(19) United States
(12) Patent Application Publication
(10) Pub. No.: US 2014/0141996 A1
(43) Pub. Date: May 22, 2014

(54) Methods and Compositions for the Treatment and Diagnosis of Cancer

(71) Applicants: Karen Chapman, Mill Valley, CA (US); Joseph Wagner, San Ramon, CA (US); Michael West, Mill Valley, CA (US); Markus Daniel Lacher, Lafayette, CA (US); Jennifer Lorie Kidd, Alameda, CA (US); Maria Prendes, Santa Cruz, CA (US)

(72) Inventors: Karen Chapman, Mill Valley, CA (US); Joseph Wagner, San Ramon, CA (US); Michael West, Mill Valley, CA (US); Markus Daniel Lacher, Lafayette, CA (US); Jennifer Lorie Kidd, Alameda, CA (US); Maria Prendes, Santa Cruz, CA (US)

(73) Assignee: ONCOCYTE CORPORATION, Alameda, CA (US)

(21) Appl. No.: 14/127,913

(22) PCT Filed: Jun. 23, 2012
(86) PCT No.: PCT/US12/43903

§ 371 Date: Dec. 23, 2013

Related U.S. Application Data
(60) Provisional application No. 61/500,132, filed on Jun. 22, 2011.
(60) Provisional application No. 61/529,500, filed on Aug. 31, 2011.

(51) Int. Cl. C12Q 1/68; G01N 33/574

(52) U.S. Cl. CPC C12Q 1/6886; G01N 33/57415
USPC 506/9; 506/18; 506/16

(57) ABSTRACT

Embodiments of the disclosure are directed to methods of diagnosis, prognosis and treatment of cancer. In some embodiments, the methods include targeting a marker that is expressed at abnormal levels in bladder cancer tissue in comparison to normal somatic tissue. Some embodiments are directed to methods of treating cancer comprising administering a composition including a therapeutic that affects the expression or function of a target marker. Some embodiments are directed to methods of detecting cancer comprising detecting a level of a target marker associated with the cancer,

This application claims priority to U.S. Provisional Application No. 61/500,132 filed Jun. 22, 2011, the entire contents of which is hereby incorporated by reference.

### FIELD OF THE INVENTION

The invention relates to the diagnosis and treatment of cancer.

### BACKGROUND

Early detection of cancer can impact treatment outcomes and disease progression. Typically, cancer detection relies on diagnostic information obtained from biopsy, x-rays, CAT scans, NMR and the like. These procedures may be invasive, time consuming and expensive. Moreover, they have limitations with regard to sensitivity and specificity. There is a need in the field of cancer diagnostics for a highly specific, highly sensitive, rapid, inexpensive, and relatively non-invasive method of diagnosing cancer. Various embodiments of the invention described below meet this need as well as other needs in the field of diagnosing and treating cancer.

### SUMMARY OF THE INVENTION

Embodiments of the disclosure are directed to methods of diagnosis, prognosis and treatment of cancer. The methods and compositions described herein can be used for a any type of cancer because the markers and genes described herein are abnormally expressed in, for example, all cancers.

In certain embodiments the invention provides a method of detecting cancer in sample comprising comparing the expression levels of a panel of markers in a sample suspected of being cancerous with the expression level of the panel of markers in a normal sample wherein elevated expression levels of the panel of markers in the sample suspected of being cancerous compared to the normal sample indicates that the sample is cancerous, wherein the panel of markers comprises one or more of the following markers: LCN2, REG4, REG1b, OLFM4, UBD, NMU, MMP11, and WNT10A.

In certain embodiments the invention provides a method of detecting cancer in sample comprising comparing the expression levels of a panel of markers in a sample suspected of being cancerous with the expression level of the panel of markers in a normal sample wherein elevated expression levels of the panel of markers in the sample suspected of being cancerous compared to the normal sample indicates that the sample is cancerous wherein the panel of markers comprises one or more of the following markers: NMU, KRT6A, ASCL1, C1orf64, FLJ23152, C2orf70, C12orf56, SLC35D, OBP2A, MMP12, MMP11, IGSF1, ZCCH12, SFTPB, FLJ30058, DSCR8, AMH, LY6G6D, SPINK4, L1TD1, DKK4.

Suitable cancers that can be diagnosed or screened for using the methods of the present invention include cancers classified by site or by histological type. Cancers classified by site include cancer of the oral cavity and pharynx (lip, tongue, salivary gland, floor of mouth, gum and other mouth, nasopharynx, tonsil, oropharynx, hypopharynx, other oral/pharynx); cancers of the digestive system (esophagus; stomach; small intestine; colon and rectum; anus, anal canal, and anorectum; liver; intrahepatic bile duct; gallbladder; other biliary; pancreas; retroperitoneum; peritoneum, omentum, and mesentery; other digestive); cancers of the respiratory system (nasal cavity, middle ear, and sinuses; larynx; lung and bronchus; pleura; trachea, mediastinum, and other respiratory); cancers of the mesothelioma; bones and joints; and soft tissue, including heart; skin cancers, including melanomas and other non-epithelial skin cancers; Kaposi's sarcoma and breast cancer; cancer of the female genital system (cervix uteri; corpus uteri; uterus, nos; ovary; vagina; vulva; and other female genital); cancers of the mate genital system (prostate gland; testis; penis; and other male genital); cancers of the urinary system (urinary bladder; kidney and renal pelvis; ureter; and other urinary); cancers of the eye and orbit; cancers of the brain and nervous system (brain; and other nervous system); cancers of the endocrine system (thyroid gland and other endocrine, including thymus); lymphomas (Hodgkin's disease and non-Hodgkin's lymphoma), multiple myeloma, and leukemias (lymphocytic leukemia; myeloid leukemia; monocytic leukemia; and other leukemias).

Other type of cancers, classified by histological type, that may be associated with the sequences of the invention include, but are not limited to, Neoplasm, malignant; Carcinoma, NOS; Carcinoma, undifferentiated, NOS; Giant and spindle cell carcinoma; Small cell carcinoma, NOS; Papillary carcinoma, NOS; Squamous cell carcinoma, NOS; Lymphoepithelial carcinoma; Basal cell carcinoma, NOS; Pilomatrix carcinoma; Transitional cell carcinoma, NOS; Papillary transitional cell carcinoma; Adenocarcinoma, NOS; Gastrinoma, malignant; Cholangiocarcinoma; Hepatocellular carcinoma, NOS; Combined hepatocellular carcinoma and cholangiocarcinoma; Trabecular adenocarcinoma; Adenoid cystic carcinoma; Adenocarcinoma in adenomatous polyp; Adenocarcinoma, familial polyposis coli; Solid carcinoma, NOS; Carcinoid tumor, malignant; Bronchiolo-alveolar adenocarcinoma; Papillary adenocarcinoma, NOS; Chromophobe carcinoma; Acidophil carcinoma; Oxyphilic adenocarcinoma; Basophil carcinoma; Clear cell adenocarcinoma, NOS; Granular cell carcinoma; Follicular adenocarcinoma, NOS; Papillary and follicular adenocarcinoma; Nonencapsulating sclerosing carcinoma; Adrenal cortical carcinoma; Endometroid carcinoma; Skin appendage carcinoma; Apocrine adenocarcinoma; Sebaceous adenocarcinoma; Ceruminous adenocarcinoma; Mucoepidermoid carcinoma; Cystadenocarcinoma, NOS; Papillary cystadenocarcinoma, NOS; Papillary serous cystadenocarcinoma; Mucinous cystadenocarcinoma, NOS; Mucinous adenocarcinoma; Signet ring cell carcinoma; Infiltrating duct carcinoma; Medullary carcinoma, NOS; Lobular carcinoma; Inflammatory carcinoma; Paget's disease, mammary; Acinar cell carcinoma; Adenosquamous carcinoma; Adenocarcinoma w/squamous metaplasia; Thymoma, malignant; Ovarian stromal tumor, malignant; Thecoma, malignant; Granulosa cell tumor, malignant; Androblastoma, malignant; Sertoli cell carcinoma; Leydig cell tumor, malignant; Lipid cell tumor, malignant; Paraganglioma, malignant; Extra-mammary paraganglioma, malignant; Pheochromocytoma; Glomangiosarcoma; Malignant melanoma, NOS; Amelanotic melanoma; Superficial spreading melanoma; Malig melanoma in giant pigmented nevus; Epithelioid cell melanoma; Blue nevus, malignant; Sarcoma, NOS; Fibrosarcoma, NOS; Fibrous histiocytoma, malignant; Myxosarcoma; Liposarcoma, NOS; Leiomyosarcoma, NOS; Rhabdomyosarcoma, NOS; Embryonal rhabdomyosarcoma; Alveolar rhabdomyosarcoma; Stromal sarcoma, NOS; Mixed tumor, malignant, NOS; Mullerian mixed tumor; Nephroblastoma; Hepatoblastoma; Carcinosarcoma, NOS; Mesenchymoma, malignant; Brenner tumor, malignant; Phyllodes tumor, malignant; Synovial sarcoma, NOS; Mesothelioma, malignant; Dysgerminoma; Embryonal carcinoma, NOS; Teratoma, malignant, NOS; Struma ovarli, malignant; Choriocarcinoma; Mesonephroma, malignant; Hemangiosarcoma; Hemangioendothelioma, malignant; Kaposi's sarcoma; Hemangiopericytoma, malignant; Lymphangiosarcoma; Osteosarcoma, NOS; Juxtacortical osteosarcoma; Chondrosarcoma, NOS; Chondroblastoma, malignant; Mesenchymal chondrosarcoma; Giant cell tumor of bone; Ewing's sarcoma; Odontogenic tumor, malignant; Ameloblastic odontosarcoma; Ameloblastoma, malignant; Ameloblastic fibrosarcoma; Pinealoma, malignant; Chordoma; Glioma, malignant; Ependymoma, NOS; Astrocytoma, NOS; Protoplasmic astrocytoma; Fibrillary astrocytoma; Astroblastoma; Glioblastoma, NOS; Oligodendroglioma, NOS; Oligodendroblastoma; Primitive neuroectodermal; Cerebellar sarcoma, NOS; Ganglioneuroblastoma; Neuroblastoma, NOS; Retinoblastoma, NOS; Olfactory neurogenic tumor; Meningioma, malignant; Neurofibrosarcoma; Neurilemmoma, malignant; Granular cell tumor, malignant; Malignant lymphoma, NOS; Hodgkin's disease, NOS; Hodgkin's; paragranuloma, NOS; Malignant lymphoma, small lymphocytic; Malignant lymphoma, large cell, diffuse; Malignant lymphoma, follicular, NOS; Mycosis fungoides; Other specified non-Hodgkin's lymphomas; Malignant histiocytosis; Multiple myeloma; Mast cell sarcoma; Immunoproliferative small intestinal disease; Leukemia, NOS; Lymphoid leukemia, NOS; Plasma cell leukemia; Erythroleuikemia; Lymphosarcoma cell leukemia; Myeloid leukemia, NOS; Basophilic leukemia; Eosinophilic leukemia; Monocytic leukemia, NOS; Mast cell leukemia; Megakaryoblastic leukemia; Myeloid sarcoma; and Hairy cell leukemia.

In some embodiments, the methods comprise targeting a marker that is expressed at abnormal levels in bladder cancer tissue in comparison to normal tissue. In some embodiments, the marker may include one or more of the sequences described herein or any combination thereof.

In some embodiments, methods for the treatment of cancer and related pharmaceutical preparations and kits are provided. Some embodiments are directed to methods of treating cancer comprising administering a composition including a therapeutic that affects the expression, abundance or activity of a target marker. In some embodiments, the target marker may include a sequence described herein or in the accession numbers described herein or encoded by the same.

In some embodiments, the therapeutic may be an antibody,

Some embodiments are directed to methods of detecting cancer comprising detecting a level of a target marker associated with the cancer. In some embodiments, the target marker may include a sequence described herein or in the accession numbers described herein or encoded by the same.

Some embodiments herein provide antigens (cancer-associated polypeptides) associated with a variety of cancers as targets for diagnostic and/or therapeutic antibodies. In some embodiments, these antigens may be useful for drug discovery (e.g., small molecules) and for further characterization of cellular regulation, growth, and differentiation.

In addition to the incorporation of the sequences disclosed in the accession numbers found in the Gene Expression Table and the other tables provided herein, in some embodiments, the sequence comprises a sequence or fragment thereof that is disclosed herein.

### DESCRIPTION OF DRAWINGS

For a fuller understanding of the nature and advantages of the present invention, reference should be had to the following detailed description taken in connection with the accompanying drawings, in which:

FIG. 1 shows the expression of the genes C2orf70 (accession number NM_{—}001105519.1) in normal somatic cells, normal tissues, malignant tumors, and cancer cell lines.

FIG. 2 shows the expression of the genes PCSK1 (accession number NM_{—}000439.3) in normal somatic cells, normal tissues, malignant tumors, and cancer cell lines. For the purpose of illustration, samples truncated with the wavy line display actual RFU values at the top of the line.

FIG. 3 shows the expression of the genes SLC35D3 (accession number NM_{—}001008783.1) in normal somatic cells, normal tissues, malignant tumors, and cancer cell

FIG. 4 shows the expression of the genes TP53TG3 (accession number NM_{—}016212.2) in normal somatic cells, normal tissues, malignant tumors, and cancer cell lines.

FIG. 5 shows the expression of the gene SNAR-A1 also known as IMAGE:6563923 5 (accession number BU536065) in normal somatic cells, normal tissues, malignant tumors, and cancer cell lines.

FIG. 6 shows the expression of the gene DSCR8 (accession number NM_{—}203428.1) in normal somatic cells, normal tissues, malignant tumors, and cancer cell lines.

FIG. 7 shows the expression of the gene SEZ6L (Accession Number NM_{—}021115.3) in normal somatic cells, normal tissues, malignant tumors, and cancer cell lines.

FIG. 8 shows the serum levels of CXCL10 in breast cancer and normal human subjects as well as patients with benign breast tumors.

FIG. 9 shows the serum levels of CXCL9 in breast cancer and normal human subjects as well as patients with benign breast tumors.

FIG. 10 shows the serum levels of CXCL9 in colon cancer and normal human subjects.

FIG. 11 shows the serum levels of MMP7 in breast cancer and normal human subjects as well as patients with benign breast tumors.

FIG. 12 shows the serum levels of MMP7 in colon cancer and normal human subjects.

FIG. 13 shows the serum levels of MMP7 in pancreatic cancer and normal human subjects,

FIG. 14 shows the serum levels of MMP12 in breast cancer and normal human subjects as well as patients with benign breast tumors.

FIG. 15 shows the serum levels of MMP12 in colon cancer and normal human subjects.

FIG. 16 shows the serum levels of MMP12 in pancreatic cancer and normal human subjects.

FIG. 17 shows the serum levels of MMP9 in breast cancer and normal human subjects as well as patients with benign breast tumors.

FIG. 18 shows the serum levels of MMP9 in colon cancer and normal human subjects.

FIG. 19 shows the serum levels of MMP9 in pancreatic cancer and normal human subjects.

FIG. 20 shows the serum levels of EPYC in breast cancer and normal human subjects,

FIG. 21 shows the serum levels of IL8 in breast cancer and normal human subjects.

FIG. 22 shows the serum levels of LAMC2 in pancreatic cancer and normal human subjects.

FIG. 23 shows the serum levels of CLCA1 in colon cancer and normal human subjects.

FIG. 24 shows the serum levels of LCN2 in colon cancer and normal human subjects.

FIG. 25 shows the serum levels of LCN2 in pancreatic cancer and normal human subjects.

FIG. 26 shows the serum levels of REG4 in colon cancer, benign colon tumors and normal human subjects.

FIG. 27 shows the serum levels of REG4 in pancreatic cancer and normal human subjects.

FIG. 28 shows the serum levels of REG1b in pancreatic cancer and normal human subjects.

FIG. 29 shows the serum levels of OLFM4 in colon cancer, benign colon tumors and normal human subjects.

FIG. 30 shows the serum levels of UBD in colon cancer and normal human subjects.

FIG. 31 shows the serum levels of UBD in pancreatic cancer and normal human subjects.

FIG. 32 shows the serum levels of NMU in breast cancer and normal human subjects.

FIG. 33 shows the serum levels of NMU in colon cancer and normal human subjects.

FIG. 34 shows the serum levels of MMP11 in breast cancer and normal human subjects as well as patients with benign breast tumors.

FIG. 35 shows the serum levels of MMP11 in colon cancer, benign colon tumors and normal human subjects.

FIG. 36 shows the serum levels of MMP11 in pancreatic cancer and normal human subjects.

FIG. 37 shows the serum levels of MMP11 in bladder cancer and normal human subjects.

FIG. 38 shows the serum levels of WNT10A in breast cancer and normal human subjects.

FIG. 39 shows the serum levels of WNT10A in colon cancer and normal human subjects.

FIG. 40 shows the levels of KRT6A is elevated in bladder cancer relative to normal human bladder tissue as measured by qPCR.

FIG. 41 shows the levels of ASCL1 is elevated in breast cancer relative to normal human breast tissue as measured by qPCR.

FIG. 42 shows the levels of C1orf64 is elevated in breast cancer relative to normal human breast tissue as measured by qPCR.

FIG. 43 shows the levels of FLJ23152 is elevated in breast cancer relative to normal human breast tissue as measured by qPCR.

FIG. 44 is a graph showing the expression levels of C2orf70 in various tumor and normal tissues as measured by qPCR.

FIG. 45 is a graph showing the expression levels of C12orf56 in various tumor and normal tissues as measured by qPCR.

FIG. 46 shows the levels of SLC35D is elevated in colon cancer relative to normal human colon tissue as measured by qPCR.

FIG. 47 shows the levels of OBP2A is elevated in ovarian cancer relative to normal human ovarian tissue as measured by qPCR.

FIG. 48 shows the levels of MMP12 is elevated in bladder cancer relative to normal human bladder tissue as measured by qPCR.

FIG. 49 shows the levels of MMP11 is elevated in bladder cancer relative to normal human bladder tissue as measured by qPCR.

FIG. 50 shows the levels of IGSF1 is elevated in thyroid cancer relative to normal human thyroid tissue as measured by qPCR.

FIG. 51 shows the levels of ZCCHC12 is elevated in thyroid cancer relative to normal human thyroid tissue as measured by qPCR,

FIG. 52 shows the levels of SFTPB is elevated in thyroid cancer relative to normal human thyroid tissue as measured by qPCR.

FIG. 53 shows the levels of FLJ30058 is elevated in thyroid cancer relative to normal human thyroid tissue as measured by qPCR.

FIG. 54 is a graph showing the expression levels of DSCR8 in various tumor and normal tissues as measured by qPCR.

FIG. 55 is a graph showing the expression levels of AMH in various tumor and normal tissues as measured by qPCR.

FIG. 56 shows the levels of NMU is elevated in thyroid cancer relative to normal human thyroid tissue as measured by qPCR.

FIG. 57 shows the levels of LY6G6D is elevated in colon cancer relative to normal human colon tissue as measured by qPCR,

FIG. 58 shows the levels of SPINK4 is elevated in colon cancer relative to normal human colon tissue as measured by qPCR.

FIG. 59 shows the levels of L1TD1 is elevated in colon cancer relative to normal human colon tissue as measured by qPCR.

FIG. 60 shows the levels of DKK4 is elevated in colon cancer relative to normal human colon tissue as measured by qPCR.

FIG. 61 shows the levels of S100A2 is elevated in bladder cancer relative to normal human bladder tissue as measured by qPCR.

FIG. 62 shows the levels of S100A7A is elevated in bladder cancer relative to normal human bladder tissue as measured by qPCR.

FIG. 63 shows expression levels of NMU can distinguish between malignant thyroid carcinoma and benign thyroid adenoma.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art, Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

As used herein, the singular forms “a,” “an,” and “the” include plural reference unless the context clearly dictates otherwise. Thus, for example, reference to a “therapeutic” is a reference to one or more therapeutics and equivalents thereof known to those skilled in the art, and so forth.

As used herein, the term “about” means plus or minus 10% of the numerical value of the number with which it is being used. Therefore, about 50% means in the range of 45% to 55%.

“Administering,” when used in conjunction with a therapeutic, means to administer a therapeutic directly into or onto a target tissue or to administer a therapeutic to a patient whereby the therapeutic positively impacts the tissue to which it is targeted. Thus, as used herein, the term “administering”, when used in conjunction with elastin digest, can include, but is not limited to, providing an elastin digest into or onto the target tissue; providing an elastin digest systemically to a patient by, e.g., intravenous injection whereby the therapeutic reaches the target tissue; providing an elastin digest in the form of the encoding sequence thereof to the target tissue (e.g., by so-called gene-therapy techniques). “Administering” a composition may be accomplished by oral administration, intravenous injection, intraperitoneal injection, intramuscular injection, subcutaneous injection, transdermal diffusion or electrophoresis, local injection, extended release delivery devices including locally implanted extended release devices such as bioerodible or reservoir-based implants, as protein therapeutics or as nucleic acid therapeutic via gene therapy vectors, topical administration, or by any of these methods in combination with other known techniques. Such combination techniques include heating, radiation and ultrasound.

The term “animal,” “patient” or “subject” as used herein includes, but is not limited to, humans and non-human vertebrates such as wild, domestic and farm animals. Preferably, the term “subject,” “patient” or “animal” refers to humans.

The term “inhibiting” includes the administration of a compound of the present invention to prevent the onset of the symptoms, alleviating the symptoms, or eliminating the disease, condition or disorder.

By “pharmaceutically acceptable”, it is meant the carrier, diluent or excipient must be compatible with the other ingredients of the formulation and not deleterious to the recipient thereof.

In some embodiments, the present disclosure provides for nucleic acid and protein sequences that are associated with cancer, herein termed “cancer associated” or “CA” sequences. In some embodiments, the present disclosure provides nucleic acid and protein sequences that are associated with cancers or carcinomas that originate in any cancer including one or more of any combination thereof of the cancers described herein.

The term “pluripotent stem cells” refers to animal cells capable of differentiating into more than one differentiated cell type. Such cells include hES cells, hED cells, hEG cells, hEC cells, and adult-derived cells including mesenchymal stem cells, neuronal stem cells, and bone marrow-derived stem cells. Pluripotent stem cells may be genetically modified or not genetically modified. Genetically modified cells may include markers such as fluorescent proteins to facilitate their identification within the egg.

The term “embryonic stem cells” (ES cells) refers to cells derived from the inner cell mass of blastocysts, blastomeres, or morulae that have been serially passaged as cell lines while maintaining an undifferentiated state (e.g. expressing TERT, OCT4, and SSEA and TRA antigens specific for ES cells of the species). The ES cells may be derived from fertilization of an egg cell with sperm or DNA, nuclear transfer, parthenogenesis, or by means to generate hES cells with hemizygosity or homozygosity in the MHC region. The term “human embryonic stem cells” (hES cells) refers to human ES cells.

The term “human embryonic germ cells” (hEG cells) refer to pluripotent stem cells derived from the primordial germ cells of fetal tissue or maturing or mature germ cells such as oocytes and spermatogonial cells, that can differentiate into various tissues in the body. The hEG cells may also be derived from pluripotent stem cells produced by gynogenetic or androgenetic means, i.e., methods wherein the pluripotent cells are derived from oocytes containing only DNA of male or female origin and therefore will comprise all female-derived or male-derived DNA (see U.S. application nos. 60/161,987, filed Oct. 28, 1999; Ser. No. 09/697,297, filed Oct. 27, 2000; Ser. No. 09/995,659, filed Nov. 29, 2001; Ser. No. 10/374,512, filed Feb. 27, 2003; PCT application no. PCT/US/00/29551, filed Oct. 27, 2000; the disclosures of which are incorporated herein in their entirety).

The term human iPS cells refers to cells with properties similar to hES cells, including the ability to form all three germ layers when translanted into immunocompromised mice wherein said iPS cells are derived from cells of varied somatic cell lineages following exposure to hES cell-specific transcription factors such as KLF4, SOX2, MYC, and OCT4 or the factors SOX2, OCT4, NANOG, and LIN28. Said iPS cells may be produced by the expression of these gene through vectors such as retrovial vectors as is known in the art, or through the introduction of these factors by permeabilization or other technologies taught by PCT application number PCT/US2006/030632 (WO2007/019398).

The term “differentiated cells” when used in reference to cells made by methods of this invention from pluripotent stem cells refer to cells having reduced potential to differentiate when compared to the parent pluripotent stem cells. The differentiated cells of this invention comprise cells that could differentiate further (i.e., they may not be terminally differentiated).

The term embryonal carcinoma (“EC”) cells, including human EC cells, refers to embryonal carcinoma cells such as TERA-1, TERA-2, and NTera-2.

As used herein, the term “naturally occurring” refers to sequences or structures that may be in a form normally found in nature. “Naturally occurring” may include sequences in a form normally found in any animal.

As used herein, the term “cancer associated sequences” refers to nucleotide or protein sequences are either differentially expressed, activated, inactivated or altered in cancers as compared to normal tissue. Cancer associated sequences may include those that are up-regulated (i.e. expressed at a higher level), as well as those that are down-regulated (i.e. expressed at a lower level), in cancers. Cancer associated sequences can also include sequences that have been altered (i.e., translocations, truncated sequences or sequences with substitutions, deletions or insertions, including, but not limited to, point mutations) and show either the same expression profile or an altered profile. In some embodiments, the cancer associated sequences are from humans; however, as will be appreciated by those in the art, cancer associated sequences from other organisms may be useful in animal models of disease and drug evaluation; thus, other cancer associated sequences may be useful such as, without limitation, sequences from vertebrates, including mammals, including rodents (rats, mice, hamsters, guinea pigs, etc.), primates, and farm animals (including sheep, goats, pigs, cows, horses, etc). Cancer associated sequences from other organisms may be obtained using the techniques outlined below.

In some embodiments, the markers described herein can be used to treat, diagnose, determine prognosis, and/or detect cancer in one or more of the following cancers, or any combination thereof. The cancers include, but are not limited to the cancers described herein.

For example, suitable cancers that can be diagnosed or screened for using the methods of the present invention include cancers classified by site or by histological type. Cancers classified by site include cancer of the oral cavity and pharynx (lip, tongue, salivary gland, floor of mouth, gum and other mouth, nasopharynx, tonsil, oropharynx, hypopharynx, other oral/pharynx); cancers of the digestive system (esophagus; stomach; small intestine; colon and rectum; anus, anal canal, and anorectum; liver; intrahepatic bile duct; gallbladder; other biliary; pancreas; retroperitoneum; peritoneum, omentum, and mesentery; other digestive); cancers of the respiratory system (nasal cavity, middle ear, and sinuses; larynx; lung and bronchus; pleura; trachea, mediastinum, and other respiratory); cancers of the mesothelioma; bones and joints; and soft tissue, including heart; skin cancers, including melanomas and other non-epithelial skin cancers; Kaposi's sarcoma and breast cancer; cancer of the female genital system (cervix uteri; corpus uteri; uterus, nos; ovary; vagina; vulva; and other female genital); cancers of the male genital system (prostate gland; testis; penis; and other male genital); cancers of the urinary system (urinary bladder; kidney and renal pelvis; ureter; and other urinary); cancers of the eye and orbit; cancers of the brain and nervous system (brain; and other nervous system); cancers of the endocrine system (thyroid gland and other endocrine, including thymus); lymphomas (Hodgkin's disease and non-Hodgkin's lymphoma), multiple myeloma, and leukemias (lymphocytic leukemia; myeloid leukemia; monocytic leukemia; and other leukemias).

Other type of cancers, classified by histological type, that may be associated with the sequences of the invention include, but are not limited to, Neoplasm, malignant; Carcinoma, NOS; Carcinoma, undifferentiated, NOS; Giant and spindle cell carcinoma; Small cell carcinoma, NOS; Papillary carcinoma, NOS; Squamous cell carcinoma, NOS; Lymphoepithelial carcinoma; Basal cell carcinoma, NOS; Pilomatrix carcinoma; Transitional cell carcinoma, NOS; Papillary transitional cell carcinoma; Adenocarcinoma, NOS; Gastrinoma, malignant; Cholangiocarcinoma; Hepatocellular carcinoma, NOS; Combined hepatocellular carcinoma and cholangiocarcinoma; Trabecular adenocarcinoma; Adenoid cystic carcinoma; Adenocarcinoma in adenomatous polyp; Adenocarcinoma, familial polyposis coli; Solid carcinoma, NOS; Carcinoid tumor, malignant; Bronchiolo-alveolar adenocarcinoma; Papillary adenocarcinoma, NOS; Chromophobe carcinoma; Acidophil carcinoma; Oxyphilic adenocarcinoma; Basophil carcinoma; Clear cell adenocarcinoma, NOS; Granular cell carcinoma; Follicular adenocarcinoma, NOS; Papillary and follicular adenocarcinoma; Nonencapsulating sclerosing carcinoma; Adrenal cortical carcinoma; Endometroid carcinoma; Skin appendage carcinoma; Apocrine adenocarcinoma; Sebaceous adenocarcinoma; Ceruminous adenocarcinoma; Mucoepidermoid carcinoma; Cystadenocarcinoma, NOS; Papillary cystadenocarcinoma, NOS; Papillary serous cystadenocarcinoma; Mucinous cystadenocarcinoma, NOS; Mucinous adenocarcinoma; Signet ring cell carcinoma; Infiltrating duct carcinoma; Medullary carcinoma, NOS; Lobular carcinoma; Inflammatory carcinoma; Paget's disease, mammary; Acinar cell carcinoma; Adenosquamous carcinoma; Adenocarcinoma w/squamous metaplasia; Thymoma, malignant; Ovarian stromal tumor, malignant; Thecoma, malignant; Granulosa cell tumor, malignant; Androblastoma, malignant; Sertoli cell carcinoma; Leydig cell tumor, malignant; Lipid cell tumor, malignant; Paraganglioma, malignant; Extra-mammary paraganglioma, malignant; Pheochromocytoma; Glomangiosarcoma; Malignant melanoma, NOS; Amelanotic melanoma; Superficial spreading melanoma; Malig melanoma in giant pigmented nevus; Epithelioid cell melanoma; Blue nevus, malignant; Sarcoma, NOS; Fibrosarcoma, NOS; Fibrous histiocytoma, malignant; Myxosarcoma; Liposarcoma, NOS; Leiomyosarcoma, NOS; Rhabdomyosarcoma, NOS; Embryonal rhabdomyosarcoma; Alveolar rhabdomyosarcoma; Stromal sarcoma, NOS; Mixed tumor, malignant, NOS; Mullerian mixed tumor; Nephroblastoma; Hepatoblastoma; Carcinosarcoma, NOS; Mesenchymoma, malignant; Brenner tumor, malignant; Phyllodes tumor, malignant; Synovial sarcoma, NOS; Mesothelioma, malignant; Dysgerminoma; Embryonal carcinoma, NOS; Teratoma, malignant, NOS; Struma ovarii, malignant; Choriocarcinoma; Mesonephroma, malignant; Hemangiosarcoma; Hemangioendothelioma, malignant; Kaposi's sarcoma; Hemangiopericytoma, malignant; Lymphangiosarcoma; Osteosarcoma, NOS; Juxtacortical osteosarcoma; Chondrosarcoma, NOS; Chondroblastoma, malignant; Mesenchymal chondrosarcoma; Giant cell tumor of bone; Ewing's sarcoma; Odontogenic tumor, malignant; Ameloblastic odontosarcoma; Ameloblastoma, malignant; Ameloblastic fibrosarcoma; Pinealoma, malignant; Chordoma; Glioma, malignant; Ependymoma, NOS; Astrocytoma, NOS; Protoplasmic astrocytoma; Fibrillary astrocytoma; Astroblastoma; Glioblastoma, NOS; Oligodendroglioma, NOS; Oligodendroblastoma; Primitive neuroectodermal; Cerebellar sarcoma, NOS; Ganglioneuroblastoma; Neuroblastoma, NOS; Retinoblastoma, NOS; Olfactory neurogenic tumor; Meningioma, malignant; Neurofibrosarcoma; Neurilemmoma, malignant; Granular cell tumor, malignant; Malignant lymphoma, NOS; Hodgkin's disease, NOS; Hodgkin's; paragranuloma, NOS; Malignant lymphoma, small lymphocytic; Malignant lymphoma, large cell, diffuse; Malignant lymphoma, follicular, NOS; Mycosis fungoides; Other specified non-Hodgkin's lymphomas; Malignant histiocytosis; Multiple myeloma; Mast cell sarcoma; Immunoproliferative small intestinal disease; Leukemia, NOS; Lymphoid leukemia, NOS; Plasma cell leukemia; Erythroleuikemia; Lymphosarcoma cell leukemia; Myeloid leukemia, NOS; Basophilic leukemia; Eosinophilic leukemia; Monocytic leukemia, NOS; Mast cell leukemia; Megakaryoblastic leukemia; Myeloid sarcoma; and Hairy cell leukemia.

The term “homology,” as used herein, refers to a degree of complementarity. There may be partial homology or complete homology. The word “identity” may substitute for the word “homology.” A partially complementary nucleic acid sequence that at least partially inhibits an identical sequence from hybridizing to a target nucleic acid is referred to as “substantially homologous.” The inhibition of hybridization of the completely complementary sequence to the target sequence may be examined using a hybridization assay (Southern or northern blot, solution hybridization, and the like) under conditions of reduced stringency. A substantially homologous sequence or hybridization probe will compete for and inhibit the binding of a completely homologous sequence to the target sequence under conditions of reduced stringency. This is not to say that conditions of reduced stringency are such that non-specific binding is permitted, as reduced stringency conditions require that the binding of two sequences to one another be a specific (i.e., a selective) interaction. The absence of non-specific binding may be tested by the use of a second target sequence which lacks even a partial degree of complementarity (e.g., less than about 30% homology or identity). In the absence of non-specific binding, the substantially homologous sequence or probe will not hybridize to the second non-complementary target sequence.

The phrases “percent homology,” “% homology,” “percent identity” or “% identity” refer to the percentage of sequence similarity found in a comparison of two or more amino acid or nucleic acid sequences. Percent identity can be determined electronically, e.g., by using the MEGALIGN program (LASERGENE software package, DNASTAR). The MEGALIGN program can create alignments between two or more sequences according to different methods, e.g., the Clustal Method. (Higgins, D. G, and P. M. Sharp (1988) Gene 73:237-244.) The Clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage similarity. Percent identity between nucleic acid sequences can also be calculated by the Clustal Method, or by other methods known in the art, such as the Jotun Hein Method. (See, e.g., Hein, J. (1990) Methods Enzymol. 183:626-645.) Identity between sequences can also be determined by other methods known in the art, e.g., by varying hybridization conditions.

In some embodiments, cancer associated sequences may include both nucleic acid and amino acid sequences. In some embodiments, the cancer associated sequences may include sequences having at least about 60% homology with the disclosed sequences. In some embodiments, the cancer associated sequences may have at least about 65%, about 70%, about 75%, about 80%, about 85%, about 90%, about 95%, about 97%, about 99%, about 99.8% homology with the disclosed sequences. In some embodiments, the cancer associated sequences may be “mutant nucleic acids”. As used herein, “mutant nucleic acids” refers to deletion mutants, insertions, point mutations, substitutions, translocations.

In some embodiments, the cancer associated sequences may be recombinant nucleic acids. By the term “recombinant nucleic acid” herein refers to nucleic acid molecules, originally formed in vitro, in general, by the manipulation of nucleic acid by polymerases and endonucleases, in a form not normally found in nature. Thus a recombinant nucleic acid may also be an isolated nucleic acid, in a linear form, or cloned in a vector formed in vitro by ligating DNA molecules that are not normally joined, are both considered recombinant for the purposes of this invention. It is understood that once a recombinant nucleic acid is made and reintroduced into a host cell or organism, it can replicate using the in vivo cellular machinery of the host cell rather than in vitro manipulations; however, such nucleic acids, once produced recombinantly, although subsequently replicated in vivo, are still considered recombinant or isolated for the purposes of the invention. As used herein, a “polynucleotide” or “nucleic acid” is a polymeric form of nucleotides of any length, either ribonucleotides or deoxyribonucleotides. This term includes double- and single-stranded DNA and RNA. It also includes known types of modifications, for example, labels which are known in the art, methylation, “caps”, substitution of one or more of the naturally occurring nucleotides with an analog, internucleotide modifications-such as, for example, those with uncharged linkages (e.g., phosphorothioates, phosphorodithioates, etc.), those containing pendant moieties, such as, for example proteins (including e.g., nucleases, toxins, antibodies, signal peptides, poly-L-lysine, etc.), those with intercalators (e.g., acridine, psoralen, etc.), those containing chelators (e.g., metals, radioactive metals, etc.), those containing alkylators, those with modified linkages (e.g., alpha anomeric nucleic acids, etc.), as well as unmodified forms of the polynucleotide.

As used herein, a polynucleotide “derived from” a designated sequence refers to a polynucleotide sequence which is comprised of a sequence of approximately at least about 6 nucleotides, preferably at least about 8 nucleotides, more preferably at least about 10-12 nucleotides, and even more preferably at least about 15-20 nucleotides corresponding to a region of the designated nucleotide sequence. “Corresponding” means homologous to or complementary to the designated sequence. Preferably, the sequence of the region from which the polynucleotide is derived is homologous to or complementary to a sequence that is unique to a cancer associated gene.

In the broadest sense, use of “nucleic acid,” “polynucleotide” or “oligonucleotide” or equivalents herein means at least two nucleotides covalently linked together. In some embodiments, an oligonucleotide is an oligomer of 6, 8, 10, 12, 20, 30 or up to 100 nucleotides. In some embodiments, an oligonucleotide is an oligomer of at least 6, 8, 10, 12, 20, 30, 40, 50, 60, 70, 80, 90, 100, 150, 200, 300, 400, or 500 nucleotides. A “polynucleotide” or “oligonucleotide” may comprise DNA, RNA, PNA or a polymer of nucleotides linked by phosphodiester and/or any alternate bonds.

As used herein, the term “fragment” refers to a portion of a sequence that is less than the whole. In some embodiments, the fragment is about 10-1000, 10-500, 10-400, 10-300, 10-200, 10-100, or 10-100 nucleotides and/or amino acid residues. In some embodiments, the fragment is at least 10, 20, 30, 40, 50, 60, 70, 80, 90, 100, 200, 300, 400, or 500 nucleotides and/or residues.

Similarly, a “recombinant protein” is a protein made using recombinant techniques, for example, but not limited to, through the expression of a recombinant nucleic acid as depicted above. A recombinant protein may be distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises about 50-75%, about 80%, or 90% by weight of the total protein. In some embodiments, a substantially pure protein comprises about 80-99%, 85-99%, 90-99%, 95-99%, or 97-99% by weight of the total protein. A recombinant protein can also include the production of a cancer associated protein from one organism (e.g. human) in a different organism (e.g. yeast, E. coli, and the like) or host cell (e.g. yeast, E. coli, and the like). Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed herein.

In some embodiments, the cancer associated sequences are nucleic acids. As will be appreciated by those skilled in the art and is described herein, cancer associated sequences of embodiments herein may be useful in a variety of applications including diagnostic applications to detect nucleic acids or their expression levels in a subject, therapeutic applications or a combination thereof. Further, the cancer associated sequences of embodiments herein may be used in screening applications; for example, generation of biochips comprising nucleic acid probes to the cancer associated sequences.

A nucleic acid of the present invention may include phosphodiester bonds, although in some cases, as outlined below (for example, in antisense applications or when a nucleic acid is a candidate drug agent); nucleic acid analogs may have alternate backbones, comprising, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpey et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference. These modifications of the ribose-phosphate backbone may be done for a variety of reasons, for example to increase the stability and half-life of such molecules in physiological environments for use in anti-sense applications or as probes on a biochip.

As will be appreciated by those skilled in the art, such nucleic acid analogs can be used in some embodiments. In addition, mixtures of naturally occurring nucleic acids and analogs can be made; alternatively, mixtures of different nucleic acid analogs, and mixtures of naturally occurring nucleic acids and analogs may be made.

In some embodiments, the nucleic acids may be single stranded or double stranded or may contain portions of both double stranded or single stranded sequence. As will be appreciated by those skilled in the art, the depiction of a single strand also defines the sequence of the other strand; thus the sequences described herein also includes the complement of the sequence. The nucleic acid may be DNA, both genomic and cDNA, RNA, or a hybrid, where the nucleic acid contains any combination of deoxyribo- and ribo-nucleotides, and any combination of bases, including uracil, adenine, thymine, cytosine, guanine, inosine, xanthine, hypoxanthine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus, for example, the subject units of a peptide nucleic acid, each containing a base, are referred to herein as a nucleoside.

As used herein, the term “tag,” “sequence tag” or “primer tag sequence” refers to an oligonucleotide with specific nucleic acid sequence that serves to identify a batch of polynucleotides bearing such tags therein. Polynucleotides from the same biological source are covalently tagged with a specific sequence tag so that in subsequent analysis the polynucleotide can be identified according to its source of origin. The sequence tags also serve as primers for nucleic acid amplification reactions.

A “microarray” is a linear or two-dimensional array of, for example, discrete regions, each having a defined area, formed on the surface of a solid support. The density of the discrete regions on a microarray is determined by the total numbers of target polynucleotides to be detected on the surface of a single solid phase support, preferably at least about 50/cm², more preferably at least about 100/cm², even more preferably at least about 500/cm², and still more preferably at least about 1,000/cm². As used herein, a DNA microarray is an array of oligonucleotide primers placed on a chip or other surfaces used to identify, amplify, detect, or clone target polynucleotides. Since the position of each particular group of primers in the array is known, the identities of the target polynucleotides can be determined based on their binding to a particular position in the microarray.

The term “label” refers to a composition capable of producing a detectable signal indicative of the presence of the target polynucleotide in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by a device or method, such as but not limited to, a spectroscopic, photochemical, biochemical, immunochemical, electrical, optical, chemical detection device or any other appropriate device. The label can also be detectable visually without the aid of a device. The term “label” is used to refer to any chemical group or moiety having a detectable physical property or any compound capable of causing a chemical group or moiety to exhibit a detectable physical property, such as an enzyme that catalyzes conversion of a substrate into a detectable product. The term “label” also encompasses compounds that inhibit the expression of a particular physical property. The label may also be a compound that is a member of a binding pair, the other member of which bears a detectable physical property.

The term “support” refers to conventional supports such as beads, particles, dipsticks, fibers, filters, membranes, and silane or silicate supports such as glass slides.

The term “amplify” is used in the broad sense to mean creating an amplification product which may include, for example, additional target molecules, or target-like molecules or molecules complementary to the target molecule, which molecules are created by virtue of the presence of the target molecule in the sample. In the situation where the target is a nucleic acid, an amplification product can be made enzymatically with DNA or RNA polymerases or reverse transcriptases, or any combination thereof.

As used herein, a “biological sample” refers to a sample of tissue or fluid isolated from a subject, including but not limited to, for example, blood, plasma, serum, spinal fluid, lymph fluid, skin, respiratory, intestinal and genitourinary tracts, tears, saliva, milk, cells (including but not limited to blood cells), tumors, organs, and also samples of in vitro cell culture constituents.

The term “biological sources” as used herein refers to the sources from which the target polynucleotides may be derived. The source can be of any form of “sample” as described above, including but not limited to, cell, tissue or fluid. “Different biological sources” can refer to different cells/tissues/organs of the same individual, or cells/tissues/organs from different individuals of the same species, or cells/tissues/organs from different species.

As used herein, the term “therapeutic” or “therapeutic agent” means an agent that can be used to treat, combat, ameliorate, prevent or improve an unwanted condition or disease of a patient. In part, embodiments of the present invention are directed to the treatment of cancer or the decrease in proliferation of cells. In some embodiments, the tem “therapeutic” or “therapeutic agent” may refer to any molecule that associates with or affects the target marker, its expression or its function. In various embodiments, such therapeutics may include molecules such as, for example, a therapeutic cell, a therapeutic peptide, a therapeutic gene, a therapeutic compound, or the like, that associates with or affects the target marker, its expression or its function.

A “therapeutically effective amount” or “effective amount” of a composition is a predetermined amount calculated to achieve the desired effect, i.e., to inhibit, block, or reverse the activation, migration; or proliferation of cells. In some embodiments, the effective amount is a prophylactic amount. In some embodiments, the effective amount is an amount used to medically treat the disease or condition. The specific dose of a composition administered according to this invention to obtain therapeutic and/or prophylactic effects will, of course, be determined by the particular circumstances surrounding the case, including, for example, the composition administered, the route of administration, and the condition being treated. It will be understood that the effective amount administered will be determined by the physician in the light of the relevant circumstances including the condition to be treated, the choice of composition to be administered, and the chosen route of administration. A therapeutically effective amount of composition of this invention is typically an amount such that when it is administered in a physiologically tolerable composition, it is sufficient to achieve an effective systemic concentration or local concentration in the targeted tissue.

The terms “treat,” “treated,” or “treating” as used herein can refer to both therapeutic treatment or prophylactic or preventative measures, wherein the object is to prevent or slow down (lessen) an undesired physiological condition, disorder or disease, or to obtain beneficial or desired clinical results. In some embodiments, it refers to both treating and preventing. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

Generally speaking, the term “tissue” refers to any aggregation of similarly specialized cells that are united in the performance of a particular function.

“Optional” or “optionally” means that the subsequently described structure, event or circumstance may or may not occur, and that the description includes instances where the event occurs and instances where it does not.

### Cancer Associated Sequences

Some embodiments herein are directed to one or more of sequences associated with cancers. In some embodiments, the sequences are the sequences incorporated by reference in the Expression Data Table. In some embodiments, the sequences comprise a sequence disclosed herein or a homolog thereof, or a fragment thereof, or any combination thereof. The use of microarray analysis of gene expression allows the identification of sequences associated with cancer. These sequences may then be used in a number of different ways, including diagnosis, prognosis, screening for modulators (including both agonists and antagonists), antibody generation (for immunotherapy and imaging), etc. However, as will be appreciated by those skilled in the art, sequences that are identified in one type of cancer may have a strong likelihood of being involved in other types of cancers as well. Thus, while the sequences outlined herein are initially identified as correlated with cancer.

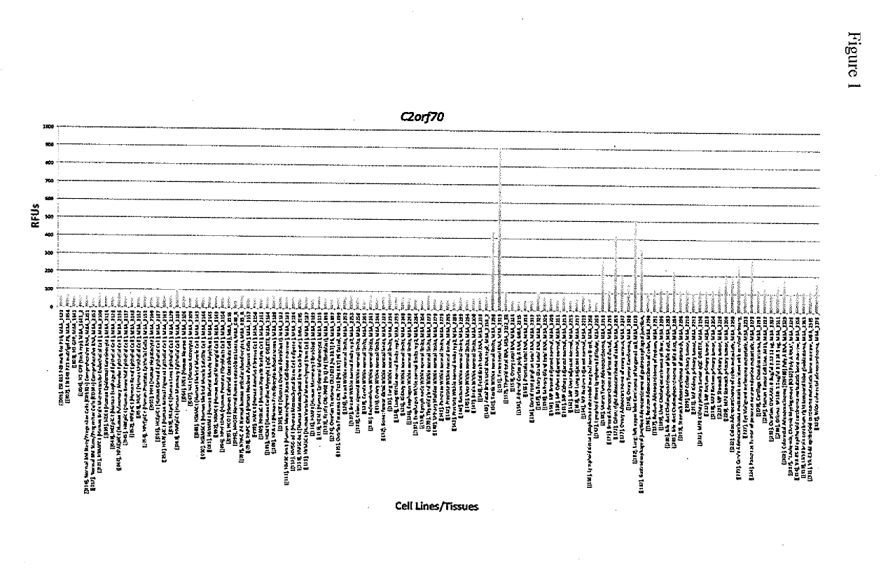

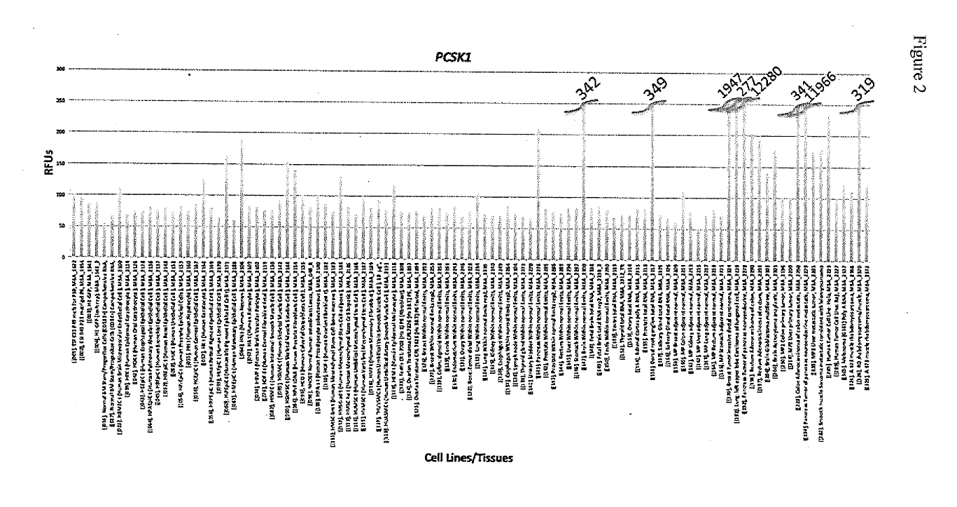

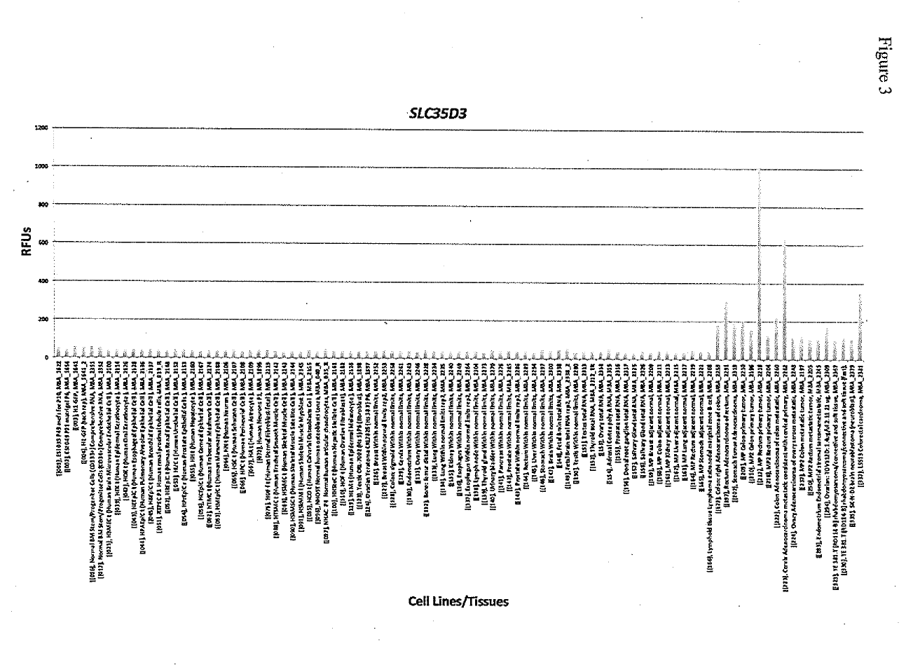

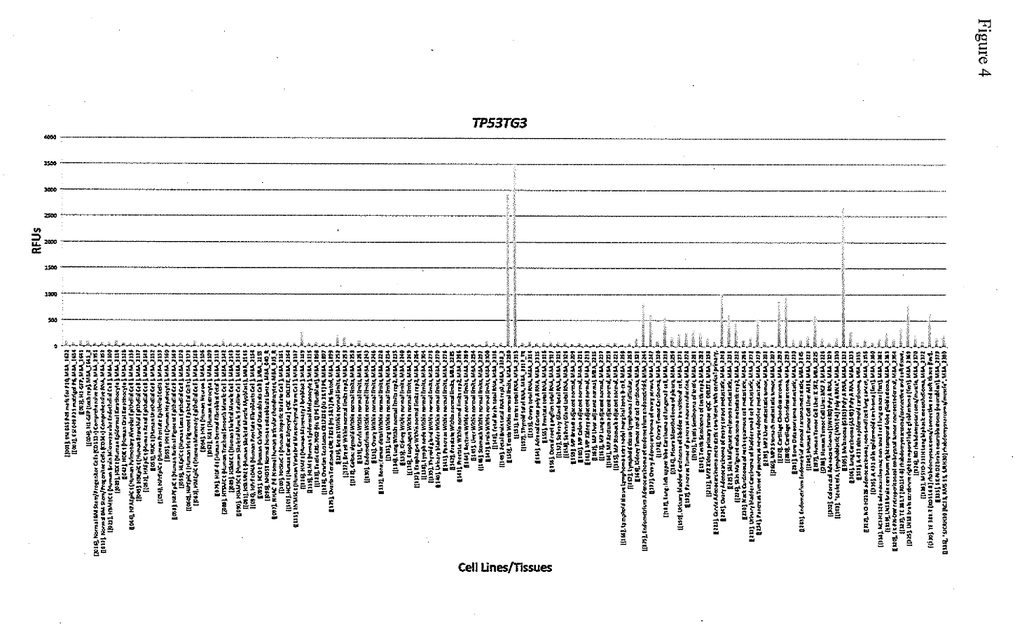

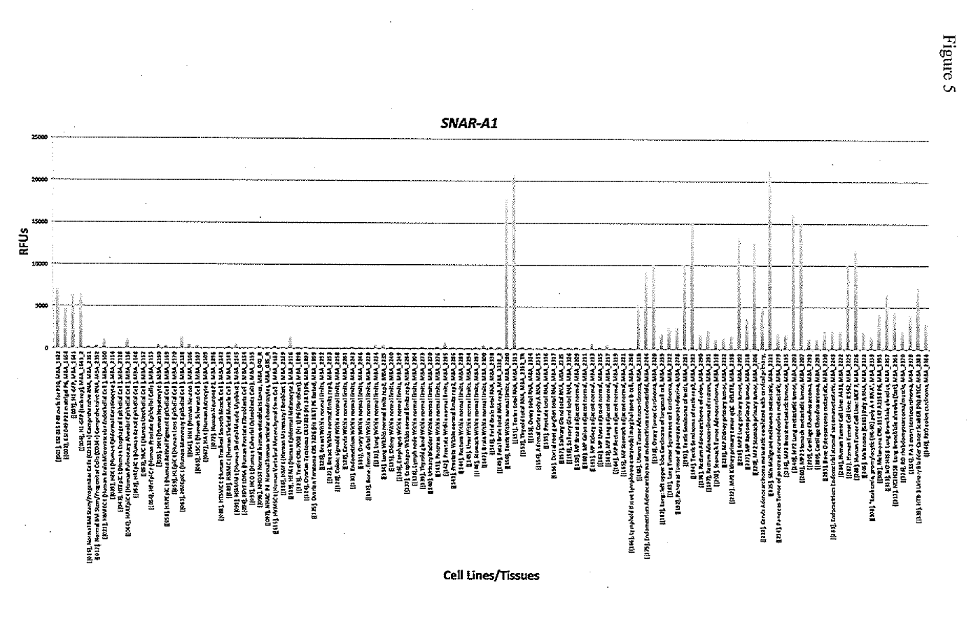

Diagnosing a patient with cancer can be difficult due to technical difficulties. The markers identified herein, which include the nucleic acid sequences and the peptides encoded by the same can be used to diagnose and then used to treat the patient with cancer. For example, therapeutic antibodies can be made against the cancer associated sequences. Examples of therapeutic antibodies and how to make such antibodies are known ill the art and can be adapted to the proteins or peptides encoded by the sequences described herein. The expression data that is provided herein has identified genes that can be used as markers and targets for therapy. In some embodiments, the data is the data provided in the table entitled, “Expression Data Table.” The expression data can be graphed for easier analysis and visualization. For example, when the data is graphed, figures such as those shown in FIGS. 1-7 are produced. The differential expression of specific genes can be easily be identified and the gene marker can then be used to identify whether or not a sample comprises a cancer overexpressing that gene. If the gene is overexpressed as compared to a normal sample, then the test sample is said to have cancer and the subject from which the sample was derived from is also said to have cancer. The type of cancer will depend upon the tissues or tumors where the specific gene is overexpressed as compared to a normal sample. The type of cancer can be identified can be determined using the data provided in the Expression Data Table.

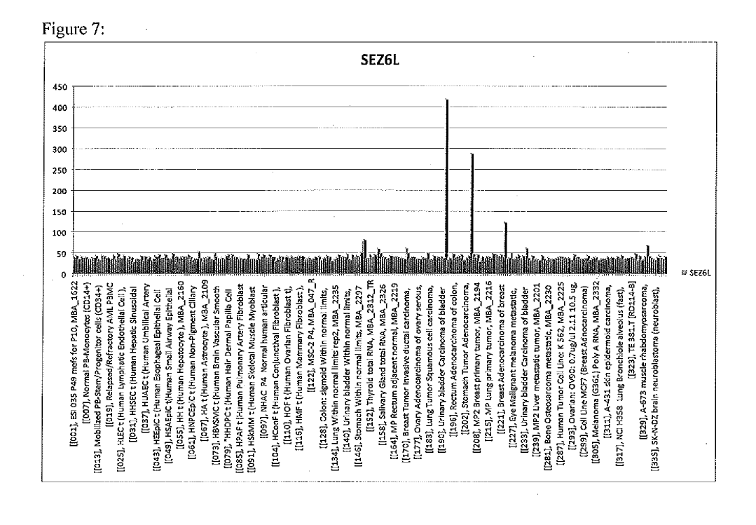

For example, SEZ6L (FIG. 7) is seen overexpressed in several types of cancers when it is compared to a normal sample (e.g. cell). Therefore, if a subject is tested and the expression of SEZ6L is determined and found to be overexpressed then the overexpression is indicative of the individual having cancer. The specific type of cancer can be confirmed by measuring other gene expression profiles or through other diagnostic tests. For other genes the overexpression profile is limited to a specific type of tissue or tumor. Therefore, those genes can be used to identify specific types of cancer. The data in the Expression Data Table contains genes that have a tissue specific overexpression profile. For example, the expression of certain genes, such as EMR1 that are overexpressed in bladder cancer. And, for example, LOC641738 is overexpressed in melanoma. The specific expression is taken from the expression data contained herein. The table describes the specificity of the cancer due to the measurement of the expression table.

The pattern of gene expression in a particular living cell may be characteristic of its current state. Nearly all differences in the state or type of a cell are reflected in the differences in RNA levels of one or more genes. Comparing expression patterns of uncharacterized genes may provide clues to their function. High throughput analysis of expression of hundreds or thousands of genes can help in (a) identification of complex genetic diseases, (b) analysis of differential gene expression over time, between tissues and disease states, and (c) drug discovery and toxicology studies. Increase or decrease in the levels of expression of certain genes correlate with cancer biology. For example, oncogenes are positive regulators of tumorigenesis, while tumor suppressor genes are negative regulators of tumorigenesis. (Marshall, Cell, 64: 313-326 (1991); Weinberg, Science, 254: 1138-1146 (1991)). Accordingly, some embodiments herein provide for polynucleotide and polypeptide sequences involved in cancer and, in particular, in oncogenesis.

Oncogenes are genes that can cause cancer. Carcinogenesis can occur by a wide variety of mechanisms, including infection of cells by viruses containing oncogenes, activation of protooncogenes in the host genome, and mutations of protooncogenes and tumor suppressor genes. Carcinogenesis is fundamentally driven by somatic cell evolution (i.e. mutation and natural selection of variants with progressive loss of growth control). The genes that serve as targets for these somatic mutations are classified as either protooncogenes or tumor suppressor genes, depending on whether their mutant phenotypes are dominant or recessive, respectively.

Some embodiments of the invention are directed to target markers for cancer. Some embodiments are directed to methods of identifying novel targets useful in the diagnosis and treatment of cancer wherein expression levels of mRNAs, miRNAs, proteins, or protein post translational modifications including but not limited to phosphorylation and sumoylation are compared between five categories of cell types: (1) immortal pluripotent stem cells (such as embryonic stem (“ES”) cells, induced pluripotent stem (“iPS”) cells, and germ-line cells such as embryonal carcinoma (“EC”) cells) or gonadal tissues; (2) ES, iPS, or EC-derived clonal embryonic progenitor (“EP”) cell lines, (3) nucleated blood cells including but not limited to CD34+ cells and CD133+ cells; (4) normal mortal somatic adult-derived tissues and cultured cells including: skin fibroblasts, vascular endothelial cells, normal non-lymphoid and non-cancerous tissues, and the like, and (5) malignant cancer cells including cultured cancer cell lines or human tumor tissue. mRNAs, miRNAs, or proteins that are generally expressed (or not expressed) in categories 1, 3, and 5, or categories 1 and 5 but not expressed (or expressed) in categories 2 and 4 are candidate targets for cancer diagnosis and therapy. Some embodiments herein are directed to human applications, non-human veterinary applications, or a combination thereof.

In some embodiments, a method of identifying a target marker comprises the steps of: 1) obtaining a molecular profile of the mRNAs, miRNAs, proteins, or protein modifications of immortal pluripotent stem cells (such as embryonic stem (“ES”) cells, induced pluripotent stem (“iPS”) cells, and germ-line cells such as embryonal carcinoma (“EC”) cells); 2) ES, iPS, or EC-derived clonal embryonic progenitor (“EP”) cell lines malignant cancer cells including cultured cancer cell lines or human tumor tissues, and comparing those molecules to those present in mortal somatic cell types such as cultured clonal human embryonic progenitors, cultured somatic cells from fetal or adult sources, or normal tissue counterparts to malignant cancer cells. Target markers that are shared between pluripotent stem cells such as hES cells and malignant cancer cells, but are not present in a majority of somatic cell types may be candidate diagnostic markers and therapeutic targets.

Cancer associated sequences associated with cancer are disclosed in the table below. The expression data is provided in the table entitled “Expression Data Table.” These sequences were identified by microarray expression analysis. Once expression was determined, the gene sequence results were further filtered by considering fold-change in a cancer sample vs. a normal sample; general specificity; secreted or not, level of expression in cancer; and signal to noise ratio. The cancer associated polynucleotide sequences include the sequences described herein or the associated accession numbers. Each of the sequences described in the disclosed accession numbers are hereby incorporated by reference in its entirety. In some embodiments, the polynucleotide sequences may be mRNA sequences selected from the accession numbers in the following table. In some embodiments, the sequences are DNA sequences that are complementary to the mRNA sequences. In some embodiments, the sequences are peptides encoded by the sequences described in the accession numbers. In some embodiments, the sequences are fragments. Sequences were found to be differentially expressed in cancer samples when compared to a normal sample. Therefore, the sequences can be used alone or in combination to determine whether an individual has cancer. The sequences can also be referred to as the gene symbol as indicated in the table below.

| GenBank | | |
|---|---|---|
| Accession | | |
| No. | Description of Sequence | Symbol |
| NM_006681.1 or | Homo sapiens neuromedin U | NMU |
| NM_006681.2 | (NMU), mRNA. | |
| NM_206955.1 | Homo sapiens preferentially | PRAME |
| | expressed antigen in | |
| | melanoma (PRAME), transcript | |
| | variant 4, mRNA. | |
| NM_206955.1 | Homo sapiens preferentially | PRAME |
| | expressed antigen in | |
| | melanoma (PRAME), transcript | |
| | variant 4, mRNA. | |
| NM_014471.1 | Homo sapiens serine | SPINK4 |
| | peptidase inhibitor, Kazal type | |
| | 4 (SPINK4), mRNA. | |
| NM_000439.3 | Homo sapiens proprotein | PCSK1 |
| | convertase subtilisin/kexin | |
| | type 1 (PCSK1), mRNA. | |
| NM_000439.3 | Homo sapiens proprotein | PCSK1 |
| | convertase subtilisin/kexin | |
| | type 1 (PCSK1), mRNA. | |
| NM_003381.2 | Homo sapiens vasoactive | VIP |
| | intestinal peptide (VIP), | |
| | transcript variant 1, mRNA. | |
| NM_001105519.1 | Homo sapiens chromosome 2 | C2orf70 |
| | open reading frame 70 | |
| | (C2orf70), mRNA. | |
| NM_020436.2 | Homo sapiens sal-like 4 | SALL4 |
| | (Drosophila) (SALL4), mRNA. | |
| NM_001008783.1 | Homo sapiens solute carrier | SLC35D3 |
| | family 35, member D3 | |
| | (SLC35D3), mRNA. | |
| NM_021246.2 | Homo sapiens lymphocyte | LY6G6D |
| | antigen 6 complex, locus G6D | |
| | (LY6G6D), mRNA. | |
| XM_001133677.1 | PREDICTED: Homo sapiens | LOC729264 |
| | similar to TP53TG3 protein, | |
| | transcript variant 2 | |
| | (LOC729264), mRNA. | |
| NM_001555.2 | Homo sapiens immunoglobulin | IGSF1 |
| | superfamily, member 1 | |
| | (IGSF1), transcript variant 1, | |
| | mRNA. | |
| NM_005940.3 | Homo sapiens matrix | MMP11 |
| | metallopeptidase 11 | |
| | (stromelysin 3) (MMP11), | |
| | mRNA. | |
| NR_006882.1 | Homo sapiens small nucleolar | SNORD3D |
| | RNA, C/D box 3D | |
| | (SNORD3D), small nucleolar | |
| | RNA. | |
| NM_000479.2 | Homo sapiens anti-Mullerian | AMH |
| | hormone (AMH), mRNA. | |
| NM_013404.3 | Homo sapiens mesothelin | MSLN |
| | (MSLN), transcript variant 2, | |
| | mRNA. | |
| BU536065 | AGENCOURT_10229596 | |
| | NIH_MGC_141 Homo sapiens | |
| | cDNA clone | |
| | IMAGE: 6563923 5, | |
| | mRNA sequence | |
| NR_002739.1 | Homo sapiens small nucleolar | SNORD56 |
| | RNA, C/D box 56 (SNORD56), | |
| | small nuclear RNA. | |
| NM_144668.4 | Homo sapiens WD repeat | WDR66 |
| | domain 66 (WDR66), mRNA. | |
| NR_006881.1 | Homo sapiens small nucleolar | SNORD3C |
| | RNA, C/D box 3C | |
| | (SNORD3C), | |
| | small nucleolar RNA. | |
| NR_006880.1 | Homo sapiens small nucleolar | SNORD3A |
| | RNA, C/D box 3A | |
| | (SNORD3A), small nucleolar | |
| | RNA. | |
| NM_144594.1 | Homo sapiens gametocyte | GTSF1 |
| | specific factor 1 (GTSF1), | |
| | mRNA. | |
| NM_016212.2 | Homo sapiens TP53 target 3 | TP53TG3 |
| | (TP53TG3), mRNA. | |
| NM_002594.2 | Homo sapiens proprotein | PCSK2 |
| | convertase subtilisin/kexin | |
| | type 2 (PCSK2), mRNA. | |
| NM_203428.1 | Homo sapiens Down syndrome | DSCR8 |
| | critical region gene 8 (DSCR8), | |
| | transcript variant 2, mRNA. | |
| NM_006183.3 | Homo sapiens neurotensin | NTS |
| | (NTS), mRNA. | |
| NM_001001888.1 | Homo sapiens variably charged | VCX-C |
| | X-C (VCX-C), mRNA. | |
| NM_203429.1 | Homo sapiens Down syndrome | DSCR8 |
| | critical region gene 8 (DSCR8), | |
| | transcript variant 3, mRNA. | |
| NM_021115.3 | Homo sapiens seizure related | c |
| | 6 homolog (mouse)-like | |
| | (SEZ6L), mRNA. | |
| NM_004535.2 | Homo sapiens myelin | MYT1 |
| | transcription factor 1 (MYT1), | |
| | mRNA. | |
| NM_152224.1 | Homo sapiens protein | PPEF1 |
| | phosphatase, EF-hand calcium | |
| | binding domain 1 (PPEF1), | |
| | transcript variant 1b, mRNA. | |
| NM_000295.3 | Homo sapiens Serpin | SERP1NA1 |
| | peptidase inhibitor, clade A | |
| | (alpha-1 antiproteinase, | |
| | antitrypsin), member 1 | |
| | (SERPINA1), transcript variant | |
| | 1, mRNA. | |
| NM_014420.2 | Homo sapiens dickkopf | DKK4 |
| | homolog 4 (Xenopus laevis) | |
| | (DKK4), mRNA. | |
| NM_013452.2 | Homo sapiens variable charge, | VCX |
| | X-linked (VCX), mRNA. | |
| NM_052959.2 | Homo sapiens pannexin 3 | PANX3 |
| | (PANX3), mRNA. | |
| NM_144967.2 | Homo sapiens hypothetical | FLJ30058 |
| | protein FLJ30058 (FLJ30058), | |
| | mRNA. | |
| NM_016379.2 | Homo sapiens variable charge, | VCX3A |
| | X-linked 3A (VCX3A), mRNA. | |
| NM_001001552.3 | Homo sapiens LEM domain | LEMD1 |
| | containing 1 (LEMD1), mRNA. | |
| XR_041261.1 | PREDICTED: Homo sapiens | LOC730081 |
| | misc_RNA (LOC730081), | |
| | miscRNA. | |
| NM_024923.2 | Homo sapiens nucleoporin | NUP210 |
| | 210 kDa (NUP210), mRNA, | |
| NM_001926.2 | Homo sapiens defensin, alpha | DEFA6 |
| | 6, Paneth cell-specific (DEFA6), | |
| | mRNA. | |
| NM_002299.2 | Homo sapiens lactase (LCT), | LCT |
| | mRNA. | |
| NM_182980.2 | Homo sapiens oxidative stress | OSGIN1 |
| | induced growth inhibitor 1 | |
| | (OSGIN1), transcript variant 2, | |
| | mRNA. | |
| NR_002581.1 | Homo sapiens small nucleolar | SNORA72 |
| | RNA, H/ACA box 72 | |
| | (SNORA72), small nucleolar | |
| | RNA. | |
| NM_004950.3 | Homo sapiens epiphycan | EPYC |
| | (EPYC), mRNA. | |
| NM_016249.2 | Homo sapiens melanoma | MAGEC2 |
| | antigen family C, 2 (MAGEC2), | |
| | mRNA. | |
| NM_207339.2 | Homo sapiens P antigen | PAGE2 |
| | family, member 2 (prostate | |
| | associated) (PAGE2), mRNA. | |
| NM_001015038.1 | Homo sapiens P antigen | PAGE2B |
| | family, member 2B (PAGE2B), | |
| | mRNA. | |
| NM_130467.3 | Homo sapiens P antigen | PAGE5 |
| | family, member 5 (prostate | |
| | associated) (PAGE5), transcript | |
| | variant 1, mRNA. | |
| NM_004988.3 | Homo sapiens melanoma | MAGEA1 |
| | antigen family A, 1 (directs | |
| | expression of antigen MZ2-E) | |
| | (MAGEA1), mRNA. | |
| NM_173798.2 | Homo sapiens zinc finger, | ZCCHC12 |
| | CCHC domain containing 12 | |
| | (ZCCHC12), mRNA. | |
| NM_001080466.1 | Homo sapiens BTB (POZ) | BTBD17 |
| | domain containing 17 | |
| | (BTBD17), mRNA. | |
| NM_021010.1 | Homo sapiens defensin, alpha | DEFA5 |
| | 5, Paneth cell-specific (DEFA5), | |
| | mRNA. | |
| XM_941629.1 | PREDICTED: Homo sapiens | LOC652235 |
| | hypothetical protein | |
| | LOC652235 (LOC652235), | |
| | mRNA. | |
| NM_014582.2 | Homo sapiens odorant binding | OBP2A |
| | protein 2A (OBP2A), mRNA. | |
| NM_001004317.2 | Homo sapiens lin-28 homolog | LIN28B |
| | B (C. elegans) | |
| | (LIN28B), mRNA. | |
| NM_001001933.1 | Homo sapiens LIM homeobox | LHX8 |
| | 8 (LHX8), mRNA. | |
| NM_002362.4 | Homo sapiens melanoma | MAGEA4 |
| | antigen family A, 4 (MAGEA4), | |
| | transcript variant 2, mRNA. | |
| NM_002196.2 | Homo sapiens insulinoma- | INSM1 |
| | associated 1 (INSM1), mRNA. | |
| NM_175901.3 | Homo sapiens hypothetical | LOC283932 |
| | protein LOC283932 | |
| | (LOC283932), mRNA. | |
| NM_014581.2 | Homo sapiens odorant binding | OBP2B |
| | protein 2B (OBP2B), mRNA. | |
| NM_001042600.1 | Homo sapiens mitogen- | MAP4K1 |
| | activated protein kinase kinase | |
| | kinase kinase 1 (MAP4K1), | |
| | transcript variant 1, mRNA. | |
| NM_001042600.1 | Homo sapiens mitogen- | MAP4K1 |
| | activated protein kinase kinase | |
| | kinase kinase 1 (MAP4K1), | |
| | transcript variant 1, mRNA. | |
| NM_002240.2 | Homo sapiens potassium | KCNJ6 |
| | inwardly-rectifying channel, | |
| | subfamily J, member 6 | |
| | (KCNJ6), mRNA. | |
| NM_199048.1 | Homo sapiens T1560 protein | T1560 |
| | (T1560), mRNA. | |
| NM_014509.3 | Homo sapiens serine | SERHL2 |
| | hydrolase-like 2 (SERHL2), | |
| | mRNA. | |
| NM_080614.1 | Homo sapiens WAP four- | WFDC3 |
| | disulfide core domain 3 | |
| | (WFDC3), mRNA. | |
| NM_203400.1 | Homo sapiens reprimo-like | RPRML |
| | (RPRML), mRNA. | |
| NM_001001663.1 | Homo sapiens transmembrane | TMEM211 |
| | protein 211 (TMEM211), | |
| | mRNA. | |
| NM_020826.1 | Homo sapiens synaptotagmin | SYT13 |
| | XIII (SYT13), mRNA. | |
| NM_018044.2 | Homo sapiens | NSUN5 |
| | NOL1/NOP2/Sun domain | |
| | family, member 5 (NSUN5), | |
| | transcript variant 2, mRNA. | |
| NM_018936.2 | Homo sapiens protocadherin | PCDHB2 |
| | beta 2 (PCDHB2), mRNA. | |
| XM_927237.1 | PREDICTED: Homo sapiens | LOC653219 |
| | similar to G antigen, family D, | |
| | 2 isoform 1a, transcript variant | |
| | 1 (LOC653219), mRNA. | |
| NM_203311.1 | Homo sapiens CSAG family, | CSAG3A |
| | member 3A (CSAG3A), mRNA. | |
| NM_004918.2 | Homo sapiens T-cell, | TCL1B |
| | leukemia/lymphoma 1B | |
| | (TCL1B), transcript variant 1, | |
| | mRNA. | |
| NM_001099676.1 | Homo sapiens chromosome 12 | C12orf56 |
| | open reading frame 56 | |
| | (C12orf56), mRNA. | |
| NM_005310.2 | Homo sapiens growth factor | GRB7 |
| | receptor-bound protein 7 | |
| | (GRB7), transcript variant 1, | |
| | mRNA. | |
| NM_021951.2 | Homo sapiens doublesex and | DMRT1 |
| | mab-3 related transcription | |
| | factor 1 (DMRT1), mRNA. | |
| NM_153479.1 | Homo sapiens | CSAG1 |
| | chondrosarcoma associated | |
| | gene 1 (CSAG1), transcript | |
| | variant b, mRNA, | |
| NM_005634.2 | Homo sapiens SRY (sex | SOX3 |
| | determining region Y)-box 3 | |
| | (SOX3), mRNA. | |
| NM_001017436.1 | Homo sapiens cancer/testis | CT45A4 |
| | antigen family 45, member A4 | |
| | (CT45A4), mRNA, | |
| NM_001017436.1 | Homo sapiens cancer/testis | CT45A4 |
| | antigen family 45, member A4 | |
| | (CT45A4), mRNA. | |
| NM_001017436.1 | Homo sapiens cancer/testis | CT45A4 |
| | antigen family 45, member A4 | |
| | (CT45A4), mRNA. | |
| NM_019079.2 | Homo sapiens LINE-1 type | L1TD1 |
| | transposase domain | |
| | containing 1 (L1TD1), mRNA. | |
| NM_133431.1 | Homo sapiens X antigen | XAGE1 |
| | family, member 1 (XAGE1), | |
| | transcript variant 2, mRNA. | |
| NM_014258.2 | Homo sapiens synaptonemal | SYCP2 |
| | complex protein 2 (SYCP2), | |
| | mRNA. | |
| NM_001017361.2 | Homo sapiens chromosome 6 | C6orf221 |
| | open reading frame 221 | |
| | (C6orf221), mRNA. | |
| XR_017850.1 | PREDICTED: Homo sapiens | PART1 |
| | prostate androgen-regulated | |
| | transcript 1 (PART1), misc | |
| | RNA. | |
| NM_014592.2 | Homo sapiens Kv channel | KCNIP1 |
| | interacting protein 1 (KCNIP1), | |
| | transcript variant 2, mRNA. | |
| NM_002846.2 | Homo sapiens protein tyrosine | PTPRN |
| | phosphatase, receptor type, N | |
| | (PTPRN), mRNA. | |
| NM_000735.2 | Homo sapiens glycoprotein | CGA |
| | hormones, alpha polypeptide | |
| | (CGA), mRNA. | |
| NM_002701.4 | Homo sapiens POU class 5 | POU5F1 |
| | homeobox 1 (POU5F1), | |
| | transcript variant 1, mRNA. | |
| NM_001079530.1 | Homo sapiens cripto, FRL-1, | CFC1B |
| | cryptic family 1B (CFC1B), | |
| | mRNA. | |
| NM_004884.3 | Homo sapiens immunoglobulin | IGDCC3 |
| | superfamily, DCC subclass, | |
| | member 3 (IGDCC3), mRNA. | |
| NM_199286.2 | Homo sapiens developmental | DPPA3 |
| | pluripotency associated 3 | |
| | (DPPA3), mRNA. | |
| NM_001097595.1 | Homo sapiens X antigen | XAGE1B |
| | family, member 1B (XAGE1B), | |
| | transcript variant 1, mRNA. | |
| NM_002379.2 | Homo sapiens matrilin 1, | MATN1 |
| | cartilage matrix protein | |
| | (MATN1), mRNA. | |
| NM_080618.2 | Homo sapiens CCCTC-binding | CTCFL |
| | factor (zinc finger protein)-like | |
| | (CTCFL), mRNA. | |
| NM_031950.2 | Homo sapiens fibroblast | FGFBP2 |
| | growth factor binding protein | |
| | 2 (FGFBP2), mRNA. | |
| AK024399 | Homo sapiens cDNA FLJ14337 | |
| | fis, clone PLACE4000494 | |
| NM_004861.1 | Homo sapiens galactose-3-O- | GAL3ST1 |
| | sulfotransferase 1 (GAL3ST1), | |
| | mRNA. | |
| CA849062 | ir69h03.y1 HR85 islet | |
| | Homo sapiens cDNA clone | |
| | IMAGE: 66079025, mRNA | |
| | sequence | |
| NM_001010874.3 | Homo sapiens steroid 5 alpha- | SRD5A2L2 |
| | reductase 2-like 2 (SRD5A2L2), | |
| | mRNA. | |
| NM_198152.2 | Homo sapiens urotensin 2 | UTS2D |
| | domain containing (UTS2D), | |
| | mRNA. | |
| NM_173509.2 | Homo sapiens family with | FAM163A |
| | sequence similarity 163, | |
| | member A (FAM163A), mRNA. | |
| NM_006998.3 | Homo sapiens secretagogin, | SCGN |
| | EF-hand calcium binding | |
| | protein (SCGN), mRNA. | |
| NM_001025290.1 | Homo sapiens developmental | DPPA5 |
| | pluripotency associated 5 | |
| | (DPPA5), mRNA. | |
| NM_032132.3 | Homo sapiens HORMA domain | HORMAD1 |
| | containing 1 (HORMAD1), | |
| | mRNA. | |
| NM_001097597.1 | Homo sapiens X antigen | XAGE1C |
| | family, member 1C (XAGE1C), | |
| | transcript variant 2, mRNA. | |
| XM_936973.2 | PREDICTED: Homo sapiens | LOC338579 |
| | hypothetical protein | |
| | LOC338579, transcript variant | |
| | 2 (LOC338579), mRNA. | |
| NM_001034838.1 | Homo sapiens Kv channel | KCNIP1 |
| | interacting protein 1 (KCNIP1), | |
| | transcript variant 3, mRNA. | |
| NM_030592.1 | Homo sapiens matrilin 4 | MATN4 |
| | (MATN4), transcript variant 3, | |
| | mRNA. | |
| NR_002304.1 | Homo sapiens POU class 5 | POU5F1P1 |
| | homeobox 1 pseudogene 1 | |
| | (POU5F1P1), non-coding RNA. | |
| NR_002304.1 | Homo sapiens POU class 5 | POU5F1P1 |
| | homeobox 1 pseudogene 1 | |
| | (POU5F1P1), non-coding RNA. | |
| NM_173092.1 | Homo sapiens potassium | KCNH6 |
| | voltage-gated channel, | |
| | subfamily H (eag-related), | |
| | member 6 (KCNH6), transcript | |
| | variant 2, mRNA. | |
| XR_017655.1 | PREDICTED: Homo sapiens | LOC645682 |
| | hypothetical LOC645682 | |
| | (LOC645682), mRNA. | |
| NM_148674.3 | Homo sapiens structural | SMC1B |
| | maintenance of chromosomes | |
| | 1B (SMC1B), mRNA. | |
| NM_178550.3 | Homo sapiens chromosome 1 | C1orf110 |
| | open reading frame 110 | |
| | (C1orf110), mRNA. | |
| XM_945048.1 | PREDICTED: Homo sapiens | LOC651957 |
| | hypothetical protein | |
| | LOC651957 (LOC651957), | |
| | mRNA. | |
| NM_203347.1 | Homo sapiens lipocalin 15 | LCN15 |
| | (LCN15), mRNA. | |
| NM_170694.1 | Homo sapiens serine | SERHL |
| | hydrolase-like (SERHL), | |
| | mRNA. | |
| NM_001097593.1 | Homo sapiens X antigen | XAGE1A |
| | family, member 1A (XAGE1A), | |
| | transcript variant 3, mRNA. | |
| NM_177524.1 | Homo sapiens mesoderm | MEST |
| | specific transcript homolog | |
| | (mouse) (MEST), transcript | |
| | variant 2, mRNA. | |
| NM_033043.1 | Homo sapiens chorionic | CGB5 |
| | gonadotropin, beta | |
| | polypeptide 5 (CGB5), mRNA. | |
| NM_002851.2 | Homo sapiens protein tyrosine | PTPRZ1 |
| | phosphatase, receptor-type, Z | |
| | polypeptide 1 (PTPRZ1), | |
| | mRNA. | |
| NM_006418.3 | Homo sapiens olfactomedin 4 | OLFM4 |
| | (OLFM4), mRNA. | |
| NM_006418.3 | Homo sapiens olfactomedin 4 | OLFM4 |
| | (OLFM4), mRNA. | |
| NM_001017417.1 | Homo sapiens cancer/testis | CT45A1 |
| | antigen family 45, member A1 | |
| | (CT45A1), mRNA. | |
| BX537518 | Homo sapiens mRNA; cDNA | |
| | DKFZp686N1989 (from clone | |
| | DKFZp686N1989) | |
| NM_001080848.1 | Homo sapiens CSAG family, | CSAG3B |
| | member 3B (CSAG3B), | |
| | mRNA. | |
| NM_003655.2 | Homo sapiens chromobox | CBX4 |
| | homolog 4 (Pc class homolog, | |
| | Drosophila) | |
| | (CBX4), mRNA. | |
| NM_003483.4 | Homo sapiens high mobility | HMGA2 |
| | group AT-hook 2 (HMGA2), | |
| | transcript variant 1, mRNA. | |
| NM_032545.2 | Homo sapiens cripto, FRL-1, | CFC1 |
| | cryptic family 1 (CFC1), | |
| | mRNA. | |
| XM_001719794.1 | PREDICTED: Homo sapiens | LOC100133542 |
| | hypothetical protein | |
| | LOC100133542 | |
| | (LOC100133542), partial | |
| | mRNA. | |
| NM_004316.2 | Homo sapiens achaete-scute | ASCL1 |
| | complex homolog 1 | |
| | (Drosophila) | |
| | (ASCL1), mRNA. | |
| NM_001017361.1 | Homo sapiens ES cell | ECAT1 |
| | associated transcript 1 | |
| | (ECAT1), mRNA. | |
| NM_198965.1 | Homo sapiens parathyroid | PTHLH |
| | hormone-like hormone | |
| | (PTHLH), transcript variant 1, | |
| | mRNA. | |
| NM_002277.2 | Homo sapiens keratin 31 | KRT31 |
| | (KRT31), mRNA. | |
| NM_024070.3 | Homo sapiens poliovirus | PVRIG |
| | receptor related | |
| | immunoglobulin domain | |
| | containing (PVRIG), mRNA. | |
| NM_175056.1 | Homo sapiens zona pellucida- | ZPLD1 |
| | like domain containing 1 | |
| | (ZPLD1), mRNA. | |
| NM_001013734.2 | Homo sapiens ret finger | RFPL4B |
| | protein-like 4B (RFPL4B), | |
| | mRNA. | |
| XM_001724554.1 | PREDICTED: Homo sapiens | LOC100134331 |
| | similar to hCG1812074 | |
| | (LOC100134331), mRNA. | |
| NM_020209.2 | Homo sapiens Src homology 2 | SHD |
| | domain containing | |
| | transforming protein D (SHD), | |
| | mRNA. | |
| NR_024418.1 | Homo sapiens hypothetical | LOC389332 |
| | LOC389332 (LOC389332), non- | |
| | coding RNA. | |
| NM_030672.2 | Homo sapiens Rho GTPase | ARHGAP28 |
| | activating protein 28 | |
| | (ARHGAP28), transcript variant | |
| | 2, mRNA. | |
| NM_033377.1 | Homo sapiens chorionic | CGB1 |
| | gonadotropin, beta | |
| | polypeptide 1 (CGB1), mRNA. | |
| NM_012284.1 | Homo sapiens potassium | KCNH3 |
| | voltage-gated channel, | |
| | subfamily H (eag-related), | |
| | member 3 (KCNH3), mRNA. | |
| NM_001844.3 | Homo sapiens collagen, type II, | COL2A1 |
| | alpha 1 (primary osteoarthritis, | |
| | spondyloepiphyseal dysplasia, | |
| | congenital) (COL2A1), | |
| | transcript variant 1, mRNA. | |
| NM_005752.2 | Homo sapiens C-type lectin | CLEC3A |
| | domain family 3, member A | |
| | (CLEC3A), mRNA. | |
| NM_182562.2 | Homo sapiens family with | FAM169B |
| | sequence similarity 169, | |
| | member B (FAM169B), mRNA. | |
| NM_002407.1 | Homo sapiens secretoglobin, | SCGB2A1 |
| | family 2A, member 1 | |
| | (SCGB2A1), mRNA. | |
| NM_001252.3 | Homo sapiens CD70 molecule | CD70 |
| | (CD70), mRNA. | |
| NM_030812.1 | Homo sapiens actin-like 8 | ACTL8 |
| | (ACTL8), mRNA. | |
| NM_006237.3 | Homo sapiens POU class 4 | POU4F1 |
| | homeobox 1 (POU4F1), mRNA. | |
| XM_001716834.1 | PREDICTED: Homo sapiens | LOC642131 |
| | similar to hCG1812074 | |
| | (LOC642131), mRNA. | |
| NM_006152.2 | Homo sapiens lymphoid- | LRMP |
| | restricted membrane protein | |
| | (LRMP), mRNA. | |
| NM_152439.2 | Homo sapiens bestrophin 3 | BEST3 |
| | (BEST3), transcript variant 2, | |
| | mRNA. | |
| NM_002411.2 | Homo sapiens secretoglobin, | SCGB2A2 |
| | family 2A, member 2 | |
| | (SCGB2A2), mRNA. | |
| XM_498560.3 | PREDICTED: Homo sapiens | LOC440132 |
| | hypothetical LOC440132 | |
| | (LOC440132), mRNA. | |
| NM_018674.3 | Homo sapiens amiloride- | ACCN4 |
| | sensitive cation channel 4, | |
| | pituitary (ACCN4), transcript | |
| | variant 1, mRNA. | |
| NM_015011.1 | Homo sapiens myosin XVI | MYO16 |
| | (MYO16), mRNA. | |
| NM_001819.1 | Homo sapiens chromogranin B | CHGB |
| | (secretogranin 1) (CHGB), | |
| | mRNA. | |
| NM_004852.2 | Homo sapiens one cut | ONECUT2 |
| | homeobox 2 (ONECUT2), | |
| | mRNA. | |
| NM_004852.2 | Homo sapiens one cut | ONECUT2 |
| | homeobox 2 (ONECUT2), | |
| | mRNA. | |
| XM_928495.1 | PREDICTED: Homo sapiens | LOC645464 |
| | similar to vasoactive intestinal | |
| | peptide receptor 2 | |
| | (LOC645464), mRNA. | |
| NM_004679.2 | Homo sapiens variable charge, | VCY |
| | Y-linked (VCY), mRNA. | |
| NM_080681.2 | Homo sapiens collagen, type | COL11A2 |
| | XI, alpha 2 (COL11A2), | |
| | transcript variant 2, mRNA. | |
| NM_031282.1 | Homo sapiens Fc receptor-like | FCRL4 |
| | 4 (FCRL4), mRNA. | |
| XR_037048.1 | PREDICTED: Homo sapiens | LOC651397 |
| | misc_RNA (LOC651397), | |
| | miscRNA. | |
| NM_001532.2 | Homo sapiens solute carrier | SLC29A2 |
| | family 29 (nucleoside | |
| | transporters), member 2 | |
| | (SLC29A2), mRNA. | |
| NM_080429.2 | Homo sapiens aquaporin 10 | AQP10 |
| | (AQP10), mRNA. | |
| NM_001010905.1 | Homo sapiens chromosome 6 | C6orf58 |
| | open reading frame 58 | |
| | (C6orf58), mRNA. | |
| NM_153046.1 | Homo sapiens tudor domain | TDRD9 |
| | containing 9 (TDRD9), mRNA. | |
| NM_000369.2 | Homo sapiens thyroid | TSHR |
| | stimulating hormone receptor | |
| | (TSHR), transcript variant 1, | |
| | mRNA. | |
| NM_002854.2 | Homo sapiens parvalbumin | PVALB |
| | (PVALB), mRNA. | |
| NM_173698.1 | Homo sapiens family with | FAM133A |
| | sequence similarity 133, | |
| | member A (FAM133A), mRNA. | |
| XM_939163.2 | PREDICTED: Homo sapiens | FLJ23152 |
| | hypothetical protein | |
| | LOC401236 (FLJ23152), | |
| | mRNA. | |
| NR_004390.1 | Homo sapiens small nucleolar | SNORA57 |
| | RNA, H/ACA box 57 | |
| | (SNORA57), small nucleolar | |
| | RNA. | |
| XM_930694.1 | PREDICTED: Homo sapiens | LOC642477 |
| | hypothetical protein | |
| | LOC642477, transcript variant | |
| | 2 (LOC642477), mRNA. | |
| NM_020708.3 | Homo sapiens solute carrier | SLC12A5 |
| | family 12, (potassium-chloride | |
| | transporter) member 5 | |
| | (SLC12A5), mRNA. | |
| NM_144647.3 | Homo sapiens calcyphosine- | CAPSL |
| | like (CAPSL), transcript variant | |
| | 1, mRNA. | |
| NM_199161.1 | Homo sapiens serum amyloid | SAA1 |
| | A1 (SAA1), transcript variant 2, | |
| | mRNA. | |
| NM_145754.2 | Homo sapiens kinesin family | KIFC2 |
| | member C2 (KIFC2), mRNA. | |
| NM_001010925.2 | Homo sapiens ankyrin repeat | ANKRD19 |
| | domain 19 (ANKRD19), | |
| | mRNA. | |
| XM_001131823.1 | PREDICTED: Homo sapiens | ANKRD30A |
| | ankyrin repeat domain 30A | |
| | (ANKRD30A), mRNA. | |
| NR_003059.1 | Homo sapiens small nucleolar | SNORD71 |
| | RNA, C/D box 71 (SNORD71), | |
| | small nucleolar RNA. | |
| NM_004833.1 | Homo sapiens absent in | AIM2 |
| | melanoma 2 (AIM2), | |
| | mRNA. | |
| NM_019106.4 | Homo sapiens septin 3 | 3-Sep |
| | (SEPT3), transcript variant B, | |
| | mRNA. | |
| NM_001042496.1 | Homo sapiens solute carrier | SLC12A6 |
| | family 12 (potassium/chloride | |
| | transporters), member 6 | |
| | (SLC12A6), transcript variant 5, | |
| | mRNA. | |
| XR_038222.1 | PREDICTED: Homo sapiens | LOC100133312 |
| | misc_RNA (LOC100133312), | |
| | miscRNA. | |
| NM_001010985.1 | Homo sapiens myosin binding | MYBPHL |
| | protein H-like (MYBPHL), | |
| | mRNA. | |
| NR_002987.1 | Homo sapiens small nucleolar | SNORA61 |
| | RNA, H/ACA box 61 | |
| | (SNORA61), small nucleolar | |
| | RNA. | |
| NM_003378.2 | Homo sapiens VGF nerve | VGF |
| | growth factor inducible (VGF), | |
| | mRNA. | |
| NM_052900.2 | Homo sapiens CUB and Sushi | CSMD3 |
| | multiple domains 3 (CSMD3), | |
| | transcript variant c, mRNA. | |
| NM_053283.2 | Homo sapiens dermcidin | DCD |
| | (DCD), mRNA. | |
| NM_172004.2 | Homo sapiens C-type lectin- | CLECL1 |
| | like 1 (CLECL1), mRNA. | |
| NM_016378.2 | Homo sapiens variable charge, | VCX2 |
| | X-linked 2 (VCX2), mRNA. | |
| XR_037336.1 | PREDICTED: Homo sapiens | LOC100131139 |
| | misc_RNA (LOC100131139), | |
| | miscRNA. | |
| NM_014224.1 | Homo sapiens pepsinogen 5, | PGA5 |
| | group 1 (pepsinogen A) (PGA5), | |
| | mRNA. | |
| NM_207033.1 | Homo sapiens endothelin 3 | EDN3 |
| | (EDN3), transcript variant 3, | |
| | mRNA. | |
| NM_138768.2 | Homo sapiens myeloma | MYEOV |
| | overexpressed (in a subset of | |
| | t(11; 14) positive multiple | |
| | myelomas) (MYEOV), mRNA. | |
| NR_023371.1 | Homo sapiens RNA, 5S | RN5S9 |
| | ribosomal 9 (RN5S9), | |
| | ribosomal RNA. | |
| XM_001713808.1 | PREDICTED: Homo sapiens | LOC100132564 |
| | hypothetical protein | |
| | LOC100132564 | |
| | (LOC100132564), mRNA. | |
| NM_000482.3 | Homo sapiens apolipoprotein | APOA4 |
| | A-IV (APOA4), mRNA. | |
| NM_006658.2 | Homo sapiens chromosome 7 | C7orf16 |
| | open reading frame 16 | |
| | (C7orf16), mRNA. | |
| NR_015379.2 | Homo sapiens urothelial | UCA1 |
| | cancer associated 1 (non- | |
| | protein coding) (UCA1), non- | |
| | coding RNA. | |
| NM_024877.1 | Homo sapiens cyclin N- | CNTD2 |
| | terminal domain containing 2 | |
| | (CNTD2), mRNA. | |
| NM_032738.3 | Homo sapiens Fc receptor-like | FCRLA |
| | A (FCRLA), mRNA. | |
| NM_000316.2 | Homo sapiens parathyroid | PTH1R |
| | hormone 1 receptor (PTH1R), | |
| | mRNA. | |
| NM_000316.2 | Homo sapiens parathyroid | PTH1R |
| | hormone 1 receptor (PTH1R), | |
| | mRNA, | |
| NM_000756.1 | Homo sapiens corticotropin | CRH |
| | releasing hormone (CRH), | |
| | mRNA. | |
| NM_004306.2 | Homo sapiens annexin A13 | ANXA13 |
| | (ANXA13), transcript variant 1, | |
| | mRNA. | |
| NM_000894.2 | Homo sapiens luteinizing | LHB |
| | hormone beta polypeptide | |
| | (LHB), mRNA. | |
| NM_004291.2 | Homo sapiens CART | CARTPT |
| | prepropeptide (CARTPT), | |
| | mRNA. | |
| NM_001093770.1 | Homo sapiens surfactant | SFTPA1 |
| | protein A1 (SFTPA1), mRNA. | |
| | XM_934600 | |
| XM_935867.1 | PREDICTED: Homo sapiens | LOC641738 |
| | hypothetical protein | |
| | LOC641738 (LOC641738), | |
| | mRNA. | |
| NM_000125.2 | Homo sapiens estrogen | ESR1 |
| | receptor 1 (ESR1), mRNA. | |
| NM_001974.3 | Homo sapiens egf-like module | EMR1 |
| | containing, mucin-like, | |
| | hormone receptor-like 1 | |
| | (EMR1), mRNA. | |
| NM_005247.2 | Homo sapiens fibroblast | FGF3 |
| | growth factor 3 (murine | |
| | mammary tumor virus | |
| | integration site (v-int-2) | |
| | oncogene homolog) (FGF3), | |
| | mRNA. | |
| XM_944750.2 | PREDICTED: Homo sapiens | LOC646360 |
| | hCG25653 (LOC646360), | |
| | mRNA. | |
| XM_927939.2 | PREDICTED: Homo sapiens | LOC644844 |
| | hypothetical LOC644844 | |
| | (LOC644844), mRNA. | |
| NM_018656.2 | Homo sapiens solute carrier | SLC35E3 |
| | family 35, member E3 | |
| | (SLC35E3), mRNA. | |
| NM_000583.2 | Homo sapiens group-specific | GC |
| | component (vitamin D binding | |
| | protein) (GC), mRNA. | |
| NM_004617.2 | Homo sapiens transmembrane | TM4SF4 |
| | 4 L six family member 4 | |
| | (TM4SF4), mRNA. | |
| NM_006365.1 | Homo sapiens chromosome 1 | C1orf61 |
| | open reading frame 61 | |
| | (C1orf61), mRNA. | |
| NM_022573.2 | Homo sapiens testis specific | TSPY2 |
| | protein, Y-linked 2 (TSPY2), | |
| | mRNA. | |
| NM_203395.1 | Homo sapiens iodotyrosine | IYD |
| | deiodinase (IYD), mRNA. | |
| NM_004190.1 | Homo sapiens lipase, gastric | LIPF |
| | (LIPF), mRNA. | |
| NM_002252.3 | Homo sapiens potassium | KCNS3 |
| | voltage-gated channel, | |
| | delayed-rectifier, subfamily S, | |
| | member 3 (KCNS3), mRNA. | |
| NM_002252.3 | Homo sapiens potassium | KCNS3 |
| | voltage-gated channel, | |
| | delayed-rectifier, subfamily S, | |
| | member 3 (KCNS3), mRNA. | |
| NM_004518.2 | Homo sapiens potassium | KCNQ2 |
| | voltage-gated channel, KQT- | |
| | like subfamily, member 2 | |
| | (KCNQ2), transcript variant 3, | |
| | mRNA. | |
| NM_005832.3 | Homo sapiens potassium large | KCNMB2 |
| | conductance calcium-activated | |
| | channel, subfamily M, beta | |
| | member 2 (KCNMB2), | |
| | transcript variant 2, mRNA. | |
| NR_002728.2 | Homo sapiens KCNQ1 | KCNQ1OT1 |
| | overlapping transcript 1 (non- | |
| | protein coding) (KCNQ1OT1), | |
| | non-coding RNA. | |
| NM_172109.1 | Homo sapiens potassium | KCNQ2 |
| | voltage-gated channel, KQT- | |
| | like subfamily, member 2 | |
| | (KCNQ2), transcript variant 5, | |
| | mRNA. | |
| NM_031460.3 | Homo sapiens potassium | KCNK17 |
| | channel, subfamily K, member | |
| | 17 (KCNK17), transcript variant | |
| | 1, mRNA. | |
| NM_022358.2 | Homo sapiens potassium | KCNK15 |
| | channel, subfamily K, member | |
| | 15 (KCNK15), mRNA. | |
| NM_001622.1 | Homo sapiens alpha-2-HS- | AHSG |
| | glycoprotein (AHSG), mRNA. | |
| NM_002250.2 | Homo sapiens potassium | KCNN4 |
| | intermediate/small | |
| | conductance calcium-activated | |
| | channel, subfamily N, member | |
| | 4 (KCNN4), mRNA. | |
| NM_032115.2 | Homo sapiens potassium | KCNK16 |
| | channel, subfamily K, member | |
| | 16 (KCNK16), mRNA. | |
| NM_178863.2 | Homo sapiens potassium | KCTD13 |
| | channel tetramerisation | |
| | domain containing 13 | |
| | (KCTD13), mRNA. | |
| NM_171829.1 | Homo sapiens potassium large | KCNMB3 |
| | conductance calcium-activated | |
| | channel, subfamily M beta | |
| | member 3 (KCNMB3), | |
| | transcript variant 2, mRNA. | |

It will be appreciated that there are various methods of obtaining expression data and uses of the expression data. For example, the expression data that can be used to detect or diagnose a subject with cancer can be obtained experimentally. In some embodiments, obtaining the expression data comprises obtaining the sample and processing the sample to experimentally determine the expression data. The expression data can comprise expression data for one or more of the markers described herein. The expression data can be experimentally determined by, for example, using a microarray or quantitative amplification method such as, but not limited to those described herein. In some embodiments, obtaining expression data associated with a sample comprises receiving the expression data from a third party that has processed the sample to experimentally determine the expression data.

In some embodiments, the expression of one or more sequences is compared to a normal sample and an increase in expression in the cancer sample or suspected of having cancer indicates that the sample has cancer. In some embodiments, the expression of the sequence is at least 10, 20, 30, 40, 50, 60, 70, 80 90, 100, 200, or 300 percent increased as compared to the normal sample. In some embodiments, the expression of the sequence is the expression of the mRNA, and, in some embodiments, the expression of the sequence is the expression of the encoded protein. The nucleic acid sequence can be detected by any method, including, but not limited to the methods described herein. The methods of detection include, for example, PCR, southern hybridization, northern hybridization, microarray, biochip, and the like, In some embodiments, the sequence is a gene sequence for or gene sequence encoding for a gene selected from the group consisting of NMU, PRAME, PRAME, SPINK4, PCSK1, PCSK1, VIP, C2orf70, SALL4, SLC35D3, LY6G6D, LOC729264, IGSF1, MMP11, SNORD3D, AMH, MSLN, SNORD56, WDR66, SNORD3C, SNORD3A, GTSF1, TP53TG3, PCSK2, DSCR8, NTS, VCX-C, DSCR8, SEZ6L, MYT1, PPEF1, SERPINA1, DKK4, VCX, PANX3, FLJ30058, VCX3A, LEMD1, LOC730081, NUP210, DEFA6, LCT, OSGIN1, SNORA72, EPYC, MAGEC2, PAGE2, PAGE2B, PAGE5, MAGEA1, ZCCHC12, BTBD17, DEFA5, LOC652235, OBP2A, LIN28B, LHX8, MAGEA4, INSM1, LOC283932, OBP2B, MAP4K1, MAP4K1, KCNJ6, T1560, SERHL2, WFDC3, RPRML, TMEM211, SYT13, NSUN5, PCDHB2, LOC653219, CSAG3A, TCL1B, C12orf56, GRB7, DMRT1, CSAG1, SOX3, CT45A4, CT45A4, CT45A4, L1TD1, XAGE1, SYCP2, C6orf221, PART1, KCNIP1, PTPRN, CGA, POU5F1, CFC1B, IGDCC3, DPPA3, XAGE1B, MATN1, CTCFL, FGFBP2, GAL3ST1, SRD5A2L2, UTS2D, FAM163A, SCGN, DPPA5, HORMAD1, XAGE1C, LOC338579, KCNIP1, MATN4, POU5F1P1, POU5F1P1, KCNH6, LOC645682, SMC113, C1orf110, LOC651957, LCN15, SERHL, XAGE1A, MEST, CGB5, PTPRZ1, OLFM4, OLFM4, CT45A1, CSAG3B, CBX4, HMGA2, CFC1, LOC100133542, ASCL1, ECAT1, PTHLH, KRT31, PVRIG, ZPLD1, RFPL4B, LOC100134331, SHD, LOC389332, ARHGAP28, CGB1, KCNH3, COL2A1, CLEC3A, FAM169B, SCGB2A1, CD70, ACTL8, POU4F1, LOC642131, LRMP, BEST3, SCGB2A2, LOC440132, ACCN4, MYO16, CHGB, ONECUT2, ONECUT2, LOC645464, VCY, COL11A2, FCRL4, LOC651397, SLC29A2, AQP10, C6orf58, TDRD9, TSHR, PVALB, FAM133A, FLJ23152, SNORA57, LOC642477, SLC12A5, CAPSL, SAA1, KIFC2, ANKRDI9, ANKRD30A, SNORD71, AIM2, 3-Sep, SLC12A6, LOC100133312, MYBPHL, SNORA61, VGF, CSMD3, DCD, CLECL1, VCX2, LOC100131139, PGA5, EDN3, MYEOV, RN5S9, LOC100132564, APOA4, C7orf16, UCA1, CNTD2, FCRLA, PTH1R, PTH1R, CRH, ANXA13, LHB, CARTPT, SFTPA1, LOC641738, ESR1, EMR1, FGF3, LOC646360, LOC644844, SLC35E3, GC, TM4SF4, C1orf61, TSPY2, IYD, LIPF, KCNS3, KCNS3, KCNQ2, KCNMB2, KCNQ1OT1, KCNQ2, KCNK17, KCNK15, AHSG, KCNN4, KCNK16, KCTD13, and KCNMB3, or homolog thereof, or fragment thereof, or variant thereof.

Examples of probes for the cancer associated sequences include, but are not limited to the following:

Probe Sequence
GCTGCAGCTCGTTCCTCACCTGCATGAGAGAAGAATGAAGAGATTCAGAG
TGGGGACAGAACCTTCTATGACCCGGAGCCCATCCTGTGCCCCTGTTTCA
GACCCACGTGCTGTATCCTGTCCCCCTGGAGAGTTATGAGGACATCCATG
GCGGCACTGATGGGCTCACATATACGAATGAATGCCAGCTCTGCTTGGCC
GTAGAGGGTGTTTGCAGAGCAATGCCCGTAATGCTTAGAGAATGTTCTCC
GTAGCTGAGTTTAACATGTGTGGTCTTGGTATTCTTAAGGGAACTTCCAC
CTTCGGCATGGCCTCTTTACAGGGCACCTTCTGCTCTCAGGTTGGGTGAC
CCTGATGCCTGAGATCCTGGGTCGTGACCAGCCTGGCTTGCTTGGAATAA
GCGGTCAGCTAAGGGAGAACTTGCGTGGAAGGAGCAATGCAGACACAGTG
ACTGAAACCCAGCCAGAAGAGGGACCACCTGTAAAGCAAGTCCTTTCAAG
TGCAGCAGCTACCGCCCTGACCTGTCTCTTGCCAGGACTGTGGAGCGGAT
TCACGTGTCTTCACGCATCTCTTGAATTGGAAATTGTGCCCTGGAGACTG
CCCTGCAAGTCAGCCCCATCTGCTGTTCCTTGGTCTCTAATCACCTGAGC
CAGGTCTTGGTAGGTGCCTGCATCTGTCTGCCTTCTGGCTGACAATCCTG
GTAGAGCACCGAAAACCCCGAGGAAGAGAGGTAGCGTTTTCTCCTGAGCG
CTCATCAGCCTGTCGGAGGAACGCATCAGCGCGCACCACGTGCCCAACAT
TTCCACCCCAAGAGAACTCGCGCTCAGTAAACGGGAACATGCCCCCTGCA
TTCCAGGGCACGAGTTCGAGGCCAGCCTGGTCCACATGGGTCGGaaaaaa
TTCGTCAACAGCAGTTCACCTAGTGAGTGTTGAGACTCTGGGTCTGAGTG
TCCCGAGGGATGGAAATCCGAGCCTGCAACCTGCTCCGTCAAAGGTTCAG
GAAGCCGGCTTTCTGGCGTTGCTTGGCTGCAACTGCCGTCAGCCATTGAT
CTGCAACTGCCGTCAGCCATTGATGATCGTTCTTCTCTCCGTATTGGGGA
GGGGCACAACTCACTACTCTGACAACAACAGCCCTGCGAGCAACATAGTT
TCACGTGTCTTCACGCATCCCTTGAATTGGAAATTGTGCCCTGGAGACTG
GGCCAGTGGAAATTCAGGTGAAAATGTTCATCAATTCCCATTGCATCACC
TCCCACTTGGCAGGGGCCGTCTTGTCCACTCGTTTCTGTAAACATGGGTG
CCACAAAATCTGTCACAGCAGGGCTTTTCAACACTGGGAGTTAATCCAGG
GGTGGAGGAACCACTGAGTCAGGAGAGCGAGATGGAAGAACCACTGAGTC
GAAGGCTGGCTCATACATTTTCCCAGACAGGAATTTGGCTGCCAACAGGG
GATGTCGCTACTATTCCAACCTCCGCCTGCCTCTGATGTACTCCCACCCC
CAGATGTGTGTGCTTGGGCGTGTTTCTCGTGTCGCGTTTGCGTGTCGGCT
TGGGTTGGACCTAGTGGTGTTGTCGTGAGTGCCACCTAACCAGGAGGCCA
AGTGGACTTAGCCCCTGTTTGCTCCTCCGATAACTGGGGTGACCTTGGTT
GACACTGCTCAAGCTCCAGAAATCTTCCAGCGTTGCGACTGTGGCCCTGG
GAACCACTGAGTCAGGAGAGCCAGGTGGAGGAACCACCGAGTCAGGAGAG
GGCTGGCTTAGAACCCTCAAAACCCAAACACCTCACCAACTCGGCATGTG
GTACAGTTTTGCTCAGGTCACGCCAACAGGGAAACCTCAAGTGTAGGTCT
GCCAGGTGGAGGAACCACTGAGTCAGGAGAGCGAGATGGAAGAACTACCG
CGAGAGCTGGAGAGAAGAAGGTTTCCCAGTGGGCTTGAAGCTTGCTGTGC
TGCAGCCATGTAAGCAAGTACCTTTCACCTCCCGCCATGATTCTGAGGCC
TGGGGGAGGAGACCCTTGGAAAAGTCCTCTCTTCCCAGCTCCTGATTCTG
AGAGCTTTGGGCTCAACAAGGGCTTTCACTTGCCATTGCAGAAGGTCCTG
AGTAGCTCTTGCGGAAACGTGTAGATACTGGTCTAGTGGGTCTGTGAACC
TGCTAAGGAAGGAGACCAGGAAGCCACCCTAACACTCGGCCAGACCCGCT
GACCATGCATGTGTCCCCAAACCTAGTTCTTTCCCTAGGTCTGGTTTCAT
CCACATCCCTCTGCCACTCCCAGAAAATCTACGAGCCCTTCACCTCCAGA
TAGTGGAACAAAATTGAAGGGTGGTCAGTAGTTTCATTTCCTTGTCCTGC
GCAGTGCCTGCTTTTCAAGGGCCTGACATGGAAGCTTTTCAACAGGAACT
CTTTCCGCCATCTTGATTCTTTGTCACTGACCGAGACTCAGCCGTGGGAA
GGGACTCTGCCCACTTTTGATCCCACTAAAGTGCTGGAAGCAGGTGAAGG
GCGGTCAGTGTTCTCAGTAGTAGGTTTCTGTTCTATTGGGTGACTTGGAG
CCCTGCAGCCTACGGGTCTGTTTTCTGTGTGTGCCCATTTCCTTGACAGC
GGAGTCCAGGTGAGGCGTGGTAGACAAGATGCCTGGTTTCAAGAGCTGAT
TTGTGCTACCCGTGAGTCCCTCTCCGGGGTGTGTGAAATCAGTGGCCGCC
CAGCAGCTGTTTTTGAGCACCTTGTCCTTTGTGTGTCCGTGGTGTGCAAC
GACTACGTCTTTTACTGCAAAGACCAGCGCCGTGGGGGCCTGCGCTACAT
CTCGCATGCAGTCATcTGGAGGGACTGAAGCACTGTTTGCCTTTCTGTAC
GGCTTATTCTGCCTACGTGCCCCAAGATGGAACGATGTTAACTGCGCTGC
GCCAGTGCATCTAACAGCCCTGTGCAGCAGCTTCCCTTGCCTCGTGTAAC
CTGCCCCCTCCCCACCCTGCCACTCTTGACATTCCACTGTGCGTTTTAGA
GTGGTACCGCCAAAATTCAAACCCCAGACTCCCGAACTCATGCTCAGAAC
GCCCAGTGACCTGCCGAGGTCGGCAGCACAGAGCTCTGGAGATGAAGACC
TGGAGGCCGTGGCTATGGTTGGAGGTCAGCTTCAGGCCTTCTGGAAGCAT
GATGATCCTACTGCTCCCAGCAACCTCTACATCCAGGAATGAGTCCCTAG
CCCATGTGGGTGTGGCAGTTACAGGGCCCAGGTGAGCTGAAGACAAACCA
CTCAGGGAACTCGGTTTTTGGAGACATGAGTCTGCTGATGCTCCTGACTG
CATGATAGACACGATGAAATCCACCCTCAAAGAGCAGTTCCAGTTTGTGG
TGTGGCCGCTTCTGTGTCCCACCAGTCCTGCCCCCAAAACTGACCATGAA
GCGTCAGAGTCGCTGAGCTTGTTGGCCTGATCTTGCGTTTGGAAAGAAAT
AGCTGGCCCCACACAACCAAGGTCCAAGGGAGGACCATCATCTTCTCCAG
CTGGGCACAGAGAATCAGCTAGGAGACCAGTTATTCAGGGTCCATTTCTC
ACGTGCTCCCTCTGCCAGGAGGAGAATGAAGACGTGGTGCGAGATGCGCT
TTGTGGAAAGTCCTTTTTTACTGCTTTGCCCATTGGAGGTGTCTCCTTTT
GACACACACAAACACAGAACCACACAGCCAGTCCCAGGAGCCCAGTAATG
GCCACGAATAAGGCCATCACCAGAAGCCAACCCCGCCAGTCCTTGATCTA
CACAGTGGGGAGCATGGAGGGATGGGTTTGGCCTGTGCTTCTGCTTATTC
TGCCAGCCTTGCAGAAAAGGCTCCCATTGTGTTACCCCATCACTCAACCT
TTGCCTCCCTCAGATAGAAAACAGCCCCCACTCCAGTCCACTCCTGACCC
AAAGTGACCTTAGTGATGCAGAGTTCCTGAGTGGTGTTTGTAGAATGAGT
AGGAGACCACCGCCTTCTCCAGTGCTTCCTTGGGCAGCCAGTAATTCCCA
GCCAATGTTAATTTATAGCCAGGTGTGCGTGTGTCTCCCGCCTCGCCGCC
AATGGAGCAGGATATTGCTGAAGTCTCCTGGCATATGTTACCGAATCAAA
TGCTGAAGTCTCCTGGCATATGTTACCGAATCAAATAGCCTTCCAGAGGC
TGAGAATACTTGTCCCTGGAGGATTATCACACCCCAAATGCATAATCTCG
CTTCTACCCAGAAGGATGGACAGCTAATAGCGTACTTGGGGATGAGGAGC
GGAACGCGGCGGAGCTGTGAGCCGGCGACTCGGGTCCCTGAGGTCTGGAT
GGATGAGAGGGAACCACTATAACATGAGTCCAAGCCCAGAAGACTTCTGT
GCCTCACTTAAGTCCAGGAAGCTGGGGTGGCGAGGAAGGATGATGTGGAT
CACAGTGCCAGCCTCTACCTAAGGAAACCCTAGACCTTGGAACCAGTTTC
GAGGTCTCTCCAGCTGTTTCAAAATGTCATGTAACTGGTGACACTCAGCC
CTGGGAGTTCCCTGAACATCTGTGTGTGTCCCCCTATGCTCCAGTATGGA
ACAGGGTCACAGTAATGGGGGGTTTCAAAGTGGAGAACCACACGGCGTGC
GTCACCCCTGGTGCCGTGAAGCTGGAGAAGGAGAAGCTGGAGCAAAACCC
CCCCAGCCAGGTCTCATAGAGGGAATTGTTCTTCAGGCTCGGAGGGGCCT
CTGGGCTGACGCCCCCTTGCCTCTGCCTGGTACCCACATGACTTGGAACT
CTCTATCGGAAGCTTTACTCCGTCGAGAGTCTGTAGGAGCAGCAGTCCTC
TGCGCGACATGGAAGGTGATCTGCAAGAGCTGCATCAGTCAAACACCGGG
GAGTGTGAGAGTGATAATGCAGGGGTGAGTGTGAGAGGGACTGCGTTTGC
GCCAGTTGACAAGATTTTTCCACCCTCGAGCAGCGTGAGAGATGCCTCTT
GCGCCTTTCTCATCAGCTTCTTCCGAGGGTGACAGGTGAAAGACCCCTAC
GAGTATCAGAATCACCTGGAAGGGCTTTTACAGATTGCTGGCCCCACCCC
CTGTGGGTCACCAAGCTCTGGAAGTTCATTCGCGATTTCCTGCGGTGGTG
TGTCCTCGCTATGAGATTTCCCACCACCCACGCTGGTGTTCACATCTAGC
ACAGAGGATACTACAGGCTGGGAAAGCTGCAGCAAAGGGAGACTAGAGAG
GGAAGGCGCCTGTCCATATGGATAAGATAGGGTTGTAAACGTCCTCATCT
TCCGAAAAAGATCTGCCGGCCCAGAGCACTCCCTCTTGCCCTAATCCTGG
CTCCCAAAGACTCAGCTCCCCTGTTAGATGGCTCTGCCTGTCCTTCCCCA
GCCGAAGCCATGAATGCCCTCGAACTAGGCCCTTGGATGAAGTGAACCAG
TGTTCTGCAGGCTTGCAGAGTTCTTCTCACCATTTAAACTGAAGGACCCT
AGTAATGGAGAGCCCCAAAAAGAAGAACCAGCAGCTGAAAGTCGGGATCC
TTGGTGGAAAAACCACCTGATGAGGCTGCACCCTTGGTGGAGGGAACAGC
AGCCATTGCCCAGTGGTCCATATCTCCACCACATCCCCTGCTTGAGCCCA
ACAGAGCTTCGGAGCCCATGCGAATGCGAAAGCCTCGTGGAGTTCCAGGG
GGCAAGCGATCAAGCAGCGACTATGCACAACGAGAGGATTTTGAGGCTGC
GCAGTGCCCGAAACCCACACTGCAGATCAGCCACATCGCCCAGCAGCTTG
ACATCCCCTGGAAGTACAAGGACTCATCTGTGGTCCCTGCTTCTCCTCCC
CAGGTTGGAGTGCGGCTAGTGCCCCAAGGCGGCTTGGAGACCTCTCAGCC
ACTCACACAGCTCCTCCACAGGAGACTTCTGGAGCAAGCAGGACCAGCCT
GTCAGCAGCTCTATCTCACCAATGACAGCCAGAATAGCAAGCAACCACTG
GAGAACATCACCACCCTGAAGCCAGAGACTAACACTGCAGGACTCAGCAA
CTCCACCCCCCGCCTGTGGGATGCCTTGTGGGACGTCTCTTTCTATTCAA
GCCCAGCTGTAGCTCTGGGCCTGGAACTATGAAGACCTAGTGCTCCCAGA
AAAGAAGAACCAGCAGCTGAAAGTCGGGATCCTACACCTGGGCAGCAGAC
GCGCAACCGGTTCTCCGAAACATGGAGTCCTGTAGGCAAGGTCTTACCTG
CGCCGTGGCTCTCAGCTGTCAATGTGCACTCTGCCGCCGCAGCACCACTG
CAAGGCAGGAAGAGAATCCATCCACCTCTCTGGACAGTAATGGTGCAGCA
TGTTCAAGTCCTAGTCTATAGGATTGGCAGTTTAAATGCTTTACTCCCCC
GAAGATTAGAACCAGACTTACTAACCAATTCCACCCCCCACCAACCCCCT
GGAGATGACCTAGAATGCAGAGAAACAGCCTCCTCTCCCAAAAGCCAACG
GGCCACCTATTTTGAAACGCACACCTTTGCCATGAAGTCTGTTGTTGCAT
AAAAATGCACTGTGAGTTTCATGCCTGCTGGCCTGCCTTCACTGTCCTGG
GAAATGTATTGTTTGAGCTCAAAAGGCCCGACCACCCCCCTTCGGGCTGC
CCCAGGGGAAGACCCAAAGGCAGCAAAAACAAGAGTCCCTCTAAAGCAGC
GCACTAAGAGAAGGAGACTCTCAAACCAAAAATGACCTGGAGGCACCATG
ACTAACGAGGACGCCGTCCAGGGCATCGCTAACGAGGACGCCGTCCACAG
CTCCTCATAGGTGAGATCAAGAGGCCACCAGTTGTACTTCAGCACCAATG
GGAGCCAGAGCCAGTCAGGGGTTAAAGTGAAAGCCCGTATTTCCGCCCAG
CCACCCCGTCCGATTTGGGTCTGATGATGAGGGCAGATACCTAACTCAGG
CCAGGACTCCAGAGCTGTGACCTGGCTCTGGTTCAACAAAAGGGGCCTGA
CTTTAATTCTTGGGCCTCCAATAAGTGTCCCATAGGTGTCTGGCCAGGCC
GGAACGGCTGCTGTTTCAGCTGTTGGCCTTTTCAATTCTATACTCCATTT
GCAAGCCCTCGCCTTCGCCGTGTGGGAATTTTCCTGGATGCTGACTTAGA
GGAAGGGCCTGGAGTGGATTGGGTACATCTATTACAGTGGGAGCACCTAC
CTTTTGAGTCCTCGGGCCCAGAATCGTATCCCAAAGCCCTCCCATGGCCT
TAAGGACCCAGTTTCTTCTGTGAGCACGGGCAGTGGGAGGCCGGTGGAAA
CGACAGAGACCAACAGGAGCCCCAAACATGTATTCCTCTTCACTATTGGC
CGTCTCCCGGGGCCCTAGGACACCCCGATCCTCCCACAATAAAGGCTTCT
GACAAGGAAGAGCTTTGCCATCCCCTGCATGTGCCCCTGCCTCTACCTGT
GACCTGATGTCCATTCATCCCACCCTCTCACAGTTCGGACTTTTCTCCCC
CTCCTGGTGGGACTTGTATCTTGTCTGCCATATCAGAACACAAACCCCTG
GGAGAGTGAAGTCTACAGCCTAGAGGGTGCCCTGTGTGTGGTGTCAGATG
CCCAAGGCGTTTGGCTCAGAGGGCTACAGACTATGGCCAGAACTCATCTG
GAGGGGACACACTCTGCACCAACCTCACTGGGACACTTTTGCCTTCCCGA
GCAGGGGACAGTTTTTCCAGGGTGGCCTATCATTGGGGTATGAGTGGCTG
GACCGTTTTGTGGCTAGTGCGATTTCACAGTCTACTGCCTGTTTCCACTG
CTGGGTCCGCCAGCCCCCAGGGAAGGGGCTGGAGTGGATTGGGGAAATCT
TACCAGACTCCGACACAATGGGCCACCACCAGTGTGACAGCAGGACATCC
CCTCCCCAAAAGCAGCATATTAGGCCCAGGACATACCTGAAGGCTGGTCG
CTTTCTGCAAGACCTTTGGCTCACAGAACTGCAGGGTATGGTGAGAAACC
GCATAGCGACTTGCCTTTCCGTCTTGTTCAGTCGTCACCAGTCAGCCGTC
TCTTGGCAGGGGGAGAGGATGGCCCAGCAGGCCTGGCCCAGCTCCCAGTT
CAAATGAGCACGCTGCCATGTACTGGTTTGAGCAGTAGGGGCTGCATCGC
TCAGCCAAAGGGGCTGACTGTCATTGGAGCGGTGGGCACTGTTAAGAAGC
CCTGTGAATACCTCAGCTTCAACTGGGCCTCCATACAGTCAGTTGGTGGG
ACCACAGTGCAGGGAAAACAAAAGTATCCCAGCATCTTCATCCTGTACAC
ATCGGGGTTGGCGTTGGCCGCAGATGCCTCTCGGTCCCTCCCTGTACTTA
CCTGGAGTTAGTCGACCGTTGCGAGACGTTGAGCTGCGGCAGATGAGTCC
ACTGCCTGCTACTTGAGGGCCAGGATGCTCCCCCAGGGAAGAAACGGAAT
GGTCTCCTGATGGAAGTGTACAATACCACCTACAAATTATCCATGCCCCA
GTTAGGAATGAAAGCATCCCTGGAGAACAGCCTGGAGGAGACCAAAGGCC
GGTGACAAAACTGAGTCCTGGGGAAAGTGACTGGTCCGTGGTAGAGCCGG
CCCTGCAGAGGATGCTCGTTTTGCAGAGAAGGCAGTGTTCCTCTATTCCC
GGGTACTGGCTGTGGATCATTTAGCTGCAGTCCTCTTTCCTACAACCTTG
CCAGGCTCCCTCCGCAGACTGACTTTCCTCTGTGTCTGGGTGTTACAGTC
CATAGACCCTGATGCCCTCAAAGAGCTCCCCCTCCTAAAGTTCCTTGGCA
GAGCTGGGATTCATCCTAAAAGGCTTCTCCCCAGATGCCAGAGACCTGTC
CCAAATGGCATACTTACAAGACGGATGCAACCTGGGTCCTTAGGTCGCTG
ATCCTTCCTGTTTACTCCTGCCTTGTGGCGGGGTCCATCCTCTGCTGCTC
ATGTCTTCGGTGCTGGCGGCTTCCCATCCGCTGGTTCTATCCTCAAACGC
GTAGGAGGCAGGTCTCCGCGGTTCATCTGTGTTGCTCTAAATGACACTGT
AGGCCTGCCTTTAATTTTCAGTGTAAGTGTTCAGTATGCCGCATCCTGCC
CATGATGAGAACCGCCTGGAAGCTTTAAGCACATGACCTGGGGACCAGGC
TGATCAGGCTGCCAATGAATGGGGCAGGAGTGGCAAAGACCCCAATCACT
GTGACGGCTGGTGACTGATGGATGGGTAGTGGGCTGAGAAGAGGGGACTA
TCTTTTGGCCTTTCCCCAATGCTTGAAAGAAGATCACCACTGCTGGAGGC
GAGAGTCTCTGTGAGACTGTTTCACAGAAGGATGTGTGTTTACCCAAGGC
TGTTGGAGGATGAAAGTACGGAGTGATCCATCGGCTAAGTGTCTTGTCAC
GCTGGTGAAACCCCGAAGATCAACACGCTTCAAACTCAGCCCCTTGGAAC
CCGTTTCTTAAATGTTACCAGTCCCAGCCAATCTTACGGTGACATTACAG
TCCCTCAACATCAGAACAATGCTCAAGTCTTTCAAGCCACGTCTGAGCAG
TTCTGGACCTCAGTCCTTCACCTAGTCACCTAGTCACAGGGTGGATCGCC
AGGAAGCCCGCCAGTGGTGAGACTGTGTCCTCAAGGAGCTTCGGCACCAT
CTCCTGATCCCTTTCCCATCGGATCTGAACACTGGTCTTGGTGGTCGTAA
TAATTGTGTGAAGTGTGTCTGTCTCCAGCCCTTCGGGCCTCCCACGAGCC
AGAGTGCCTAAGAGAAACCCTTGCACCTGGGAGCGCTGCTTGGCTCTATC
CCAGGGTTAGCCAGACAGGCACCAAAGCCAAGGAAGCAGAGATCCAGCCT
CGCTGACATCAAAACTGTTCGGACTTCCCCGTTAGAACTCGCGTTTCCAC
GGTATATACAGGGAGGCCAGGCAGCCTGGAGTTAGTCGACCGTTGCGAGA
ACTTTAGGAGACGGAGGCGGGAGGGTCACTTGAGCCCAGGAATTGTGAGA
TTAAGCCTAAGTCTCTTCAGCCACCTCCCAGGAAGATCTGGCCTCCGTCC
GAGGGCCACGAGGCGTCGGCTTTAGACACAGATCATAGCTCTACAGGAGT
TGCTGGGTGTGTTGCATGGCCCTCCCAACCAATTCAGTATTTTTCTCCCC
GTCTGATCTCGGAAGCTAAGCAGGGTCGGGCCTGGTTAGTACTTGGACGG
GAGCAGCTCCCTCGCTGCGATCTATTGAAAGTCAGATCTCCACACAAGGG
GAAGGCCACTTGAGCTTCCTGGAGAAGGACCTGAGGGACAAGGTCAACTC
TGGATGCCCAGGGGAAGGCATACAGGCCTCATCCACCAGGCAATAGACAG
TCCTCGGCTTAGTGGCTGAAGACTGATGCTGCCCGATCGCCTCAGAAGCC
CGACGCTTCTTCTCAGCCTCCAGTCTCCAGTTCCAAGGATGGGTCATCTC
AAGGCCCCAGAGAGCTAACTCACCCTTCCACCATATGAGGACGTGGCAAG
GCGCTGGGGGCTGGACCTGCTGACATAGTGGATGGACAGATGGACCAAAA
ACTGGCACTGGACTTCAAGCGAAAGGCACGCAGCGGGAGCAGCAGCTATA
GAGGGAGAGAGGGAGAGAGCCTATACCCCTTACTTAGCATGCACAAAGTG
CAGGGCAATAGGAACACAGGGTGGAACCGCCTTTGTCAAGAGCACATTCC
GTGGACCCCGTGGTCTCCTTCCCTGTGGCTCTCAGCTGTCGCTGTGGACC
AGTGTGCAGTGAGGAAAGGGGCAAGGATCGGGAAGCTGTGTGACTGTCCC
GGAATAAGATACTTGTTGCTGTCACAGTTATTACCATCCCCCCAGCTACC
CTGTGTCGGAGTCAGCGCAGCTTTTGAAGCTGGAGAGCATCATATTTTAG
GCTGTGCACCCTAGAAACAACATATTGTCCCATGAGCAGGTGCCTGAGAC
TCTCAGCTTAACATGGAAATGAGGATCCCACCAGCCCCAGAACCCTCTGG
TTGAGCCCTTGCCCCTGCGTCCCGCGTCTGGGTTCTCAGCTATTTCCAGA
GAGATGAGTGCGGGGCTCATCTATCCCTGGAATTGTCTTTCCCACAATCC
CCTGCCCCTGCTCTGCACTCTCAGGTATTCCCTGCTCTTACTCCAAAAAG
AGGAAGGACACACAGTAGCTCTCTGCTTGCTGATAGATGGTTTCCCAGTG
ACTTTGACCAGAGTTGGAGCCACCCAGGGGAATGATCTCTGATGACCTAA
GCTGCCTTGCTTGAGTTGCTTGTGACTGATCTTTTGAGGCTGTCATCATG
CAGGAAGCCCACTGTACCTGGAGCCATCTGGGATAAGACTTTGACCCATG
GTTACAGCCATATGCAGGACAGCAGTACTCAGCATGGTCTTATGCACAGG
GGGCATGAGTCCTTGACAATAGTAAATAGCACCTCTGTTCCCTTATTGGG
TTATGACTGGGGAAGCCCAGTTCAGAATAGGATGCACTATGATCAGTCCC
CCAGTCTCTCTTCTGTAGGCATTGTGGTGAGCGATCCTGACTCCACAGAT
CTGACAAGTAGAATCAAAGGTGCAGCTGACTGAGACGACATGCATGTAAG
TACTCACACGGACAGGTTGATGCCAGAGCCGTAAGAATGCGCCAGTGCGG
AACTGAGAGAAAGAGCAACAAAGCGGCGAGTGGTGTGAGAGGGCAGCACG
GGTATGTCTGGAGGGTGAAGAGAGGTTGGTTAGTGGGTAAAAATACACAG
ACCGCCGCCGGGCACCTGCCACCAAGCAACTGTTTCATTTTTTATTTTCC
ACATGTCCTGGGTGACATGGGATGTGACTTTCGGGTGTCGGGGCAGCATG
AGGGTCGAATCTGGAATGGGAGGGTCTGGCTTCAGCTATCAGGGCACCCT
TCCTCACAGGACAGAAGCAGAGTGGGTGGTGGTTATGTTTGACAGAAGGC
TGGAGTGGGTTGGCTTGCTGATGGCTGCTGGAGGGGACGCTGGCTAAAGT
AAGGACAGAGGTGTGGGAGACCCAAGGTGGTCTTGAGATTGACAGACAGC
TCGTCCATGCCAAAGTGTGCGGCCCTTCCTGACATCACCACAGTCTGAGC
TGGCCTTCACTGACTCTGCTAGGTGGTGCCCTGATTGTTGGCATGGTGAG

In some embodiments, the probes are specific to a cancer associated sequence. In some embodiments, the probes are specific to the sequence described in the accession number and its associated gene symbol (which can be found in the tables described herein) as described in the following table:

| GenBank | |
|---|---|
| Accession No. | Probe Sequence |
| NM_006681.1 | GCTGCAGCTCGTTCCTCACCTGCATGAGAGAAGAATGAAGAGATTCAGAG |
| NM_206955.1 | TGGGGACAGAACGTTCTATGACCCGGAGCCCATCCTGTGCCCCTGTTTCA |
| NM_206955.1 | GACCCACGTGCTGTATCCTGTCCCCCTGGAGAGTTATGAGGACATCCATG |
| NM_014471.1 | GCGGCACTGATGGGCTCACATATACGAATGAATGCCAGCTCTGCTTGGCC |
| NM_000439.3 | GTAGAGGGTGTTTGCAGAGCAATGCCCGTAATGCTTAGAGAATGTTCTCC |
| NM_000439.3 | GTAGCTGAGTTTAACATGTGTGGTCTTGGTATTCTTAAGGGAACTTCCAC |
| NM_003381.2 | CTTCGGCATGGCCTCTTTACAGGGCACCTTCTGCTCTCAGGTTGGGTGAC |
| NM_001105519.1 | CCTGATGCCTGAGATCCTGGGTCGTGACCAGCCTGGCTTGCTTGGAATAA |
| NM_020436.2 | GCGGTCAGCTAAGGGAGAACTTGCGTGGAAGGAGCAATGCAGACACAGTG |
| NM_001008783.1 | ACTGAAACCCAGCCAGAAGAGGGACCACCTGTAAAGCAAGTCCTTTCAAG |
| NM_021246.2 | TGCAGCAGCTACCGCCCTGACCTGTCTCTTGCCAGGACTGTGGAGCGGAT |
| XM_001133677.1 | TCACGTGTCTTCACGCATCTCTTGAATTGGAAATTGTGCCCTGGAGACTG |
| NM_001555.2 | CCCTGCAAGTCAGCCCCATCTGCTGTTCCTTGGTCTCTAATCACCTGAGC |
| NM_005940.3 | CAGGTCTTGGTAGGTGCCTGCATCTGTCTGCCTTCTGGCTGACAATCCTG |
| NR_006882.1 | GTAGAGCACCGAAAACCCCGAGGAAGAGAGGTAGCGTTTTCTCCTGAGCG |
| NM_000479.2 | CTCATCAGCCTGTCGGAGGAACGCATCAGCGCGCACCACGTGCCCAACAT |
| NM_013404.3 | TTCCACCCCAAGAGAACTCGCGCTCAGTAAACGGGAACATGCCCCCTGCA |
| B0536065 | TTCCAGGGCACGAGTTCGAGGCCAGCCTGGTCCACATGGGTCGGaaaaaa |
| NR_002739.1 | TTCGTCAACAGCAGTTCACCTAGTGAGTGTTGAGACTCTGGGTCTGAGTG |
| NM_144668.4 | TCCCGAGGGATGGAAATCCGAGCCTGCAACCTGCTCCGTCAAAGGTTCAG |
| NR_006881.1 | GAAGCCGGCTTTCTGGCGTTGCTTGGCTGCAACTGCCGTCAGCCATTGAT |
| NR_006880.1 | CTGCAACTGCCGTCAGCCATTGATGATCGTTCTTCTCTCCGTATTGGGGA |
| NM_144594.1 | GGGGCACAACTCACTACTCTGACAACAACAGCCCTGCGAGCAACATAGTT |
| NM_016212.2 | TCACGTGTCTTCACGCATCCCTTGAATTGGAAATTGTGCCCTGGAGACTG |
| NM_002594.2 | GGCCAGTGGAAATTCAGGTGAAAATGTTCATCAATTCCCATTGCATCACC |
| NM_203428.1 | TCCCACTTGGCAGGGGCCGTCTTGTCCACTCGTTTCTGTAAACATGGGTG |
| NM_006183.3 | CCACAAAATCTGTCACAGCAGGGCTTTTCAACACTGGGAGTTAATCCAGG |
| NM_001001888.1 | GGTGGAGGAACCACTGAGTCAGGAGAGCGAGATGGAAGAACCACTGAGTC |
| NM_203429.1 | GAAGGCTGGCTCATACATTTTCCCAGACAGGAATTTGGCTGCCAACAGGG |
| NM_021115.3 | GATGTCGCTACTATTCCAACCTCCGCCTGCCTCTGATGTACTCCCACCCC |
| NM_004535.2 | CAGATGTGTGTGCTTGGGCGTGTTTCTCGTGTCGCGTTTGCGTGTCGGCT |
| NM_152224.1 | TGGGTTGGACCTAGTGGTGTTGTCGTGAGTGCCACCTAACCAGGAGGCCA |
| NM_000295.3 | AGTGGACTTAGCCCCTGTTTGCTCCTCCGATAACTGGGGTGACCTTGGTT |
| NM_014420.2 | GACACTGCTCAAGCTCCAGAAATCTTCCAGCGTTGCGACTGTGGCCCTGG |
| NM_013452.2 | GAACCACTGAGTCAGGAGAGCCAGGTGGAGGAACCACCGAGTCAGGAGAG |
| NM_052959.2 | GGCTGGCTTAGAACCCTCAAAACCCAAACACCTCACCAACTCGGCATGTG |
| NM_144967.2 | GTACAGTTTTGCTCAGGTCACGCCAACAGGGAAACCTCAAGTGTAGGTCT |
| NM_016379.2 | GCCAGGTGGAGGAACCACTGAGTCAGGAGAGCGAGATGGAAGAACTACCG |
| NM_001001552.3 | CGAGAGCTGGAGAGAAGAAGGTTTCCCAGTGGGCTTGAAGCTTGCTGTGC |
| XR_041261.1 | TGCAGCCATGTAAGCAAGTACCTTTCACCTCCCGCCATGATTCTGAGGCC |
| NM_024923.2 | TGGGGGAGGAGACCCTTGGAAAAGTCCTCTCTTCCCAGCTCCTGATTCTG |
| NM_001926.2 | AGAGCTTTGGGCTCAACAAGGGCTTTCACTTGCCATTGCAGAAGGTCCTG |
| NM_002299.2 | AGTAGCTCTTGCGGAAACGTGTAGATACTGGTCTAGTGGGTCTGTGAACC |
| NM_182980.2 | TGCTAAGGAAGGAGACCAGGAAGCCACCCTAACACTCGGCCAGACCCGCT |
| NR_002581.1 | GACCATGCATGTGTCCCCAAACCTAGTTCTTTCCCTAGGTCTGGTTTCAT |
| NM_004950.3 | CCACATCCCTCTGCCACTCCCAGAAAATCTACGAGCCCTTCACCTCCAGA |
| NM_016249.2 | TAGTGGAACAAAATTGAAGGGTGGTCAGTAGTTTCATTTCCTTGTCCTGC |
| NM_207339.2 | GCAGTGCCTGCTTTTCAAGGGCCTGACATGGAAGCTTTTCAACAGGAACT |
| NM_001015038.1 | CTTTCCGCCATCTTGATTCTTTGTCACTGACCGAGACTCAGCCGTGGGAA |
| NM_130467.3 | GGGACTCTGCCCACTTTTGATCCCACTAAAGTGCTGGAAGGAGGTGAAGG |
| NM_004988.3 | GCGGTCAGTGTTCTCAGTAGTAGGTTTCTGTTCTATTGGGTGACTTGGAG |
| NM_173798.2 | CCCTGCAGCCTACGGGTCTGTTTTCTGTGTGTGCCCATTTCCTTGACAGC |
| NM_001080466.1 | GGAGTCCAGGTGAGGCGTGGTAGACAAGATGCCTGGTTTCAAGAGCTGAT |
| NM_021010.1 | TTGTGCTACCCGTGAGTCCCTCTCCGGGGTGTGTGAAATCAGTGGCCGCC |
| XM_941629.1 | CAGGAGCTGTTTTTGAGCACCTTGTCCTTTGTGTGTCCGTGGTGTGCAAC |
| NM_014582.2 | GACTACGTCTTTTACTGCAAAGACCAGCGCCGTGGGGGCCTGCGCTACAT |
| NM_001004317.2 | CTCGCATGCAGTCATCTGGAGGGACTGAAGCACTGTTTGCCTTTCTGTAC |
| NM_001001933.1 | GGCTTATTCTGCCTACGTGCCCCAAGATGGAACGATGTTAACTGCGCTGC |
| NM_002362.4 | GCCAGTGCATCTAACAGCCCTGTGCAGCAGCTTCCCTTGCCTCGTGTAAC |
| NM_002196.2 | CTGCCCCCTCCCCACCCTGCCACTCTTGACATTCCACTGTGCGTTTTAGA |
| NM_175901.3 | GTGGTACCGCCAAAATTCAAACCCCAGACTCCCGAACTCATGCTCAGAAC |
| NM_014581.2 | GCCCAGTGACCTGCCGAGGTCGGCAGCACAGAGCTCTGGAGATGAAGACC |
| NM_001042600.1 | TGGAGGCCGTGGCTATGGTTGGAGGTCAGCTTCAGGCCTTCTGGAAGCAT |
| NM_001042600.1 | GATGATCCTACTGCTCCCAGCAACCTCTACATCCAGGAATGAGTCCCTAG |
| NM_002240.2 | CCCATGTGGGTGTGGCAGTTACAGGGCCCAGGTGAGCTGAAGACAAACCA |
| NM_199048.1 | CTCAGGGAACTCGGTTTTTGGAGACATGAGTCTGCTGATGCTCCTGACTG |
| NM_014509.3 | CATGATAGACACGATGAAATCCACCCTCAAAGAGCAGTTCCAGTTTGTGG |
| NM_080614.1 | TGTGGCCGCTTCTGTGTCCCACCAGTCCTGCCCCCAAAACTGACCATGAA |
| NM_203400.1 | GCGTCAGAGTCGCTGAGCTTGTTGGCCTGATCTTGCGTTTGGAAAGAAAT |
| NM_001001663.1 | AGCTGGCCCCACACAACCAAGGTCCAAGGGAGGACCATCATCTTCTCCAG |
| NM_020826.1 | CTGGGCACAGAGAATCAGCTAGGAGACCAGTTATTCAGGGTCCATTTCTC |
| NM_018044.2 | ACGTGCTCCCTCTGCCAGGAGGAGAATGAAGACGTGGTGCGAGATGCGCT |
| NM_018936.2 | TTGTGGAAAGTCCTTTTTTACTGCTTTGCCCATTGGAGGTGTCTCCTTTT |
| XM_927237.1 | GACACACACAAACACAGAACCACACAGCCAGTCCCAGGAGCCCAGTAATG |
| NM_203311.1 | GCCACGAATAAGGCCATCACCAGAAGCCAACCCCGCCAGTCCTTGATCTA |
| NM_004918.2 | CACAGTGGGGAGCATGGAGGGATGGGTTTGGCCTGTGCTTCTGCTTATTC |
| NM_001099676.1 | TGCCAGCCTTGCAGAAAAGGCTCCCATTGTGTTACCCCATCACTCAACCT |
| NM_005310.2 | TTGCCTCCCTCAGATAGAAAACAGCCCCCACTCCAGTCCACTCCTGACCC |
| NM_021951.2 | AAAGTGACCTTAGTGATGCAGAGTTCCTGAGTGGTGTTTGTAGAATGAGT |
| NM_153479.1 | AGGAGACCACCGCCTTCTCCAGTGCTTCCTTGGGCAGCCAGTAATTCCCA |
| NM_005634.2 | GCCAATGTTAATTTATAGCCAGGTGTGCGTGTGTCTCCCGCCTCGCCGCC |
| NM_001017436.1 | AATGGAGCAGGATATTGCTGAAGTCTCCTGGCATATGTTACCGAATCAAA |
| NM_001017436.1 | TGCTGAAGTCTCCTGGCATATGTTACCGAATCAAATAGCCTTCCAGAGGC |
| NM_001017436.1 | TGAGAATACTTGTCCCTGGAGGATTATCACACCCCAAATGCATAATCTCG |
| NM_019079.2 | CTTCTACCCAGAAGGATGGACAGCTAATAGCGTACTTGGGGATGAGGAGC |
| NM_133431.1 | GGAACGCGGCGGAGCTGTGAGCCGGCGACTCGGGTCCCTGAGGICTGGAT |
| NM_014258.2 | GGATGAGAGGGAACCACTATAACATGAGTCCAAGCCCAGAAGACTTCTGT |
| NM_001017361.2 | GCCTCACTTAAGTCCAGGAAGCTGGGGTGGCGAGGAAGGATGATGTGGAT |
| XR_017850.1 | CACAGTGCCAGCCTCTACCTAAGGAAACCCTAGACCTTGGAACCAGTTTC |
| NM_014592.2 | GAGGTCTCTCCAGCTGTTTCAAAATGTCATGTAACTGGTGACACTCAGCC |
| NM_002846.2 | CTGGGAGTTCCCTGAACATCTGTGTGTGTCCCCCTATGCTCCAGTATGGA |
| NM_000735.2 | ACAGGGTCACAGTAATGGGGGGTTTCAAAGTGGAGAACCACACGGCGTGC |
| NM_002701.4 | GTCACCCCTGGTGCCGTGAAGCTGGAGAAGGAGAAGCTGGAGCAAAACCC |
| NM_001079530.1 | CCCCAGCCAGGTCTCATAGAGGGAATTGTTCTTCAGGCTCGGAGGGGCCT |
| NM_004884.3 | CTGGGCTGACGCCCCCTTGCCTCTGCCTGGTACCCACATGACTTGGAACT |
| NM_199286.2 | CTCTATCGGAAGCTTTACTCCGTCGAGAGTCTGTAGGAGCAGCAGTCCTC |
| NM_001097595.1 | TGCGCGACATGGAAGGTGATCTGCAAGAGCTGCATCAGTCAAACACCGGG |
| NM_002379.2 | GAGTGTGAGAGTGATAATGCAGGGGTGAGTGTGAGAGGGACTGCGTTTGC |
| NM_080618.2 | GCCAGTTGACAAGATTTTTCCACCCTCGAGCAGCGTGAGAGATGCCTCTT |
| NM_031950.2 | GCGCCTTTCTCATCAGCTTCTTCCGAGGGTGACAGGTGAAAGACCCCTAC |
| AK024399 | GAGTATCAGAATCACCTGGAAGGGCTTTTACAGATTGCTGGCCCCACCCC |
| NM_004861.1 | CTGTGGGTCACCAAGCTCTGGAAGTTCATTCGCGATTTCCTGCGGTGGTG |
| CA849062 | TGTCCTCGCTATGAGATTTCCCACCACCCACGCTGGTGTTCACATCTAGC |
| NM_001010874.3 | ACAGAGGATACTACAGGCTGGGAAAGCTGCAGCAAAGGGAGACTAGAGAG |
| NM_198152.2 | GGAAGGCGCCTGTCCATATGGATAAGATAGGGTTGTAAACGTCCTCATCT |
| NM_173509.2 | TCCGAAAAAGATCTGCCGGCCCAGAGCACTCCCTCTTGCCCTAATCCTGG |
| NM_006998.3 | CTCCCAAAGACTCAGCTCCCCTGTTAGATGGCTCTGCCTGTCCTTCCCCA |
| NM_001025290.1 | GCCGAAGCCATGAATGCCCTCGAACTAGGCCCTTGGATGAAGTGAACCAG |
| NM_032132.3 | TGTTCTGCAGGCTTGCAGAGTTCTTCTCACCATTTAAACTGAAGGACCCT |
| NM_001097597.1 | AGTAATGGAGAGCCCCAAAAAGAAGAACCAGGAGCTGAAAGTCGGGATCC |
| XM_936973.2 | TTGGTGGAAAAACCACCTGATGAGGCTGCACCCTTGGTGGAGGGAACAGC |
| NM_001034838.1 | AGCCATTGCCCAGTGGTCCATATCTCCACCACATCCCCTGCTTGAGCCCA |
| NM_030592.1 | ACAGAGCTTCGGAGCCCATGCGAATGCGAAAGCCTCGTGGAGTTCCAGGG |
| NR_002304.1 | GGCAAGCGATCAAGCAGCGACTATGCACAACGAGAGGATTTTGAGGCTGC |
| NR_002304.1 | GCAGTGCCCGAAACCCACACTGCAGATCAGCCACATCGCCCAGCAGCTTG |
| NM_173092.1 | ACATCCCCTGGAAGTACAAGGACTCATCTGTGGTCCCTGCTTCTCCTCCC |
| XR_017655.1 | CAGGTTGGAGTGCGGCTAGTGCCCCAAGGCGGCTTGGAGACCTCTCAGCC |
| NM_148674.3 | ACTCACACAGCTCCTCCACAGGAGACTTCTGGAGCAAGCAGGACCAGCCT |
| NM_178550.3 | GTCAGCAGCTCTATCTCACCAATGACAGCCAGAATAGCAAGCAACCACTG |
| XM_945048.1 | GAGAACATCACCACCCTGAAGCCAGAGACTAACACTGCAGGACTCAGCAA |
| NM_203347.1 | CTCCACCCCCCGCCTGTGGGATGCCTTGTGGGACGTCTCTTTCTATTCAA |
| NM_170694.1 | GCCCAGCTGTAGCTCTGGGCCTGGAACTATGAAGACCTAGTGCTCCCAGA |
| NM_001097593.1 | AAAGAAGAACCAGCAGCTGAAAGTCGGGATCCTACACCTGGGCAGCAGAC |
| NM_177524.1 | GCGCAACCGGTTCTCCGAAACATGGAGTCCTGTAGGCAAGGTCTTACCTG |
| NM_033043.1 | CGCCGTGGCTCTCAGCTGTCAATGTGCACTCTGCCGCCGCAGCACCACTG |
| NM_002851.2 | CAAGGCAGGAAGAGAATCCATCCACCTCTCTGGACAGTAATGGTGCAGCA |
| NM_006418.3 | TGTTCAAGTCCTAGTCTATAGGATTGGCAGTTTAAATGCTTTACTCCCCC |
| NM_006418.3 | GAAGATTAGAACCAGACTTACTAACCAATTCCACCCCCCACCAACCCCCT |
| NM_001017417.1 | GGAGATGACCTAGAATGCAGAGAAACAGCCTCCTCTCCCAAAAGCCAACG |
| BX537518 | GGCCACCTATTTTGAAACGCACACCTTTGCCATGAAGTCTGTTGTTGCAT |
| NM_001080848.1 | AAAAATGCACTGTGAGTTTCATGCCTGCTGGCCTGCCTTCACTGTCCTGG |
| NM_003655.2 | GAAATGTATTGTTTGAGCTCAAAAGGCCCGACCACCCCCCTTCGGGCTGC |
| NM_003483.4 | CCCAGGGGAAGACCCAAAGGCAGCAAAAACAAGAGTCCCTCTAAAGCAGC |
| NM 032545.2 | GCACTAAGAGAAGGAGACTCTCAAACCAAAAATGACCTGGAGGCACCATG |
| XM_001719794.1 | ACTAACGAGGACGCCGTCCAGGGCATCGCTAACGAGGACGCCGTCCACAG |
| NM_004316.2 | CTCCTCATAGGTGAGATCAAGAGGCCACCAGTTGTACTTCAGCACCAATG |
| NM_001017361.1 | GGAGCCAGAGCCAGTCAGGGGTTAAAGTGAAAGCCCGTATTTCCGCCCAG |
| NM_198965.1 | CCACCCCGTCCGATTTGGGTCTGATGATGAGGGCAGATACCTAACTCAGG |
| NM_002277.2 | CCAGGACTCCAGAGCTGTGACCTGGCTCTGGTTCAACAAAAGGGGCCTGA |
| NM_024070.3 | CTTTAATTCTTGGGCCTCCAATAAGTGTCCCATAGGTGTCTGGCCAGGCC |
| NM_175056.1 | GGAACGGCTGCTGTTTCAGCTGTTGGCCTTTTCAATTCTATACTCCATTT |
| NM_001013734.2 | GCAAGCCCTCGCCTTCGCCGTGTGGGAATTTTCCTGGATGCTGACTTAGA |
| XM_001724554.1 | GGAAGGGCCTGGAGTGGATTGGGTACATCTATTACAGTGGGAGCACCTAC |
| NM_020209.2 | CTTTTGAGTCCTCGGGCCCAGAATCGTATCCCAAAGCCCTCCCATGGCCT |
| NR_024418.1 | TAAGGACCCAGTTTCTTCTGTGAGCACGGGCAGTGGGAGGCCGGTGGAAA |
| NM_030672.2 | CGACAGAGACCAACAGGAGCCCCAAACATGTATTCCTCTTCACTATTGGC |
| NM_033377.1 | CGTCTCCCGGGGCCCTAGGACACCCCGATCCTCCCACAATAAAGGCTTCT |
| NM_012284.1 | GACAAGGAAGAGCTTTGCCATCCCCTGCATGTGCCCCTGCCTCTACCTGT |
| NM_001844.3 | GACCTGATGTCCATTCATCCCACCCTCTCACAGTTCGGACTTTTCTCCCC |
| NM_005752.2 | CTCCTGGTGGGACTTGTATCTTGTCTGCCATATCAGAACACAAACCCCTG |
| NM_182562.2 | GGAGAGTGAAGTCTACAGCCTAGAGGGTGCCCTGTGTGTGGTGTCAGATG |
| NM_002407.1 | CCCAAGGCGTTTGGCTCAGAGGGCTACAGACTATGGCCAGAACTCATCTG |
| NM_001252.3 | GAGGGGACACACTCTGCACCAACCTCACTGGGACACTTTTGCCTTCCCGA |
| NM_030812.1 | GCAGGGGACAGTTTTTCCAGGGTGGCCTATCATTGGGGTATGAGTGGCTG |
| NM_006237.3 | GACCGTTTTGTGGCTAGTGCGATTTCACAGTCTACTGCCTGTTTCCACTG |
| XM_001716834.1 | CTGGGTCCGCCAGCCCCCAGGGAAGGGGCTGGAGTGGATTGGGGAAATCT |
| NM_006152.2 | TACCAGACTCCGACACAATGGGCCACCACCAGTGTGACAGCAGGACATCC |
| NM_152439.2 | CCTCCCCAAAAGCAGCATATTAGGCCCAGGACATACCTGAAGGCTGGTCG |
| NM_002411.2 | CTTTCTGCAAGACCTTTGGCTCACAGAACTGCAGGGTATGGTGAGAAACC |
| XM_498560.3 | GCATAGCGACTTGCCTTTCCGTCTTGTTCAGTCGTCACCAGTCAGCCGTC |
| NM_018674.3 | TCTTGGCAGGGGGAGAGGATGGCCCAGCAGGCCTGGCCCAGCTCCCAGTT |
| NM_015011.1 | CAAATGAGCACGCTGCCATGTACTGGTTTGAGCAGTAGGGGCTGCATCGC |
| NM_001819.1 | TCAGCCAAAGGGGCTGACTGTCATTGGAGCGGTGGGCACTGTTAAGAAGC |
| NM_004852.2 | CCTGTGAATACCTCAGCTTCAACTGGGCCTCCATACAGTCAGTTGGTGGG |
| NM_004852.2 | ACCACAGTGCAGGGAAAACAAAAGTATCCCAGCATCTTCATCCTGTACAC |
| XM_928495.1 | ATCGGGGTTGGCGTTGGCCGCAGATGCCTCTCGGTCCCTCCCTGTACTTA |
| NM_004679.2 | CCTGGAGTTAGTCGACCGTTGCGAGACGTTGAGCTGCGGCAGATGAGTCC |
| NM_080681.2 | ACTGCCTGCTACTTGAGGGCCAGGATGCTCCCCCAGGGAAGAAACGGAAT |
| NM_031282.1 | GGTCTCCTGATGGAAGTGTACAATACCACCTACAAATTATCCATGCCCCA |
| XR_037048.1 | GTTAGGAATGAAAGCATCCCTGGAGAACAGCCTGGAGGAGACCAAAGGCC |
| NM_001532.2 | GGTGACAAAACTGAGTCCTGGGGAAAGTGACTGGTCCGTGGTAGAGCCGG |
| NM_080429.2 | CCCTGCAGAGGATGCTCGTTTTGCAGAGAAGGCAGTGTTCCTCTATTCCC |
| NM_001010905.1 | GGGTACTGGCTGTGGATCATTTAGCTGCAGTCCTCTTTCCTACAACCTTG |
| NM_153046.1 | CCAGGCTCCCTCCGCAGACTGACTTTCCTCTGTGTCTGGGTGTTACAGTC |
| NM_000369.2 | CATAGACCCTGATGCCCTCAAAGAGCTCCCCCTCCTAAAGTTCCTTGGCA |
| NM_002854.2 | GAGCTGGGATTCATCCTAAAAGGCTTCTCCCCAGATGCCAGAGACCTGTC |
| NM_173698.1 | CCAAATGGCATACTTACAAGACGGATGCAACCTGGGTCCTTAGGTCGCTG |
| XM_939163.2 | ATCCTTCCTGTTTACTCCTGCCTTGTGGCGGGGTCCATCCTCTGCTGCTC |
| NR_004390.1 | ATGTCTTCGGTGCTGGCGGCTTCCCATCCGCTGGTTCTATCCTCAAACGC |
| XM_930694.1 | GTAGGAGGCAGGTCTCCGCGGTTCATCTGTGTTGCTCTAAATGACACTGT |
| NM_020708.3 | AGGCCTGCCTTTAATTTTCAGTGTAAGTGTTCAGTATGCCGCATCCTGCC |
| NM_144647.3 | CATGATGAGAACCGCCTGGAAGCTTTAAGCACATGACCTGGGGACCAGGC |
| NM_199161.1 | TGATCAGGCTGCCAATGAATGGGGCAGGAGTGGCAAAGACCCCAATCACT |
| NM_145754.2 | GTGACGGCTGGTGACTGATGGATGGGTAGTGGGCTGAGAAGAGGGGACTA |
| NM_001010925.2 | TCTTTTGGCCTTTCCCCAATGCTTGAAAGAAGATCACCACTGCTGGAGGC |
| XM_001131823.1 | GAGAGTCTCTGTGAGACTGTTTCACAGAAGGATGTGTGTTTACCCAAGGC |
| NR_003059.1 | TGTTGGAGGATGAAAGTACGGAGTGATCCATCGGCTAAGTGTCTTGTCAC |
| NM_004833.1 | GCTGGTGAAACCCCGAAGATCAACACGCTTCAAACTCAGCCCCTTGGAAC |
| NM_019106.4 | CCGTTTCTTAAATGTTACCAGTCCCAGCCAATCTTACGGTGACATTACAG |
| NM_001042496.1 | TCCCTCAACATCAGAACAATGCTCAAGTCTTTCAAGCCACGTCTGAGCAG |
| XR_038222.1 | TTCTGGACCTCAGTCCTTCACCTAGTCACCTAGTCACAGGGTGGATCGCC |
| NM_001010985.1 | AGGAAGCCCGCCAGTGGTGAGACTGTGTCCTCAAGGAGCTTCGGCACCAT |
| NR_002987.1 | CTCCTGATCCCTTTCCCATCGGATCTGAACACTGGTCTTGGTGGTCGTAA |
| NM_003378.2 | TAATTGTGTGAAGTGTGTCTGTCTCCAGCCCTTCGGGCCTCCCACGAGCC |
| NM_052900.2 | AGAGTGCCTAAGAGAAACCCTTGCACCTGGGAGCGCTGCTTGGCTCTATC |
| NM_053283.2 | CCAGGGTTAGCCAGACAGGCACCAAAGCCAAGGAAGCAGAGATCCAGCCT |
| NM_172004.2 | CGCTGACATCAAAACTGTTCGGACTTCCCCGTTAGAACTCGCGTTTCCAC |
| NM_016378.2 | GGTATATACAGGGAGGCCAGGCAGCCTGGAGTTAGTCGACCGTTGCGAGA |
| XR_037336.1 | ACTTTAGGAGACGGAGGCGGGAGGGTCACTTGAGCCCAGGAATTGTGAGA |
| NM_014224.1 | TTAAGCCTAAGTCTCTTCAGCCACCTCCCAGGAAGATCTGGCCTCCGTCC |
| NM_207033.1 | GAGGGCCACGAGGCGTCGGCTTTAGACACAGATCATAGCTCTACAGGAGT |
| NM_138768.2 | TGCTGGGTGTGTTGCATGGCCCTCCCAACCAATTCAGTATTTTTCTCCCC |
| NR_023371.1 | GTCTGATCTCGGAAGCTAAGCAGGGTCGGGCCTGGTTAGTACTTGGACGG |
| XM_001713808.1 | GAGGAGCTCCCTCGCTGCGATCTATTGAAAGTCAGATCTCCACACAAGGG |
| NM_000482.3 | GAAGGCCACTTGAGCTTCCTGGAGAAGGACCTGAGGGACAAGGTCAACTC |
| NM_006658.2 | TGGATGCCCAGGGGAAGGCATACAGGCCTCATCCACCAGGCAATAGACAG |
| NR_015379.2 | TCCTCGGCTTAGTGGCTGAAGACTGATGCTGCCCGATCGCCTCAGAAGCC |
| NM_024877.1 | CGACGCTTCTTCTCAGCCTCCAGTCTCCAGTTCCAAGGATGGGTCATCTC |
| NM_032738.3 | AAGGCCCCAGAGAGCTAACTCACCCTTCCACCATATGAGGACGTGGCAAG |
| NM_000316.2 | GCGCTGGGGGCTGGACCTGCTGACATAGTGGATGGACAGATGGACCAAAA |
| NM_000316.2 | ACTGGCACTGGACTTCAAGCGAAAGGCACGCAGCGGGAGCAGCAGCTATA |
| NM_000756.1 | GAGGGAGAGAGGGAGAGAGCCTATACCCCTTACTTAGCATGCACAAAGTG |
| NM_004306.2 | CAGGGCAATAGGAACACAGGGTGGAACCGCCTTTGTCAAGAGCACATTCC |
| NM_000894.2 | GTGGACCCCGTGGTCTCCTTCCCTGTGGCTCTCAGCTGTCGCTGTGGACC |
| NM_004291.2 | AGTGTGCAGTGAGGAAAGGGGCAAGGATCGGGAAGCTGTGTGACTGTCCC |
| NM_001093770.1 | GGAATAAGATACTTGTTGCTGTCACAGTTATTACCATCCCCCCAGCTACC |
| XM_935867.1 | CTGTGTCGGAGTCAGCGCAGCTTTTGAAGCTGGAGAGCATCATATTTTAG |
| NM_000125.2 | GCTGTGCACCCTAGAAACAACATATTGTCCCATGAGGAGGTGCCTGAGAC |
| NM_001974.3 | TCTCAGCTTAACATGGAAATGAGGATCCCACCAGCCCCAGAACCCTCTGG |
| NM_005247.2 | TTGAGCCCTTGCCCCTGCGTCCCGCGTCTGGGTTCTCAGCTATTTCCAGA |
| XM_944750.2 | GAGATGAGTGCGGGGCTCATCTATCCCTGGAATTGTCTTTCCCACAATCC |
| XM_927939.2 | CCTGCCCCTGCTCTGCACTCTCAGGTATTCCCTGCTCTTACTCCAAAAAG |
| NM_018656.2 | AGGAAGGACACACAGTAGCTCTCTGCTTGCTGATAGATGGTTTCCCAGTG |
| NM_000583.2 | ACTTTGACCAGAGTTGGAGCCACCCAGGGGAATGATCTCTGATGACCTAA |
| NM_004617.2 | GCTGCCTTGCTTGAGTTGCTTGTGACTGATCTTTTGAGGCTGTCATCATG |
| NM_006365.1 | CAGGAAGCCCACTGTACCTGGAGCCATCTGGGATAAGACTTTGACCCATG |
| NM_022573.2 | GTTACAGCCATATGCAGGACAGCAGTACTCAGCATGGTCTTATGCACAGG |
| NM_203395.1 | GGGCATGAGTCCTTGACAATAGTAAATAGCACCTCTGTTCCCTTATTGGG |
| NM_004190.1 | TTATGACTGGGGAAGCCCAGTTCAGAATAGGATGCACTATGATCAGTCCC |
| NM_002252.3 | CCAGTCTCTCTTCTGTAGGCATTGTGGTGAGCGATCCTGACTCCACAGAT |
| NM_002252.3 | CTGACAAGTAGAATCAAAGGTGCAGCTGACTGAGACGACATGCATGTAAG |
| NM_004518.2 | TACTCACACGGACAGGTTGATGCCAGAGCCGTAAGAATGCGCCAGTGCGG |
| NM_005832.3 | AACTGAGAGAAAGAGCAACAAAGCGGCGAGTGGTGTGAGAGGGCAGCACG |
| NR_002728.2 | GGTATGTCTGGAGGGTGAAGAGAGGTTGGTTAGTGGGTAAAAATACACAG |
| NM_172109.1 | ACCGCCGCCGGGCACCTGCCACCAAGCAACTGTTTCATTTTTTATTTTCC |
| NM_031460.3 | ACATGTCCTGGGTGACATGGGATGTGACTTTCGGGTGTCGGGGCAGCATG |
| NM_022358.2 | AGGGTCGAATCTGGAATGGGAGGGTCTGGCTTCAGCTATCAGGGCACCCT |
| NM_001622.1 | TCCTCACAGGACAGAAGCAGAGTGGGTGGTGGTTATGTTTGACAGAAGGC |
| NM_002250.2 | TGGAGTGGGTTGGCTTGCTGATGGCTGCTGGAGGGGACGCTGGCTAAAGT |
| NM_032115.2 | AAGGACAGAGGTGTGGGAGACCCAAGGTGGTCTTGAGATTGACAGACAGC |
| NM_178863.2 | TCGTCCATGCCAAAGTGTGCGGCCCTTCCTGACATCACCACAGTCTGAGC |
| NM_171829.1 | TGGCCTTCACTGACTCTGCTAGGTGGTGCCCTGATTGTTGGCATGGTGAG |

As described herein the sequences can be homologs of the sequences described herein. In some embodiments, the sequences are at least 80% homologous. In some embodiments, the length of the sequence is at least 10, 20, 30, 40, 50, 60, 70, 80, or 90% of the sequences described herein or incorporated by reference herein.

In some embodiments, the cancer associated sequences may be DNA sequences encoding the above mRNA or the cancer associated protein or cancer associated polypeptide expressed by the above mRNA. In some embodiments, the cancer associated sequence may be a mutant nucleic acid of the above disclosed sequences. In some embodiments, the cancer associated protein or polypeptide sequence may be selected from sequences described herein or in the accession numbers described herein or a homolog thereof. In some embodiments, the homolog may have at least about 60%, at least about 65%, at least about 70%, at least about 75%, at least about 80%, at least about 85%, at least about 90%, at least about 95%, at least about 97%, at least about 98%, at least about 99%, at least about 99.5% identity with the disclosed polypeptide sequence. In some embodiments, the cancer associated DNA sequences may be selected from the sequences described herein.

In some embodiments, antigen presenting cells (APCs) may used to activate T lymphocytes in vivo or ex vivo, to elicit an immune response against cells expressing a cancer associated sequence. APCs are highly specialized cells and may include, without limitation, macrophages, monocytes, and dendritic cells (DCs). APCs may process antigens and display their peptide fragments on the cell surface together with molecules required for lymphocyte activation. In some embodiments, the APCs may be dendritic cells. DCs may be classified into subgroups, including, e.g., follicular dendritic cells, Langerhans dendritic cells, and epidermal dendritic cells.

Some embodiments are directed to the use of cancer associated polypeptides and polynucleotides encoding a cancer associated sequence, a fragment thereof, or a mutant thereof, and antigen presenting cells (such as, without limitation, dendritic cells), to elicit an immune response against cells expressing a cancer-associated polypeptide sequence, such as, without limitation, cancer cells, in a subject. In some embodiments, the method of eliciting an immune response against cells expressing a cancer associated sequence comprises (1) isolating a hematopoietic stem cell, (2) genetically modifying the cell to express a cancer associated sequence, (3) differentiating the cell into DCs; and (4) administering the DCs to the subject (e.g., human patient). In some embodiments, the method of eliciting an immune response includes (1) isolating DCs (or isolation and differentiation of DC precursor cells), (2) pulsing the cells with a cancer associated sequence, and; (3) administering the DCs to the subject. These approaches are discussed in greater detail, infra. In some embodiments, the pulsed or expressing DCs may be used to activate T lymphocytes ex vivo. These general techniques and variations thereof may be within the skill of those in the art (see, e.g., WO97/29182; WO 97/04802; WO 97/22349; WO 96/23060; WO 98/01538; Hsu et al., 1996, Nature Med. 2:52-58), and that still other variations may be discovered in the future. In some embodiments, the cancer associated sequence is contacted with a subject to stimulate an immune response. In some embodiments, the immune response is a therapeutic immune response. In some embodiments, the immune response is a prophylactic immune response. For example, the cancer associated sequence can be contacted with a subject under conditions effective to stimulate an immune response. The cancer associated sequence can be administered as, for example, a DNA molecule (e.g. DNA vaccine), RNA molecule, or polypeptide, or any combination thereof. Administering sequence to stimulate an immune responses are known, but the identity of which sequences to use was not known prior to the present disclosure. Any sequence or combination of sequences disclosed herein or a homolog thereof can be administered to a subject to stimulate an immune response.

In some embodiments, dendritic cell precursor cells are isolated for transduction with a cancer associated sequence, and induced to differentiate into dendritic cells. The genetically modified DCs express the cancer associated sequence, and may display peptide fragments on the cell surface.

In some embodiments, the cancer associated sequence comprises a sequence of a naturally occurring protein. In some embodiments, the cancer associate sequence does not comprise a naturally occurring sequence. As already noted, fragments of naturally occurring proteins may be used; in addition, the expressed polypeptide may comprise mutations such as deletions, insertions, or amino acid substitutions when compared to a naturally occurring polypeptide, so long as at least one peptide epitope can be processed by the DC and presented on a MHC class I or II surface molecule. In some embodiments, it may be desirable to use sequences other than “wild type,” in order to for example, increase antigenicity of the peptide or to increase peptide expression levels. In some embodiments, the introduced cancer associated sequences may encode variants such as polymorphic variants (e.g., a variant expressed by a particular human patient) or variants characteristic of a particular cancer (e.g., a cancer in a particular subject).

In some embodiments, the sequences described herein comprises a sequence that encodes a gene product that is referred to as NMU, PRAME, PRAME, SPINK4, PCSK1, PCSK1, VIP, C2orf70, SALL4, SLC35D3, LY6G6D, LOC729264, IGSF1, MMP11, SNORD3D, AMH, MSLN, SNORD56, WDR66, SNORD3C, SNORD3A, GTSF1, TP53TG3, PCSK2, DSCR8, NTS, VCX-C, DSCR8, SEZ6L, MYT1, PPEF1, SERPINA1, DKK4, VCX, PANX3, FLJ30058, VCX3A, LEMD1, LOC730081, NUP210, DEFA6, LCT, OSGIN1, SNORA72, EPYC, MAGEC2, PAGE2, PAGE2B, PAGE5, MAGEA1, ZCCHC12, BTBD17, DEFA5, LOC652235, OBP2A, LIN28B, LHX8, MAGEA4, INSM1, LOC283932, OBP2B, MAP4K1, MAP4K1, KCNJ6, T1560, SERHL2, WFDC3, RPRML, TMEM211, SYT13, NSUN5, PCDHB2, LOC653219, CSAG3A, TCL1B, C12orf56, GRB7, DMRT1, CSAG1, SOX3, CT45A4, CT45A4, CT45A4, L1TD1, XAGE1, SYCP2, C6orf221, PART1, KCNIP1, PTPRN, CGA, POU5F1, CFC1B, IGDCC3, DPPA3, XAGE1B, MATN1, CTCFL, FGFBP2, GAL3ST1, SRD5A2L2, UTS2D, FAM163A, SCGN, DPPA5, HORMAD1, XAGE1C, LOC338579, KCNIP1, MATN4, POU5F1P1, POU5F1P1, KCNH6, LOC645682, SMC1B, C1orf110, LOC651957, LCN15, SERHL, XAGE1A, MEST, CGB5, PTPRZ1, OLFM4, OLFM4, CT45A1, CSAG3B, CBX4, HMGA2, CFC1, LOC100133542, ASCL1, ECAT1, PTHLH, KRT31, PVRIG, ZPLD1, RFPL4B, LOC100134331, SHD, LOC389332, ARHGAP28, CGB1, KCNH3, COL2A1, CLEC3A, FAM169B, SCGB2A1, CD70, ACTL8, POU4F1, LOC642131, LRMP, BEST3, SCGB2A2, LOC440132, ACCN4, MYO16, CHGB, ONECUT2, ONECUT2, LOC645464, VCY, COL11A2, FCRL4, LOC651397, SLC29A2, AQP10, C6orf58, TDRD9, TSHR, PVALB, FAM133A, F1123152, SNORA57, LOC642477, SLC12A5, CAPSL, SAA1, KIFC2, ANKRD19, ANKRD30A, SNORD71, AIM2, 3-Sep, SLC12A6, LOC100133312, MYBPHL, SNORA61, VGF, CSMD3, DCD, CLECL1, VCX2, LOC100131139, PGA5, EDN3, MYEOV, RN5S9, LOC100132564, APOA4, C7orf16, UCA1, CNTD2, FCRLA, PTH1R, PTH1R, CRH, ANXA13, LHB, CARTPT, SFTPA1, LOC641738, ESR1, EMR1, FGF3, LOC646360, LOC644844, SLC35E3, GC, TM4SF4, C1orf61, TSPY2, IYD, LIPF, KCNS3, KCNS3, KCNQ2, KCNMB2, KCNQ1OT1, KCNQ2, KCNK17, KCNK15, AHSG, KCNN4, KCNK16, KCTD13, and/or KCNMB3, or homolog thereof, or fragment thereof, or variant thereof. The homolog can have a percent homology either at the nucleic acid level or the amino acid sequence level. The percent homology can be as described herein.

In some embodiments, a cancer associated expression sequence may be introduced (transduced) into DCs or stem cells in any of a variety of standard methods, including transfection, recombinant vaccinia viruses, adeno-associated viruses (AAVs), retroviruses, etc.

In some embodiments, the transformed DCs of the invention may be introduced into the subject (e.g., without limitation, a human patient) where the DCs may induce an immune response. Typically, the immune response includes a cytotoxic T-lymphocyte (CTL) response against target cells bearing antigenic peptides (e.g., in a MHC class I/peptide complex). These target cells are typically cancer cells.

In some embodiments, when the DCs of the invention are to be administered to a subject, they may preferably isolated from, or derived from precursor cells from, that subject (i.e., the DCs may administered to an autologous subject). However, the cells may be infused into HLA-matched allogeneic, or HLA-mismatched allogeneic subject. In the latter case, immunosuppressive drugs may be administered to the subject.

In some embodiments, the cells may be administered in any suitable manner. In some embodiments, the cell may be administered with a pharmaceutically acceptable carrier (e.g., saline). In some embodiments, the cells may be administered through intravenous, infra-articular, intramuscular, intradermal, intraperitoneal, or subcutaneous routes. Administration (i.e., immunization) may be repeated at time intervals. Infusions of DC may be combined with administration of cytokines that act to maintain DC number and activity (e.g., GM-CSF, IL-12).

In some embodiments, the dose administered to a subject may be a dose sufficient to induce an immune response as detected by assays which measure T cell proliferation, T lymphocyte cytotoxicity, and/or effect a beneficial therapeutic response in the patient over time, e.g., to inhibit growth of cancer cells or result in reduction in the number of cancer cells or the size of a tumor.

In some embodiments, DCs are obtained (either from a patient or by in vitro differentiation of precursor cells) and pulsed with antigenic peptides having a cancer associated sequence. The pulsing results in the presentation of peptides onto the surface MHC molecules of the cells. The peptide/MHC complexes displayed on the cell surface may be capable of inducing a MHC-restricted cytotoxic T-lymphocyte response against target cells expressing cancer associated polypeptides (e.g., without limitations, cancer cells).

In some embodiments, cancer associated sequences used for pulsing may have at least about 6 or 8 amino acids and fewer than about 30 amino acids or fewer than about 50 amino acid residues in length. In some embodiments, an immunogenic peptide sequence may have from about 8 to about 12 amino acids. In some embodiments, a mixture of human protein fragments may be used; alternatively a particular peptide of defined sequence may be used. The peptide antigens may be produced by de novo peptide synthesis, enzymatic digestion of purified or recombinant human peptides, by purification of the peptide sequence from a natural source (e.g., a subject or tumor cells from a subject), or expression of a recombinant polynucleotide encoding a human peptide fragment.

In some embodiments, the amount of peptide used for pulsing DC may depend on the nature, size and purity of the peptide or polypeptide. In some embodiments, an amount of from about 0.05 ug/ml to about 1 mg/ml, from about 0.05 ug/ml to about 500 ug/ml, from about 0.05 ug/ml to about 250 ug/ml, from about 0.5 ug/ml to about 1 mg/ml, from about 0.5 ug/ml to about 500 ug/ml, from about 0.5 ug/ml to about 250 ug/ml, or from about 1 ug/ml to about 100 ug/ml of peptide may be used. After adding the peptide antigen(s) to the cultured DC, the cells may then be allowed sufficient time to take up and process the antigen and express antigen peptides on the cell surface in association with either class I or class II MHC. In some embodiments, the time to take up and process the antigen may be about 18 to about 30 hours, about 20 to about 30 hours, or about 24 hours.

Numerous examples of systems and methods for predicting peptide binding motifs for different MHC Class I and II molecules have been described. Such prediction could be used for predicting peptide motifs that will bind to the desired MHC Class I or II molecules. Examples of such methods, systems, and databases that those of ordinary skill in the art might consult for such purpose include: Peptide Binding Motifs for MHC Class I and II Molecules; William E. Biddison, Roland Martin, Current Protocols in Immunology, Unit 1I (DOI: 10.1002/0471142735.ima01is36; Online Posting Date: May, 2001)

The Biddison Reference above, provides an overview of the use of peptide-binding motifs to predict interaction with a specific MHC class I or II allele, and gives examples for the use of MHC binding motifs to predict T-cell recognition.

The table below provides an exemplary result for a HLA peptide motif search at the NIH Center for Information Technology website, Bioinformatics and Molecular Analysis Section (http://www-bimas.citnih.gov/cgi-bin/molbio/ken_parker_comboform),

TABLE
User Parameters and Scoring Information

| | |
|---|---|
| method selected to limit number of results | explicit number |
| number of results requested | 20 |
| HLA molecule type selected | A_0201 |
| length selected for subsequences to be | 9 |
| scored | |
| echoing mode selected for input sequence | Y |
| echoing format | numbered lines |
| length of user's input peptide sequence | 369 |
| number of subsequence scores calculated | 361 |
| number of top-scoring subsequences repotted | 20 |
| back in scaling output table | |

Scoring Results

| | | | Score (Estimate of |
|---|---|---|---|
| | | | Half Time of Disas- |
| | | Subsequence | sociation of a |
| | Start | Residue | Molecule Containing |
| Rank | Position | Listing | This Subsequence) |
| 1 | 310 | SLLKFLAKV | 2249.173 |
| 2 | 183 | HLLVFGIDV | 1662.432 |
| 3 | 137 | KVTDLVQFL | 339.313 |
| 4 | 254 | GLYDGMEHL | 315.870 |
| 5 | 228 | ILILSIIFI | 224.357 |
| 6 | 296 | FLWGPRAHA | 189.678 |
| 7 | 245 | VIWEALHMM | 90.891 |
| 8 | 308 | KMSLLKFLA | 72.836 |
| 9 | 166 | KNYEDHFPL | 37.140 |
| 10 | 201 | FVLVTSLGL | 31.814 |
| 11 | 174 | LLFSEASEC | 31.249 |
| 12 | 213 | GMLSDVQSM | 30.534 |
| 13 | 226 | ILILILSII | 16.725 |
| 14 | 225 | GILILILSI | 12.208 |
| 15 | 251 | NMMGLYDGH | 9.750 |
| 16 | 88 | QIACSSPSV | 9.563 |
| 17 | 66 | LIPSTPEEV | 7.966 |
| 18 | 220 | SMPKTGILI | 7.535 |
| 19 | 233 | IIFIEGYCT | 6.445 |
| 20 | 247 | WEALNMMGL | 4.395 |

One skilled in the art of peptide-based vaccination may determine which peptides would work best in individuals based on their HLA alleles (e.g., due to “MHC restriction”). Different HLA alleles will bind particular peptide motifs (usually 2 or 3 highly conserved positions out of 8-10) with different energies which can be predicted theoretically or measured as dissociation rates. Thus, a skilled artisan may be able to tailor the peptides to a subject's HLA profile.

In some embodiments, implementation of an immunotherapy strategy for treating, reducing the symptoms of, or preventing cancer or neoplasms, (e.g., a vaccine) may be achieved using many different techniques available to the skilled artisan.

Immunotherapy, or the use of antibodies for therapeutic purposes has been used in recent years to treat cancer. Passive immunotherapy involves the use of monoclonal antibodies in cancer treatments. See for example, Cancer: Principles and Practice of Oncology, 6 th Edition (2001) Chapt. 20 pp. 495-508. Inherent therapeutic biological activity of these antibodies include direct inhibition of tumor cell growth or survival, and the ability to recruit the natural cell killing activity of the body's immune system. These agents may be administered alone or in conjunction with radiation or chemotherapeutic agents. Rituxan® and Herceptin®, approved for treatment of lymphoma and breast cancer, respectively, are two examples of such therapeutics. Alternatively, antibodies may be used to make antibody conjugates where the antibody is linked to a toxic agent and directs that agent to the tumor by specifically binding to the tumor. Mylotarg® is an example of an approved antibody conjugate used for the treatment of leukemia.

Some embodiments also provide for antigens (cancer-associated polypeptides) associated with a variety of cancers as targets for diagnostic and/or therapeutic antibodies. These antigens may also be useful for drug discovery (e.g., small molecules) and for further characterization of cellular regulation, growth, and differentiation.

Electroporation may be used to introduce the cancer associated nucleic acids described herein into mammalian cells (Neumann, E. et al. (1982) EMBO J. 1, 841-845), plant and bacterial cells, and may also be used to introduce proteins (Marrero, M. B. et al. (1995) J. Biol. Chem. 270, 15734-15738; Nolkrantz, K. et al. (2002) Anal. Chem. 74, 4300-4305; Rui, M. et al. (2002) Life Sci. 71, 1771-1778). Cells (such as the cells of this invention) suspended in a buffered solution of the purified protein of interest are placed in a pulsed electrical field. Briefly, high-voltage electric pulses result in the formation of small (nanometer-sized) pores in the cell membrane. Proteins enter the cell via these small pores or during the process of membrane reorganization as the pores close and the cell returns to its normal state. The efficiency of delivery may be dependent upon the strength of the applied electrical field, the length of the pulses, temperature and the composition of the buffered medium. Electroporation is successful with a variety of cell types, even some cell lines that are resistant to other delivery methods, although the overall efficiency is often quite low. Some cell lines may remain refractory even to electroporation unless partially activated.

Microinjection may be used to introduce femtoliter volumes of DNA directly into the nucleus of a cell (Capecchi, M. R. (1980) Cell 22, 470-488) where it can be integrated directly into the host cell genome, thus creating an established cell line bearing the sequence of interest. Proteins such as antibodies (Abarzua, P. et al. (1995) Cancer Res. 55, 3490-3494; Theiss, C. and Metier, K. (2002) Exp. Cell Res. 281, 197-204) and mutant proteins (Naryanan, A. et al. (2003) J. Cell Sci. 116, 177-186) can also be directly delivered into cells via microinjection to determine their effects on cellular processes firsthand. Microinjection has the advantage of introducing macromolecules directly into the cell, thereby bypassing exposure to potentially undesirable cellular compartments such as low-pH endosomes.

Several proteins and small peptides have the ability to transduce or travel through biological membranes independent of classical receptor-mediated or endocytosis-mediated pathways. Examples of these proteins include the HIV-1 TAT protein, the herpes simplex virus 1 (HSV-1) DNA-binding protein VP22, and the Drosophila Antennapedia (Antp) homeotic transcription factor. In some embodiments, protein transduction domains (PTDs) from these proteins may be fused to other macromolecules, peptides or proteins such as, without limitation, a cancer associated polypepdtide to successfully transport the polypeptide into a cell (Schwarze, S. R. et al. (2000) Trends Cell Biol. 10, 290-295). Exemplary advantages of using fusions of these transduction domains is that protein entry is rapid, concentration-dependent and appears to work with difficult cell types (Fenton, M. et al. (1998) J. Immunol. Methods 212, 41-48.).

In some embodiments, liposomes may be used as vehicles to deliver oligonucleotides, DNA (gene) constructs and small drug molecules into cells (Zabner, J. et al. (1995) J. Biol. Chem. 270, 18997-19007; Feigner, P. L. et al. (1987) Proc. Natl. Acad. Sci. USA 84, 7413-7417). Certain lipids, when placed in an aqueous solution and sonicated, form closed vesicles consisting of a circularized lipid bilayer surrounding an aqueous compartment. The vesicles or liposomes of embodiments herein may be formed in a solution containing the molecule to be delivered. In addition to encapsulating DNA in an aqueous solution, cationic liposomes may spontaneously and efficiently form complexes with DNA, with the positively charged head groups on the lipids interacting with the negatively charged backbone of the DNA. The exact composition and/or mixture of cationic lipids used can be altered, depending upon the macromolecule of interest and the cell type used (Feigner, J. H. et al. (1994) J. Biol. Chem. 269, 2550-2561). The cationic liposome strategy has also been applied successfully to protein delivery (Zelphati, O. et al. (2001) J. Biol. Chem. 276, 35103-35110). Because proteins are more heterogeneous than DNA, the physical characteristics of the protein, such as its charge and hydrophobicity, may influence the extent of its interaction with the cationic lipids.

In some embodiments, a method of screening drug candidates includes comparing the level of expression of the cancer-associated sequence in the absence of the drug candidate to the level of expression in the presence of the drug candidate.

Some embodiments are directed to a method of screening for a therapeutic agent capable of binding to a cancer-associated sequence (nucleic acid or protein), the method comprising combining the cancer-associated sequence and a candidate therapeutic agent, and determining the binding of the candidate agent to the cancer-associated sequence.

Further provided herein is a method for screening for a therapeutic agent capable of modulating the activity of a cancer-associated sequence. In some embodiments, the method comprises combining the cancer-associated sequence and a candidate therapeutic agent, and determining the effect of the candidate agent on the bioactivity of the cancer-associated sequence. An agent that modulates the bioactivity of the cancer associate sequence is said to be a therapeutic agent capable of modulating the activity of the cancer-associated sequence

In some embodiments, a method of evaluating the effect of a candidate cancer drug may comprise administering the drug to a patient and removing a cell sample from the patient. The expression profile of the cell is then determined. In some embodiments, the method may further comprise comparing the expression profile of the patient to an expression profile of a healthy individual. In some embodiments, the expression profile comprises measuring the expression of one or more or any combination thereof of the sequences disclosed herein. In some embodiments, where the expression profile of one or more or any combination thereof of the sequences disclosed herein is modified (increased or decreased) the candidate cancer drug is said to be effective.

Some embodiments are directed to a biochip comprising a nucleic acid segment which encodes a cancer associated protein, for example, but not limited to, selected from the sequences described herein or encoded by the sequences described in the accession numbers listed herein.

Also provided herein is a method for diagnosing or determining the propensity to cancers. The method of diagnosing may comprise measuring the level of expression of a cancer associated marker disclosed herein.

In some embodiments, an isolated nucleic acid comprises at least 10, 12, 15, 20 or 30 contiguous nucleotides of a sequence selected from the group consisting of the cancer associated polynucleotide sequences disclosed in sequences described herein or in the accession numbers described herein

In some embodiments, the polynucleotide, or its complement or a fragment thereof, further comprises a detectable label, is attached to a solid support, is prepared at least in part by chemical synthesis, is an antisense fragment, is single stranded, is double stranded or comprises a microarray.

In some embodiments, the invention provides an isolated polypeptide, encoded within an open reading frame of a cancer associated sequence selected from the polynucleotide sequences of sequences described herein or in the accession numbers described herein, or its complement. In some embodiments, the invention provides an isolated polypeptide, wherein said polypeptide comprises the amino acid sequence encoded by a polynucleotide selected from the group consisting of sequences described herein or in the accession numbers described herein. In some embodiments, the invention provides an isolated polypeptide, wherein said polypeptide comprises the amino acid sequence encoded by a polypeptide selected from the group consisting of sequences described herein or in the accession numbers described herein.

In some embodiments, the invention further provides an isolated polypeptide, comprising the amino acid sequence of an epitope of the amino acid sequence of a cancer associated polypeptide selected from the group consisting of sequences described herein or in the accession numbers described herein and shown in the tables, wherein the polypeptide or fragment thereof may be attached to a solid support. In some embodiments the invention provides an isolated antibody (monoclonal or polyclonal) or antigen binding fragment thereof, that binds to such a polypeptide. The isolated antibody or antigen binding fragment thereof may be attached to a solid support, or further comprises a detectable label.

In some embodiments, the invention provides a kit for diagnosing the presence of cancer in a test sample, said kit comprising at least one polynucleotide that selectively hybridizes to a cancer associated polynucleotide sequence shown in sequences described herein or in the accession numbers described herein, or its complement. In another embodiment the invention provides an electronic library comprising a cancer associated polynucleotide, a cancer associated polypeptide, or fragment thereof, shown sequences described herein or in the accession numbers described herein.

In some embodiments, the invention provides a method of screening for anticancer activity comprising: (a) providing a cell that expresses a cancer associated gene encoded by a nucleic acid sequence selected from the group consisting of the cancer associated sequences shown in sequences described herein or in the accession numbers described herein, or fragment thereof, (b) contacting the cell, which can be derived from a cancer cell with an anticancer drug candidate; (c) monitoring an effect of the anticancer drug candidate on an expression of the cancer associated sequence in the cell sample, and optionally (d) comparing the level of expression in the absence of said drug candidate to the level of expression in the presence of the drug candidate. The drug candidate may be an inhibitor of transcription, a G-protein coupled receptor antagonist, a growth factor antagonist, a serine-threonine kinase antagonist, a tyrosine kinase antagonist. In some embodiments, where the candidate modulates the expression of the cancer associated sequence the candidate is said to have anticancer activity. In some embodiments, the anticancer activity is determined by measuring cell growth. In some embodiments, the candidate inhibits or retards cell growth and is said to have anticancer activity. In some embodiments, the candidate causes the cell to die, and thus, the candidate is said to have anticancer activity.

In some embodiments, the invention provides a method for detecting a cancer associated sequence with the expression of a polypeptide in a test sample, comprising detecting a level of expression of at least one polypeptide selected from the group consisting of sequences described herein or in the accession numbers described herein, or a fragment thereof. In some embodiments, the method comprises comparing the level of expression of the polypeptide in the test sample with a level of expression of polypeptide in a normal sample, wherein an altered level of expression of the polypeptide in the test sample relative to the level of polypeptide expression in the normal sample is indicative of the presence of cancer in the test sample. In some embodiments, the polypeptide expression is compared to a cancer sample, wherein the level of expression is at least the same as the cancer is indicative of the presence of cancer in the test sample. In some embodiments, the sample is a cell sample.

Detecting a level of expression or similar steps that are described herein can be done experimentally or provided by a third-party as is described herein. Therefore, for example, “detecting a level of expression” can refer to experimentally measuring the data and/or having the data provided by another party who has processed a sample to determine and detect a level of expression data. In some embodiments, the expression data is detected experimentally and provided by a third party.

In some embodiments, the invention provides a method for detecting cancer by detecting the presence of an antibody in a test serum sample. In some embodiments, the antibody recognizes a polypeptide or an epitope thereof disclosed herein. In some embodiments, the antibody recognizes a polypeptide or epitope thereof encoded by a nucleic acid sequence disclosed herein. In some embodiments, the method comprises detecting a level of an antibody against an antigenic polypeptide selected from the group consisting of sequences described herein or in the accession numbers described herein, or antigenic fragment thereof. In some embodiments, the method comprises comparing the level of the antibody in the test sample with a level of the antibody in the control sample, wherein an altered level of antibody in said test sample relative to the level of antibody in the control sample is indicative of the presence of cancer in the test sample. In some embodiments, the control sample is a sample derived from a normal cell or non-cancerous sample. In some embodiments, the control is derived from a cancer sample, and, therefore, in some embodiments, the method comprises comparing the levels of binding and/or the amount of antibody in the sample, wherein when the levels or amount are the same as the cancer control sample is indicative of the presence of cancer in the test sample. As used herein, the term “level of antibody” refers to either the binding activity or affinity or the amount of the antibody in the sample. In some embodiments, the affinity of the antibody to its binding partner is increased and, therefore, the level of the antibody is said to altered. In some embodiments, the affinity of the antibody for its binding partner is decreased, and, therefore, the level of the antibody is said to altered. In some embodiments, the concentration of the antibody or the absolute amount of the antibody is increased or decreased in the sample as compared to a normal sample, and, either would be considered to be altered. In some embodiments, the binding affinity and the amount or concentration of the antibody are both different when compared to the normal sample. In some embodiments, an altered level of antibody refers to changes in one or the other, or both.

In some embodiments, the invention provides a method for screening for a therapeutic agent capable of modulating the activity of a cancer associated sequence, wherein said sequence can be encoded by a nucleic acid comprising a nucleic acid sequence selected from the group consisting of the polynucleotide sequences described herein or in the accession numbers described herein, said method comprising: a) combining said cancer associated sequence and a candidate therapeutic agent; and b) determining the effect of the candidate agent on the bioactivity of said cancer associated sequence. According to the method the therapeutic agent: affects the expression of the cancer associated sequence; and/or affects the activity of the cancer associated sequence, wherein such activity is selected from the activities of the protein. Examples of the activity include, but are not limited to, enzymatic (e.g. kinase, phosphatase, reductase, protease, transcriptase, polymerase) and the like. Binding activity can also be affected by the compounds. In some embodiments, the cancer associated sequence is a cancer associate protein (CAP). In some embodiments, the cancer associated sequence is a cancer associate nucleic acid molecule.

In some embodiments, a method for diagnosing cancer comprises a) determining the expression of one or more genes comprising a nucleic acid sequence selected from the group consisting of the human genomic and mRNA sequences described in sequences described herein or in the accession numbers described herein, in a first sample type. (e.g. tissue) of a first individual; and b) comparing said expression of said gene(s) from a second normal sample type from said first individual or a second unaffected individual; wherein a difference in said expression indicates that the first individual has cancer. In some embodiments, the expression is increased as compared to the normal sample. In some embodiments, the expression is decreased as compared to the normal sample.

In some embodiments, a method for treating cancer comprises administering to a subject in need thereof a therapeutic agent modulating the activity of a cancer associated protein (CAP), wherein said CAP is encoded by a nucleic acid comprising a nucleic acid sequence selected from the group consisting of the human nucleic acid sequences described herein or in the accession numbers described herein. In some embodiments, the therapeutic agent binds to the cancer associated protein; wherein the cancer associated protein is selected from the group consisting of the sequences described herein or in the accession numbers described herein.

In some embodiments, a method of treating cancer comprises administering an antibody (e.g. monoclonal antibody, human antibody, humanized antibody, chimeric antibody, and the like) that specifically binds to a cancer associated protein (CAP) that is expressed on a cell surface, wherein the cancer associated protein is selected from the group consisting of a protein encoded by a sequence described herein or in the accession numbers described herein. In some embodiments, the antibody binds to an extracellular domain of the cancer associated protein. In some embodiments, the antibody binds to a cancer associated protein differentially expressed on a cancer cell surface relative to a normal cell surface, or, in some embodiments, to at least one human cancer cell line. In some embodiments, the antibody is linked to a therapeutic agent. Kits and pharmaceutical compositions for detecting a presence or an absence of cancer cells in a subject, and comprising such antibodies are also provided.

In some embodiments, the invention also provides a method for detecting presence or absence of cancer cells in a subject. In some embodiments, the method comprises contacting one or more cells from the subject with an antibody as described herein. In some embodiments, the method comprises detecting a complex of a CAP and the antibody, wherein detection of the complex indicates with the presence of cancer cells in the subject. In some embodiments the invention provides a method for inhibiting growth of cancer cells in a subject. In some embodiments, the method comprises administering to the subject an effective amount of a pharmaceutical composition as described herein. In some embodiments the invention provides a method for delivering a therapeutic agent to cancer cells in a subject, the method comprising: administering to the subject an effective amount of a pharmaceutical composition according to according to the invention.

In some embodiments, the cancer cell can be targeted specifically with a therapeutic based upon the differentially expressed gene or gene product. For example, in some embodiments, the differentially expressed gene product is an enzyme, which can convert a anticancer prodrug into its active form. Therefore, in normal cells, where the differentially expressed gene product is not expressed or expressed at significantly lower levels, the prodrug is either not activated or activated in a lesser amount, and is, therefore less toxic to normal cells. Therefore, the cancer prodrug can, in some embodiments, be given in a higher dosage so that the cancer cells can metabolize the prodrug, which will, for example, kill the cancer cell, and the normal cells will not metabolize the prodrug or not as well, and, therefore, be less toxic to the patient. An example of this is where tumor cells overexpress a metalloprotease, which is described in Atkinson et al., British Journal of Pharmacology (2008) 153, 1344-1352, which is hereby incorporated by reference in its entirety and for the method of specifically targeting cancer cells. Using proteases to target cancer cells is also described in Carl et al., PNAS, Vol. 77, No. 4, pp. 2224-2228, April 1980, which is hereby incorporated by reference in its entirety and for the method of specifically targeting cancer cells. For example, doxorubicin or other type of chemotherapeutic can be linked to a peptide sequence that is specifically cleaved or recognized by the differentially expressed gene product. The doxorubicin or other type of chemotherapeutic is then cleaved from the peptide sequence and is activated such that it can kill or inhibit the growth of the cancer cell whereas in the normal cell the chemotherapeutic is never internalized into the cell or is not metabolized as efficiently, and is, therefore, less toxic. An example of this type of method is described in the Examples.

Additionally cells can be target based upon the proteins expressed on the surface of a cell. For example, the vascular endothelium and lymphatic endothelium, perivascular cells such as cell that express RGS5. Example of such cells include, but are not limited to, EP cell lines 4D20.9, CM02, E33, E111, E164, EN13, and U31. Other cells that can be targeted or used are mesenchymal stem cells, tumor stromal cells, tumor infiltrating lymphocytes; monocytes, and macrophages. As described herein a cell can be contacted with a prodrug composition (e.g. a linker that is cleaved by a protease made by the cancer cell where the cleaved linker sequence release the activated prodrug.) In some embodiments, the sequence has an inducible promoter such that the gene product is only expressed upon induction. The inducible promoter can be an x-ray inducible promoter or other chemically or otherwise inducible promoter.

Examples of linkers that can be cleaved to convert a prodrug into an active drug are described in, for example, but not limited to, JBC, Vol. 268, No. 3, Issue of January 25, pp. 1763-1769, 1993; Molecular Endocrinology, Vol. 6, 1441-1450, (1992). Examples of where cancers overexpress a protease or other enzyme that can be used to specifically target a cancer cell are described in, for example, but not limited to, Journal of Molecular Endocrinology (2001) 26, 95-105. Clin Cancer Res 2009; 15:274-283. An example of a gene product that can be used to specifically target cancer cells is described in, for example, but not limited to, JBC, Vol. 268, No. 8, Issue of March 15, pp. 5615-5623, 1993; Biochem. J. (1993) 292, 891-900. These references also describe various recognition sequences that can be used as linkers for prodrugs. These papers and other references show that specific enzymes that are differentially expressed in a cell can be used to target the cell with a prodrug so that when the prodrug is contacted with the cell that has increased expression (e.g. differentially expressed) the cell activates the prodrug whereas a normal cell does not. The sequences and gene products encoded by the same that are described herein can be adapted in a similar fashion based on the routine knowledge of one of skill in the art now that the present application has described the genes and gene products that are differentially expressed and can be used as targeting molecules for cancer treatments.

In some embodiments, the present invention provides methods of treating cancer. Embodiments are described herein. In some embodiments, the method comprise gene knockdown of one or more cancer associated sequences described herein. Gene knockdown refers to techniques by which the expression of one or more of an organism's genes is reduced, either through genetic modification (a change in the DNA of one of the organism's chromosomes such as, without limitation, chromosomes encoding cancer associated sequences) or by treatment with a reagent such as a short DNA or RNA oligonucleotide with a sequence complementary to either an mRNA transcript or a gene. In some embodiments, the oligonucleotide used may be selected from RNase-H competent antisense, such as, without limitation, ssDNA oligos, ssRNA oligos, phosphorothioate oligos, or chimeric oligos; RNase-independent antisense, such as morpholino oligos, 2′-O-methyl phosphorothioate oligos, locked nucleic acid oligos, or peptide nucleic acid oligos; RNAi Egos, such as, without limitation, siRNA duplex oligos, or shRNA oligos; or any combination thereof. In some embodiments, a plasmid may be introduced into a cell, wherein the plasmid expresses either an antisense RNA transcript or an shRNA transcript. The oligo introduced or transcript expressed may interact with the target mRNA (ex. SEQ ID NOs. 1-55) by complementary base pairing (a sense-antisense interaction).

The specific mechanism of gene knockdown may vary with the oligo chemistry. In some embodiments, the binding of a oligonucleotide described herein to the active gene or its transcripts may cause decreased expression through blocking of transcription, degradation of the mRNA transcript (e.g. by small interfering RNA (siRNA) or RNase-H dependent antisense) or blocking either mRNA translation, pre-mRNA splicing sites or nuclease cleavage sites used for maturation of other functional RNAs such as miRNA (e.g. by Morpholino oligos or other RNase-H independent antisense). For example, RNase-H competent antisense oligos (and antisense RNA transcripts) may form duplexes with RNA that are recognized by the enzyme RNase-H, which cleaves the RNA strand. As another example, RNase-independent oligos may bind to the mRNA and block the translation process application In some embodiments, the oligos may bind in the 5′-UTR and halt the initiation complex as it travels from the 5′-cap to the start codon, preventing ribosome assembly. A single strand of RNAi oligos may be loaded into the RISC complex, which catalytically cleaves complementary sequences and inhibits translation of some mRNAs bearing partially-complementary sequences. The oligos may be introduced into a cell by any technique including, without limitation, electroporation, microinjection, salt-shock methods such as, for example, CaCl2 shock; transfection of anionic oligo by cationic lipids such as, for example, Lipofectamine; transfection of uncharged oligos by endosomal release agents such as, for example, Endo-Porter; or any combination thereof. In some embodiments, the oligos may be delivered from the blood to the cytosol using techniques selected from nanoparticle complexes, virally-mediated transfection, oligos linked to octaguanidinium dendrimers (Morpholino oligos), or any combination thereof.

In some embodiments, a method of treating cancer may comprise treating cells to knockdown or inhibit expression of a gene encoding the mRNA disclosed herein. The method may comprise culturing hES cell-derived clonal embryonic progenitor cell lines CM02 and EN13 (see U.S. Patent Publication 20080070303, entitled “Methods to accelerate the isolation of novel cell strains from pluripotent stem cells and cells obtained thereby”; and U.S. patent application Ser. No. 12/504,630 filed on Jul. 16, 2009 and titled “Methods to Accelerate the Isolation of Novel Cell Strains from Pluripotent Stem Cells and Cells Obtained Thereby”, each of which is incorporated by reference herein in its entirety) with a retrovirus expressing silencing RNA directed to a cancer-associated sequence. In some embodiments, the method may further comprise confirming down-regulation by PCR. In some embodiments, the method further comprises cryopreserving the cells. In some embodiments, the method further comprises reprogramming the cells. In some embodiments, the method comprises cryopreserving or reprogramming the cells within two days by the exogenous administration of OCT4, MYC, KLF4, and SOX2 (see Takahashi and Yamanaka 2006 Aug. 25; 126(4):663-76; U.S. patent application Ser. No. 12/086,479, published as US2009/0068742 and entitled “Nuclear Reprogramming Factor”, each of which is incorporated herein by reference) and by the method described in PCT/US06/30632, published as WO/2007/019398 and entitled “Improved Methods of Reprogramming Animal Somatic Cells”, incorporated by reference herein in its entirety. In some embodiments, the method may comprise culturing mammalian differentiated cells under conditions that promote the propagation of ES cells. In some embodiments, any convenient ES cell propagation condition may be used, e.g., on feeders or in feeder free media capable of propagating ES cells. In some embodiments, the method comprises identifying cells from ES colonies in the culture. Cells from the identified ES colony may then be evaluated for ES markers, e.g., Oct4, TRA 1-60, TRA 1-81, SSEA4, etc., and those having ES cell phenotype may be expanded. Control lines that have not been preconditioned by the knockdown may be reprogrammed in parallel to demonstrate the effectiveness of the preconditioning. In some embodiments, one or more of the genes disclosed herein are knocked down. In some embodiments, one or more genes are selected from the group consisting NMU, PRAME, PRAME, SPINK4, PCSK1, PCSK1, VIP, C2orf70, SALL4, SLC35D3, LY6G6D, LOC729264, IGSF1, MMP11, SNORD3D, AMH, MSLN, SNORD56, WDR66, SNORD3C, SNORD3A, GTSF1, TP53TG3, PCSK2, DSCR8, NTS, VCX-C, DSCR8, SEZ6L, MYT1, PPEF1, SERPINA1, DKK4, VCX, PANX3, FLJ30058, VCX3A, LEMD1, LOC730081, NUP210, DEFA6, LCT, OSGIN1, SNORA72, EPYC, MAGEC2, PAGE2, PAGE2B, PAGE5, MAGEA1, ZCCHC12, BTBD17, DEFA5, LOC652235, OBP2A, LIN28B, LHX8, MAGEA4, INSM1, LOC283932, OBP2B, MAP4K1, MAP4K1, KCNJ6, T1560, SERHL2, WFDC3, RPRML, TMEM211, SYT13, NSUN5, PCDHB2, LOC653219, CSAG3A, TCL1B, C12orf56, GRB7, DMRT1, CSAG1, SOX3, CT45A4, CT45A4, CT45A4, L1TD1, XAGE1, SYCP2, C6orf221, PART1, KCNIP1, PTPRN, CGA, POU5F1, CFC1B, IGDCC3, DPPA3, XAGE1B, MATN1, CTCFL, FGFBP2, GAL3ST1, SRD5A2L2, UTS2D, FAM163A, SCGN, DPPA5, HORMAD1, XAGE1C, LOC338579, KCNIP1, MATN4, POU5F1P1, POU5F1P1, KCNH6, LOC645682, SMC1B, C1orf110, LOC651957, LCN15, SERHL, XAGE1A, MEST, CGB5, PTPRZ1, OLFM4, OLFM4, CT45A1, CSAG3B, CBX4, HMGA2, CFC1, LOC100133542, ASCL1, ECAT1, PTHLH, KRT31, PVRIG, ZPLD1, RFPL4B, LOC100134331, SHD, LOC389332, ARHGAP28, CGB1, KCNH3, COL2A1, CLEC3A, FAM169B, SCGB2A1, CD70, ACTL8, POU4F1, LOC642131, LRMP, BEST3, SCGB2A2, LOC440132, ACCN4, MYO16, CHGB, ONECUT2, ONECUT2, LOC645464, VCY, COL11A2, FCRL4, LOC651397, SLC29A2, AQP10, C6orf58, TDRD9, TSHR, PVALB, FAM133A, FLJ23152, SNORA57, LOC642477, SLC12A5, CAPSL, SAA1, KIFC2, ANKRD19, ANKRD30A, SNORD71, AIM2, 3-Sep, SLC12A6, LOC100133312, MYBPHL, SNORA61, VGF, CSMD3, DCD, CLECL1, VCX2, LOC100131139, PGA5, EDN3, MYEOV, RN5S9, LOC100132564, APOA4, C7orf16, UCA1, CNTD2, FCRLA, PTH1R, PTH1R, CRH, ANXA13, LHB, CARTPT, SFTPA1, LOC641738, ESR1, EMR1, FGF3, LOC646360, LOC644844, SLC35E3, GC, TM4SF4, C1orf61, TSPY2, IYD, LIPF, KCNS3, KCNS3, KCNQ2, KCNMB2, KCNQ10T1, KCNQ2, KCNK17, KCNK15, AHSG, KCNN4, KCNK16, KCTD13, and KCNMB3, or homolog thereof, or fragment thereof, or variant thereof.

Embodiments illustrating the method and materials used may be further understood by reference to the following non-limiting examples.

### Example 1

RNA was obtained from cultured diverse cultured human cell types, normal human tissues, and malignant tumors and analyzed on Illumina gene expression microarrays.

### Example 2

### SNAR-A1 Expression in Diverse Cancer Types

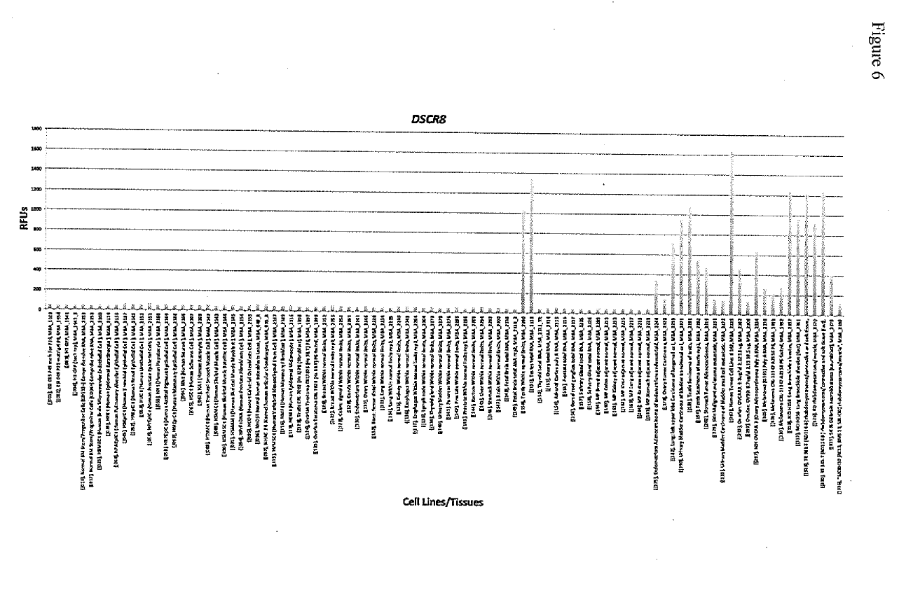

RNA was obtained from cultured diverse cultured human cell types, normal human tissues, and malignant human tumors and analyzed on Illumina gene expression microarrays. The gene encoding the RNA small nuclear ILF3/NF90-associated RNA A1 (SNAR-A1), A1 also known as IMAGE:6563923 5 (accession number BU536065) was detected as a gene expressed in relatively higher levels in hES cells, testis and diverse cancers compared to normal cultured somatic cell types and tissues. There are no reports that measurements of SNAR-A1 may be useful for screening or diagnosing a wide array of cancers. While Parrott et al, (2007) (Novel rapidly evolving hominid RNAs bind nuclear factor 90 and display tissue-restricted distribution, Nucleic Acids Res. 35:6249-6258) report that SNAR-A1 is expressed in relatively specifically in testis compared to other human tissues and report that it is expressed in a number of tumor cell lines, they do not report that the relative expression of SNAR-A1 is diagnostic of malignant tumor tissue compared to normal tissue. Parrott et al (2011) (The evolution and expression of the snaR family of small non-coding RNAs, Nucleic Acids Res. 39:1485-14500) report that the gene is overexpressed in transformed cell lines compared to their normal counterparts, but do not teach that the gene is diagnostic or prognostic for actual tumor samples. Therefore, surprisingly, as shown in FIG. 5, while diverse cultured normal somatic cell types such as brain microvascular endothelial cells, dermal fibroblasts, smooth muscle cells, esophageal epithelial cells, urothelial cells, pulmonary epithelial cells, prostate epithelial cells, hepatocytes, astrocytes, as well as others and normal tissues tested express relatively low levels of signal (i.e. either background signal of <100 RFU or low (<2,000 RFUs)), numerous samples of normal hES cells, testis, and diverse malignant tumors expressed the gene at relatively high levels (>2,000 RFU). Examples of such tumors are: adenocarcinoma of the uterus, ovarian carcinoma, small cell lung cancer, squamous cell lung cancer, neuroendocrine pancreatic cancer, seminoma of the testis, adenocarcinoma of the rectum, stomach adenocarcinoma, kidney cancer, cervical adenocarcinoma, skin melanoma, breast cancer, chondrosarcoma (which is expressed at relatively low levels but greater than that of normal chondrocytes), osteosarcoma, and endometrial sarcoma, Since sensitive technologies exist to express to detect genes such as SNAR-A1, said nucleotide probes such as PCR primers or the oligonucleotide probe used in the microarray described herein (TTCCAGGGCACGAGTTCGAGGCCAGCCTGGTCCACATGGGTCGGaaaaaa), as well as other detection techniques described herein, may be used in the unexpected manner described herein to screen for or to otherwise stage the wide array of cancers described above.

In addition, the specific expression of SNAR-A1 in varied malignancies provides novel therapeutic strategies wherein the knockdown or inhibition of the activity of the RNA encoded by SNAR-A1 are used in reducing tumor mass and treating cancer.

### Example 3

### DSCR8 Expression in Diverse Cancer Types

RNA was obtained from cultured diverse cultured human cell types, normal human tissues, and malignant human tumors and analyzed on Illumina gene expression microarrays, The gene encoding the protein down syndrome critical region gene 8 DSCR8 also known as MMA-1a (accession number NM_{—}203428.1) was detected as a gene expressed in relatively higher levels in testis and diverse cancers compared to normal cultured somatic cell types and tissues. There are reports that DSCR8 is expressed in testis and in melanoma (de Wit, N.J. et al Expression profiling of MMA-1a and splice variant MMA-1b: new cancer/testis antigens identified in human melanoma. Int. J. Cancer 98:547-553) and uterine (Risinger, J.I. et al (2007) Global expression analysis of cancer/testis genes in uterine cancers reveals a high incidence of BORIS expression. Clin. Cancer Res. 13:1713-1719) cancer. Measurements of DSCR8 may be useful for screening or diagnosing a wide array of cancers. While these previous reports suggest DSCR8 is expressed in relatively specifically in testis compared to other human tissues and report that it is expressed in uterine cancers and melanomas, they do not report that the relative expression of DSCR8 is diagnostic of the malignant tumors described herein. Surprisingly, as shown in FIG. 6, while diverse cultured normal somatic cell types such as brain microvascular endothelial cells, dermal fibroblasts, smooth muscle cells, esophageal epithelial cells, urothelial cells, pulmonary epithelial cells, prostate epithelial cells, hepatocytes, astrocytes, as well as others and normal tissues tested express relatively low levels of signal (i.e. either background signal of <100 RFU or in the case of eye-derived cells low (<250 RFUs)), samples of normal testis, and diverse malignant tumors expressed the gene at relatively high levels (>250 RFU). Examples of such tumors are: endometrial adenocarcinoma (as predicted based on the art), small cell lung cancer, bladder carcinoma, seminoma of the testis, adenocareinoma of the stomach, the myelogenous leukemia cell line K562, the ovarian cancer cell line OVCAR3, and the melanoma cell line G361 (as expected in the art). Since sensitive technologies exist to express to detect genes such as DSCR8, said nucleotide probes such as PCR primers or the oligonucleotide probe used in the microarray described herein (TCCCACTTGGCAGGGGCCGTCTTGTCCACTCGTTTCTGTAAACATGGGTG), as well as other detection techniques described herein including but not limited to the detection of the protein in tissue samples or blood using monoclonal or polyclonal antibodies, may be used in the unexpected manner described herein to screen for or to otherwise stage the wide array of cancers described above.

In addition, the specific expression of DSCR8 in varied malignancies provides novel therapeutic strategies wherein the knockdown or inhibition of the activity of the protein encoded by DSCR8 or down-regulating the expression or translation of the gene are used in reducing tumor mass and treating cancer.

### Example 4

### PCSK1 Expression in Diverse Cancer Types

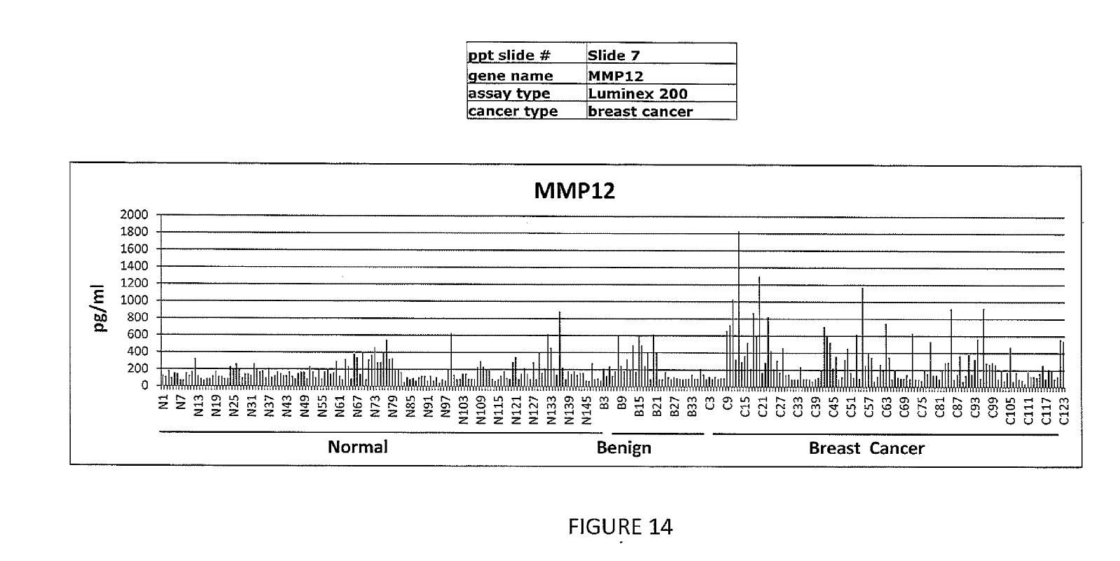

RNA was obtained from cultured diverse cultured human cell types, normal human tissues, and malignant human tumors and analyzed on Illumina gene expression microarrays. The gene encoding the protein proprotein convertase subtilisin/kexin type I (PCSK1) also known as PC1, accession number NM_{—}000439.3 was detected as a gene expressed in relatively higher levels in diverse cancers compared to normal cultured somatic cell types and tissues. There are limited reports that measurements of PCSK1 may be useful for screening or diagnosing a wide array of cancers with the exception that PCSK1 was previously reported to be up-regulated in breast cancer (Cheng M et al, 1997 Pro-protein convertase gene expression in human breast cancer. Intl. J. Cancer 71:966-971) and small cell lung cancer (Moss A.C. et al, SCG3 transcript in peripheral blood is a prognostic biomarker for REST-deficient small cell lung cancer, Clin. Cancer Res. 15: 274-283, (2009)), though Cheng et al 1997 did not report on the specific types of breast cancer in which PCSK1 was abnormally expressed. Surprisingly, as shown in FIG. 14, while diverse cultured normal somatic cell types such as brain microvascular endothelial cells, dermal fibroblasts, smooth muscle cells, esophageal epithelial cells, urothelial cells, pulmonary epithelial cells, mammary epithelial cells, prostate epithelial cells, hepatocytes, astrocytes, as well as others express no signal or low levels of signal (i.e. background signal of <100 RFU), and normal tissues with the expected exception of brain, neurons, dorsal root ganglia, and pancreas expressed no signal (i.e. background signal of <100 RFU only), numerous samples of diverse malignant tumors expressed the gene well above background levels (>100 RFU) such as ductal breast cancer (but not adenocarcinoma of the breast), small cell carcinoma of the lung as expected based on Moss et al, 2009 above, neuroendocrine cancer of the pancreas, rectal adenocarcinoma, gliobastoma multiforme, anaplastic astrocytoma, colon cancer, liver cancer, metastatic smooth muscle sarcoma, the cervical cancer cell line HeLa, and rhabdomyosarcoma. Since technologies exist to express proteins from genes such as PCSK1 and create monoclonal or polyclonal antibodies from said peptides or proteins, said antibodies or nucleotide probes such as PCR primers or the oligonucleotide probe used in the microarray described herein, as well as other detection techniques described herein, may be used in the unexpected manner described herein to screen for or to otherwise stage a wide array of cancers such as ductal cancer of the breast (as opposed to other cancers of the breast), glioblastoma multioforme, neuroendocrine cancer of the pancreas, colon cancer, liver cancer, smooth muscle sarcomas and rhabdomyosarcomas as well as cervical cancer.

Since many cancers are dependent in whole or in part on paracrine or autocrine growth factors for their growth and many growth factors such as epidermal growth factor (EGF), transforming growth factors alpha (TGFA) and beta (TGFB), insulin-like growth factors I and II, insulin-like growth factor receptor I, as well as others have been shown to be processed by proprotein convertases (Mbikay et al, 1993 From proopiomelanocortin to cancer. Possible role of convertases in neoplasia. Annals of the New York Academy of Sciences. 680:13-19), knowledge of proprotein convertases could offer novel targets for cancer therapy. While PCSK1 is not currently recognized as a gene up-regulated in neoplasia with the exception of Cheng et al, 2001 (Elevated expression of proprotein convertases alters breast cancer cell growth in response to estrogen and tamoxifen. J. Mal. Endocrinol. 26:95-105) that reported that breast cancers up-regulated PCI, PC7, PACE4, and FURIN and that exogenous expression of PCSK1 or FURIN in the breast cancer cell line MCF-7 resulted in approximately doubling the rate of proliferation. However, these researchers did not observe the up-regulation of PCSK1 in the other malignancies disclosed herein, nor the novel methods described herein for inhibiting the PCSK1 convertase or utilizing prodrug substrates that are capable of being activated by the PCSK1 convertase into a toxic molecule capable of specifically targeting tumors for destruction as described herein as well as by Carl et al (1980) Protease-activated “prodrugs” for cancer chemotherapy Proc Natl Acad Sci 77:2224-2228; Atkinson et al (2008) Tumour endoproteinases: the cutting edge of cancer drug therapy? Br. J. Pharmacol. 153:1344-1352) incorporated herein by reference.

The aforementioned prodrug substrates that may be activated by the PCSK1 proprotein convertase from a non-active to an active form may be designed by methods well known in the art but where the target site of PCSK1 is introduced into the substrate proprotein at a site wherein proteolytic cleavage at the site will activate the proprotein. The exogenously-added substrate can be utilized as a substrate of the tumor PCSK1 or other related cancer-specific proprotein convertases including but not limited to the proteins encoded by FURIN, PCSK6 (PACE4), and PCSK7, of specifically activating or deactivating protein substrates delivered to the tumor to alter the properties of these proteins such that the altered properties of the proteins is therapeutic by decreasing the growth rates or metastasis of tumors, causing a cytotoxic effect on the tumor, causing a local inhibition of angiogenesis or induction of blood clotting, or otherwise inducing effects that decrease human morbidity and mortality from cancer.

By way on nonlimiting example, the target site for the cleavage of PCSK1 proprotein convertase Lys-Ser-Val-Lys-Arg-*-Ser-Val-Ser-Glu-Ile-Gln-Leu (where the cleavage site is marked with an asterisk (“*”)) is introduced onto the amino terminus of a protein followed by an effector molecule such as desacetyl-Vinblastine as described by Atkinson et at (2008) (Tumour endoproteinases: the cutting edge of cancer drug therapy? Br. J. Pharmacol. 153:1344-1352) incorporated herein by reference. Such substrates can be introduced systemically, or more preferably locally, and most preferable within the tumor niche and expressed by an inducible promoter such as a radiation inducible promoter and expressed by living cells that target tumors, such as vascular endothelial, perivascular cells such as mesenchymal stem cells and pericytes, tumor infiltrating lymphocytes or monocyte/macrophages, or cancer stromal cells.

Other methods for targeting tumor cells are described in PCT/US03/01827, published as WO 2003/061591; and entitled “STEM CELL-DERIVED ENDOTHELIAL CELLS MODIFIED TO DISRUPT TUMOR ANGIOGENESIS” and Us app. publications: 2006/0024280 and 2004/0018178, each of which is hereby incorporated by reference in its entirety.

### Example 5

### Serum Detection of Breast Cancer and Colon Cancer Markers

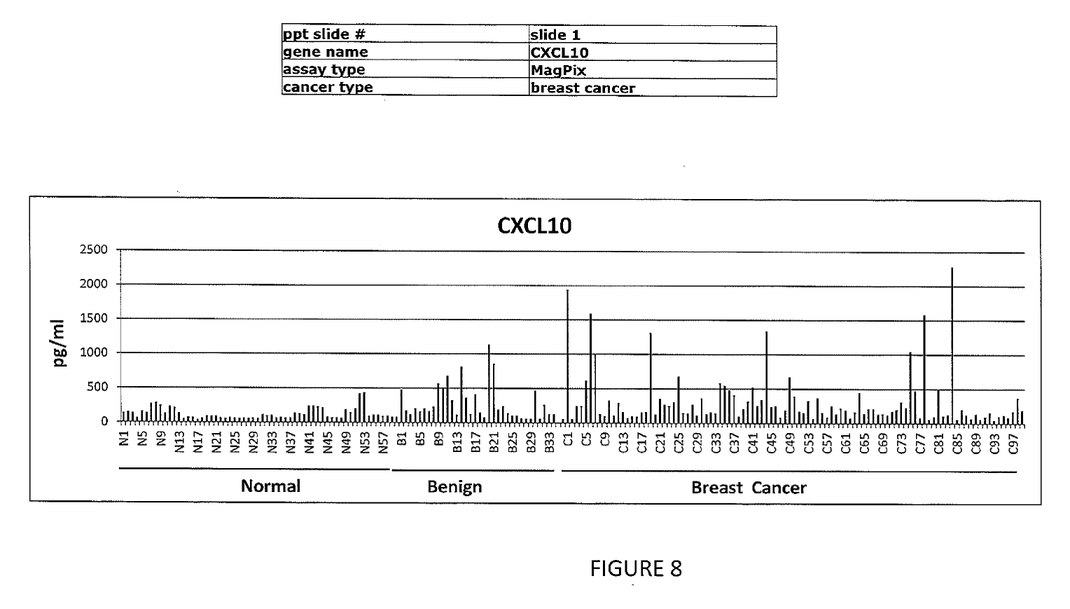

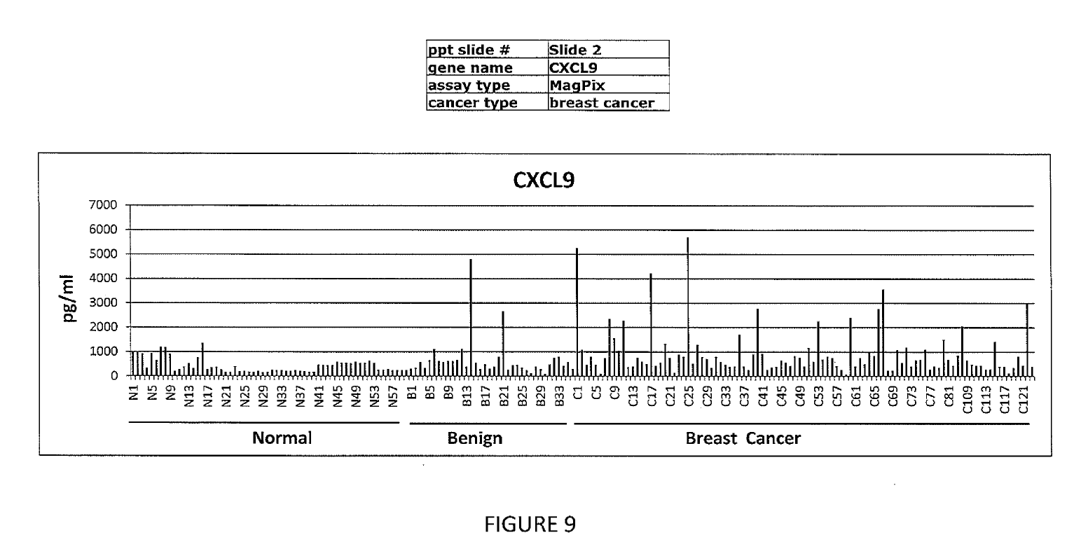

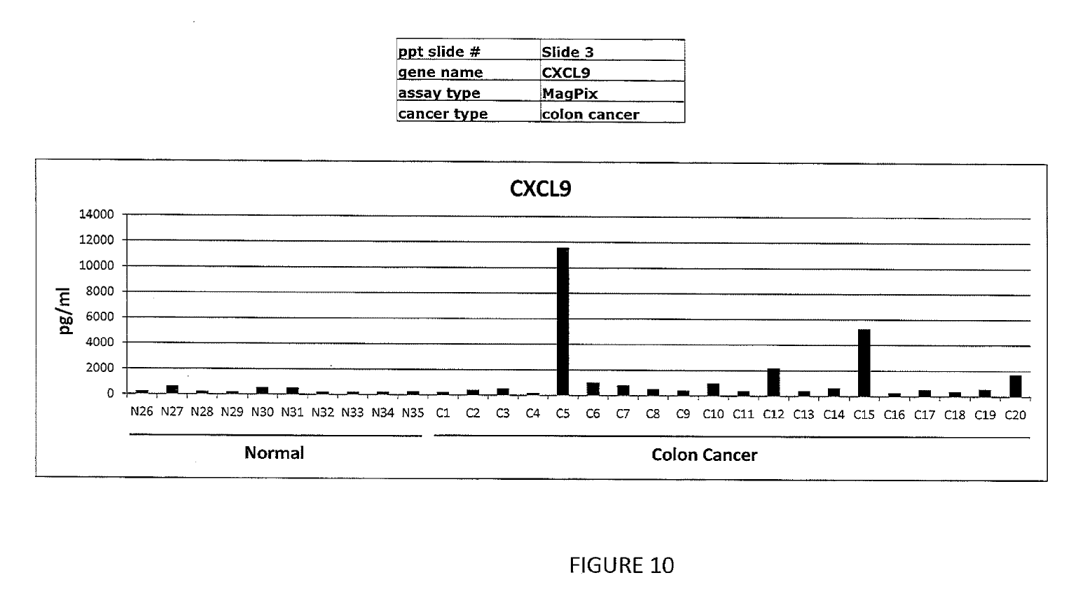

Levels of CXCL10, CXCL9 protein were assayed in human serum using a Human Cytokine/Chemokine magnetic bead panel kit (Millipore, Bedford Mass.) according to the manufacturer's instructions. In brief, 200 μL of wash buffer was added per well and shaken for 10 minutes, then decanted. Then, 25 μL of standard or control was added to the appropriate wells and 25 μL of assay buffer was added to the background and sample wells. Then, 25 μL of the appropriate matrix solution was added to the background, standards, and control wells. Then, 25 μL of serum sample were added to the sample wells and then 25 μL of beads were added to each well and incubated overnight at 4° C. with shaking. The contents of the wells were removed and washed 2× with 200 μL wash buffer and then 25 uL detection antibodies were added to each well and incubated for 1 hour at room temperature. Then, 25 μL Streptavidin-Phycoerythrin per well was added and incubated for 30 minutes at room temperature. The contents of the wells were removed and washed 2 times with 200 uL wash buffer and 150 uL of wash buffer was added per well and was read on the Luminex MagPix instrument (100 μL, 50 beads per bead set). A standard curve was derived from the standards supplied in the kit and the sample values were extrapolated from this curve. Serum samples of normal serum, subjects with benign breast or colon tumors and malignant colon and breast tumors were analyzed. The results shown in FIGS. 8-10 show that CXCL10 protein expression is elevated in malignant breast tumors compared to serum obtained from non-cancerous subjects or those with a benign breast tumor.

### Example 6

### Serum Detection of Breast Cancer, Pancreatic Cancer and Colon Cancer Markers

Levels of MMP7, MMP12 and MMP9 protein were assayed in serum using a Luminex kit (Millipore, Bedford, Mass.) according to the manufacturer's instructions. In brief, 200 μL of wash buffer was added per well, shaken for 10 minutes, then decanted. Then, 25 μL of standard or control was added to the appropriate wells and 25 μL of assay buffer was added to the background and sample wells. Then, 25 μl of the appropriate matrix solution was added to the background, standards, and control wells. Then, 25 μL of serum sample were added to the sample wells and then 25 μL of beads were added to each well and incubated overnight at 4° C. with shaking. The contents of the wells was removed and washed 2× with 200 μL wash buffer and then 25 uL detection antibodies was added to each well and incubated for 1 hour at room temperature. Then, 25 μL Streptavidin-Phycoerythrin per well was added and incubated for 30 minutes at room temperature. The contents of the wells were removed and washed 2 times with 200 uL wash buffer and 100 uL of wash buffer was added per well and samples were read on the Luminex 200 instrument (50 μL, 50 beads per bead set). A standard curve was derived from the standards supplied in the kit and the sample values were extrapolated from this curve.

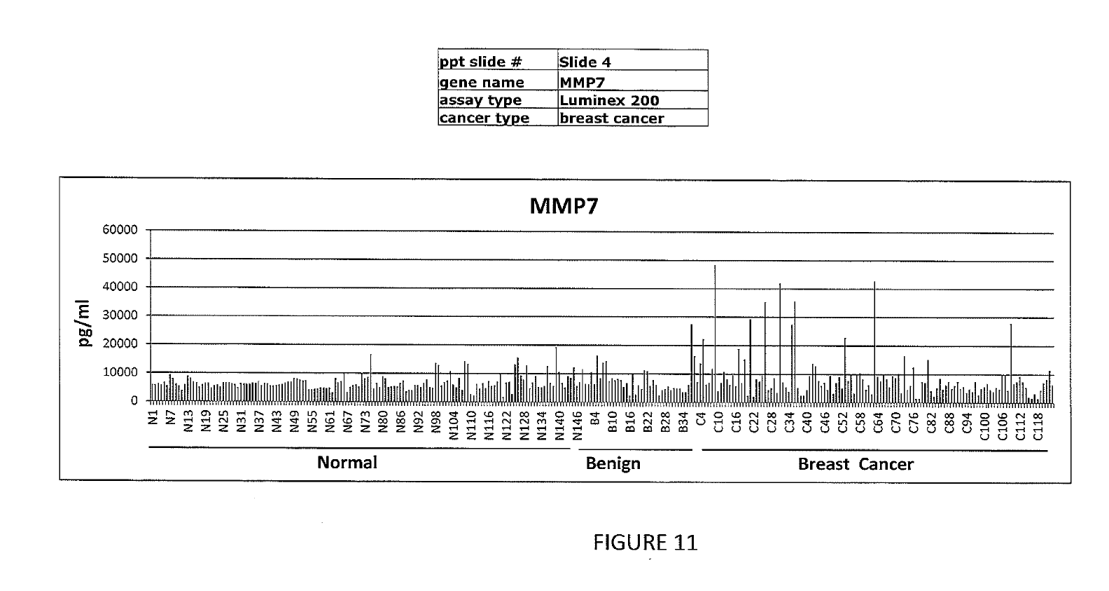

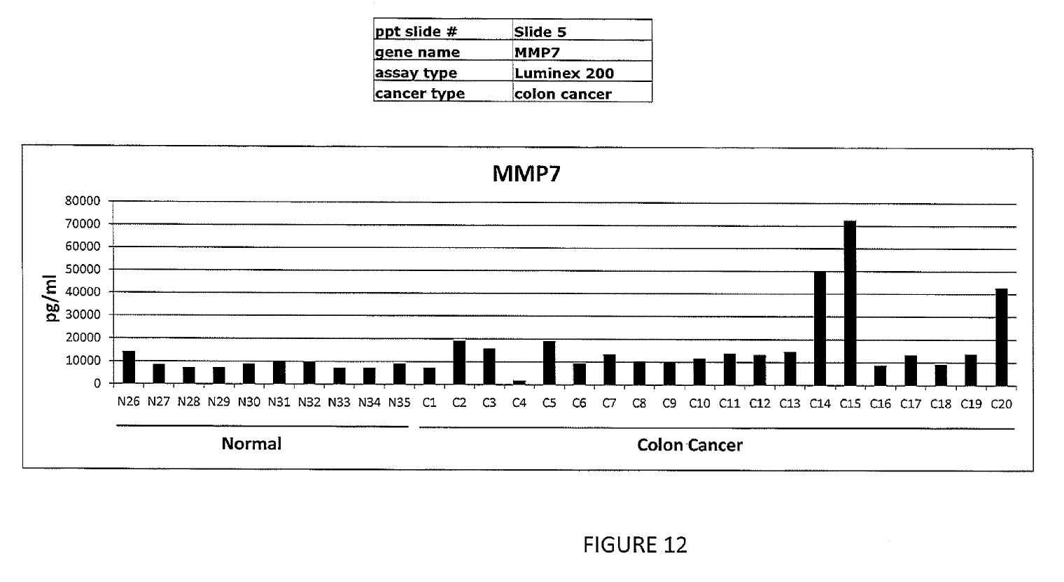

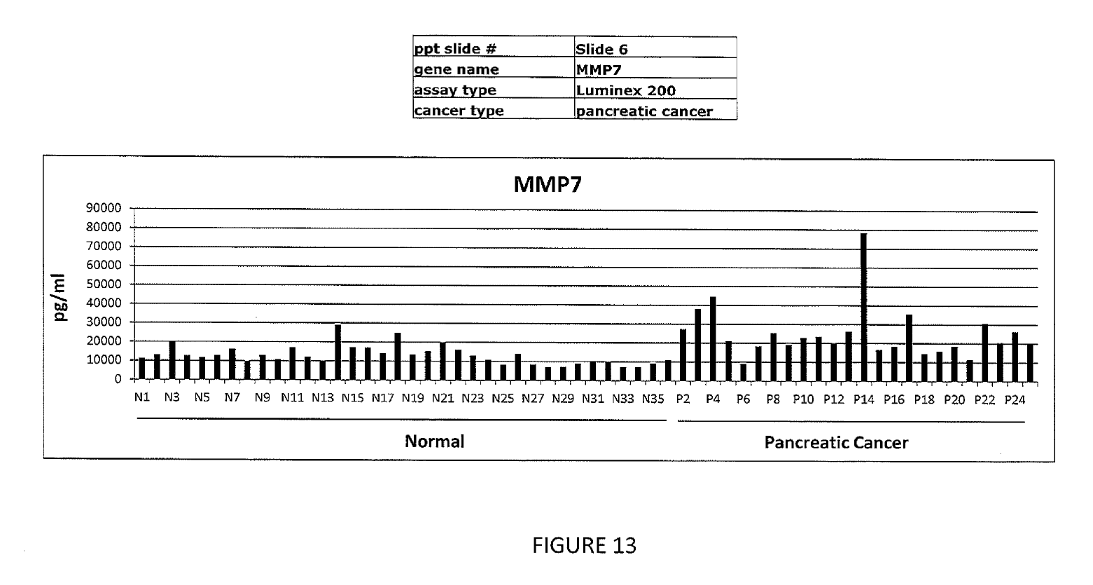

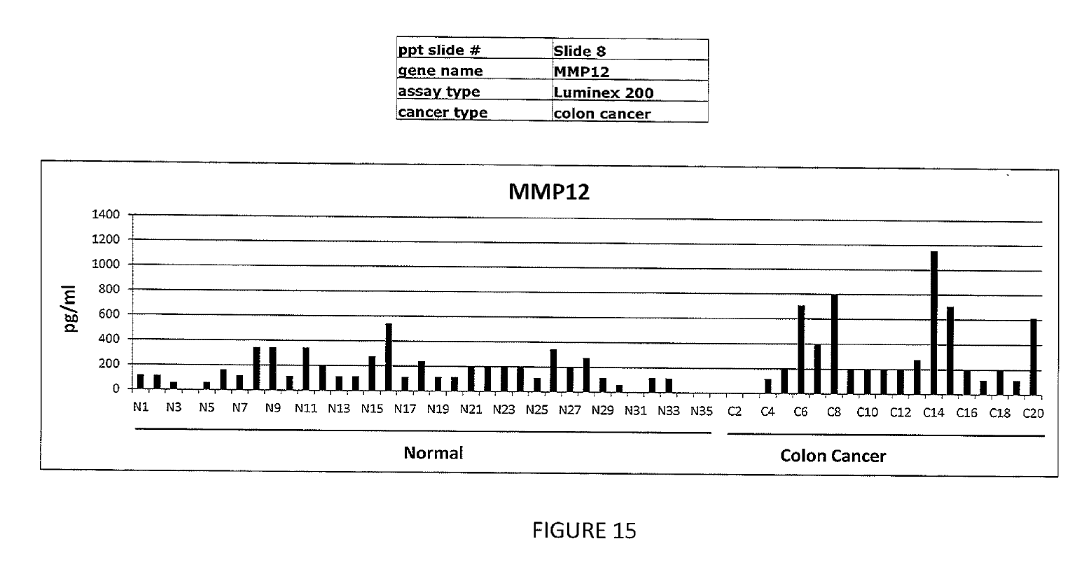

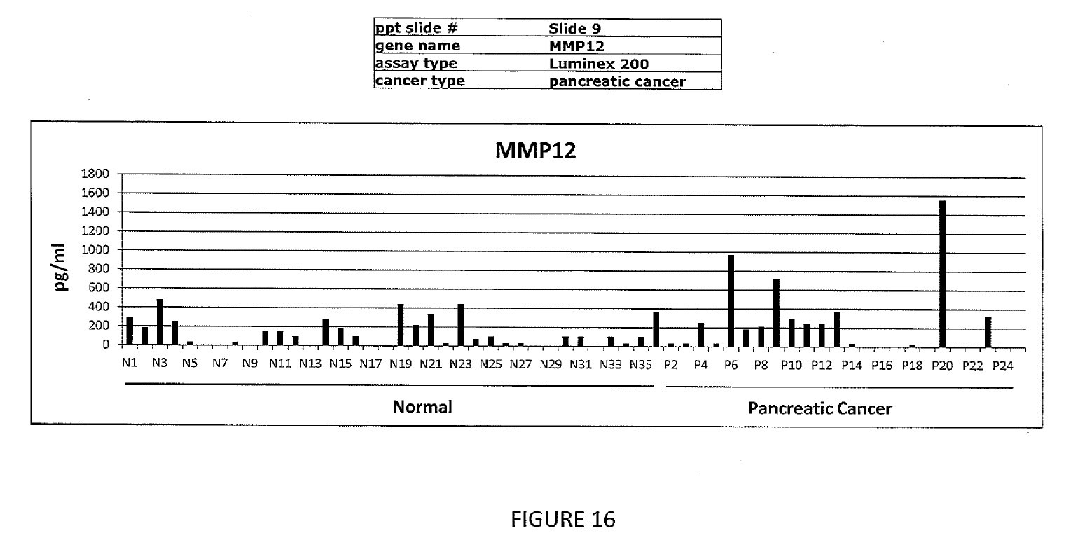

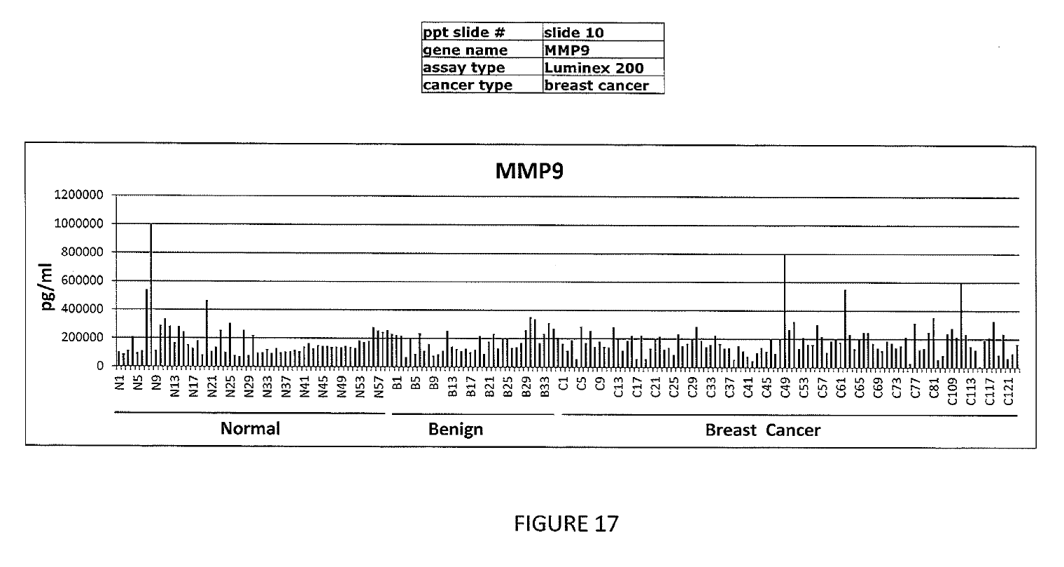

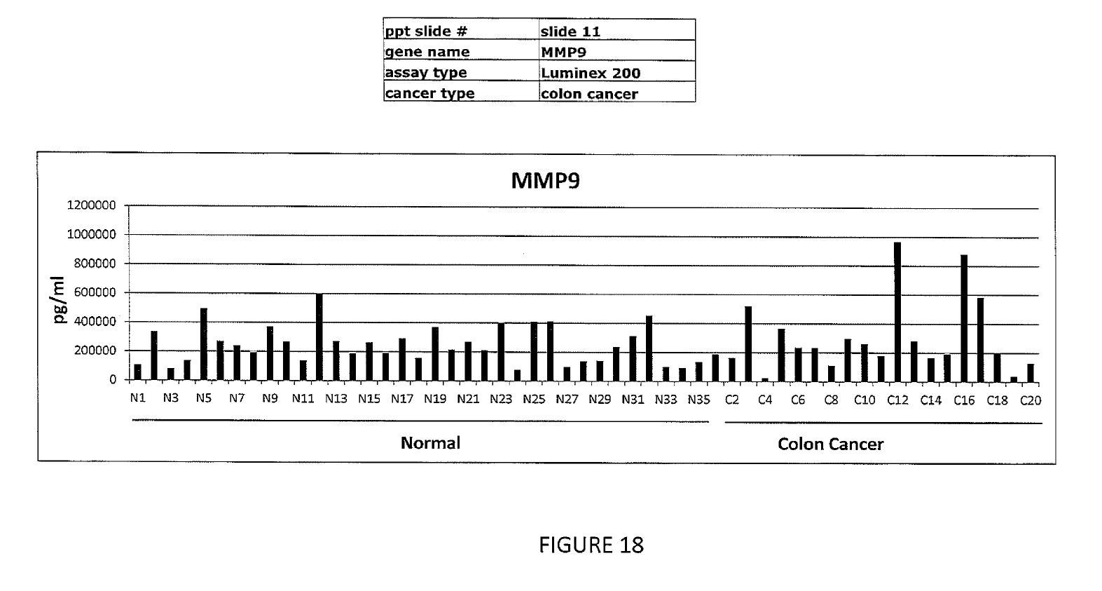

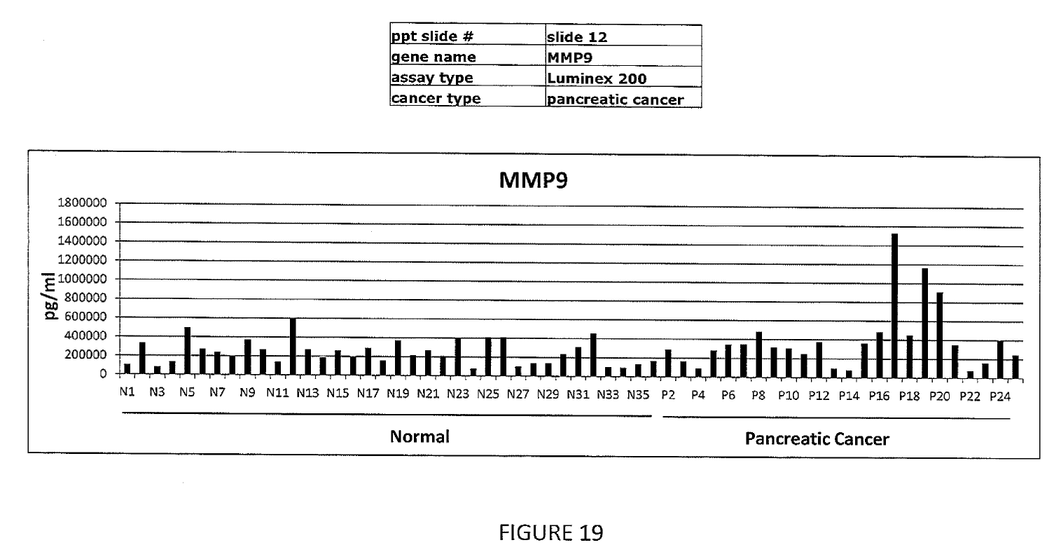

Serum samples from normal subjects, subjects and those with either pancreatic, breast or colon cancer were analyzed. In addition, subjects with benign breast tumors were also analyzed. The results, shown in FIGS. 11-19 indicate that protein expression levels of MMP9, MMP7 and MMP12 in serum of subjects with colon, pancreatic or breast cancer were elevated when compared to normal subjects.

### Example 7

### Serum Detection of Cancer Markers

Levels of the proteins EPYC, IL8, LAMC2, and CLCA1 were assayed in serum using a USCN ELISA kit (USCN) according to the manufacturer's instructions. In brief, 100 μL of the blank, standards, and samples with specified dilutions were added to the appropriate wells of a 96 well plate followed by 2 hours of incubation at 37° C. After removal of the liquid, 100 ul of Detection Reagent A was added to each well and incubated for 1 hour at 37° C. After removal of Reagent A, each well was washed 3 times with 350 uL of wash solution. 100 uL of Detection Reagent B was added to each well and then incubated for 30 minutes at 37° C. After removal of Reagent B, each well was washed 5 times with 350 uL of wash solution. 90 uL of Substrate solution was added to each well and incubated for 15-25 minutes at 37° C. 50 uL of Stop Solution was added to each well. The plate was read either on the Molecular Devices SpectraMax250 or the BioTek Synergy H1 plate reader at 450 nm. A standard curve was derived from the standards supplied in the kit and the sample values were extrapolated from this curve.

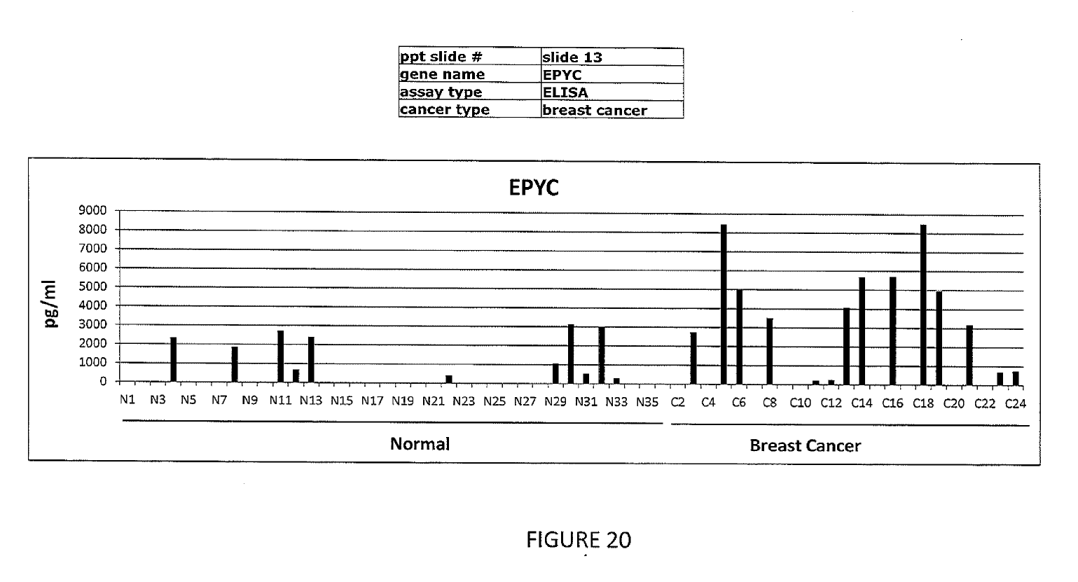

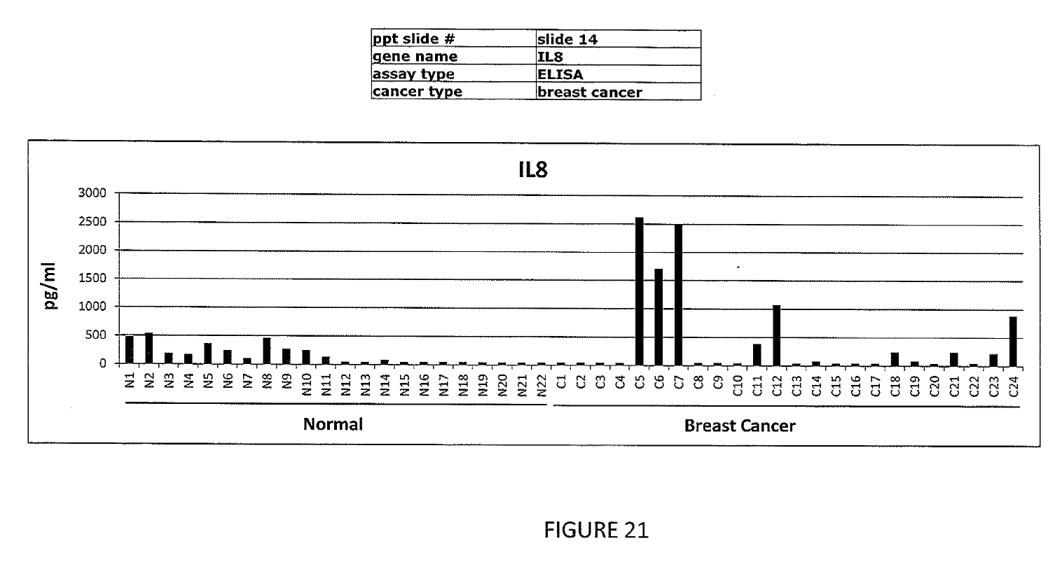

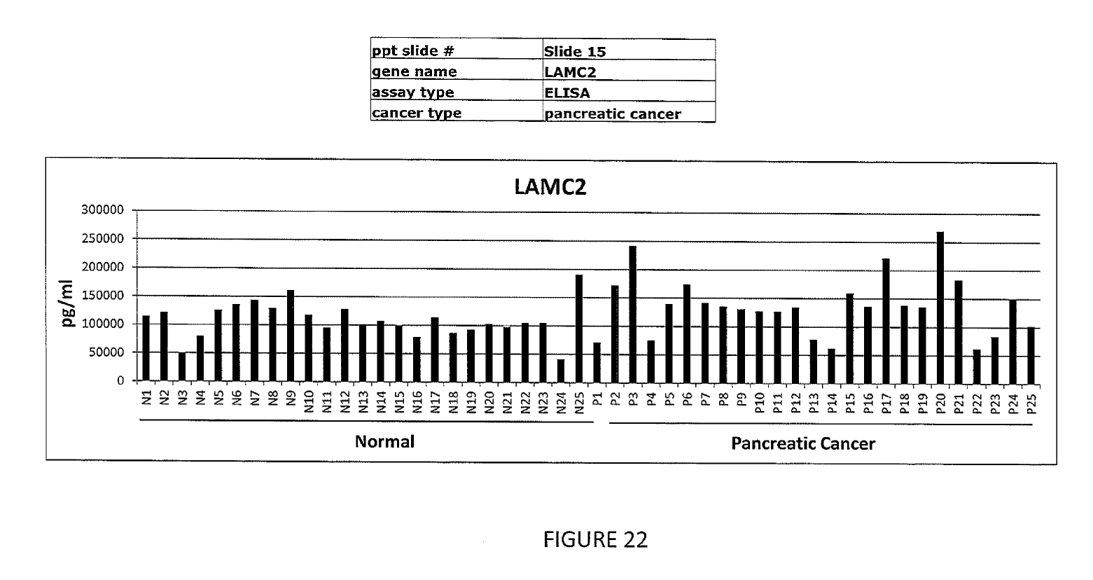

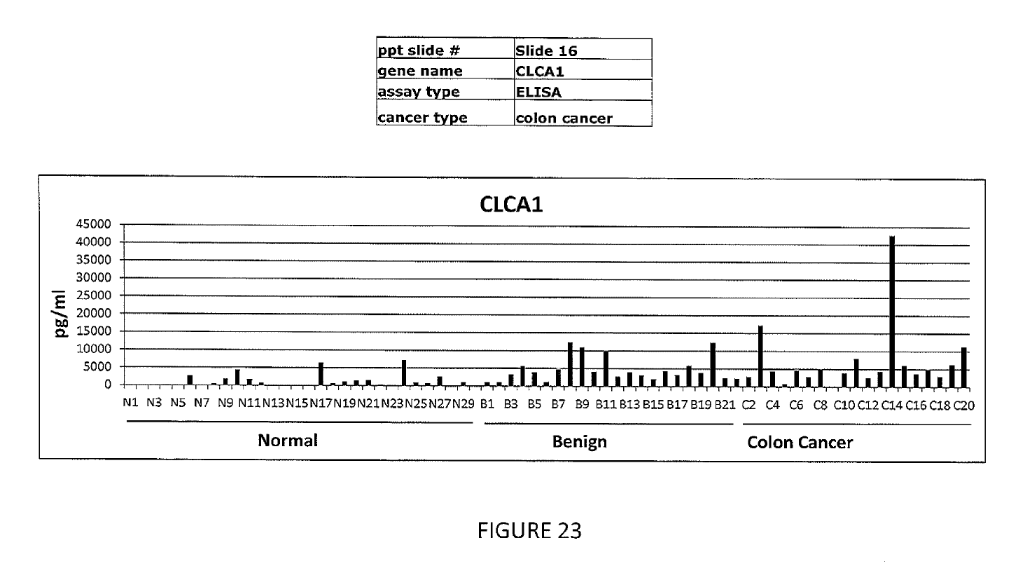

The results shown in FIGS. 20-23 indicated that EPYC and IL8 were elevated in serum obtained from subjects with breast cancer compared to normal subjects and that LAMC2 was elevated in subjects with pancreatic cancer compared to normal subjects. The results also showed that CLCA1 was elevated in subjects with colon cancer compared to normal subjects.

### Example 9

### Serum Detection Level of LCN2 in Cancer

Levels of the protein LCN2 were assayed in serum using a USCN ELISA kit (USCN) according to the manufacturer's instructions. In brief, 100 μL of the blank, standards, and samples with specified dilutions were added to the appropriate wells of a 96 well plate followed by 2 hours of incubation at 37° C. After removal of the liquid, 100 ul of Detection Reagent A was added to each well and incubated for 1 hour at 37° C. After removal of Reagent A, each well was washed 3 times with 350 uL of wash solution. 100 uL of Detection Reagent B was added to each well and then incubated for 30 minutes at 37° C. After removal of Reagent B, each well was washed 5 times with 350 uL of wash solution. 90 uL of Substrate solution was added to each well and incubated for 15-25 minutes at 37° C. 50 uL of Stop Solution was added to each well. The plate was read either on the Molecular Devices SpectraMax250 or the BioTek Synergy H1 plate reader at 450 nm. A standard curve was derived from the standards supplied in the kit and the sample values were extrapolated from this curve.

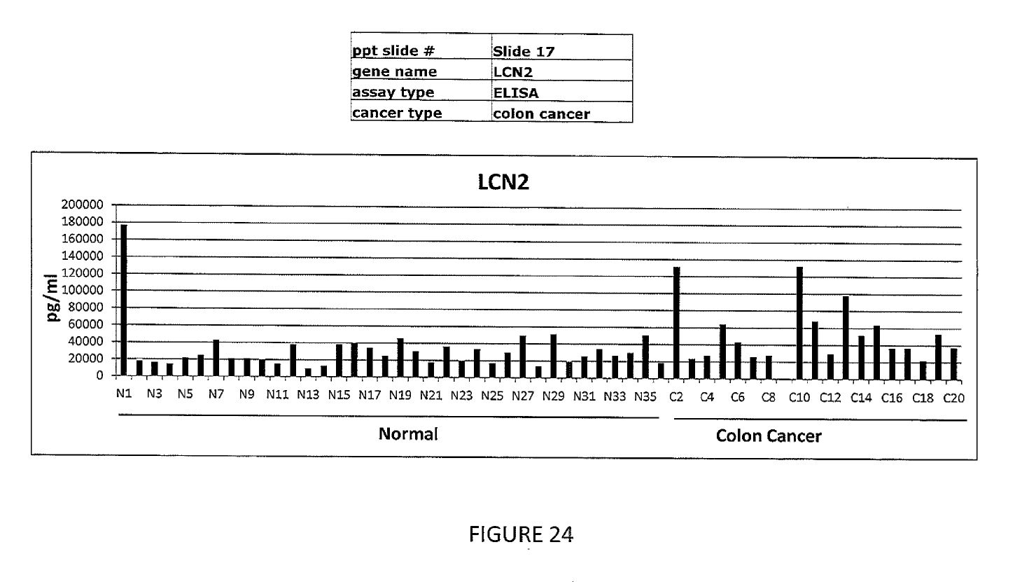

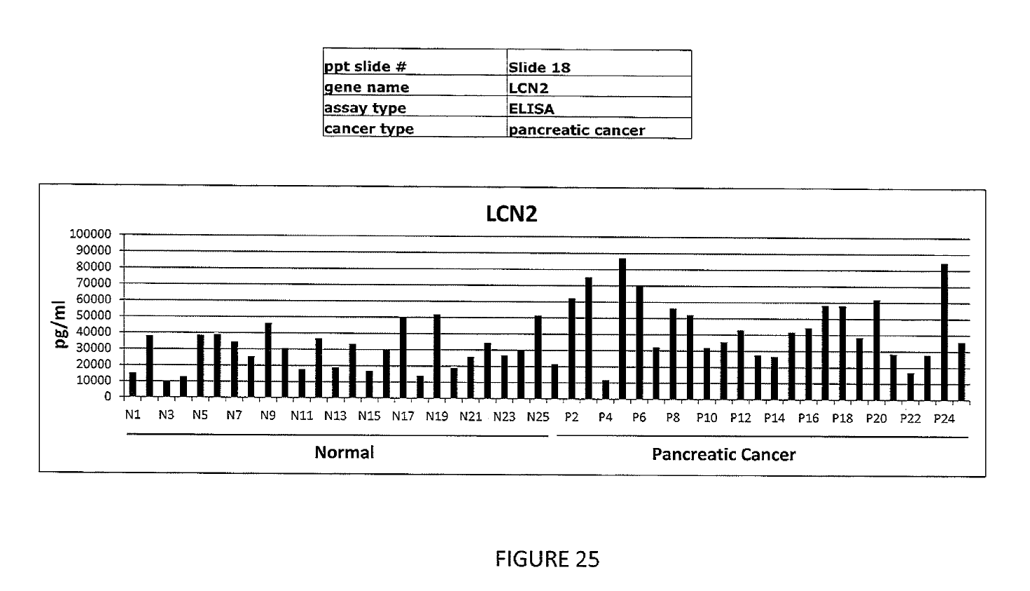

The results shown in FIGS. 24-25 indicated that LCN2 was elevated in serum obtained from subjects with colon and pancreatic cancer compared to normal subjects.

### Example 10

### Serum Detection Level of REG4, REG1b and OLFM4 in Cancer

Levels of the proteins REG4 AND REG1 b were assayed in serum using a USCN ELISA kit (USCN) according to the manufacturer's instructions. In brief, 100 μL of the blank, standards, and samples with specified dilutions were added to the appropriate wells of a 96 well plate followed by 2 hours of incubation at 37° C. After removal of the liquid, 100 ul of Detection Reagent A was added to each well and incubated for 1 hour at 37° C. After removal of Reagent A, each well was washed 3 times with 350 uL of wash solution. 100 uL of Detection Reagent B was added to each well and then incubated for 30 minutes at 37° C. After removal of Reagent B, each well was washed 5 times with 350 uL of wash solution. 90 uL of Substrate solution was added to each well and incubated for 15-25 minutes at 37° C. 50 uL of Stop Solution was added to each well. The plate was read either on the Molecular Devices SpectraMax250 or the BioTek Synergy H1 plate reader at 450 nm. A standard curve was derived from the standards supplied in the kit and the sample values were extrapolated from this curve.

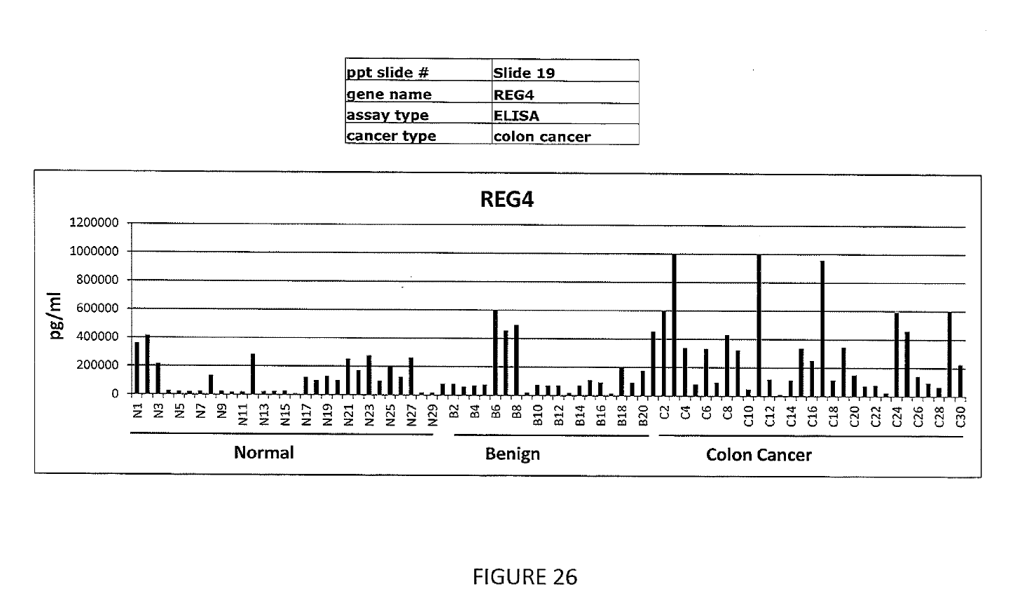

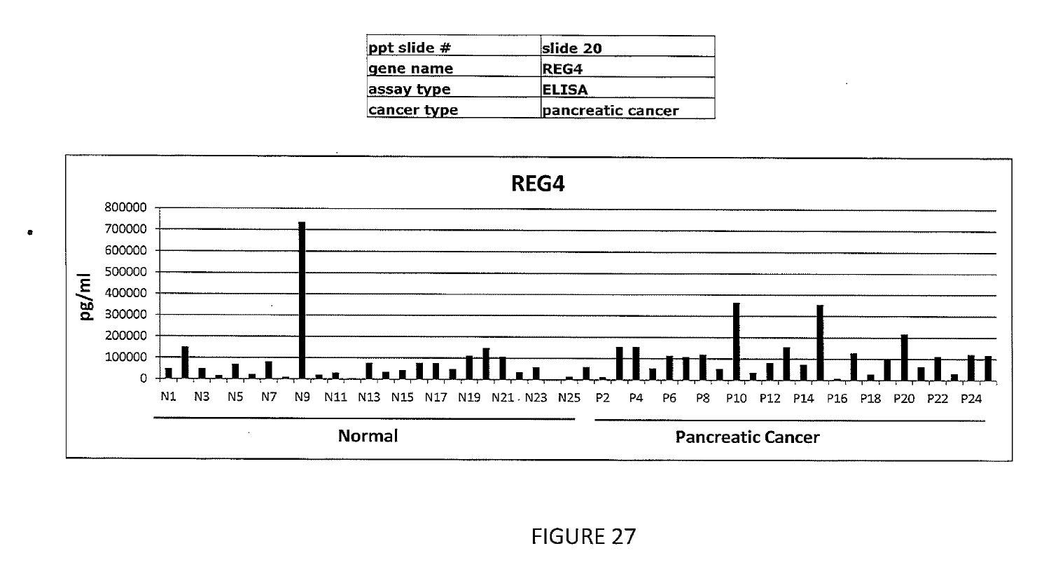

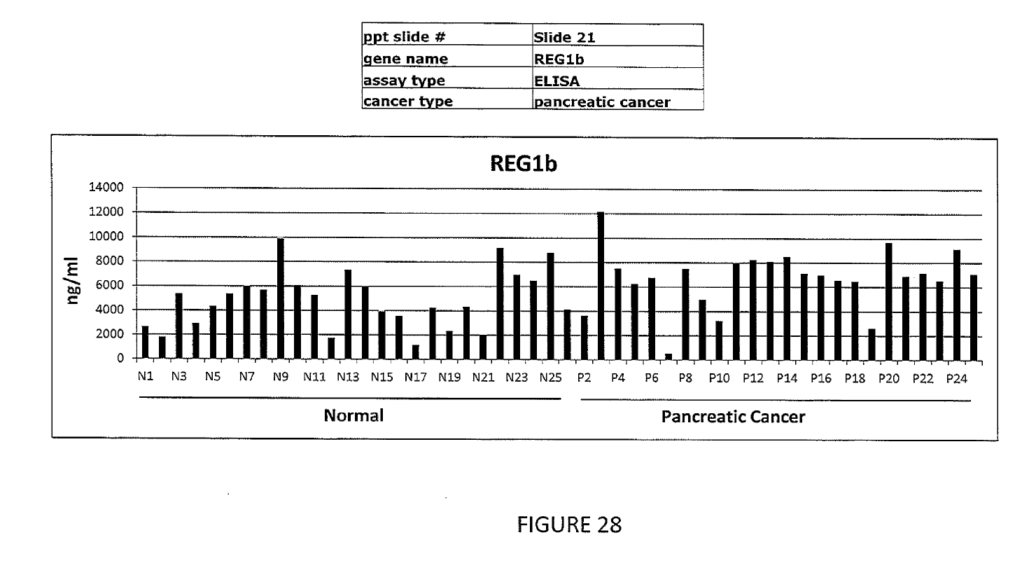

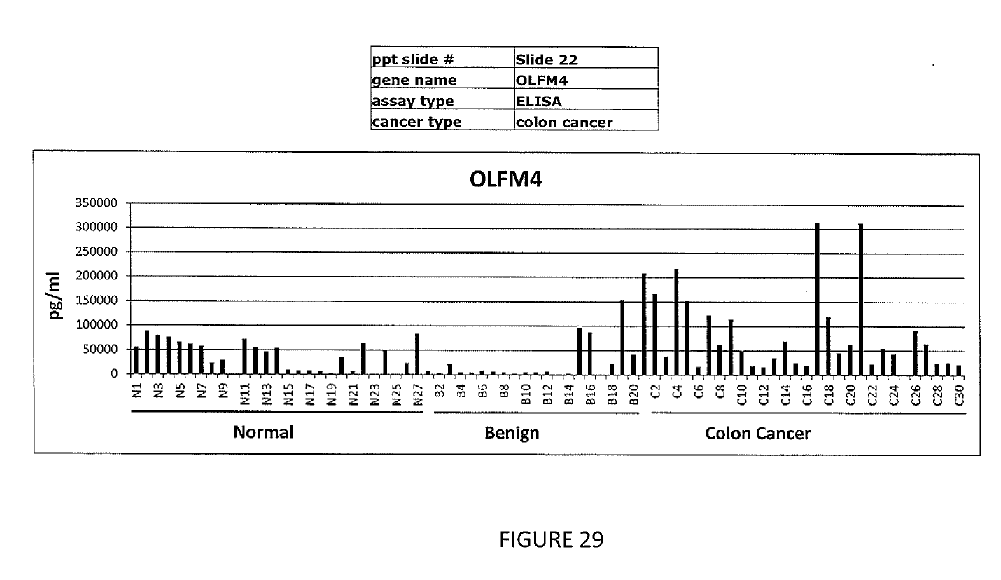

The results shown in FIGS. 26-28 indicated that REG4 was elevated in serum obtained from subjects with colon and pancreatic cancer compared to normal subjects and that REG1 b was elevated in subjects with pancreatic cancer compared to normal subjects. OLFM4 was found to be elevated in subjects with colon cancer compared to normal subjects.

### Example 11

### Serum Detection Level of UBD in Cancer

Levels of the protein UBD were assayed in serum using a USCN ELISA kit (USCN) according to the manufacturer's instructions. In brief, 100 μL of the blank, standards, and samples with specified dilutions were added to the appropriate wells of a 96 well plate followed by 2 hours of incubation at 37° C. After removal of the liquid, 100 ul of Detection Reagent A was added to each well and incubated for 1 hour at 37° C. After removal of Reagent A, each well was washed 3 times with 350 uL of wash solution. 100 uL of Detection Reagent B was added to each well and then incubated for 30 minutes at 37° C. After removal of Reagent B, each well was washed 5 times with 350 uL of wash solution. 90 uL of Substrate solution was added to each well and incubated for 15-25 minutes at 37° C. 50 uL of Stop Solution was added to each well. The plate was read either on the Molecular Devices SpectraMax250 or the BioTek Synergy H1 plate reader at 450 nm. A standard curve was derived from the standards supplied in the kit and the sample values were extrapolated from this curve.

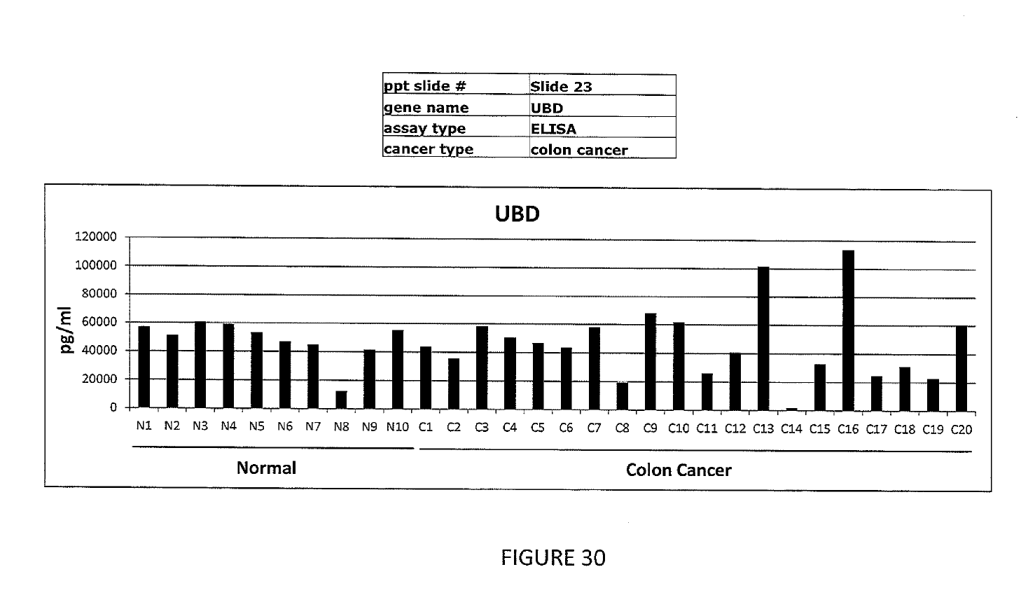

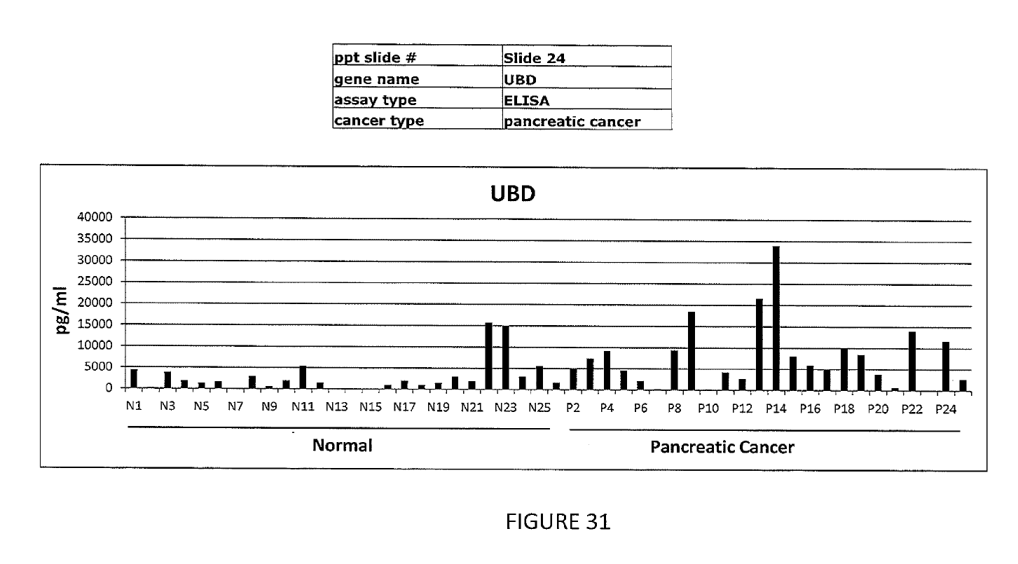

The results shown in FIGS. 30-31 indicated that UBD was elevated in serum obtained from subjects with colon and pancreatic cancer compared to normal subjects.

### Example 12

### Serum Detection Level of NMU in Cancer

Levels of the protein NMU were assayed in serum using a USCN ELISA kit (USCN) according to the manufacturer's instructions, In brief, 100 μL of the blank, standards, and samples with specified dilutions were added to the appropriate wells of a 96 well plate followed by 2 hours of incubation at 37° C. After removal of the liquid, 100 ul of Detection Reagent A was added to each well and incubated for 1 hour at 37° C., After removal of Reagent A, each well was washed 3 times with 350 uL of wash solution. 100 uL of Detection Reagent B was added to each well and then incubated for 30 minutes at 37° C. After removal of Reagent B, each well was washed 5 times with 350 uL of wash solution. 90 uL of Substrate solution was added to each well and incubated for 15-25 minutes at 37° C., 50 uL of Stop Solution was added to each well. The plate was read either on the Molecular Devices SpectraMax250 or the BioTek Synergy H1 plate reader at 450 nm. A standard curve was derived from the standards supplied in the kit and the sample values were extrapolated from this curve.

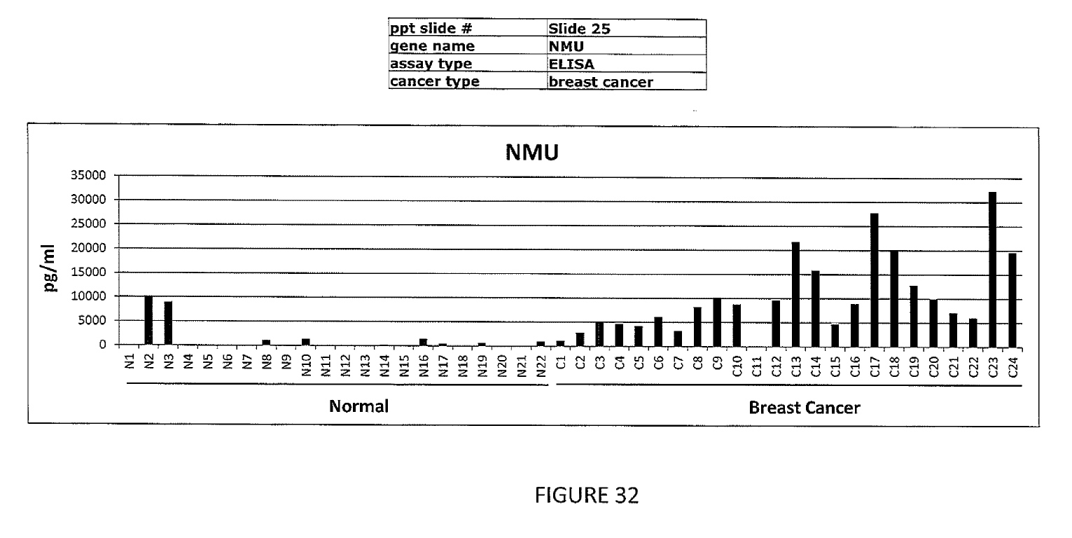

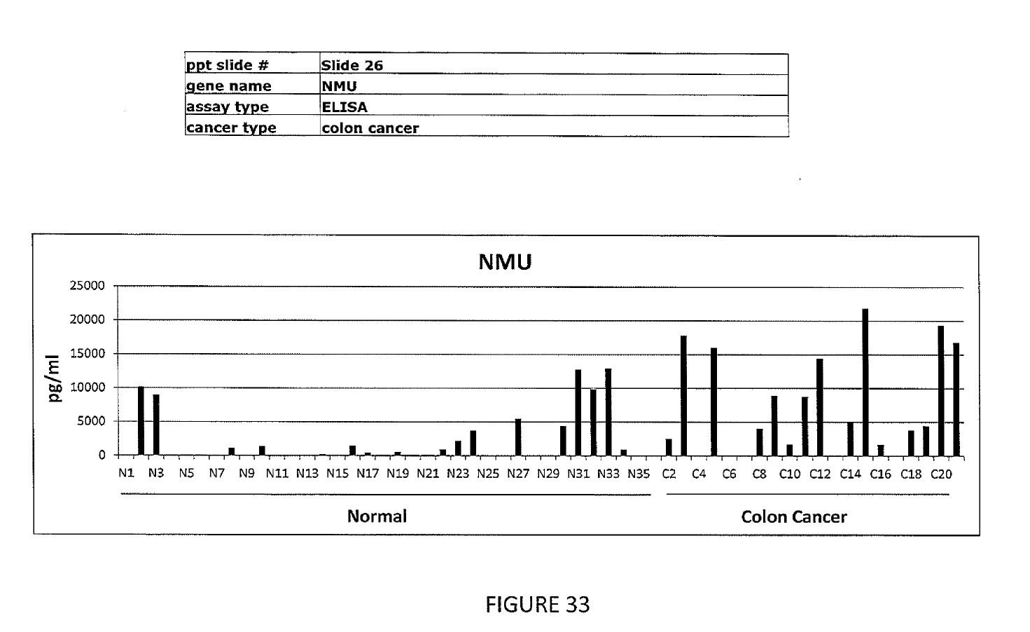

The results shown in FIGS. 32-33 indicated that UBD was elevated in serum obtained from subjects with colon and breast cancer compared to normal subjects.

### Example 13

### Serum Detection Level of MMP11 in Cancer

Levels of the protein MMP11 were assayed in serum using a USCN ELISA kit (USCN) according to the manufacturer's instructions. In brief, 100 μL of the blank, standards, and samples with specified dilutions were added to the appropriate wells of a 96 well plate followed by 2 hours of incubation at 37° C. After removal of the liquid, 100 ul of Detection Reagent A was added to each well and incubated for 1 hour at 37° C. After removal of Reagent A, each well was washed 3 times with 350 uL of wash solution, 100 uL of Detection Reagent B was added to each well and then incubated for 30 minutes at 37° C. After removal of Reagent B, each well was washed 5 times with 350 uL of wash solution. 90 uL of Substrate solution was added to each well and incubated for 15-25 minutes at 37° C. 50 uL of Stop Solution was added to each well. The plate was read either on the Molecular Devices SpectraMax250 or the BioTek Synergy H1 plate reader at 450 nm. A standard curve was derived from the standards supplied in the kit and the sample values were extrapolated from this curve.

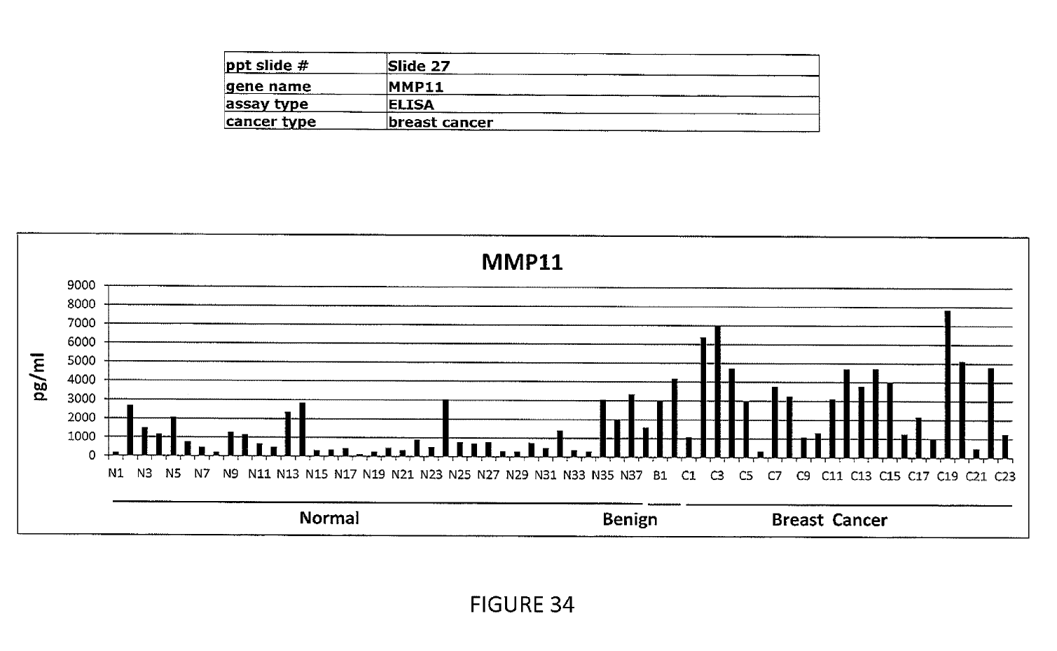

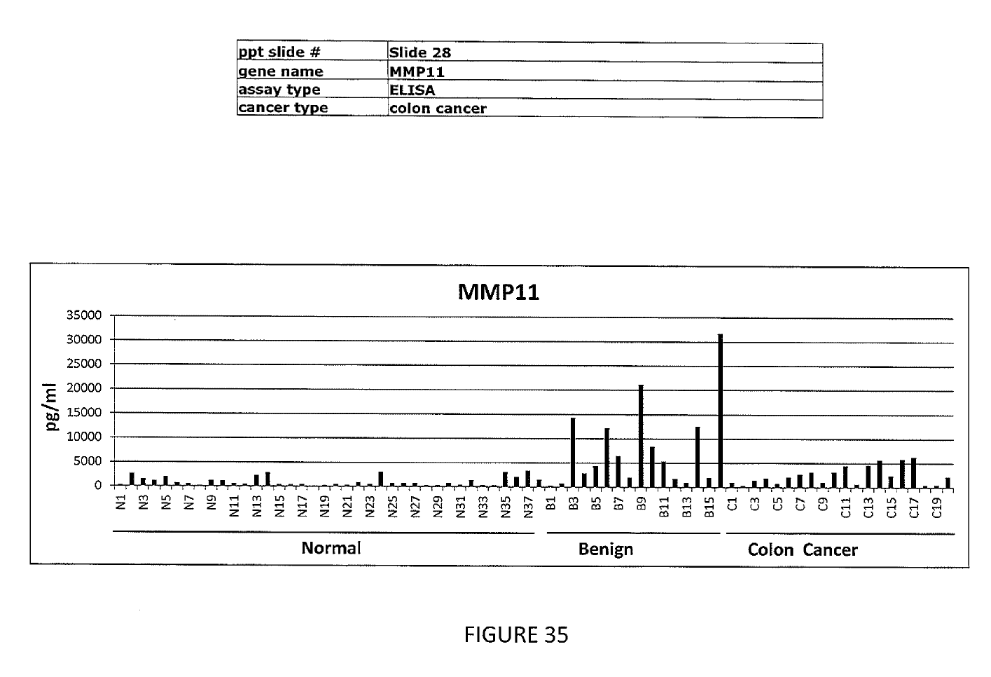

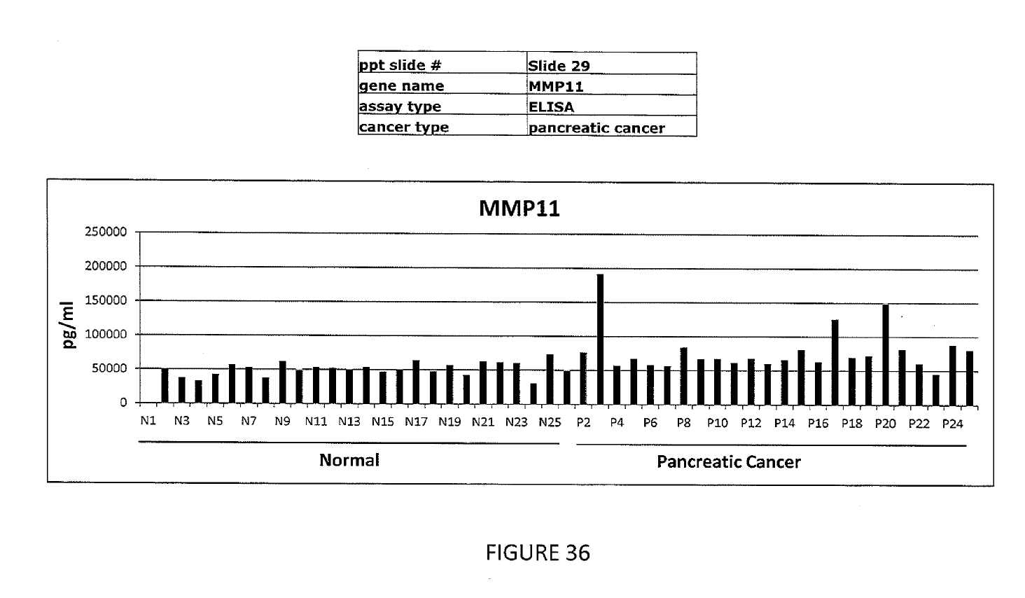

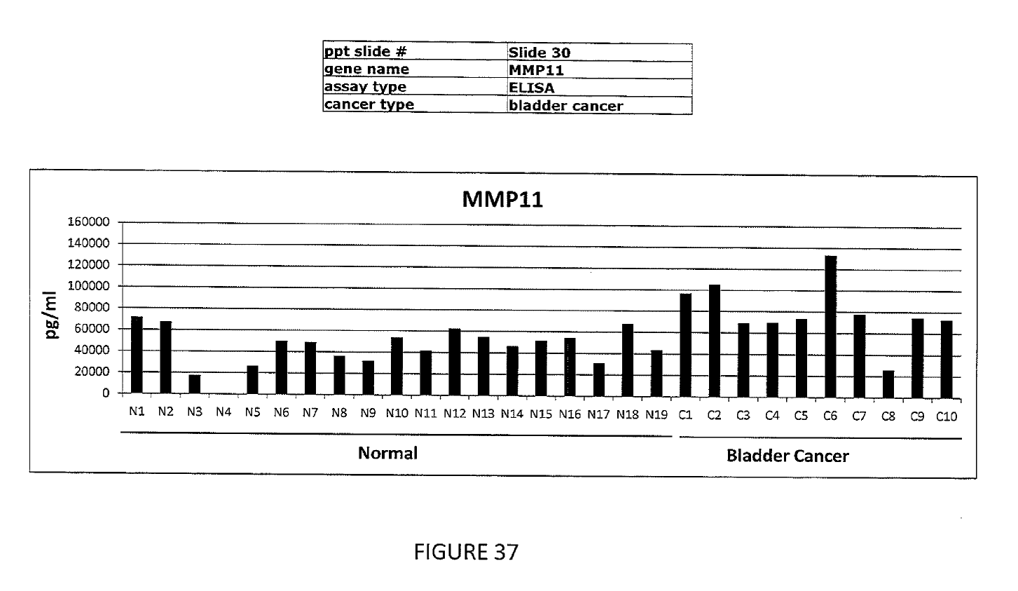

The results shown in FIGS. 34-37 indicated that MMP11 was elevated in serum obtained from subjects with colon, pancreatic, bladder and breast cancer compared to normal subjects.

### Example 14

### Serum Detection Level of WNT10A in Cancer

Levels of the protein WNT10A was assayed in serum using a USCN ELISA kit (USCN) according to the manufacturer's instructions. In brief, 100 μL of the blank, standards, and samples with specified dilutions were added to the appropriate wells of a 96 well plate followed by 2 hours of incubation at 37° C. After removal of the liquid, 100 ul of Detection Reagent A was added to each well and incubated for 1 hour at 37° C. After removal of Reagent A, each well was washed 3 times with 350 uL of wash solution. 100 uL of Detection Reagent B was added to each well and then incubated for 30 minutes at 37° C. After removal of Reagent B, each well was washed 5 times with 350 uL of wash solution. 90 uL of Substrate solution was added to each well and incubated for 15-25 minutes at 37° C. 50 uL of Stop Solution was added to each well. The plate was read either on the Molecular Devices SpectraMax250 or the BioTek Synergy H1 plate reader at 450 nm. A standard curve was derived from the standards supplied in the kit and the sample values were extrapolated from this curve.

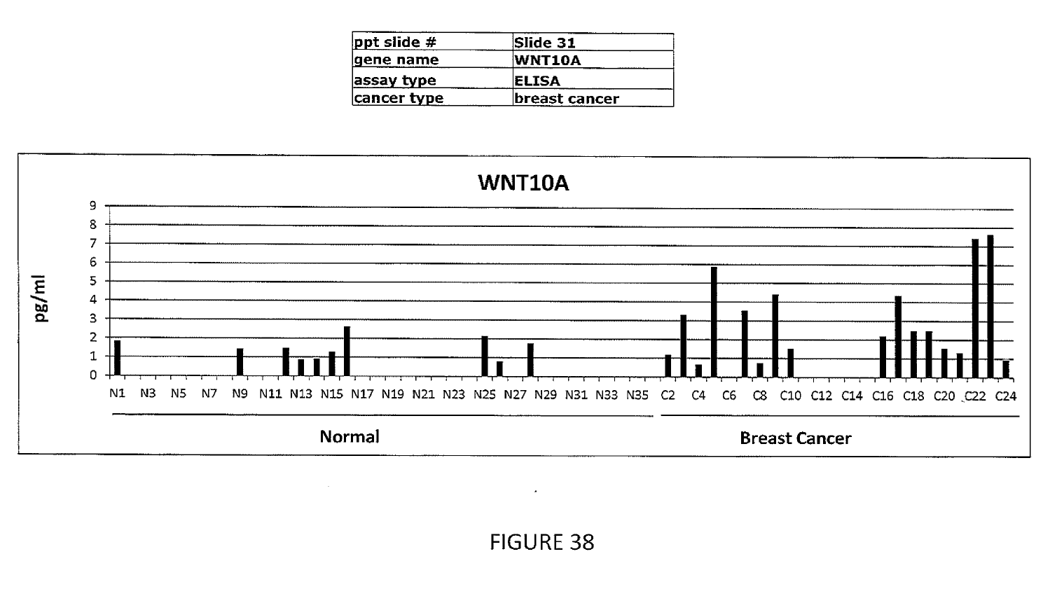

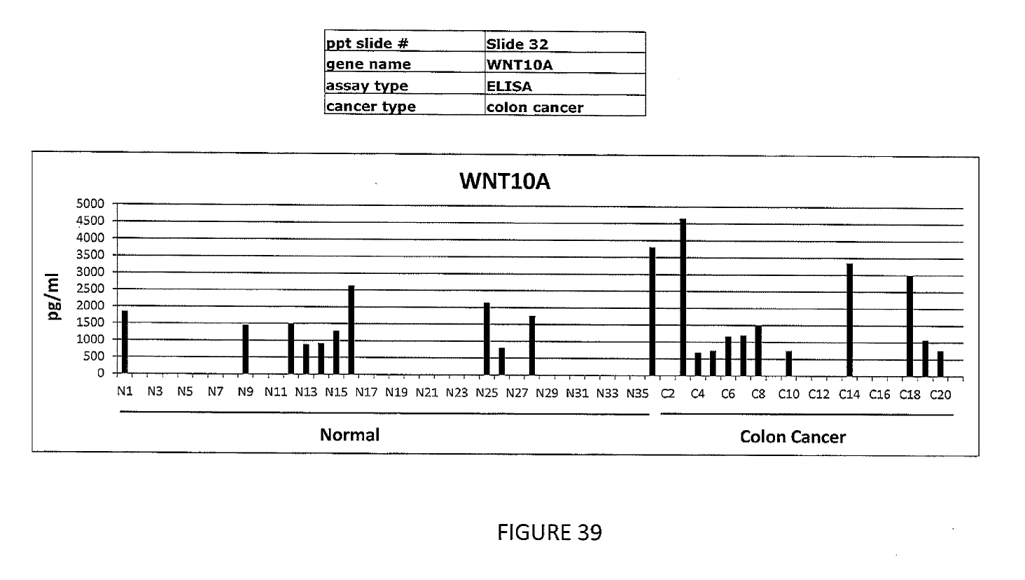

The results shown in FIGS. 38-39 indicated that WNT10A was elevated in serum obtained from subjects with colon, and breast cancer compared to normal subjects.

### Example 15

### qPCR Analysis of Tissue Samples for Cancer Markers

qPCR was performed on the following tumor tissue and normal tissue: bladder, breast, cervix, gastroesophageal, colon, skin, ovary, tonsil thyroid brain, stomach, and lung. Positive controls were specific known tumors previously assayed by microarray.

Total RNA was extracted with the RNeasy Mini Kit (Qiagen) and cDNA generated using the SuperScript III reverse transcriptase in combination with random hexamer primers alone or in combination with oligo-dT primers (all reverse transcription components from Invitrogen/Life Technologies). PCRs were carried out on a 7900HT Sequence Detection System or a 7500 Real Time PCR System (Applied Biosystems/Life Technologies) utilizing SYBR Green or TaqMan chemistries. The primers used for the PCR reactions are listed in Tables 7 and 8. PCR parameters were: activation at 50° C. for 2 minutes; denature at 95° C. for 10 minutes; followed by 40-42 cycles of 95° C. for 15 seconds and 60° C. for 1 minute (72° C. for amplicons> than 120 bp) followed by dissociation at 95° C. for 15 seconds; 60° C. for 15 seconds, and 95° C. for 15 seconds.

Primers are provided in the Table below:

| Gene | GenBank (NCBI) | FORWARD | |
|---|---|---|---|
| Symbol | Accession number | PRIMER NAME | FORWARD PRIMER (5′->3′) |
| KRT6A | NM_005554.3 | JK1186-KRT6A-F | TGAGGAGTGCAGGCTGAATGGC |
| | | | (SEQ ID NO:) |
| MMP12 | NM_002426.2 | JK1192-MMP12- | TCTGGACTACACATTCAGGAGGCAC |
| | | F | (SEQ ID NO:) |
| MMP11 | NM_005940.3 | JK1178-MMP11- | ACCGCTGGAGCCAGACGCC |
| | | F | (SEQ ID NO:) |
| COL10A1 | NM_000493.3 | ES577-COL10A1- | GGGCCTCAATGGACCCACCG |
| | | F | (SEQ ID NO:) |
| ASCL1 | NM_004316.2 | JK1095-ASCL1-F | AATGGACTTTGGAAGCAGGGTGATC |
| | | | (SEQ ID NO:) |
| C1ORF64 | NM_178840.2 | JK1089-C1orf64- | AGACCAGCTCCGGTGGGAAGC |
| | | F | (SEQ ID NO: 1) |
| FLJ23152 | NM_001190766.1 | JK1087- | TCACGTTGACTACGACTGAGAAGCC |
| | | FLJ23152-F | (SEQ ID NO: 2) |
| SALL4 | NM_020436.3 | JK1016-SALL4-F | CAAAGGCAACTTAAAGGTTCACTAC |
| | | | (SEQ ID NO: 3) |
| AMH | NM_000479.3 | JK1044-AMH-F | CGGAGAGGACTCCCGGC |
| | | | (SEQ ID NO: 4) |
| SLC35D3 | NM_001008783.1 | JK1024- | GCTATTTTGAAAATATGAGTTCTTAGC |
| | | SLC3503-F | (SEQ ID NO: 5) |
| C2orf70 | NM_001105519.1 | JK1010-C2orf70- | CCACCGTCCTGCCTCCTC |
| | | F | (SEQ ID NO: 6) |
| DSCR8 | NR_026838.1 | JK1036-DSCR8-F | ATGCCTAATCCCAGCTTCATC |
| | | | (SEQ ID NO: 7) |
| C1orf56 | NM_001099676.1 | JK1052- | ACTCTAGCTGAGTATATTAGGAATAAC |
| | | C12orf56-F | (SEQ ID NO: 8) |
| SNORD56 | NR_002739.1 | JK1028- | CCACAATGATGGCAATATTTTTC |
| | | SNORD56-F | (SEQ ID NO: 9) |
| OBP2A | NM_014582.2 | JK1070-OBP2A-F | AGCCCTGGGCGGTGGGAAC |
| | | | (SEQ ID NO: 10) |
| SNORD3 | NR_006880.1 | JK1014-SNORD-F | CTATACTTTCAGGGATCATTTCTATA |
| | | | (SEQ ID NO: 11) |
| PPEF1 | NM_152224.1 | JK1032-PPEF1-F | GACCAGATGTTACTTCCAAGATTC |
| | | | (SEQ ID NO: 12) |
| NMU | NM_006681.2 | JK1210-NMU-F | TCTTTTCTGTCCATTGATTCTCAGCCTC |
| | | | (SEQ ID NO: 13) |
| SERPINA1 | NM_000295.3 | SERPINA1-ML-F1 | GGGCAATGCCACCGCCATCT |
| | | | (SEQ ID NO: 14) |
| FLJ30058 | NM_144967.2 | FLJ30058-ML-F1 | CACAACCCCGACCGCAGGAC |
| | | | (SEQ ID NO: 15) |
| SFTPB | NM_000542.2 | JK1156-SFTPB-F | CTCTGTGGCCCAGGCACTGC |
| | | | (SEQ ID NO: 16) |
| ZCCHC12 | NM_173798.2 | JK1158- | TCCACCAGCGGAGCACAGGCC |
| | | ZCCHC12-F | (SEQ ID NO: 17) |
| IGSF1 | NM_001555.2 | JK1132-IGSF1-F | GGGCCTTCAACTACCATCCCAC |
| | | | (SEQ ID NO: 18) |
| S100A2 | NM_005978.3 | S100A2-F1 | TCTGCCACCTGGTCTGCCACA |
| | | | (SEQ ID NO: 19) |

| Gene Symbol | REVERSE PRIMER NAME | REVERSE PRIMER (5′->3′) |
|---|---|---|
| KRT6A | JK1187-KRT6A-R | CAATGGCTCTGCCACTGCTGGAAC (SEQ ID NO: 20) |
| MMP12 | JK1193-MMP12-R | GTCACAGAGAGCTGGTTCTGAATTGTC (SEQ ID NO: 21) |
| MMP11 | JK1179-MMP11-R | CGAGAGGCCAATGCTGGGTAGC (SEQ ID NO: 22) |
| COL10A1 | ES578-COL10A1-R | CTGGGCCTTTGGCCTGCCTT (SEQ ID NO: 23) |
| ASCL1 | JK1096-ASCL1-R | TAGTTGGCGATGGGGTTGGTTGAC (SEQ ID NO: 24) |
| C1ORF64 | JK1090-C1orf64-R | CCCACAGAACACGATGTAAGTCTTC (SEQ ID NO: 25) |
| FLJ23152 | JK1088-FLJ23152-R | GTCTTCCTGCGCTCCTCGGC (SEQ ID NO: 26) |
| SALL4 | JK1017-SALL4-R | AGTTCTCCCTTAGCTGACCGC (SEQ ID NO: 27) |
| AMH | JK1045-AMH-R | GGATGGCCTGGGCGGC (SEQ ID NO: 28) |
| SLC35D3 | JK1025-SLC35D3-R | CTTTACAGGTGGTCCCTCTTC (SEQ ID NO: 29) |
| C2orf70 | JK1011-C2orf70-R | CATCAGGCTCTGCTCTGAAC (SEQ ID NO: 30) |
| DSCR8 | JK1037-DSCR8-R | GAAAATGTATGAGCCAGCCTTC (SEQ ID NO: 31) |
| C1orf56 | JK1053-C12orf56-R | ATGGGGTAACACAATGGGAGC (SEQ ID NO: 32) |
| SNORD56 | JK1029-SNORD56-R | TCACTCAGACCCAGAGTC (SEQ ID NO: 33) |
| OBP2A | JK1071-OBP2A-R | TTCCTGCCCCCATAGGCGCTGA (SEQ ID NO: 34) |
| SNORD3 | JK1015-SNORD-R | CAATACGGAGAGAAGAACGATC (SEQ ID NO: 35) |
| PPEF1 | JK1033-PPEF1-R | TCACCACCTTCCCATCATGAC (SEQ ID NO: 36) |
| NMU | JK1211-NMU-R | CTCTCATGCAGGTGAGGAACGAGC (SEQ ID NO: 37) |
| SERPINA1 | SERPINA1-ML-R1 | GCCCAGTTGACCCAGGACGC (SEQ ID NO: 38) |
| FLJ30058 | FLJ30058-ML-R1 | ACAGGAAATGTCTGGCCACGAGT (SEQ ID NO: 39) |
| SFTPB | JK1157-SFTPB-R | ACACTCTTGGCATAGGTCATCGGC (SEQ ID NO: 40) |
| ZCCHC12 | JK1159-ZCCHC12-R | TGCCTTCCTATCTCAGCAGGGGAC (SEQ ID NO: 41) |
| IGSF1 | JK1133-IGSF1-R | GGCACCAAAGCGTGATGTTCTCC (SEQ ID NO: 42) |
| S100A2 | S100A2-R1 | AGTGACCAGCACAGCCAGCG (SEQ ID NO: 43) |

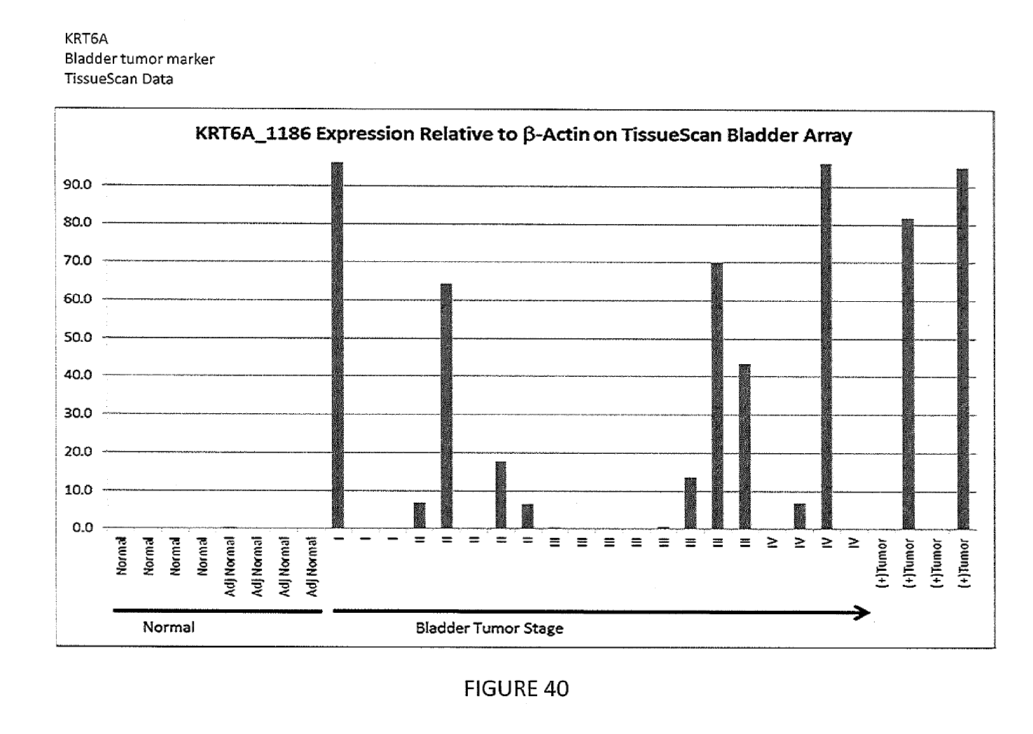

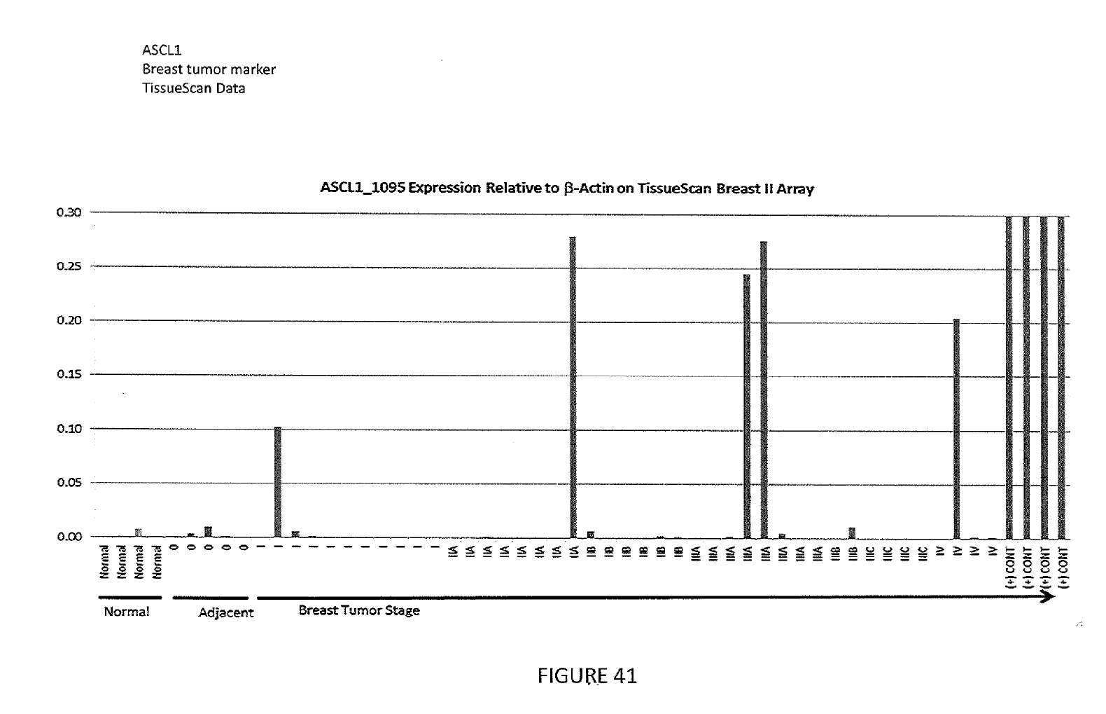

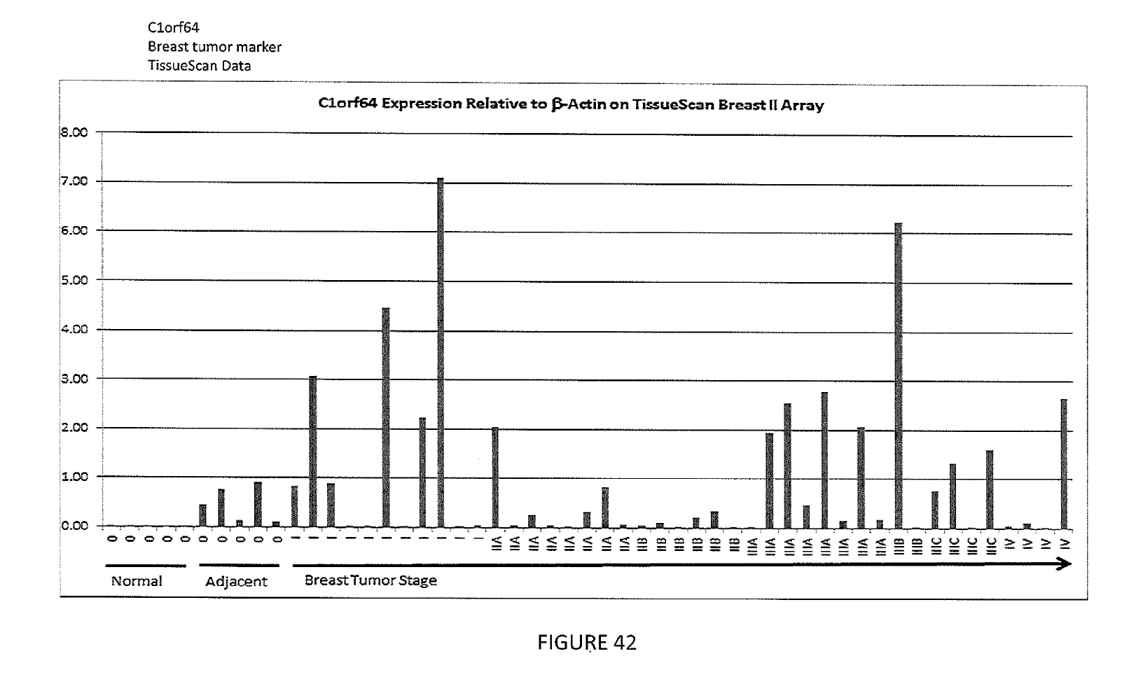

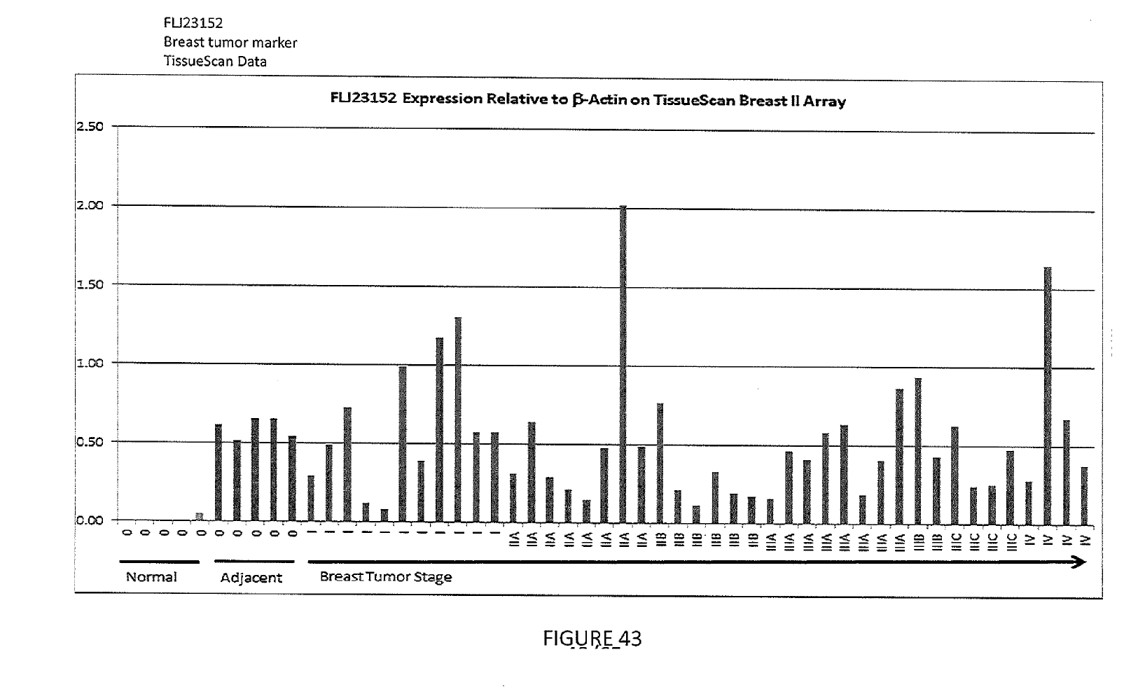

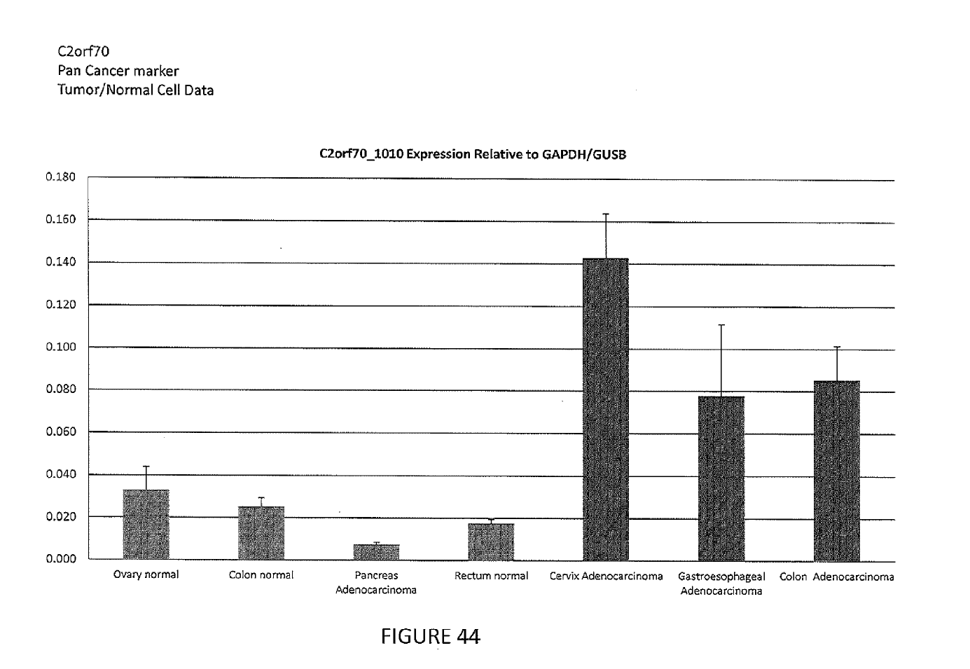

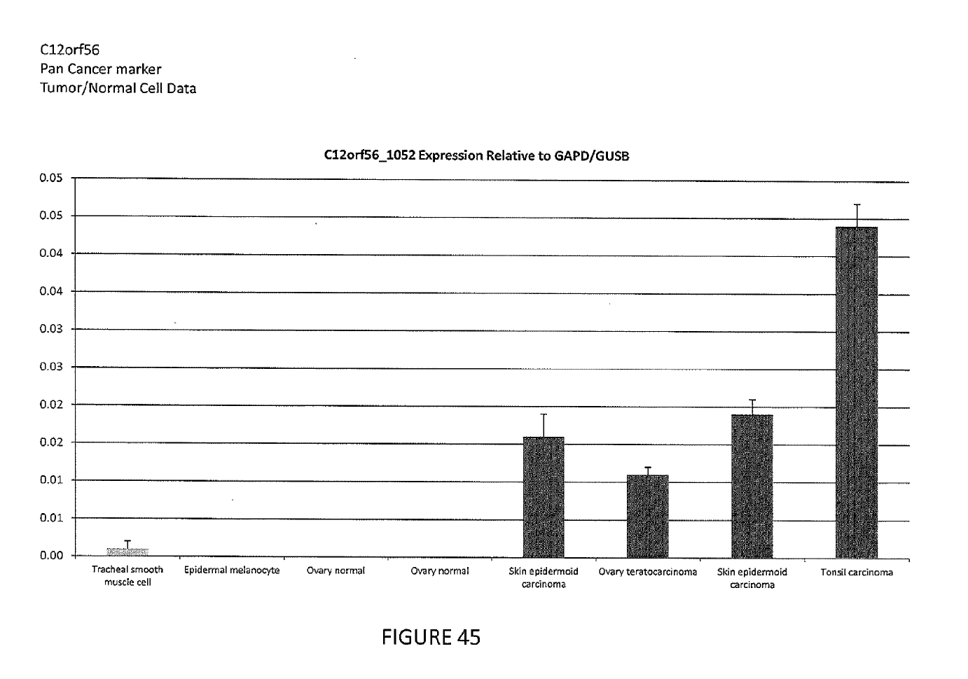

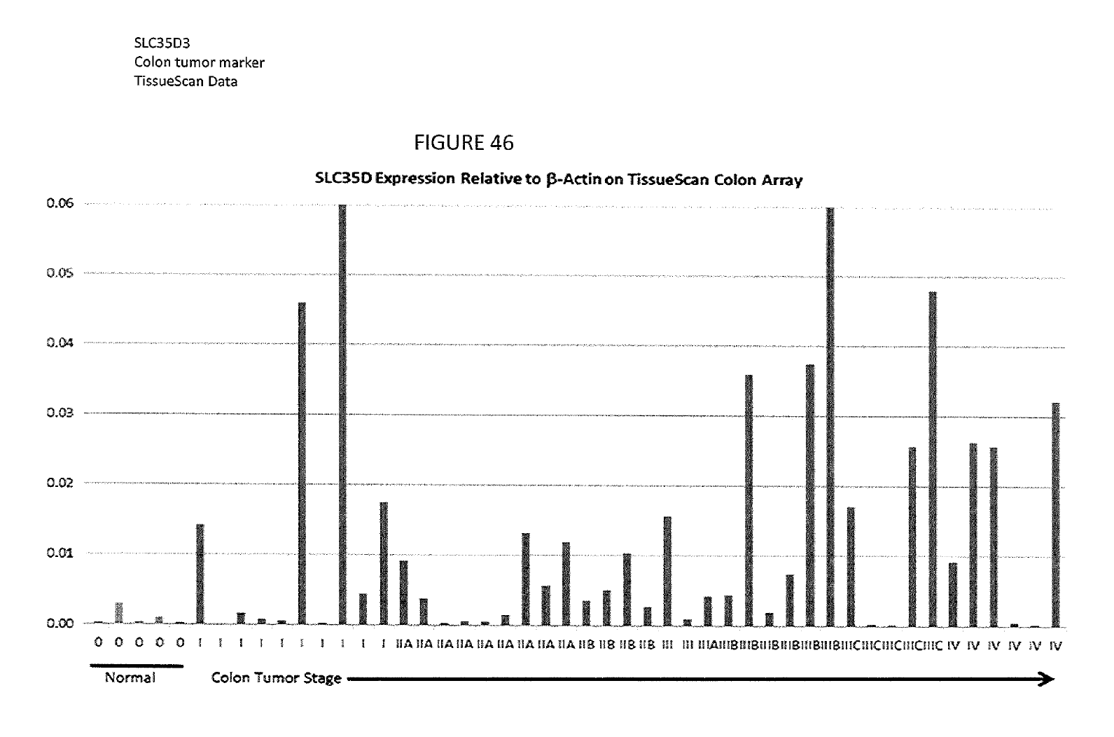

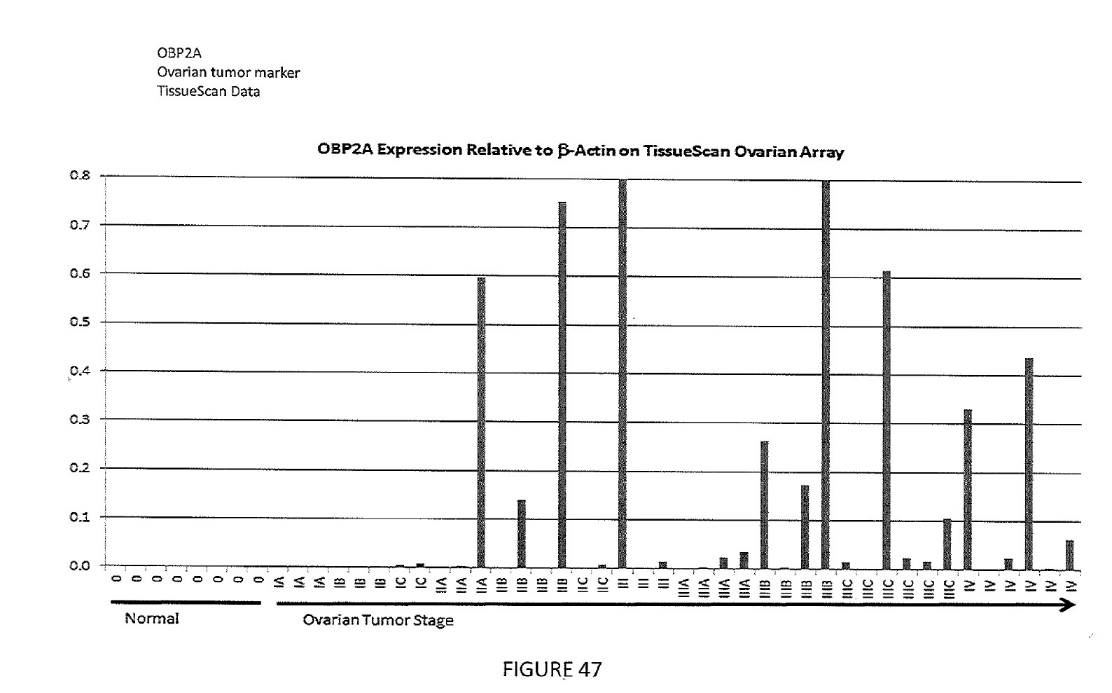

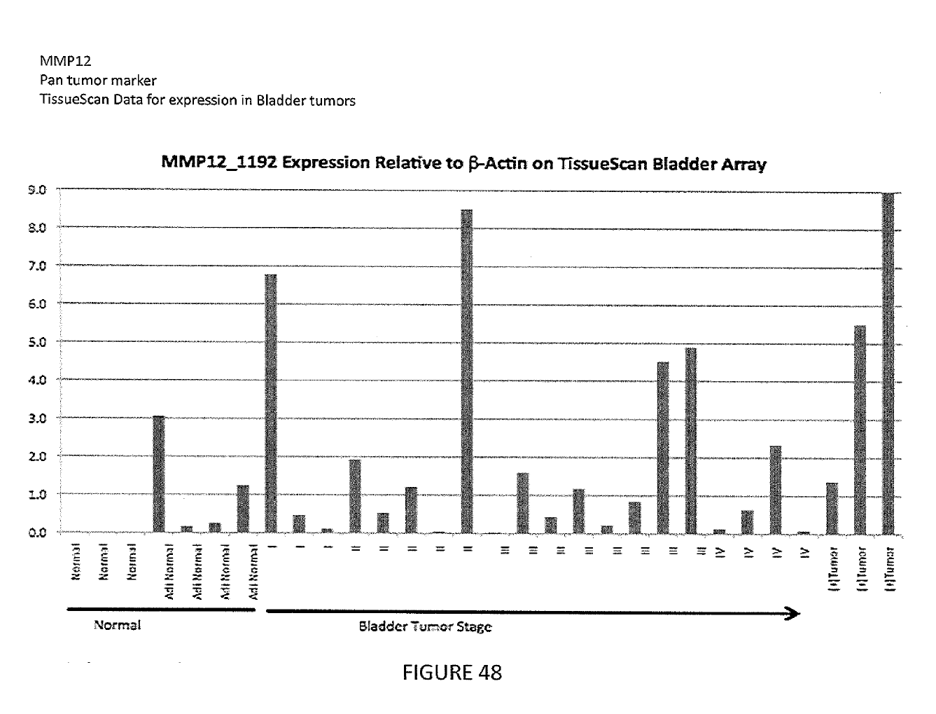

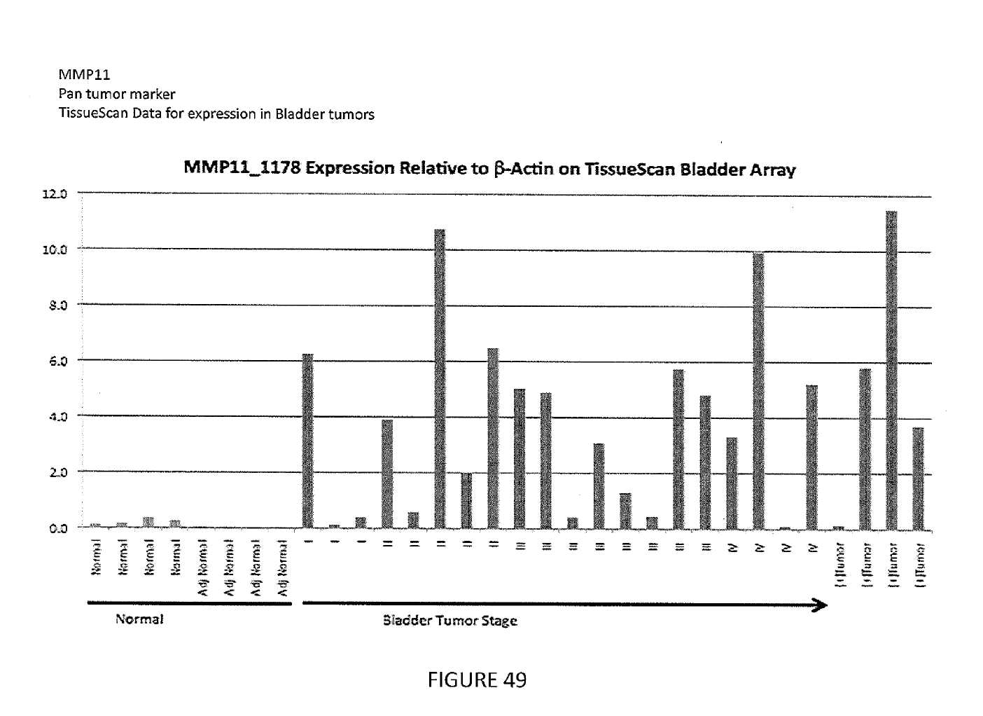

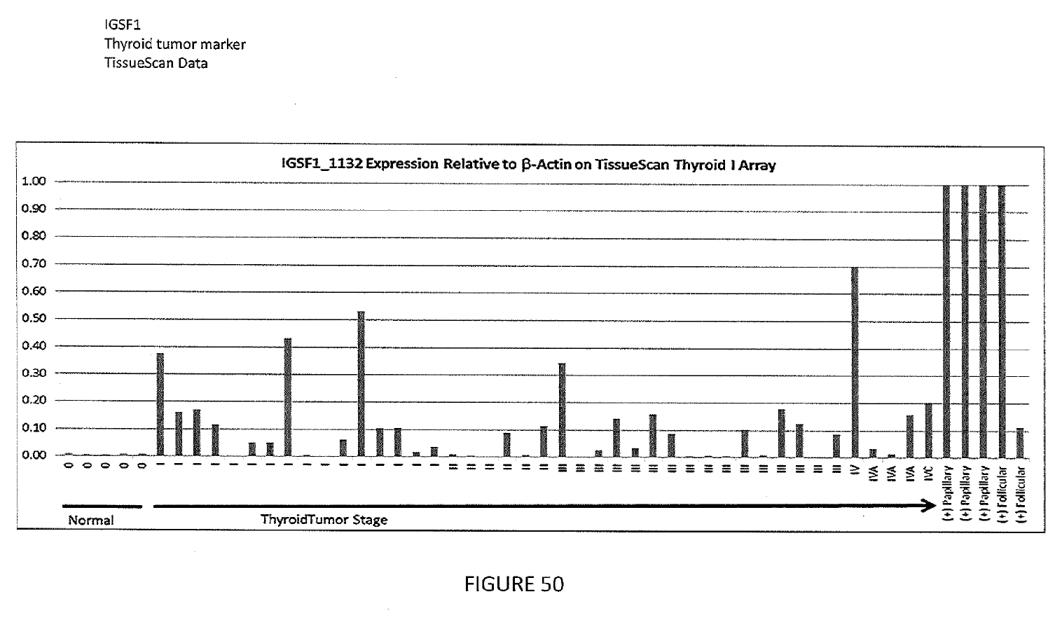

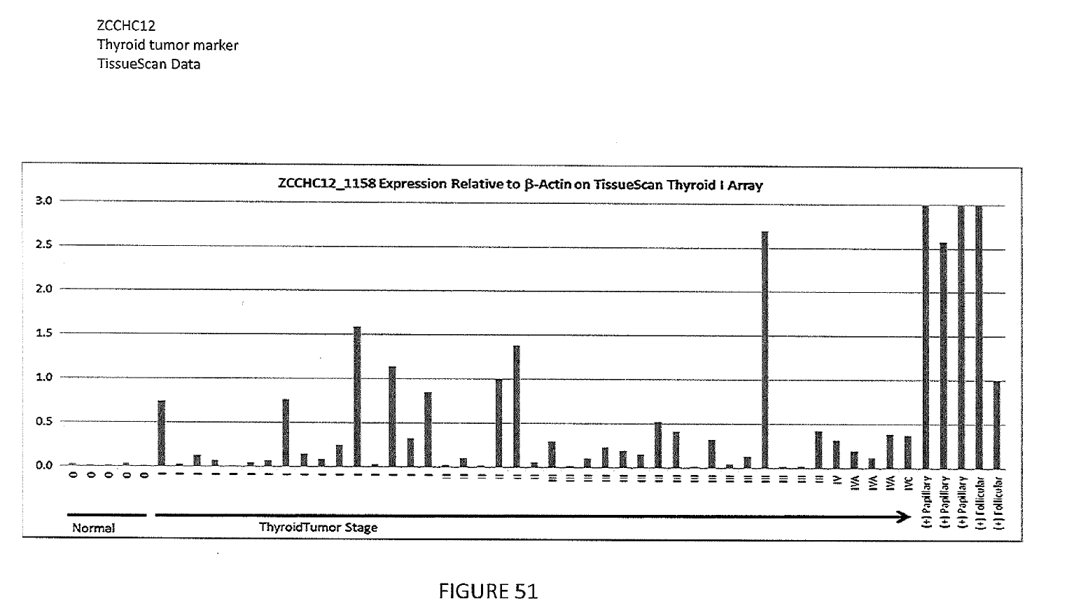

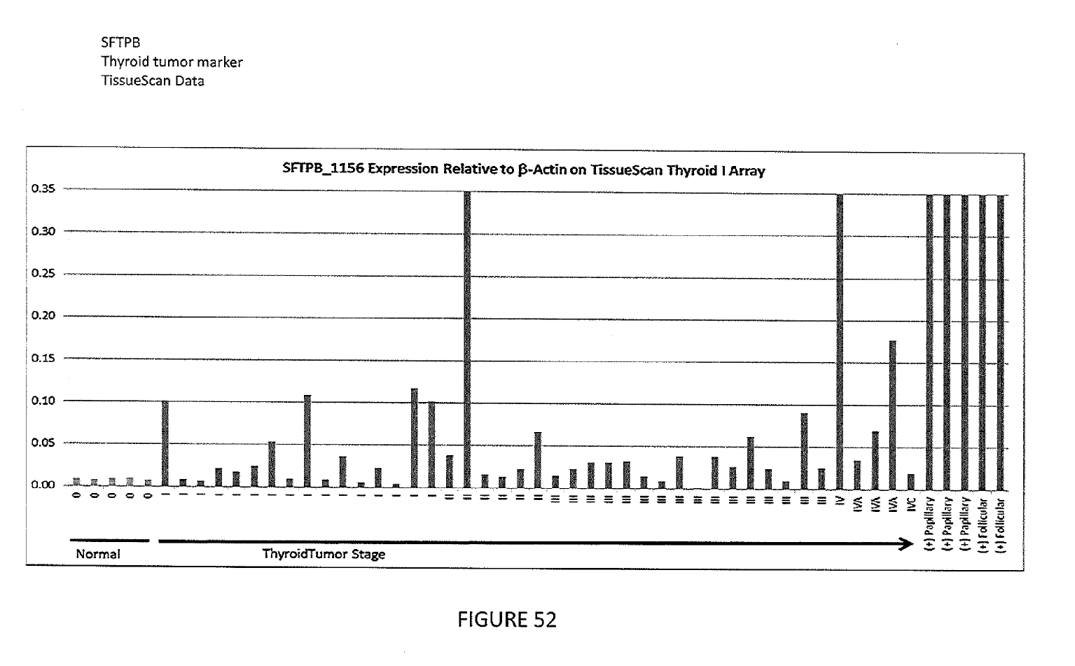

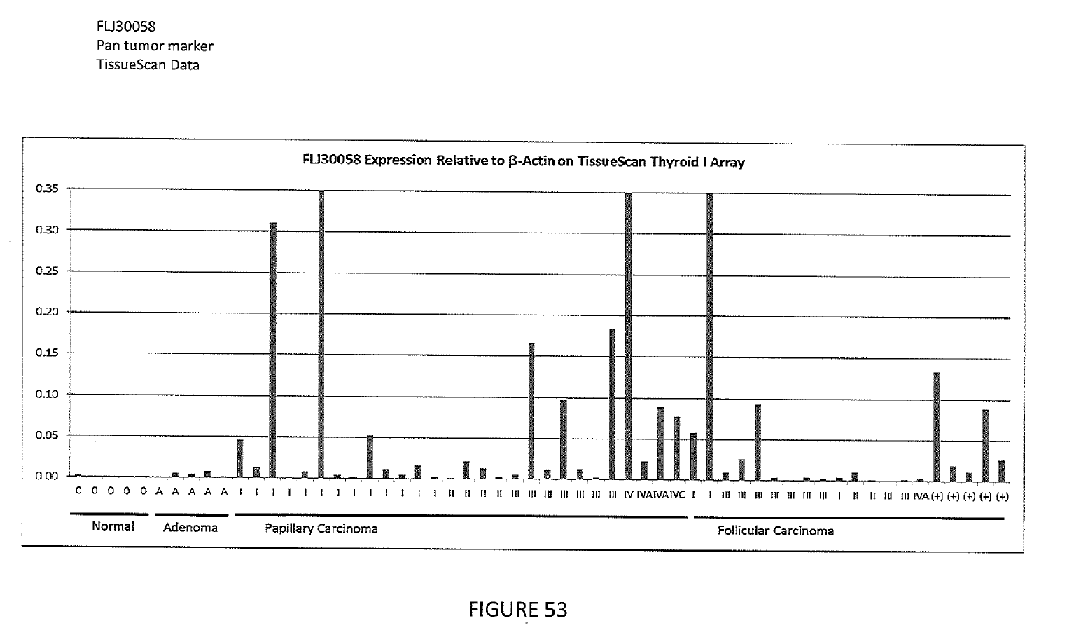

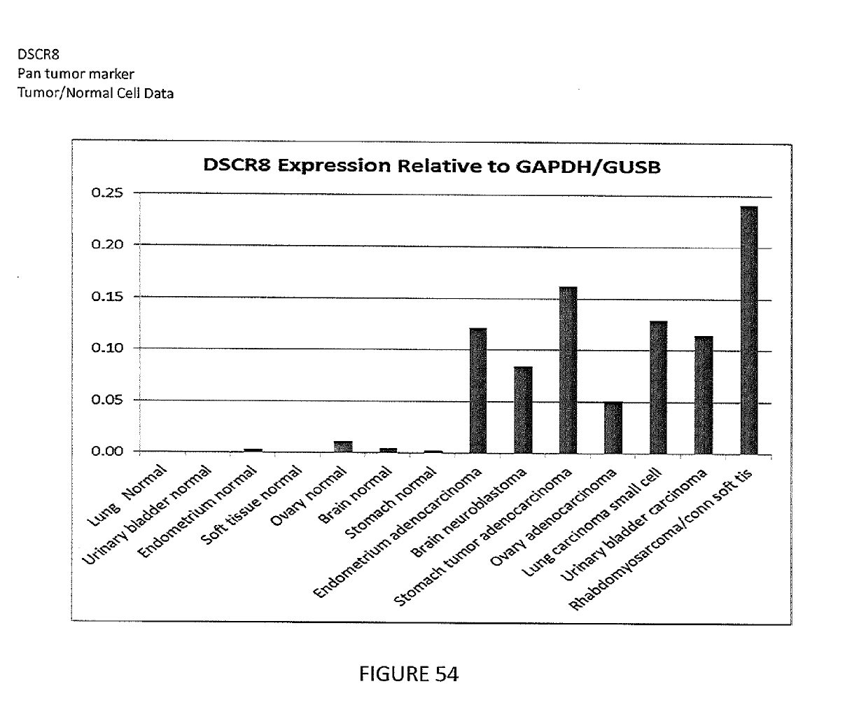

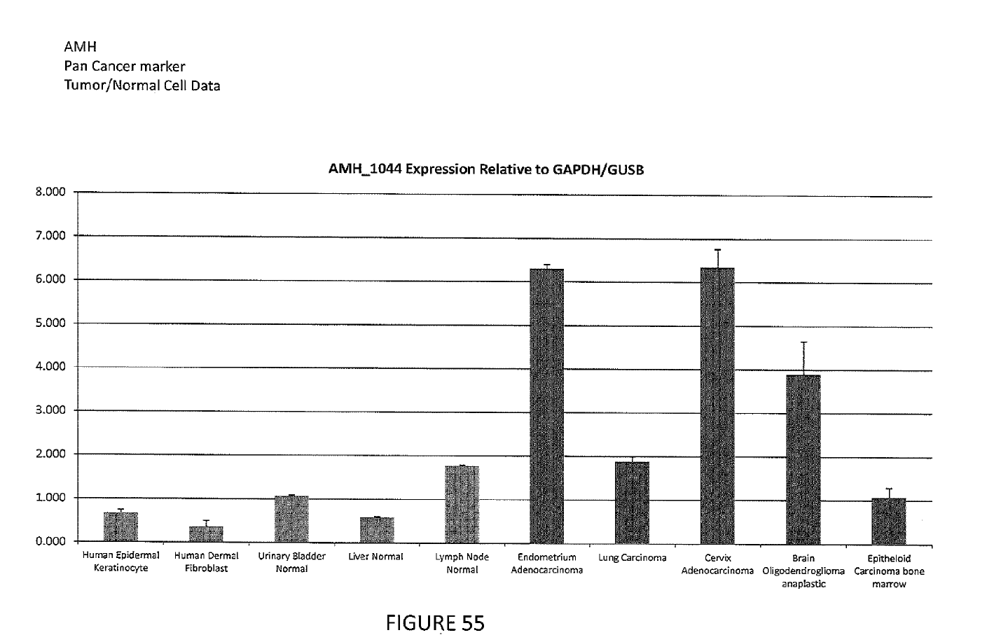

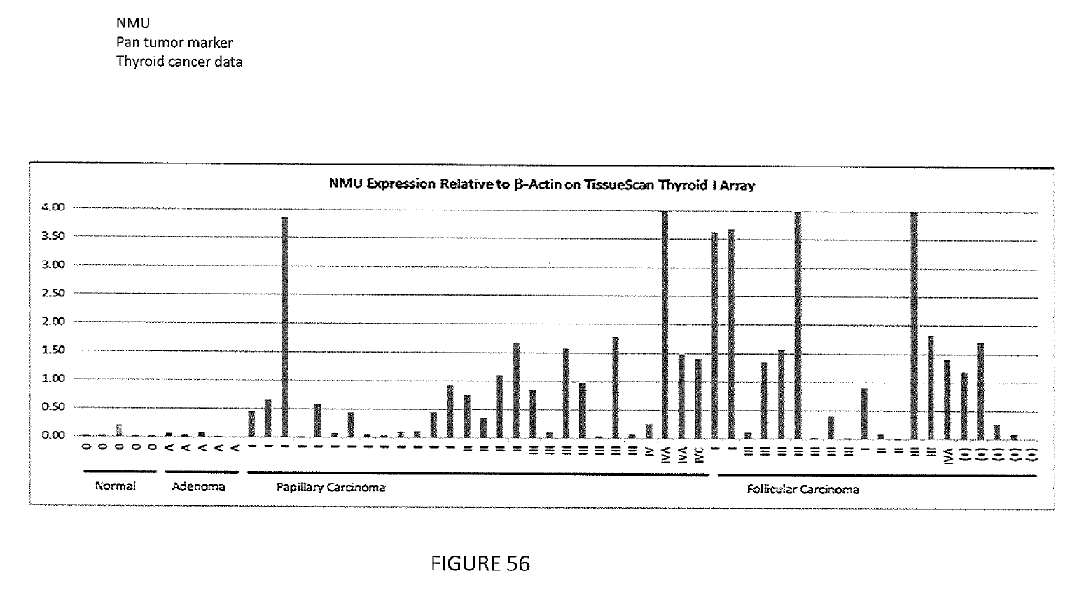

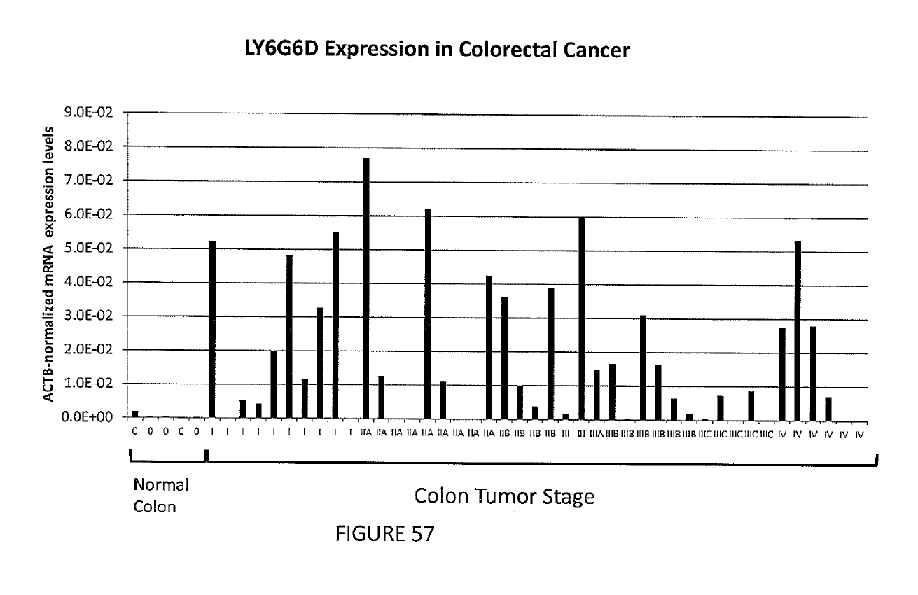

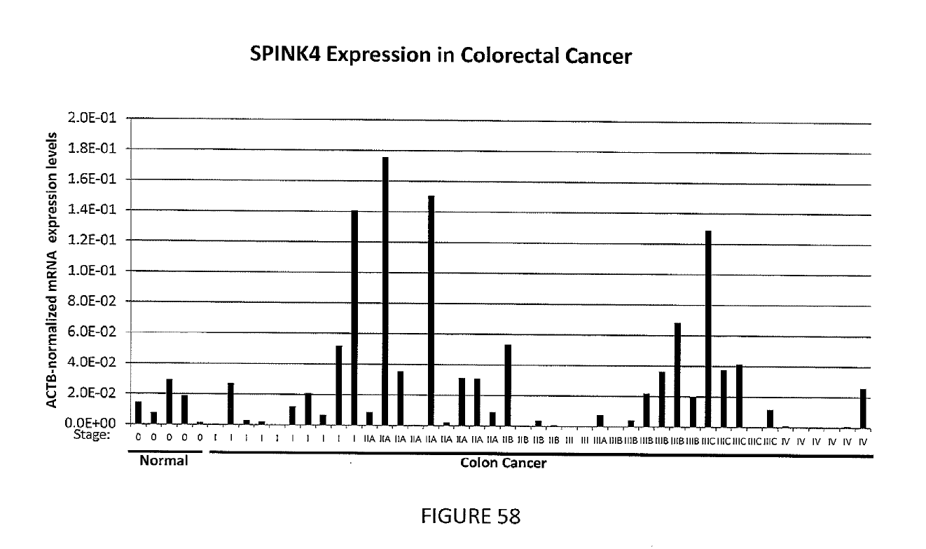

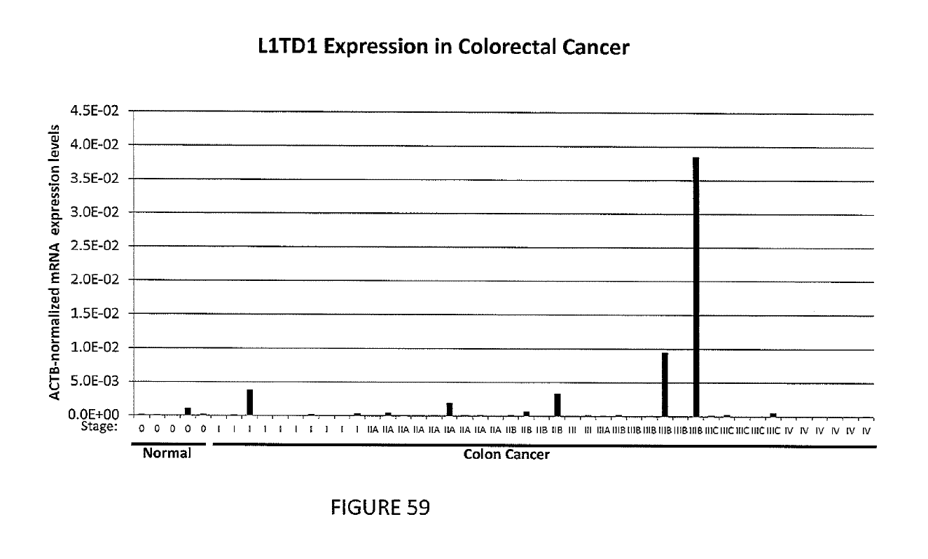

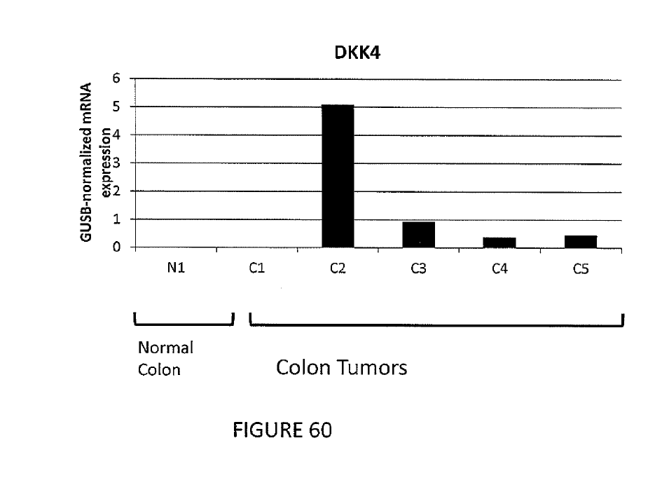

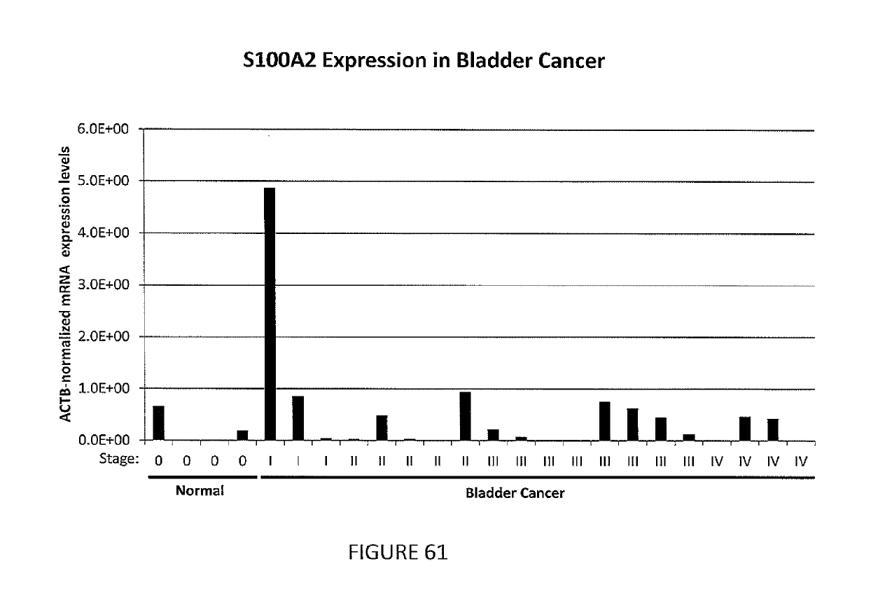

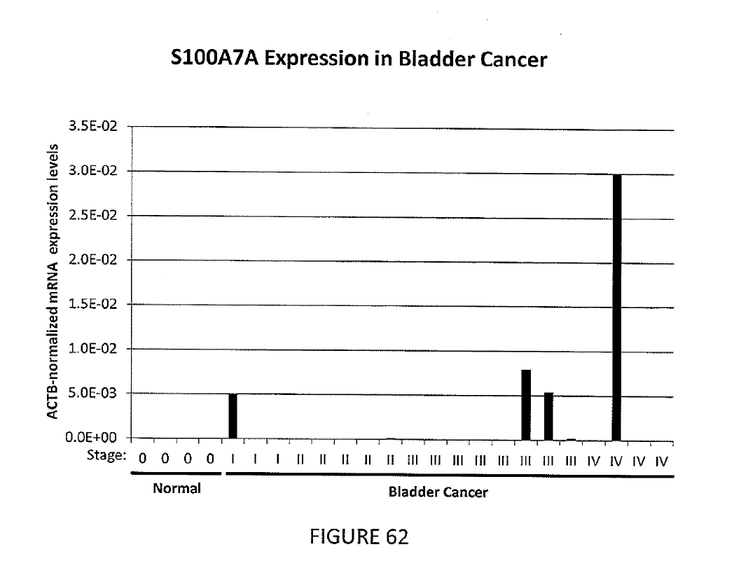

The results are provided in FIGS. 40-62 and showed that KRT6A, ASCL1, C1orf64, FLJ23152, C2orf70, C12orf56, SLC35D, OBP2A, MMP12, MMP11, IGSF, ZCCHC12, SFTPB, FLJ30058, DSCR8, AMH, NMU, LY6G6D, SPINK4, L1TD1, DKK4, S100A2 ands S100A7a were all elevated in cancer tissue relative to normal tissue (normalized to β-actin expression). Moreover, the signal pattern seen for the positive controls previously analyzed by microarray, was the same obtained by microarray confirming that the PCR reaction worked.

### Example 16

### Microarray Analysis of Thyroid Tissue

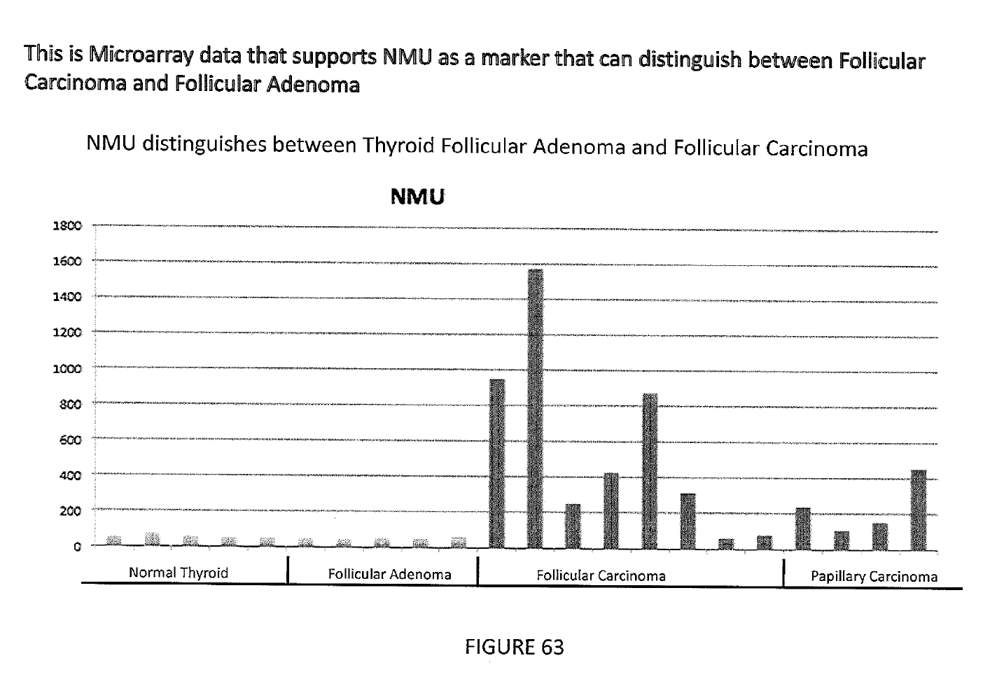

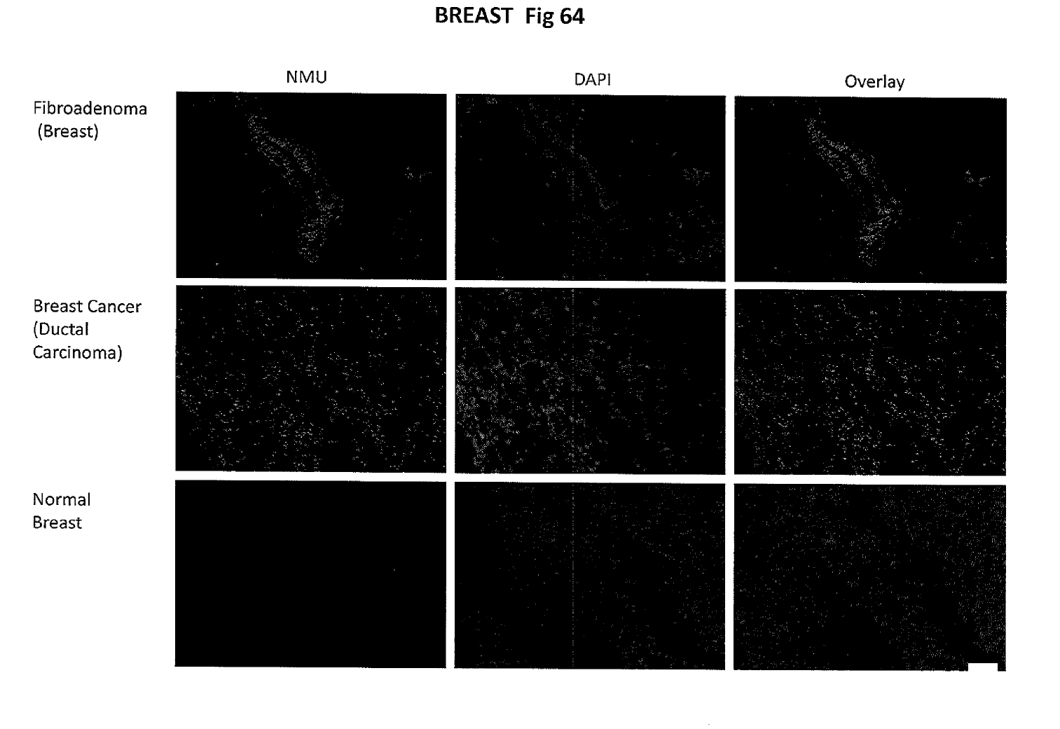

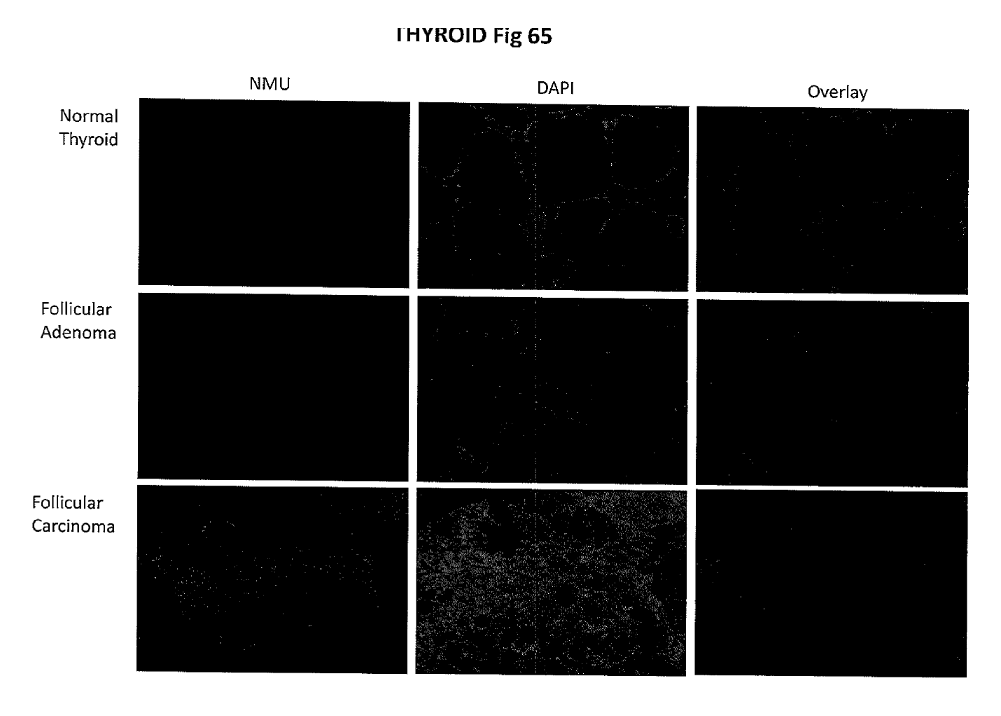

RNA was obtained from normal thyroid, thyroid carcinoma, and thyroid follicular adenoma and analyzed on Illumina gene expression microarrays. The results are shown in FIG. 63 and indicate that NMU can distinguish between malignant thyroid carcinoma and both benign thyroid follicular adenoma as well as normal thyroid tissue.

### Example 17

### Immunofluorescence Microscopy

Paraffin embedded tissue sections were obtained from Asterand (Detroit, Mich.). These specimens included: Normal breast tissue (donors with no history of cancer), fibroadenoma of the breast, breast ductal cell carcinoma, normal thyroid tissue (donors with no history of cancer), thyroid follicular adenoma and thyroid follicular carcinoma, Prior to the staining with antibodies, the sections were dewaxed in xylene and rehydrated in cycles of ethanol (100%, 95%, 70%) followed by a wash in distilled water. Antigen retrieval was performed in epitope retrieval buffer (IHC World #IW-1100) by incubating the slides at 95° C. 40 minutes using an IHC-Steamer Set (IHC World #IW-1102). Immunostaining was performed using a polyclonal rabbit anti-human NMU antibody (Abeam #ab92693) at a 1:100 dilution. The primary antibody was detected using an Alexa Fluor 594 Donkey anti-rabbit IgG (Life Sciences #A21207) at a 1:200 dilution.

Vectashield mounting medium with DAPI was used to preserve the stained samples (Vector Laboratories #H-1200). Images were taken with an exposure time of 400 milliseconds using a Nikon Eclipse TE2000-U at a magnification of 10,000 and an X-Cite 120 fluorescence illumination system (Lumen Dynamics).

The results provided in FIG. 64 how that NMU is detected in breast cancer, but not normal breast tissue. The results presented in FIG. 65 show that NMU can distinguish between malignant thyroid follicular carcinoma and benign thyroid follicular adenoma.

Although the present invention has been described in considerable detail with reference to certain preferred embodiments thereof, other versions are possible. Therefore the spirit and scope of the appended claims should not be limited to the description and the preferred versions contained within this specification.

We claim:

1-2. (canceled)

3. A kit comprising a plurality of agents that bind to a plurality of markers chosen from CXCL10, CXCL9, MMP7, MMP12, MMP9, EPYC, IL8, NMU, WNT10A, ASC11, C1orf64, and FLJ23152.

4. The kit of claim 3 comprising a plurality of agents that binds to each of the following markers CXCL10, CXCL9, MMP7, MMP12, MMP9, EPYC, IL8, NMU, WNT10A, ASC11, C1orf64, and FLJ23152.

5. The kit of claim 3 wherein the plurality of agents are proteins and/or peptides.

6. The kit of claim 5, wherein the proteins are antibodies and/or antibody fragments.

7. The kit of claim 3, wherein the one or more agents is a plurality of agents.

8. The kit of claim 3, wherein the plurality of agents are nucleic acid oligonucleotides.

9. The kit of claim 8, wherein the nucleic acid oligonucleotides are DNA oligonucleotides.

10. The kit of claim 8, wherein the nucleic acid oligonucleotides bind to a DNA sequence encoding a plurality of markers chosen from CXCL10, CXCL9, MMP7, MMP12, MMP9, EPYC, IL8, NMU, WNT10A, ASC11, C1orf64, and FLJ23152.

11. The kit of claim 4, wherein the plurality of agents are proteins and/or peptides.

12. The kit of claim 11, wherein the proteins are antibodies and/or antibody fragments.

13. The kit of claim 4, wherein the one or more agents is a plurality of agents.

14. The kit of claim 4, wherein the plurality of agents are nucleic acid oligonucleotides.

15. The kit of claim 14, wherein the nucleic acid oligonucleotides are DNA oligonucleotides.

16. The kit of claim 14, wherein the nucleic acid oligonucleotides bind to a DNA sequence encoding a plurality of markers chosen from CXCL10, CXCL9, MMP7, MMP12, MMP9, EPYC, IL8, NMU, WNT10A, ASC11, C1orf64, and FLJ23152.

17. The use of the kit of claim 3 to detect breast cancer.

18. The use of the kit of claim 4 to detect breast cancer.


SEQUENCE LISTING (555 sequences)

SEQ ID NO: 1 - DNA, 21 bp, Homo sapiens
agaccagctccggtgggaagc

SEQ ID NO: 2 - DNA, 25 bp, Homo sapiens
tcacgttgactacgactgagaagcc

SEQ ID NO: 3 - DNA, 25 bp, Homo sapiens
caaaggcaacttaaaggttcactac

SEQ ID NO: 4 - DNA, 17 bp, Homo sapiens
cggagaggactcccggc

SEQ ID NO: 5 - DNA, 27 bp, Homo sapiens
gctattttgaaaatatgagttcttagc

SEQ ID NO: 6 - DNA, 18 bp, Homo sapiens
ccaccgtcctgcctcctc

SEQ ID NO: 7 - DNA, 21 bp, Homo sapiens
atgcctaatcccagcttcatc

SEQ ID NO: 8 - DNA, 27 bp, Homo sapiens
actctagctgagtatattaggaataac

SEQ ID NO: 9 - DNA, 23 bp, Homo sapiens
ccacaatgatggcaatatttttc

SEQ ID NO: 10 - DNA, 19 bp, Homo sapiens
agccctgggcggtgggaac

SEQ ID NO: 11 - DNA, 26 bp, Homo sapiens
ctatactttcagggatcatttctata

SEQ ID NO: 12 - DNA, 24 bp, Homo sapiens
gaccagatgttacttccaagattc

SEQ ID NO: 13 - DNA, 28 bp, Homo sapiens
tcttttctgtccattgattctcagcctc

SEQ ID NO: 14 - DNA, 20 bp, Homo sapiens
gggcaatgccaccgccatct

SEQ ID NO: 15 - DNA, 20 bp, Homo sapiens
cacaaccccgaccgcaggac

SEQ ID NO: 16 - DNA, 20 bp, Homo sapiens
ctctgtggcccaggcactgc

SEQ ID NO: 17 - DNA, 21 bp, Homo sapiens
tccaccagcggagcacaggcc

SEQ ID NO: 18 - DNA, 22 bp, Homo sapiens
gggccttcaactaccatcccac

SEQ ID NO: 19 - DNA, 21 bp, Homo sapiens
tctgccacctggtctgccaca

SEQ ID NO: 20 - DNA, 24 bp, Homo sapiens
caatggctctgccactgctggaac

SEQ ID NO: 21 - DNA, 27 bp, Homo sapiens
gtcacagagagctggttctgaattgtc

SEQ ID NO: 22 - DNA, 22 bp, Homo sapiens
cgagaggccaatgctgggtagc

SEQ ID NO: 23 - DNA, 20 bp, Homo sapiens
ctgggcctttggcctgcctt

SEQ ID NO: 24 - DNA, 24 bp, Homo sapiens
tagttggcgatggggttggttgac

SEQ ID NO: 25 - DNA, 25 bp, Homo sapiens
cccacagaacacgatgtaagtcttc

SEQ ID NO: 26 - DNA, 20 bp, Homo sapiens
gtcttcctgcgctcctcggc

SEQ ID NO: 27 - DNA, 21 bp, Homo sapiens
agttctcccttagctgaccgc

SEQ ID NO: 28 - DNA, 16 bp, Homo sapiens
ggatggcctgggcggc

SEQ ID NO: 29 - DNA, 21 bp, Homo sapiens
ctttacaggtggtccctcttc

SEQ ID NO: 30 - DNA, 20 bp, Homo sapiens
catcaggctctgctctgaac

SEQ ID NO: 31 - DNA, 22 bp, Homo sapiens
gaaaatgtatgagccagccttc

SEQ ID NO: 32 - DNA, 21 bp, Homo sapiens
atggggtaacacaatgggagc

SEQ ID NO: 33 - DNA, 18 bp, Homo sapiens
tcactcagacccagagtc

SEQ ID NO: 34 - DNA, 22 bp, Homo sapiens
ttcctgcccccataggcgctga

SEQ ID NO: 35 - DNA, 22 bp, Homo sapiens
caatacggagagaagaacgatc

SEQ ID NO: 36 - DNA, 21 bp, Homo sapiens
tcaccaccttcccatcatgac

SEQ ID NO: 37 - DNA, 24 bp, Homo sapiens
ctctcatgcaggtgaggaacgagc

SEQ ID NO: 38 - DNA, 20 bp, Homo sapiens
gcccagttgacccaggacgc

SEQ ID NO: 39 - DNA, 23 bp, Homo sapiens
acaggaaatgtctggccacgagt

SEQ ID NO: 40 - DNA, 24 bp, Homo sapiens
acactcttggcataggtcatcggc

SEQ ID NO: 41 - DNA, 24 bp, Homo sapiens
tgccttcctatctcagcaggggac

SEQ ID NO: 42 - DNA, 23 bp, Homo sapiens
ggcaccaaagcgtgatgttctcc

SEQ ID NO: 43 - DNA, 20 bp, Homo sapiens
agtgaccagcacagccagcg

SEQ ID NO: 44 - DNA, 20 bp, Homo sapiens
gggcctcaatggacccaccg

SEQ ID NO: 45 - DNA, 25 bp, Homo sapiens
aatggactttggaagcagggtgatc

SEQ ID NO: 46 - DNA, 50 bp, Homo sapiens
gctgcagctcgttcctcacctgcatgagagaagaatgaagagattcagag

SEQ ID NO: 47 - DNA, 50 bp, Homo sapiens
tggggacagaaccttctatgacccggagcccatcctgtgcccctgtttca

SEQ ID NO: 48 - DNA, 50 bp, Homo sapiens
gacccacgtgctgtatcctgtccccctggagagttatgaggacatccatg

SEQ ID NO: 49 - DNA, 50 bp, Homo sapiens
gcggcactgatgggctcacatatacgaatgaatgccagctctgcttggcc

SEQ ID NO: 50 - DNA, 50 bp, Homo sapiens
gtagagggtgtttgcagagcaatgcccgtaatgcttagagaatgttctcc

SEQ ID NO: 51 - DNA, 50 bp, Homo sapiens
gtagctgagtttaacatgtgtggtcttggtattcttaagggaacttccac

SEQ ID NO: 52 - DNA, 50 bp, Homo sapiens
cttcggcatggcctctttacagggcaccttctgctctcaggttgggtgac

SEQ ID NO: 53 - DNA, 50 bp, Homo sapiens
cctgatgcctgagatcctgggtcgtgaccagcctggcttgcttggaataa

SEQ ID NO: 54 - DNA, 50 bp, Homo sapiens
gcggtcagctaagggagaacttgcgtggaaggagcaatgcagacacagtg

SEQ ID NO: 55 - DNA, 50 bp, Homo sapiens
actgaaacccagccagaagagggaccacctgtaaagcaagtcctttcaag

SEQ ID NO: 56 - DNA, 50 bp, Homo sapiens
tgcagcagctaccgccctgacctgtctcttgccaggactgtggagcggat

SEQ ID NO: 57 - DNA, 50 bp, Homo sapiens
tcacgtgtcttcacgcatctcttgaattggaaattgtgccctggagactg

SEQ ID NO: 58 - DNA, 50 bp, Homo sapiens
ccctgcaagtcagccccatctgctgttccttggtctctaatcacctgagc

SEQ ID NO: 59 - DNA, 50 bp, Homo sapiens
caggtcttggtaggtgcctgcatctgtctgccttctggctgacaatcctg

SEQ ID NO: 60 - DNA, 50 bp, Homo sapiens
gtagagcaccgaaaaccccgaggaagagaggtagcgttttctcctgagcg

SEQ ID NO: 61 - DNA, 50 bp, Homo sapiens
ctcatcagcctgtcggaggaacgcatcagcgcgcaccacgtgcccaacat

SEQ ID NO: 62 - DNA, 50 bp, Homo sapiens
ttccaccccaagagaactcgcgctcagtaaacgggaacatgccccctgca

SEQ ID NO: 63 - DNA, 50 bp, Homo sapiens
ttccagggcacgagttcgaggccagcctggtccacatgggtcggaaaaaa

SEQ ID NO: 64 - DNA, 50 bp, Homo sapiens
ttcgtcaacagcagttcacctagtgagtgttgagactctgggtctgagtg

SEQ ID NO: 65 - DNA, 50 bp, Homo sapiens
tcccgagggatggaaatccgagcctgcaacctgctccgtcaaaggttcag

SEQ ID NO: 66 - DNA, 50 bp, Homo sapiens
gaagccggctttctggcgttgcttggctgcaactgccgtcagccattgat

SEQ ID NO: 67 - DNA, 50 bp, Homo sapiens
ctgcaactgccgtcagccattgatgatcgttcttctctccgtattgggga

SEQ ID NO: 68 - DNA, 50 bp, Homo sapiens
ggggcacaactcactactctgacaacaacagccctgcgagcaacatagtt

SEQ ID NO: 69 - DNA, 50 bp, Homo sapiens
tcacgtgtcttcacgcatcccttgaattggaaattgtgccctggagactg

SEQ ID NO: 70 - DNA, 50 bp, Homo sapiens
ggccagtggaaattcaggtgaaaatgttcatcaattcccattgcatcacc

SEQ ID NO: 71 - DNA, 50 bp, Homo sapiens
tcccacttggcaggggccgtcttgtccactcgtttctgtaaacatgggtg

SEQ ID NO: 72 - DNA, 50 bp, Homo sapiens
ccacaaaatctgtcacagcagggcttttcaacactgggagttaatccagg

SEQ ID NO: 73 - DNA, 50 bp, Homo sapiens
ggtggaggaaccactgagtcaggagagcgagatggaagaaccactgagtc

SEQ ID NO: 74 - DNA, 50 bp, Homo sapiens
gaaggctggctcatacattttcccagacaggaatttggctgccaacaggg

SEQ ID NO: 75 - DNA, 50 bp, Homo sapiens
gatgtcgctactattccaacctccgcctgcctctgatgtactcccacccc

SEQ ID NO: 76 - DNA, 50 bp, Homo sapiens
cagatgtgtgtgcttgggcgtgtttctcgtgtcgcgtttgcgtgtcggct

SEQ ID NO: 77 - DNA, 50 bp, Homo sapiens
tgggttggacctagtggtgttgtcgtgagtgccacctaaccaggaggcca

SEQ ID NO: 78 - DNA, 50 bp, Homo sapiens
agtggacttagcccctgtttgctcctccgataactggggtgaccttggtt

SEQ ID NO: 79 - DNA, 50 bp, Homo sapiens
gacactgctcaagctccagaaatcttccagcgttgcgactgtggccctgg

SEQ ID NO: 80 - DNA, 50 bp, Homo sapiens
gaaccactgagtcaggagagccaggtggaggaaccaccgagtcaggagag

SEQ ID NO: 81 - DNA, 50 bp, Homo sapiens
ggctggcttagaaccctcaaaacccaaacacctcaccaactcggcatgtg

SEQ ID NO: 82 - DNA, 50 bp, Homo sapiens
gtacagttttgctcaggtcacgccaacagggaaacctcaagtgtaggtct

SEQ ID NO: 83 - DNA, 50 bp, Homo sapiens
gccaggtggaggaaccactgagtcaggagagcgagatggaagaactaccg

SEQ ID NO: 84 - DNA, 50 bp, Homo sapiens
cgagagctggagagaagaaggtttcccagtgggcttgaagcttgctgtgc

SEQ ID NO: 85 - DNA, 50 bp, Homo sapiens
tgcagccatgtaagcaagtacctttcacctcccgccatgattctgaggcc

SEQ ID NO: 86 - DNA, 50 bp, Homo sapiens
tgggggaggagacccttggaaaagtcctctcttcccagctcctgattctg

SEQ ID NO: 87 - DNA, 50 bp, Homo sapiens
agagctttgggctcaacaagggctttcacttgccattgcagaaggtcctg

SEQ ID NO: 88 - DNA, 50 bp, Homo sapiens
agtagctcttgcggaaacgtgtagatactggtctagtgggtctgtgaacc

SEQ ID NO: 89 - DNA, 50 bp, Homo sapiens
tgctaaggaaggagaccaggaagccaccctaacactcggccagacccgct

SEQ ID NO: 90 - DNA, 50 bp, Homo sapiens
gaccatgcatgtgtccccaaacctagttctttccctaggtctggtttcat

SEQ ID NO: 91 - DNA, 50 bp, Homo sapiens
ccacatccctctgccactcccagaaaatctacgagcccttcacctccaga

SEQ ID NO: 92 - DNA, 50 bp, Homo sapiens
tagtggaacaaaattgaagggtggtcagtagtttcatttccttgtcctgc

SEQ ID NO: 93 - DNA, 50 bp, Homo sapiens
gcagtgcctgcttttcaagggcctgacatggaagcttttcaacaggaact

SEQ ID NO: 94 - DNA, 50 bp, Homo sapiens
ctttccgccatcttgattctttgtcactgaccgagactcagccgtgggaa

SEQ ID NO: 95 - DNA, 50 bp, Homo sapiens
gggactctgcccacttttgatcccactaaagtgctggaagcaggtgaagg

SEQ ID NO: 96 - DNA, 50 bp, Homo sapiens
gcggtcagtgttctcagtagtaggtttctgttctattgggtgacttggag

SEQ ID NO: 97 - DNA, 50 bp, Homo sapiens
ccctgcagcctacgggtctgttttctgtgtgtgcccatttccttgacagc

SEQ ID NO: 98 - DNA, 50 bp, Homo sapiens
ggagtccaggtgaggcgtggtagacaagatgcctggtttcaagagctgat

SEQ ID NO: 99 - DNA, 50 bp, Homo sapiens
ttgtgctacccgtgagtccctctccggggtgtgtgaaatcagtggccgcc

SEQ ID NO: 100 - DNA, 50 bp, Homo sapiens
cagcagctgtttttgagcaccttgtcctttgtgtgtccgtggtgtgcaac

SEQ ID NO: 101 - DNA, 50 bp, Homo sapiens
gactacgtcttttactgcaaagaccagcgccgtgggggcctgcgctacat

SEQ ID NO: 102 - DNA, 50 bp, Homo sapiens
ctcgcatgcagtcatctggagggactgaagcactgtttgcctttctgtac

SEQ ID NO: 103 - DNA, 50 bp, Homo sapiens
ggcttattctgcctacgtgccccaagatggaacgatgttaactgcgctgc

SEQ ID NO: 104 - DNA, 50 bp, Homo sapiens
gccagtgcatctaacagccctgtgcagcagcttcccttgcctcgtgtaac

SEQ ID NO: 105 - DNA, 50 bp, Homo sapiens
ctgccccctccccaccctgccactcttgacattccactgtgcgttttaga

SEQ ID NO: 106 - DNA, 50 bp, Homo sapiens
gtggtaccgccaaaattcaaaccccagactcccgaactcatgctcagaac

SEQ ID NO: 107 - DNA, 50 bp, Homo sapiens
gcccagtgacctgccgaggtcggcagcacagagctctggagatgaagacc

SEQ ID NO: 108 - DNA, 50 bp, Homo sapiens
tggaggccgtggctatggttggaggtcagcttcaggccttctggaagcat

SEQ ID NO: 109 - DNA, 50 bp, Homo sapiens
gatgatcctactgctcccagcaacctctacatccaggaatgagtccctag

SEQ ID NO: 110 - DNA, 50 bp, Homo sapiens
cccatgtgggtgtggcagttacagggcccaggtgagctgaagacaaacca

SEQ ID NO: 111 - DNA, 50 bp, Homo sapiens
ctcagggaactcggtttttggagacatgagtctgctgatgctcctgactg

SEQ ID NO: 112 - DNA, 50 bp, Homo sapiens
catgatagacacgatgaaatccaccctcaaagagcagttccagtttgtgg

SEQ ID NO: 113 - DNA, 50 bp, Homo sapiens
tgtggccgcttctgtgtcccaccagtcctgcccccaaaactgaccatgaa

SEQ ID NO: 114 - DNA, 50 bp, Homo sapiens
gcgtcagagtcgctgagcttgttggcctgatcttgcgtttggaaagaaat

SEQ ID NO: 115 - DNA, 50 bp, Homo sapiens
agctggccccacacaaccaaggtccaagggaggaccatcatcttctccag

SEQ ID NO: 116 - DNA, 50 bp, Homo sapiens
ctgggcacagagaatcagctaggagaccagttattcagggtccatttctc

SEQ ID NO: 117 - DNA, 50 bp, Homo sapiens
acgtgctccctctgccaggaggagaatgaagacgtggtgcgagatgcgct

SEQ ID NO: 118 - DNA, 50 bp, Homo sapiens
ttgtggaaagtccttttttactgctttgcccattggaggtgtctcctttt

SEQ ID NO: 119 - DNA, 50 bp, Homo sapiens
gacacacacaaacacagaaccacacagccagtcccaggagcccagtaatg

SEQ ID NO: 120 - DNA, 50 bp, Homo sapiens
gccacgaataaggccatcaccagaagccaaccccgccagtccttgatcta

SEQ ID NO: 121 - DNA, 50 bp, Homo sapiens
cacagtggggagcatggagggatgggtttggcctgtgcttctgcttattc

SEQ ID NO: 122 - DNA, 50 bp, Homo sapiens
tgccagccttgcagaaaaggctcccattgtgttaccccatcactcaacct

SEQ ID NO: 123 - DNA, 50 bp, Homo sapiens
ttgcctccctcagatagaaaacagcccccactccagtccactcctgaccc

SEQ ID NO: 124 - DNA, 50 bp, Homo sapiens
aaagtgaccttagtgatgcagagttcctgagtggtgtttgtagaatgagt

SEQ ID NO: 125 - DNA, 50 bp, Homo sapiens
aggagaccaccgccttctccagtgcttccttgggcagccagtaattccca

SEQ ID NO: 126 - DNA, 50 bp, Homo sapiens
gccaatgttaatttatagccaggtgtgcgtgtgtctcccgcctcgccgcc

SEQ ID NO: 127 - DNA, 50 bp, Homo sapiens
aatggagcaggatattgctgaagtctcctggcatatgttaccgaatcaaa

SEQ ID NO: 128 - DNA, 50 bp, Homo sapiens
tgctgaagtctcctggcatatgttaccgaatcaaatagccttccagaggc

SEQ ID NO: 129 - DNA, 50 bp, Homo sapiens
tgagaatacttgtccctggaggattatcacaccccaaatgcataatctcg

SEQ ID NO: 130 - DNA, 50 bp, Homo sapiens
cttctacccagaaggatggacagctaatagcgtacttggggatgaggagc

SEQ ID NO: 131 - DNA, 50 bp, Homo sapiens
ggaacgcggcggagctgtgagccggcgactcgggtccctgaggtctggat

SEQ ID NO: 132 - DNA, 50 bp, Homo sapiens
ggatgagagggaaccactataacatgagtccaagcccagaagacttctgt

SEQ ID NO: 133 - DNA, 50 bp, Homo sapiens
gcctcacttaagtccaggaagctggggtggcgaggaaggatgatgtggat

SEQ ID NO: 134 - DNA, 50 bp, Homo sapiens
cacagtgccagcctctacctaaggaaaccctagaccttggaaccagtttc

SEQ ID NO: 135 - DNA, 50 bp, Homo sapiens
gaggtctctccagctgtttcaaaatgtcatgtaactggtgacactcagcc

SEQ ID NO: 136 - DNA, 50 bp, Homo sapiens
ctgggagttccctgaacatctgtgtgtgtccccctatgctccagtatgga

SEQ ID NO: 137 - DNA, 50 bp, Homo sapiens
acagggtcacagtaatggggggtttcaaagtggagaaccacacggcgtgc

SEQ ID NO: 138 - DNA, 50 bp, Homo sapiens
gtcacccctggtgccgtgaagctggagaaggagaagctggagcaaaaccc

SEQ ID NO: 139 - DNA, 50 bp, Homo sapiens
ccccagccaggtctcatagagggaattgttcttcaggctcggaggggcct

SEQ ID NO: 140 - DNA, 50 bp, Homo sapiens
ctgggctgacgcccccttgcctctgcctggtacccacatgacttggaact

SEQ ID NO: 141 - DNA, 50 bp, Homo sapiens
ctctatcggaagctttactccgtcgagagtctgtaggagcagcagtcctc

SEQ ID NO: 142 - DNA, 50 bp, Homo sapiens
tgcgcgacatggaaggtgatctgcaagagctgcatcagtcaaacaccggg

SEQ ID NO: 143 - DNA, 50 bp, Homo sapiens
gagtgtgagagtgataatgcaggggtgagtgtgagagggactgcgtttgc

SEQ ID NO: 144 - DNA, 50 bp, Homo sapiens
gccagttgacaagatttttccaccctcgagcagcgtgagagatgcctctt

SEQ ID NO: 145 - DNA, 50 bp, Homo sapiens
gcgcctttctcatcagcttcttccgagggtgacaggtgaaagacccctac

SEQ ID NO: 146 - DNA, 50 bp, Homo sapiens
gagtatcagaatcacctggaagggcttttacagattgctggccccacccc

SEQ ID NO: 147 - DNA, 50 bp, Homo sapiens
ctgtgggtcaccaagctctggaagttcattcgcgatttcctgcggtggtg

SEQ ID NO: 148 - DNA, 50 bp, Homo sapiens
tgtcctcgctatgagatttcccaccacccacgctggtgttcacatctagc

SEQ ID NO: 149 - DNA, 50 bp, Homo sapiens
acagaggatactacaggctgggaaagctgcagcaaagggagactagagag

SEQ ID NO: 150 - DNA, 50 bp, Homo sapiens
ggaaggcgcctgtccatatggataagatagggttgtaaacgtcctcatct

SEQ ID NO: 151 - DNA, 50 bp, Homo sapiens
tccgaaaaagatctgccggcccagagcactccctcttgccctaatcctgg

SEQ ID NO: 152 - DNA, 50 bp, Homo sapiens
ctcccaaagactcagctcccctgttagatggctctgcctgtccttcccca

SEQ ID NO: 153 - DNA, 50 bp, Homo sapiens
gccgaagccatgaatgccctcgaactaggcccttggatgaagtgaaccag

SEQ ID NO: 154 - DNA, 50 bp, Homo sapiens
tgttctgcaggcttgcagagttcttctcaccatttaaactgaaggaccct

SEQ ID NO: 155 - DNA, 50 bp, Homo sapiens
agtaatggagagccccaaaaagaagaaccagcagctgaaagtcgggatcc

SEQ ID NO: 156 - DNA, 50 bp, Homo sapiens
ttggtggaaaaaccacctgatgaggctgcacccttggtggagggaacagc

SEQ ID NO: 157 - DNA, 50 bp, Homo sapiens
agccattgcccagtggtccatatctccaccacatcccctgcttgagccca

SEQ ID NO: 158 - DNA, 50 bp, Homo sapiens
acagagcttcggagcccatgcgaatgcgaaagcctcgtggagttccaggg

SEQ ID NO: 159 - DNA, 50 bp, Homo sapiens
ggcaagcgatcaagcagcgactatgcacaacgagaggattttgaggctgc

SEQ ID NO: 160 - DNA, 50 bp, Homo sapiens
gcagtgcccgaaacccacactgcagatcagccacatcgcccagcagcttg

SEQ ID NO: 161 - DNA, 50 bp, Homo sapiens
acatcccctggaagtacaaggactcatctgtggtccctgcttctcctccc

SEQ ID NO: 162 - DNA, 50 bp, Homo sapiens
caggttggagtgcggctagtgccccaaggcggcttggagacctctcagcc

SEQ ID NO: 163 - DNA, 50 bp, Homo sapiens
actcacacagctcctccacaggagacttctggagcaagcaggaccagcct

SEQ ID NO: 164 - DNA, 50 bp, Homo sapiens
gagaacatcaccaccctgaagccagagactaacactgcaggactcagcaa

SEQ ID NO: 165 - DNA, 50 bp, Homo sapiens
ctccaccccccgcctgtgggatgccttgtgggacgtctctttctattcaa

SEQ ID NO: 166 - DNA, 50 bp, Homo sapiens
gcccagctgtagctctgggcctggaactatgaagacctagtgctcccaga

SEQ ID NO: 167 - DNA, 50 bp, Homo sapiens
aaagaagaaccagcagctgaaagtcgggatcctacacctgggcagcagac

SEQ ID NO: 168 - DNA, 50 bp, Homo sapiens
gcgcaaccggttctccgaaacatggagtcctgtaggcaaggtcttacctg

SEQ ID NO: 169 - DNA, 50 bp, Homo sapiens
cgccgtggctctcagctgtcaatgtgcactctgccgccgcagcaccactg

SEQ ID NO: 170 - DNA, 50 bp, Homo sapiens
gtcagcagctctatctcaccaatgacagccagaatagcaagcaaccactg

SEQ ID NO: 171 - DNA, 50 bp, Homo sapiens
caaggcaggaagagaatccatccacctctctggacagtaatggtgcagca

SEQ ID NO: 172 - DNA, 50 bp, Homo sapiens
tgttcaagtcctagtctataggattggcagtttaaatgctttactccccc

SEQ ID NO: 173 - DNA, 50 bp, Homo sapiens
gaagattagaaccagacttactaaccaattccaccccccaccaaccccct

SEQ ID NO: 174 - DNA, 50 bp, Homo sapiens
ggagatgacctagaatgcagagaaacagcctcctctcccaaaagccaacg

SEQ ID NO: 175 - DNA, 50 bp, Homo sapiens
ggccacctattttgaaacgcacacctttgccatgaagtctgttgttgcat

SEQ ID NO: 176 - DNA, 50 bp, Homo sapiens
aaaaatgcactgtgagtttcatgcctgctggcctgccttcactgtcctgg

SEQ ID NO: 177 - DNA, 50 bp, Homo sapiens
gaaatgtattgtttgagctcaaaaggcccgaccaccccccttcgggctgc

SEQ ID NO: 178 - DNA, 50 bp, Homo sapiens
cccaggggaagacccaaaggcagcaaaaacaagagtccctctaaagcagc

SEQ ID NO: 179 - DNA, 50 bp, Homo sapiens
gcactaagagaaggagactctcaaaccaaaaatgacctggaggcaccatg

SEQ ID NO: 180 - DNA, 50 bp, Homo sapiens
actaacgaggacgccgtccagggcatcgctaacgaggacgccgtccacag

SEQ ID NO: 181 - DNA, 50 bp, Homo sapiens
ctcctcataggtgagatcaagaggccaccagttgtacttcagcaccaatg

SEQ ID NO: 182 - DNA, 50 bp, Homo sapiens
ggagccagagccagtcaggggttaaagtgaaagcccgtatttccgcccag

SEQ ID NO: 183 - DNA, 50 bp, Homo sapiens
ccaccccgtccgatttgggtctgatgatgagggcagatacctaactcagg

SEQ ID NO: 184 - DNA, 50 bp, Homo sapiens
ccaggactccagagctgtgacctggctctggttcaacaaaaggggcctga

SEQ ID NO: 185 - DNA, 50 bp, Homo sapiens
ctttaattcttgggcctccaataagtgtcccataggtgtctggccaggcc

SEQ ID NO: 186 - DNA, 50 bp, Homo sapiens
ggaacggctgctgtttcagctgttggccttttcaattctatactccattt

SEQ ID NO: 187 - DNA, 50 bp, Homo sapiens
gcaagccctcgccttcgccgtgtgggaattttcctggatgctgacttaga

SEQ ID NO: 188 - DNA, 50 bp, Homo sapiens
ggaagggcctggagtggattgggtacatctattacagtgggagcacctac

SEQ ID NO: 189 - DNA, 50 bp, Homo sapiens
cttttgagtcctcgggcccagaatcgtatcccaaagccctcccatggcct

SEQ ID NO: 190 - DNA, 50 bp, Homo sapiens
taaggacccagtttcttctgtgagcacgggcagtgggaggccggtggaaa

SEQ ID NO: 191 - DNA, 50 bp, Homo sapiens
cgacagagaccaacaggagccccaaacatgtattcctcttcactattggc

SEQ ID NO: 192 - DNA, 50 bp, Homo sapiens
cgtctcccggggccctaggacaccccgatcctcccacaataaaggcttct

SEQ ID NO: 193 - DNA, 50 bp, Homo sapiens
gacaaggaagagctttgccatcccctgcatgtgcccctgcctctacctgt

SEQ ID NO: 194 - DNA, 50 bp, Homo sapiens
gacctgatgtccattcatcccaccctctcacagttcggacttttctcccc

SEQ ID NO: 195 - DNA, 50 bp, Homo sapiens
ctcctggtgggacttgtatcttgtctgccatatcagaacacaaacccctg

SEQ ID NO: 196 - DNA, 50 bp, Homo sapiens
ggagagtgaagtctacagcctagagggtgccctgtgtgtggtgtcagatg

SEQ ID NO: 197 - DNA, 50 bp, Homo sapiens
cccaaggcgtttggctcagagggctacagactatggccagaactcatctg

SEQ ID NO: 198 - DNA, 50 bp, Homo sapiens
gaggggacacactctgcaccaacctcactgggacacttttgccttcccga

SEQ ID NO: 199 - DNA, 50 bp, Homo sapiens
gcaggggacagtttttccagggtggcctatcattggggtatgagtggctg

SEQ ID NO: 200 - DNA, 50 bp, Homo sapiens
gaccgttttgtggctagtgcgatttcacagtctactgcctgtttccactg

SEQ ID NO: 201 - DNA, 50 bp, Homo sapiens
ctgggtccgccagcccccagggaaggggctggagtggattggggaaatct

SEQ ID NO: 202 - DNA, 50 bp, Homo sapiens
taccagactccgacacaatgggccaccaccagtgtgacagcaggacatcc

SEQ ID NO: 203 - DNA, 50 bp, Homo sapiens
cctccccaaaagcagcatattaggcccaggacatacctgaaggctggtcg

SEQ ID NO: 204 - DNA, 50 bp, Homo sapiens
ctttctgcaagacctttggctcacagaactgcagggtatggtgagaaacc

SEQ ID NO: 205 - DNA, 50 bp, Homo sapiens
gcatagcgacttgcctttccgtcttgttcagtcgtcaccagtcagccgtc

SEQ ID NO: 206 - DNA, 50 bp, Homo sapiens
tcttggcagggggagaggatggcccagcaggcctggcccagctcccagtt

SEQ ID NO: 207 - DNA, 50 bp, Homo sapiens
caaatgagcacgctgccatgtactggtttgagcagtaggggctgcatcgc

SEQ ID NO: 208 - DNA, 50 bp, Homo sapiens
tcagccaaaggggctgactgtcattggagcggtgggcactgttaagaagc

SEQ ID NO: 209 - DNA, 50 bp, Homo sapiens
cctgtgaatacctcagcttcaactgggcctccatacagtcagttggtggg

SEQ ID NO: 210 - DNA, 50 bp, Homo sapiens
accacagtgcagggaaaacaaaagtatcccagcatcttcatcctgtacac

SEQ ID NO: 211 - DNA, 50 bp, Homo sapiens
atcggggttggcgttggccgcagatgcctctcggtccctccctgtactta

SEQ ID NO: 212 - DNA, 50 bp, Homo sapiens
cctggagttagtcgaccgttgcgagacgttgagctgcggcagatgagtcc

SEQ ID NO: 213 - DNA, 50 bp, Homo sapiens
actgcctgctacttgagggccaggatgctcccccagggaagaaacggaat

SEQ ID NO: 214 - DNA, 50 bp, Homo sapiens
ggtctcctgatggaagtgtacaataccacctacaaattatccatgcccca

SEQ ID NO: 215 - DNA, 50 bp, Homo sapiens
gttaggaatgaaagcatccctggagaacagcctggaggagaccaaaggcc

SEQ ID NO: 216 - DNA, 50 bp, Homo sapiens
ggtgacaaaactgagtcctggggaaagtgactggtccgtggtagagccgg

SEQ ID NO: 217 - DNA, 50 bp, Homo sapiens
ccctgcagaggatgctcgttttgcagagaaggcagtgttcctctattccc

SEQ ID NO: 218 - DNA, 50 bp, Homo sapiens
gggtactggctgtggatcatttagctgcagtcctctttcctacaaccttg

SEQ ID NO: 219 - DNA, 50 bp, Homo sapiens
ccaggctccctccgcagactgactttcctctgtgtctgggtgttacagtc

SEQ ID NO: 220 - DNA, 50 bp, Homo sapiens
catagaccctgatgccctcaaagagctccccctcctaaagttccttggca

SEQ ID NO: 221 - DNA, 50 bp, Homo sapiens
gagctgggattcatcctaaaaggcttctccccagatgccagagacctgtc

SEQ ID NO: 222 - DNA, 50 bp, Homo sapiens
ccaaatggcatacttacaagacggatgcaacctgggtccttaggtcgctg

SEQ ID NO: 223 - DNA, 50 bp, Homo sapiens
atccttcctgtttactcctgccttgtggcggggtccatcctctgctgctc

SEQ ID NO: 224 - DNA, 50 bp, Homo sapiens
atgtcttcggtgctggcggcttcccatccgctggttctatcctcaaacgc

SEQ ID NO: 225 - DNA, 50 bp, Homo sapiens
gtaggaggcaggtctccgcggttcatctgtgttgctctaaatgacactgt

SEQ ID NO: 226 - DNA, 50 bp, Homo sapiens
aggcctgcctttaattttcagtgtaagtgttcagtatgccgcatcctgcc

SEQ ID NO: 227 - DNA, 50 bp, Homo sapiens
catgatgagaaccgcctggaagctttaagcacatgacctggggaccaggc

SEQ ID NO: 228 - DNA, 50 bp, Homo sapiens
tgatcaggctgccaatgaatggggcaggagtggcaaagaccccaatcact

SEQ ID NO: 229 - DNA, 50 bp, Homo sapiens
gtgacggctggtgactgatggatgggtagtgggctgagaagaggggacta

SEQ ID NO: 230 - DNA, 50 bp, Homo sapiens
tcttttggcctttccccaatgcttgaaagaagatcaccactgctggaggc

SEQ ID NO: 231 - DNA, 50 bp, Homo sapiens
gagagtctctgtgagactgtttcacagaaggatgtgtgtttacccaaggc

SEQ ID NO: 232 - DNA, 50 bp, Homo sapiens
tgttggaggatgaaagtacggagtgatccatcggctaagtgtcttgtcac

SEQ ID NO: 233 - DNA, 50 bp, Homo sapiens
gctggtgaaaccccgaagatcaacacgcttcaaactcagccccttggaac

SEQ ID NO: 234 - DNA, 50 bp, Homo sapiens
ccgtttcttaaatgttaccagtcccagccaatcttacggtgacattacag

SEQ ID NO: 235 - DNA, 50 bp, Homo sapiens
tccctcaacatcagaacaatgctcaagtctttcaagccacgtctgagcag

SEQ ID NO: 236 - DNA, 50 bp, Homo sapiens
ttctggacctcagtccttcacctagtcacctagtcacagggtggatcgcc

SEQ ID NO: 237 - DNA, 50 bp, Homo sapiens
aggaagcccgccagtggtgagactgtgtcctcaaggagcttcggcaccat

SEQ ID NO: 238 - DNA, 50 bp, Homo sapiens
ctcctgatccctttcccatcggatctgaacactggtcttggtggtcgtaa

SEQ ID NO: 239 - DNA, 50 bp, Homo sapiens
taattgtgtgaagtgtgtctgtctccagcccttcgggcctcccacgagcc

SEQ ID NO: 240 - DNA, 50 bp, Homo sapiens
agagtgcctaagagaaacccttgcacctgggagcgctgcttggctctatc

SEQ ID NO: 241 - DNA, 50 bp, Homo sapiens
ccagggttagccagacaggcaccaaagccaaggaagcagagatccagcct

SEQ ID NO: 242 - DNA, 50 bp, Homo sapiens
cgctgacatcaaaactgttcggacttccccgttagaactcgcgtttccac

SEQ ID NO: 243 - DNA, 50 bp, Homo sapiens
ggtatatacagggaggccaggcagcctggagttagtcgaccgttgcgaga

SEQ ID NO: 244 - DNA, 50 bp, Homo sapiens
actttaggagacggaggcgggagggtcacttgagcccaggaattgtgaga

SEQ ID NO: 245 - DNA, 50 bp, Homo sapiens
ttaagcctaagtctcttcagccacctcccaggaagatctggcctccgtcc

SEQ ID NO: 246 - DNA, 50 bp, Homo sapiens
gagggccacgaggcgtcggctttagacacagatcatagctctacaggagt

SEQ ID NO: 247 - DNA, 50 bp, Homo sapiens
tgctgggtgtgttgcatggccctcccaaccaattcagtatttttctcccc

SEQ ID NO: 248 - DNA, 50 bp, Homo sapiens
gtctgatctcggaagctaagcagggtcgggcctggttagtacttggacgg

SEQ ID NO: 249 - DNA, 50 bp, Homo sapiens
gagcagctccctcgctgcgatctattgaaagtcagatctccacacaaggg

SEQ ID NO: 250 - DNA, 50 bp, Homo sapiens
gaaggccacttgagcttcctggagaaggacctgagggacaaggtcaactc

SEQ ID NO: 251 - DNA, 50 bp, Homo sapiens
tggatgcccaggggaaggcatacaggcctcatccaccaggcaatagacag

SEQ ID NO: 252 - DNA, 50 bp, Homo sapiens
tcctcggcttagtggctgaagactgatgctgcccgatcgcctcagaagcc

SEQ ID NO: 253 - DNA, 50 bp, Homo sapiens
cgacgcttcttctcagcctccagtctccagttccaaggatgggtcatctc

SEQ ID NO: 254 - DNA, 50 bp, Homo sapiens
aaggccccagagagctaactcacccttccaccatatgaggacgtggcaag

SEQ ID NO: 255 - DNA, 50 bp, Homo sapiens
gcgctgggggctggacctgctgacatagtggatggacagatggaccaaaa

SEQ ID NO: 256 - DNA, 50 bp, Homo sapiens
actggcactggacttcaagcgaaaggcacgcagcgggagcagcagctata

SEQ ID NO: 257 - DNA, 50 bp, Homo sapiens
gagggagagagggagagagcctataccccttacttagcatgcacaaagtg

SEQ ID NO: 258 - DNA, 50 bp, Homo sapiens
cagggcaataggaacacagggtggaaccgcctttgtcaagagcacattcc

SEQ ID NO: 259 - DNA, 50 bp, Homo sapiens
gtggaccccgtggtctccttccctgtggctctcagctgtcgctgtggacc

SEQ ID NO: 260 - DNA, 50 bp, Homo sapiens
agtgtgcagtgaggaaaggggcaaggatcgggaagctgtgtgactgtccc

SEQ ID NO: 261 - DNA, 50 bp, Homo sapiens
ggaataagatacttgttgctgtcacagttattaccatccccccagctacc

SEQ ID NO: 262 - DNA, 50 bp, Homo sapiens
ctgtgtcggagtcagcgcagcttttgaagctggagagcatcatattttag

SEQ ID NO: 263 - DNA, 50 bp, Homo sapiens
gctgtgcaccctagaaacaacatattgtcccatgagcaggtgcctgagac

SEQ ID NO: 264 - DNA, 50 bp, Homo sapiens
tctcagcttaacatggaaatgaggatcccaccagccccagaaccctctgg

SEQ ID NO: 265 - DNA, 50 bp, Homo sapiens
ttgagcccttgcccctgcgtcccgcgtctgggttctcagctatttccaga

SEQ ID NO: 266 - DNA, 50 bp, Homo sapiens
gagatgagtgcggggctcatctatccctggaattgtctttcccacaatcc

SEQ ID NO: 267 - DNA, 50 bp, Homo sapiens
cctgcccctgctctgcactctcaggtattccctgctcttactccaaaaag

SEQ ID NO: 268 - DNA, 50 bp, Homo sapiens
aggaaggacacacagtagctctctgcttgctgatagatggtttcccagtg

SEQ ID NO: 269 - DNA, 50 bp, Homo sapiens
actttgaccagagttggagccacccaggggaatgatctctgatgacctaa

SEQ ID NO: 270 - DNA, 50 bp, Homo sapiens
gctgccttgcttgagttgcttgtgactgatcttttgaggctgtcatcatg

SEQ ID NO: 271 - DNA, 50 bp, Homo sapiens
caggaagcccactgtacctggagccatctgggataagactttgacccatg

SEQ ID NO: 272 - DNA, 50 bp, Homo sapiens
gttacagccatatgcaggacagcagtactcagcatggtcttatgcacagg

SEQ ID NO: 273 - DNA, 50 bp, Homo sapiens
gggcatgagtccttgacaatagtaaatagcacctctgttcccttattggg

SEQ ID NO: 274 - DNA, 50 bp, Homo sapiens
ttatgactggggaagcccagttcagaataggatgcactatgatcagtccc

SEQ ID NO: 275 - DNA, 50 bp, Homo sapiens
ccagtctctcttctgtaggcattgtggtgagcgatcctgactccacagat

SEQ ID NO: 276 - DNA, 50 bp, Homo sapiens
ctgacaagtagaatcaaaggtgcagctgactgagacgacatgcatgtaag

SEQ ID NO: 277 - DNA, 50 bp, Homo sapiens
tactcacacggacaggttgatgccagagccgtaagaatgcgccagtgcgg

SEQ ID NO: 278 - DNA, 50 bp, Homo sapiens
aactgagagaaagagcaacaaagcggcgagtggtgtgagagggcagcacg

SEQ ID NO: 279 - DNA, 50 bp, Homo sapiens
ggtatgtctggagggtgaagagaggttggttagtgggtaaaaatacacag

SEQ ID NO: 280 - DNA, 50 bp, Homo sapiens
accgccgccgggcacctgccaccaagcaactgtttcattttttattttcc

SEQ ID NO: 281 - DNA, 50 bp, Homo sapiens
acatgtcctgggtgacatgggatgtgactttcgggtgtcggggcagcatg

SEQ ID NO: 282 - DNA, 50 bp, Homo sapiens
agggtcgaatctggaatgggagggtctggcttcagctatcagggcaccct

SEQ ID NO: 283 - DNA, 50 bp, Homo sapiens
tcctcacaggacagaagcagagtgggtggtggttatgtttgacagaaggc

SEQ ID NO: 284 - DNA, 50 bp, Homo sapiens
tggagtgggttggcttgctgatggctgctggaggggacgctggctaaagt

SEQ ID NO: 285 - DNA, 50 bp, Homo sapiens
aaggacagaggtgtgggagacccaaggtggtcttgagattgacagacagc

SEQ ID NO: 286 - DNA, 50 bp, Homo sapiens
tcgtccatgccaaagtgtgcggcccttcctgacatcaccacagtctgagc

SEQ ID NO: 287 - DNA, 50 bp, Homo sapiens
tggccttcactgactctgctaggtggtgccctgattgttggcatggtgag

SEQ ID NO: 288 - DNA, 50 bp, Homo sapiens; identical to SEQ ID NO: 47

SEQ ID NO: 289 - DNA, 50 bp, Homo sapiens; identical to SEQ ID NO: 48

SEQ ID NO: 290 - DNA, 50 bp, Homo sapiens; identical to SEQ ID NO: 49

SEQ ID NO: 291 - DNA, 50 bp, Homo sapiens; identical to SEQ ID NO: 50

SEQ ID NO: 292 - DNA, 50 bp, Homo sapiens; identical to SEQ ID NO: 51

SEQ ID NO: 293 - DNA, 50 bp, Homo sapiens; identical to SEQ ID NO: 52

SEQ ID NO: 294 - DNA, 50 bp, Homo sapiens; identical to SEQ ID NO: 53

SEQ ID NO: 295 - DNA, 50 bp, Homo sapiens; identical to SEQ ID NO: 54

SEQ ID NO: 296 - DNA, 50 bp, Homo sapiens; identical to SEQ ID NO: 55

SEQ ID NO: 297 - DNA, 50 bp, Homo sapiens; identical to SEQ ID NO: 56

SEQ ID NO: 298 - DNA, 50 bp, Homo sapiens; identical to SEQ ID NO: 57

SEQ ID NO: 299 - DNA, 50 bp, Homo sapiens; identical to SEQ ID NO: 58

SEQ ID NO: 300 - DNA, 50 bp, Homo sapiens; identical to SEQ ID NO: 59

SEQ ID NO: 301 - DNA, 50 bp, Homo sapiens; identical to SEQ ID NO: 60

SEQ ID NO: 302 - DNA, 50 bp, Homo sapiens; identical to SEQ ID NO: 61

SEQ ID NO: 303 - DNA, 50 bp, Homo sapiens; identical to SEQ ID NO: 62

SEQ ID NO: 304 - DNA, 50 bp, Homo sapiens; identical to SEQ ID NO: 63

SEQ ID NO: 305 - DNA, 50 bp, Homo sapiens; identical to SEQ ID NO: 64

SEQ ID NO: 306 - DNA, 50 bp, Homo sapiens; identical to SEQ ID NO: 65

SEQ ID NO: 307 - DNA, 50 bp, Homo sapiens; identical to SEQ ID NO: 66

SEQ ID NO: 308 - DNA, 50 bp, Homo sapiens; identical to SEQ ID NO: 67

SEQ ID NO: 309 - DNA, 50 bp, Homo sapiens; identical to SEQ ID NO: 68

SEQ ID NO: 310 - DNA, 50 bp, Homo sapiens; identical to SEQ ID NO: 70

SEQ ID NO: 311 - DNA, 50 bp, Homo sapiens; identical to SEQ ID NO: 70

SEQ ID NO: 312 - DNA, 50 bp, Homo sapiens; identical to SEQ ID NO: 71

SEQ ID NO: 313 - DNA, 50 bp, Homo sapiens; identical to SEQ ID NO: 72

SEQ ID NO: 314 - DNA, 50 bp, Homo sapiens; identical to SEQ ID NO: 73

SEQ ID NO: 315 - DNA, 50 bp, Homo sapiens; identical to SEQ ID NO: 74

SEQ ID NO: 316 - DNA, 50 bp, Homo sapiens; identical to SEQ ID NO: 75

SEQ ID NO: 317 - DNA, 50 bp, Homo sapiens; identical to SEQ ID NO: 76

SEQ ID NO: 318 - DNA, 50 bp, Homo sapiens; identical to SEQ ID NO: 77

SEQ ID NO: 319 - DNA, 50 bp, Homo sapiens; identical to SEQ ID NO: 78

SEQ ID NO: 320 - DNA, 50 bp, Homo sapiens; identical to SEQ ID NO: 79

SEQ ID NO: 321 - DNA, 50 bp, Homo sapiens; identical to SEQ ID NO: 80

SEQ ID NO: 322 - DNA, 50 bp, Homo sapiens; identical to SEQ ID NO: 81

SEQ ID NO: 323 - DNA, 50 bp, Homo sapiens; identical to SEQ ID NO: 82

SEQ ID NO: 324 - DNA, 50 bp, Homo sapiens; identical to SEQ ID NO: 83

SEQ ID NO: 325 - DNA, 50 bp, Homo sapiens; identical to SEQ ID NO: 84

SEQ ID NO: 326 - DNA, 50 bp, Homo sapiens; identical to SEQ ID NO: 85

SEQ ID NO: 327 - DNA, 50 bp, Homo sapiens; identical to SEQ ID NO: 86

SEQ ID NO: 328 - DNA, 50 bp, Homo sapiens; identical to SEQ ID NO: 87

SEQ ID NO: 329 - DNA, 50 bp, Homo sapiens; identical to SEQ ID NO: 88

SEQ ID NO: 330 - DNA, 50 bp, Homo sapiens; identical to SEQ ID NO: 89

SEQ ID NO: 331 - DNA, 50 bp, Homo sapiens; identical to SEQ ID NO: 90

SEQ ID NO: 332 - DNA, 50 bp, Homo sapiens; identical to SEQ ID NO: 91

SEQ ID NO: 333 - DNA, 50 bp, Homo sapiens; identical to SEQ ID NO: 92

SEQ ID NO: 334 - DNA, 50 bp, Homo sapiens; identical to SEQ ID NO: 93

SEQ ID NO: 335 - DNA, 50 bp, Homo sapiens; identical to SEQ ID NO: 94

SEQ ID NO: 336 - DNA, 50 bp, Homo sapiens; identical to SEQ ID NO: 95

SEQ ID NO: 337 - DNA, 50 bp, Homo sapiens; identical to SEQ ID NO: 96

SEQ ID NO: 338 - DNA, 50 bp, Homo sapiens; identical to SEQ ID NO: 97

SEQ ID NO: 339 - DNA, 50 bp, Homo sapiens; identical to SEQ ID NO: 98

SEQ ID NO: 340 - DNA, 50 bp, Homo sapiens; identical to SEQ ID NO: 99

SEQ ID NO: 341 - DNA, 50 bp, Homo sapiens; identical to SEQ ID NO: 100

SEQ ID NO: 342 - DNA, 50 bp, Homo sapiens; identical to SEQ ID NO: 101

SEQ ID NO: 343 - DNA, 50 bp, Homo sapiens; identical to SEQ ID NO: 102

SEQ ID NO: 344 - DNA, 50 bp, Homo sapiens; identical to SEQ ID NO: 103

SEQ ID NO: 345 - DNA, 50 bp, Homo sapiens; identical to SEQ ID NO: 104

SEQ ID NO: 346 - DNA, 50 bp, Homo sapiens; identical to SEQ ID NO: 105

SEQ ID NO: 347 - DNA, 50 bp, Homo sapiens; identical to SEQ ID NO: 107

SEQ ID NO: 348 - DNA, 50 bp, Homo sapiens; identical to SEQ ID NO: 108

SEQ ID NO: 349 - DNA, 50 bp, Homo sapiens; identical to SEQ ID NO: 108

SEQ ID NO: 350 - DNA, 50 bp, Homo sapiens; identical to SEQ ID NO: 109

SEQ ID NO: 351 - DNA, 50 bp, Homo sapiens; identical to SEQ ID NO: 110

SEQ ID NO: 352 - DNA, 50 bp, Homo sapiens; identical to SEQ ID NO: 111

SEQ ID NO: 353 - DNA, 50 bp, Homo sapiens; identical to SEQ ID NO: 112

SEQ ID NO: 354 - DNA, 50 bp, Homo sapiens; identical to SEQ ID NO: 113

SEQ ID NO: 355 - DNA, 50 bp, Homo sapiens; identical to SEQ ID NO: 114

SEQ ID NO: 356 - DNA, 50 bp, Homo sapiens; identical to SEQ ID NO: 115

SEQ ID NO: 357 - DNA, 50 bp, Homo sapiens; identical to SEQ ID NO: 116

SEQ ID NO: 358 - DNA, 50 bp, Homo sapiens; identical to SEQ ID NO: 117

SEQ ID NO: 359 - DNA, 50 bp, Homo sapiens; identical to SEQ ID NO: 118

SEQ ID NO: 360 - DNA, 50 bp, Homo sapiens; identical to SEQ ID NO: 119

SEQ ID NO: 361 - DNA, 50 bp, Homo sapiens; identical to SEQ ID NO: 120

SEQ ID NO: 362 - DNA, 50 bp, Homo sapiens; identical to SEQ ID NO: 121

SEQ ID NO: 363 - DNA, 50 bp, Homo sapiens; identical to SEQ ID NO: 122

SEQ ID NO: 364 - DNA, 50 bp, Homo sapiens; identical to SEQ ID NO: 123

SEQ ID NO: 365 - DNA, 50 bp, Homo sapiens; identical to SEQ ID NO: 124

SEQ ID NO: 366 - DNA, 50 bp, Homo sapiens; identical to SEQ ID NO: 125

SEQ ID NO: 367 - DNA, 50 bp, Homo sapiens; identical to SEQ ID NO: 126

SEQ ID NO: 368 - DNA, 50 bp, Homo sapiens; identical to SEQ ID NO: 127

SEQ ID NO: 369 - DNA, 50 bp, Homo sapiens; identical to SEQ ID NO: 128

SEQ ID NO: 370 - DNA, 50 bp, Homo sapiens; identical to SEQ ID NO: 129

SEQ ID NO: 371 - DNA, 50 bp, Homo sapiens; identical to SEQ ID NO: 130

SEQ ID NO: 372 - DNA, 50 bp, Homo sapiens; identical to SEQ ID NO: 131

SEQ ID NO: 373 - DNA, 50 bp, Homo sapiens; identical to SEQ ID NO: 132

SEQ ID NO: 374 - DNA, 50 bp, Homo sapiens; identical to SEQ ID NO: 133

SEQ ID NO: 375 - DNA, 50 bp, Homo sapiens; identical to SEQ ID NO: 134

SEQ ID NO: 376 - DNA, 50 bp, Homo sapiens; identical to SEQ ID NO: 135

SEQ ID NO: 377 - DNA, 50 bp, Homo sapiens; identical to SEQ ID NO: 136

SEQ ID NO: 378 - DNA, 50 bp, Homo sapiens; identical to SEQ ID NO: 137

SEQ ID NO: 379 - DNA, 50 bp, Homo sapiens; identical to SEQ ID NO: 138

SEQ ID NO: 380 - DNA, 50 bp, Homo sapiens; identical to SEQ ID NO: 139

SEQ ID NO: 381 - DNA, 50 bp, Homo sapiens; identical to SEQ ID NO: 140

SEQ ID NO: 382 - DNA, 50 bp, Homo sapiens; identical to SEQ ID NO: 141

SEQ ID NO: 383 - DNA, 50 bp, Homo sapiens; identical to SEQ ID NO: 142

SEQ ID NO: 384 - DNA, 50 bp, Homo sapiens; identical to SEQ ID NO: 144

SEQ ID NO: 385 - DNA, 50 bp, Homo sapiens; identical to SEQ ID NO: 144

SEQ ID NO: 386 - DNA, 50 bp, Homo sapiens; identical to SEQ ID NO: 145

SEQ ID NO: 387 - DNA, 50 bp, Homo sapiens; identical to SEQ ID NO: 146

SEQ ID NO: 388 - DNA, 50 bp, Homo sapiens; identical to SEQ ID NO: 147

SEQ ID NO: 389 - DNA, 50 bp, Homo sapiens; identical to SEQ ID NO: 148

SEQ ID NO: 390 - DNA, 50 bp, Homo sapiens; identical to SEQ ID NO: 149

SEQ ID NO: 391 - DNA, 50 bp, Homo sapiens; identical to SEQ ID NO: 150

SEQ ID NO: 392 - DNA, 50 bp, Homo sapiens; identical to SEQ ID NO: 151

SEQ ID NO: 393 - DNA, 50 bp, Homo sapiens; identical to SEQ ID NO: 152

SEQ ID NO: 394 - DNA, 50 bp, Homo sapiens; identical to SEQ ID NO: 153

SEQ ID NO: 395 - DNA, 50 bp, Homo sapiens; identical to SEQ ID NO: 154

SEQ ID NO: 396 - DNA, 50 bp, Homo sapiens; identical to SEQ ID NO: 155

SEQ ID NO: 397 - DNA, 50 bp, Homo sapiens; identical to SEQ ID NO: 156

SEQ ID NO: 398 - DNA, 50 bp, Homo sapiens; identical to SEQ ID NO: 157

SEQ ID NO: 399 - DNA, 50 bp, Homo sapiens; identical to SEQ ID NO: 158

SEQ ID NO: 400 - DNA, 50 bp, Homo sapiens; identical to SEQ ID NO: 159

SEQ ID NO: 401 - DNA, 50 bp, Homo sapiens; identical to SEQ ID NO: 160

SEQ ID NO: 402 - DNA, 49 bp, Homo sapiens
acatcccctggaagtacaaggactcatctgtggtccctgcttctcctcc

SEQ ID NO: 403 - DNA, 49 bp, Homo sapiens
caggttggagtgcggctagtgccccaaggcggcttggagacctctcagc

SEQ ID NO: 404 - DNA, 49 bp, Homo sapiens
actcacacagctcctccacaggagacttctggagcaagcaggaccagcc

SEQ ID NO: 405 - DNA, 49 bp, Homo sapiens
gtcagcagctctatctcaccaatgacagccagaatagcaagcaaccact

SEQ ID NO: 406 - DNA, 50 bp, Homo sapiens; identical to SEQ ID NO: 164

SEQ ID NO: 407 - DNA, 50 bp, Homo sapiens; identical to SEQ ID NO: 166

SEQ ID NO: 408 - DNA, 50 bp, Homo sapiens; identical to SEQ ID NO: 166

SEQ ID NO: 409 - DNA, 50 bp, Homo sapiens; identical to SEQ ID NO: 167

SEQ ID NO: 410 - DNA, 50 bp, Homo sapiens; identical to SEQ ID NO: 168

SEQ ID NO: 411 - DNA, 50 bp, Homo sapiens; identical to SEQ ID NO: 169

SEQ ID NO: 412 - DNA, 50 bp, Homo sapiens; identical to SEQ ID NO: 171

SEQ ID NO: 413 - DNA, 50 bp, Homo sapiens; identical to SEQ ID NO: 172

SEQ ID NO: 414 - DNA, 50 bp, Homo sapiens; identical to SEQ ID NO: 173

SEQ ID NO: 415 - DNA, 50 bp, Homo sapiens; identical to SEQ ID NO: 174

SEQ ID NO: 416 - DNA, 50 bp, Homo sapiens; identical to SEQ ID NO: 176

SEQ ID NO: 417 - DNA, 50 bp, Homo sapiens; identical to SEQ ID NO: 177

SEQ ID NO: 418 - DNA, 50 bp, Homo sapiens; identical to SEQ ID NO: 177

SEQ ID NO: 419 - DNA, 50 bp, Homo sapiens; identical to SEQ ID NO: 178

SEQ ID NO: 420 - DNA, 50 bp, Homo sapiens; identical to SEQ ID NO: 179

SEQ ID NO: 421 - DNA, 50 bp, Homo sapiens; identical to SEQ ID NO: 180

SEQ ID NO: 422 - DNA, 50 bp, Homo sapiens; identical to SEQ ID NO: 181

SEQ ID NO: 423 - DNA, 50 bp, Homo sapiens; identical to SEQ ID NO: 182

SEQ ID NO: 424 - DNA, 50 bp, Homo sapiens; identical to SEQ ID NO: 183

SEQ ID NO: 425 - DNA, 50 bp, Homo sapiens; identical to SEQ ID NO: 184

SEQ ID NO: 426 - DNA, 50 bp, Homo sapiens; identical to SEQ ID NO: 185

SEQ ID NO: 427 - DNA, 50 bp, Homo sapiens; identical to SEQ ID NO: 186

SEQ ID NO: 428 - DNA, 50 bp, Homo sapiens; identical to SEQ ID NO: 187

SEQ ID NO: 429 - DNA, 50 bp, Homo sapiens; identical to SEQ ID NO: 188

SEQ ID NO: 430 - DNA, 50 bp, Homo sapiens; identical to SEQ ID NO: 189

SEQ ID NO: 431 - DNA, 50 bp, Homo sapiens; identical to SEQ ID NO: 190

SEQ ID NO: 432 - DNA, 50 bp, Homo sapiens; identical to SEQ ID NO: 191

SEQ ID NO: 433 - DNA, 50 bp, Homo sapiens; identical to SEQ ID NO: 192

SEQ ID NO: 434 - DNA, 50 bp, Homo sapiens; identical to SEQ ID NO: 193

SEQ ID NO: 435 - DNA, 50 bp, Homo sapiens; identical to SEQ ID NO: 194

SEQ ID NO: 436 - DNA, 50 bp, Homo sapiens; identical to SEQ ID NO: 195

SEQ ID NO: 437 - DNA, 50 bp, Homo sapiens; identical to SEQ ID NO: 196

SEQ ID NO: 438 - DNA, 50 bp, Homo sapiens; identical to SEQ ID NO: 197

SEQ ID NO: 439 - DNA, 50 bp, Homo sapiens; identical to SEQ ID NO: 199

SEQ ID NO: 440 - DNA, 50 bp, Homo sapiens; identical to SEQ ID NO: 199

SEQ ID NO: 441 - DNA, 50 bp, Homo sapiens; identical to SEQ ID NO: 200

SEQ ID NO: 442 - DNA, 50 bp, Homo sapiens; identical to SEQ ID NO: 201

SEQ ID NO: 443 - DNA, 50 bp, Homo sapiens; identical to SEQ ID NO: 202

SEQ ID NO: 444 - DNA, 50 bp, Homo sapiens; identical to SEQ ID NO: 203

SEQ ID NO: 445 - DNA, 50 bp, Homo sapiens; identical to SEQ ID NO: 204

SEQ ID NO: 446 - DNA, 50 bp, Homo sapiens; identical to SEQ ID NO: 205

SEQ ID NO: 447 - DNA, 50 bp, Homo sapiens; identical to SEQ ID NO: 206

SEQ ID NO: 448 - DNA, 50 bp, Homo sapiens; identical to SEQ ID NO: 207

SEQ ID NO: 449 - DNA, 50 bp, Homo sapiens; identical to SEQ ID NO: 208

SEQ ID NO: 450 - DNA, 50 bp, Homo sapiens; identical to SEQ ID NO: 209

SEQ ID NO: 451 - DNA, 50 bp, Homo sapiens; identical to SEQ ID NO: 210

SEQ ID NO: 452 - DNA, 50 bp, Homo sapiens; identical to SEQ ID NO: 211

SEQ ID NO: 453 - DNA, 50 bp, Homo sapiens; identical to SEQ ID NO: 212

SEQ ID NO: 454 - DNA, 50 bp, Homo sapiens; identical to SEQ ID NO: 213

SEQ ID NO: 455 - DNA, 50 bp, Homo sapiens; identical to SEQ ID NO: 214

SEQ ID NO: 456 - DNA, 50 bp, Homo sapiens; identical to SEQ ID NO: 215

SEQ ID NO: 457 - DNA, 50 bp, Homo sapiens; identical to SEQ ID NO: 216

SEQ ID NO: 458 - DNA, 50 bp, Homo sapiens; identical to SEQ ID NO: 217

SEQ ID NO: 459 - DNA, 50 bp, Homo sapiens; identical to SEQ ID NO: 218

SEQ ID NO: 460 - DNA, 50 bp, Homo sapiens; identical to SEQ ID NO: 219

SEQ ID NO: 461 - DNA, 50 bp, Homo sapiens; identical to SEQ ID NO: 220

SEQ ID NO: 462 - DNA, 50 bp, Homo sapiens; identical to SEQ ID NO: 221

SEQ ID NO: 463 - DNA, 50 bp, Homo sapiens; identical to SEQ ID NO: 222

SEQ ID NO: 464 - DNA, 50 bp, Homo sapiens; identical to SEQ ID NO: 223

SEQ ID NO: 465 - DNA, 50 bp, Homo sapiens; identical to SEQ ID NO: 224

SEQ ID NO: 466 - DNA, 50 bp, Homo sapiens; identical to SEQ ID NO: 225

SEQ ID NO: 467 - DNA, 50 bp, Homo sapiens; identical to SEQ ID NO: 226

SEQ ID NO: 468 - DNA, 50 bp, Homo sapiens; identical to SEQ ID NO: 227

SEQ ID NO: 469 - DNA, 50 bp, Homo sapiens; identical to SEQ ID NO: 228

SEQ ID NO: 470 - DNA, 50 bp, Homo sapiens; identical to SEQ ID NO: 229

SEQ ID NO: 471 - DNA, 50 bp, Homo sapiens; identical to SEQ ID NO: 230

SEQ ID NO: 472 - DNA, 50 bp, Homo sapiens; identical to SEQ ID NO: 231

SEQ ID NO: 473 - DNA, 50 bp, Homo sapiens; identical to SEQ ID NO: 232

SEQ ID NO: 474 - DNA, 50 bp, Homo sapiens; identical to SEQ ID NO: 233

SEQ ID NO: 475 - DNA, 50 bp, Homo sapiens; identical to SEQ ID NO: 234

SEQ ID NO: 476 - DNA, 50 bp, Homo sapiens; identical to SEQ ID NO: 235

SEQ ID NO: 477 - DNA, 50 bp, Homo sapiens; identical to SEQ ID NO: 236

SEQ ID NO: 478 - DNA, 50 bp, Homo sapiens; identical to SEQ ID NO: 237

SEQ ID NO: 479 - DNA, 50 bp, Homo sapiens; identical to SEQ ID NO: 238

SEQ ID NO: 480 - DNA, 50 bp, Homo sapiens; identical to SEQ ID NO: 239

SEQ ID NO: 481 - DNA, 50 bp, Homo sapiens; identical to SEQ ID NO: 240

SEQ ID NO: 482 - DNA, 50 bp, Homo sapiens; identical to SEQ ID NO: 241

SEQ ID NO: 483 - DNA, 50 bp, Homo sapiens; identical to SEQ ID NO: 242

SEQ ID NO: 484 - DNA, 50 bp, Homo sapiens; identical to SEQ ID NO: 243

SEQ ID NO: 485 - DNA, 50 bp, Homo sapiens; identical to SEQ ID NO: 244

SEQ ID NO: 486 - DNA, 50 bp, Homo sapiens; identical to SEQ ID NO: 245

SEQ ID NO: 487 - DNA, 50 bp, Homo sapiens; identical to SEQ ID NO: 246

SEQ ID NO: 488 - DNA, 50 bp, Homo sapiens; identical to SEQ ID NO: 247

SEQ ID NO: 489 - DNA, 50 bp, Homo sapiens; identical to SEQ ID NO: 248

SEQ ID NO: 490 - DNA, 50 bp, Homo sapiens; identical to SEQ ID NO: 249

SEQ ID NO: 491 - DNA, 50 bp, Homo sapiens; identical to SEQ ID NO: 250

SEQ ID NO: 492 - DNA, 50 bp, Homo sapiens; identical to SEQ ID NO: 251

SEQ ID NO: 493 - DNA, 50 bp, Homo sapiens; identical to SEQ ID NO: 252

SEQ ID NO: 494 - DNA, 50 bp, Homo sapiens; identical to SEQ ID NO: 253

SEQ ID NO: 495 - DNA, 50 bp, Homo sapiens; identical to SEQ ID NO: 254

SEQ ID NO: 496 - DNA, 50 bp, Homo sapiens; identical to SEQ ID NO: 256

SEQ ID NO: 497 - DNA, 50 bp, Homo sapiens; identical to SEQ ID NO: 256

SEQ ID NO: 498 - DNA, 50 bp, Homo sapiens; identical to SEQ ID NO: 257

SEQ ID NO: 499 - DNA, 50 bp, Homo sapiens; identical to SEQ ID NO: 258

SEQ ID NO: 500 - DNA, 50 bp, Homo sapiens; identical to SEQ ID NO: 259

SEQ ID NO: 501 - DNA, 50 bp, Homo sapiens; identical to SEQ ID NO: 260

SEQ ID NO: 502 - DNA, 50 bp, Homo sapiens; identical to SEQ ID NO: 261

SEQ ID NO: 503 - DNA, 50 bp, Homo sapiens; identical to SEQ ID NO: 262

SEQ ID NO: 504 - DNA, 50 bp, Homo sapiens; identical to SEQ ID NO: 263

SEQ ID NO: 505 - DNA, 50 bp, Homo sapiens; identical to SEQ ID NO: 264

SEQ ID NO: 506 - DNA, 50 bp, Homo sapiens; identical to SEQ ID NO: 265

SEQ ID NO: 507 - DNA, 50 bp, Homo sapiens; identical to SEQ ID NO: 266

SEQ ID NO: 508 - DNA, 50 bp, Homo sapiens; identical to SEQ ID NO: 267

SEQ ID NO: 509 - DNA, 50 bp, Homo sapiens; identical to SEQ ID NO: 268

SEQ ID NO: 510 - DNA, 50 bp, Homo sapiens; identical to SEQ ID NO: 269

SEQ ID NO: 511 - DNA, 50 bp, Homo sapiens; identical to SEQ ID NO: 270

SEQ ID NO: 512 - DNA, 50 bp, Homo sapiens; identical to SEQ ID NO: 271

SEQ ID NO: 513 - DNA, 50 bp, Homo sapiens; identical to SEQ ID NO: 272

SEQ ID NO: 514 - DNA, 50 bp, Homo sapiens; identical to SEQ ID NO: 273

SEQ ID NO: 515 - DNA, 50 bp, Homo sapiens; identical to SEQ ID NO: 274

SEQ ID NO: 516 - DNA, 50 bp, Homo sapiens; identical to SEQ ID NO: 275

SEQ ID NO: 517 - DNA, 50 bp, Homo sapiens; identical to SEQ ID NO: 276

SEQ ID NO: 518 - DNA, 50 bp, Homo sapiens; identical to SEQ ID NO: 277

SEQ ID NO: 519 - DNA, 50 bp, Homo sapiens; identical to SEQ ID NO: 278

SEQ ID NO: 520 - DNA, 50 bp, Homo sapiens; identical to SEQ ID NO: 279

SEQ ID NO: 521 - DNA, 50 bp, Homo sapiens; identical to SEQ ID NO: 280

SEQ ID NO: 522 - DNA, 50 bp, Homo sapiens; identical to SEQ ID NO: 281

SEQ ID NO: 523 - DNA, 50 bp, Homo sapiens; identical to SEQ ID NO: 282

SEQ ID NO: 524 - DNA, 50 bp, Homo sapiens; identical to SEQ ID NO: 283

SEQ ID NO: 525 - DNA, 50 bp, Homo sapiens; identical to SEQ ID NO: 284

SEQ ID NO: 526 - DNA, 50 bp, Homo sapiens; identical to SEQ ID NO: 285

SEQ ID NO: 527 - DNA, 50 bp, Homo sapiens; identical to SEQ ID NO: 286

SEQ ID NO: 528 - DNA, 50 bp, Homo sapiens; identical to SEQ ID NO: 287

SEQ ID NO: 529 - protein, 9 aa, Homo sapiens
SLLKFLAKV

SEQ ID NO: 530 - protein, 9 aa, Homo sapiens
MLLVFGIDV

SEQ ID NO: 531 - protein, 9 aa, Homo sapiens
KVTDLVQFL

SEQ ID NO: 532 - protein, 10 aa, Homo sapiens
GLYDGMMEHL

SEQ ID NO: 533 - protein, 9 aa, Homo sapiens
ILILSIIFI

SEQ ID NO: 534 - protein, 9 aa, Homo sapiens
FLWGPRAHA

SEQ ID NO: 535 - protein, 9 aa, Homo sapiens
VIWEALNMM

SEQ ID NO: 536 - protein, 9 aa, Homo sapiens
KMSILKFLA

SEQ ID NO: 537 - protein, 9 aa, Homo sapiens
KNYEDHFPL

SEQ ID NO: 538 - protein, 9 aa, Homo sapiens
FVLVTSLGL

SEQ ID NO: 539 - protein, 9 aa, Homo sapiens
ILFSEASEC

SEQ ID NO: 540 - protein, 9 aa, Homo sapiens
GMLSDVQSM

SEQ ID NO: 541 - protein, 9 aa, Homo sapiens
ILILILSII

SEQ ID NO: 542 - protein, 9 aa, Homo sapiens
GILILILSI

SEQ ID NO: 543 - protein, 9 aa, Homo sapiens
NMMGLYDGM

SEQ ID NO: 544 - protein, 9 aa, Homo sapiens
QIACSSPSV

SEQ ID NO: 545 - protein, 9 aa, Homo sapiens
LIPSTPEEV

SEQ ID NO: 546 - protein, 9 aa, Homo sapiens
SMPKTGILI

SEQ ID NO: 547 - protein, 9 aa, Homo sapiens
IIFIEGYCT

SEQ ID NO: 548 - protein, 8 aa, Homo sapiens
WEALNMGL

SEQ ID NO: 549 - DNA, 50 bp, Homo sapiens; identical to SEQ ID NO: 63

SEQ ID NO: 550 - DNA, 50 bp, Homo sapiens; identical to SEQ ID NO: 71

SEQ ID NO: 551 - protein, 12 aa, Homo sapiens
KSVKRSVSEIQL

SEQ ID NO: 552 - DNA, 22 bp, Homo sapiens
tgaggagtgcaggctgaatggc

SEQ ID NO: 553 - DNA, 22 bp, Homo sapiens
tgaggagtgcaggctgaatggc

SEQ ID NO: 554 - DNA, 19 bp, Homo sapiens
accgctggagccagacgcc

SEQ ID NO: 555 - DNA, 50 bp, Homo sapiens; identical to SEQ ID NO: 46